(19) United States
(12) Patent Application Publication
(10) Pub. No.: US 2026/0234253 A1
(43) Pub. Date: Aug. 13, 2026

(54) COMBINATION OF BISPECIFIC ANTIBODIES AND CHIMERIC ANTIGEN RECEPTOR T CELLS FOR TREATMENT

(71) Applicant: Evolvelmmune Therapeutics, Inc., Branford, CT (US)

(72) Inventors: Jeremy S. Myers, Orange, CT (US); Oksana Sergeeva, Fairfield, CT (US); Mohosin Sarkar, Woodbridge, CT (US); Eric M. Tam, Maple Valley, WA (US)

(73) Assignee: Evolvelmmune Therapeutics, Inc., Branford, CT (US)

(21) Appl. No.: 19/156,029

(22) PCT Filed: Feb. 14, 2024
(86) PCT No.: PCT/US2024/015867

§ 371 Date: Aug. 13, 2025

Related U.S. Application Data
(60) Provisional application No. 63/484,973, filed on Feb. 14, 2023.

(51) Int. Cl. C07K 16/28

(52) U.S. Cl. CPC C07K 16/2809; C07K 16/2878; C07K 16/2887; C07K 2317/31

(57) ABSTRACT

The present disclosure provides bispecific antibodies comprising a first antigen binding region that binds to a modified T cell or CAR domain expressed on a modified T cell, a second antigen binding region that binds to the tumor cell, and an at least third antigen binding region that binds to any other antigen (e.g., a biological molecule, e.g., a cell surface antigen, e.g., a disease associated antigen). Also included are methods of use thereof, including T-cell re-targeting, and treating cancer in a subject in need thereof.

### RELATED APPLICATIONS

This application claims priority to, and the benefit of, U.S. Provisional Application No. 63/484,973, filed on Feb. 14, 2023. The contents of this application are incorporated herein by reference in its entirety.

### BACKGROUND

Over the past decade, Chimeric Antigen Receptor (CAR) T cell therapy has demonstrated remarkable efficacy against B-lineage leukemias, lymphomas and multiple myeloma and held promise for the treatment of all malignancies which are otherwise incurable with conventional therapies. Across multiple clinical trials, CAR T cells targeting the CD19 antigen have induced complete remission (CR) in 70-90% of patients with multiply-relapsed and/or refractory acute lymphoblastic leukemia (ALL). However, 30-60% of patients relapse after CAR treatment, and among those, 10-20% are CD19-negative relapse. The eligible treatment options for post-CAR relapse are limited, making it more difficult to achieve CR and improve survival rate; thus, treatments for post-CAR relapse are particularly significant. Post-CAR relapses present a clinical challenge as conventional chemotherapy, antibody-based therapies (blinatumomab and inotuzumab) and retreatment with the same CAR T cells have been found to infrequently be capable of reinducing patients into remissions or achieving complete remission (CR).

CD19-directed CAR T cell therapy for relapse and/or refractory B-lineage lymphomas has demonstrated similar results, with Objective Response Rates (ORR) of 52-82%, and 40-54% of patients achieving a Complete Response (CR), yet disease recurrence and/or progression after CAR T cell therapy remains common with less than 40% of patients remaining progression-free 1 year later. Similarly. CD19-directed CAR T cell therapies have demonstrated consistently high anti-tumor efficacy in children and adult populations with relapsed B-cell acute lymphoblastic leukemia (B-ALL), chronic lymphocytic leukemia (CLL), and B-cell non-Hodgkin lymphoma (B-NHL). Similar to ALL results, there are no established therapies which are effective for lymphoma and leukemia patients whose disease relapsed and/or progressed after CAR T cells and reinfusion of the same CAR T cells has been largely ineffective.

Relapses after CAR therapy occur through a variety of mechanisms. In B cell lineage leukemias treated with CD19-directed CAR T cells, upfront treatment failures and relapses in which the leukemia continues to express the CD19 antigen are highly correlated to low levels of CAR T cell expansion and a short duration of CAR T cell persistence in the patient, and it is generally held that improving CAR T cell expansion and persistence would improve outcomes by preventing relapses of antigen-positive leukemias. Another major mechanism of relapse after CAR T cell therapy is the modulation of the targeted antigen on the malignant cells as a means of escaping CAR T cell detection. In B cell leukemias, this has been mostly observed as the emergence of CD19-negative leukemia cells upon relapse. Similarly, decreased surface expression of the CD19 antigen on B-cell lineage lymphomas has been implicated in refractoriness to and relapse after treatment with CD19-directed CAR T cells. Further, B cell lineage lymphomas with a lineage switch provides another therapeutic challenge. Here loss of CD19 antigens associated with one lineage is combined with the gain of a tumor associated antigen associated with another lineage. In either antigen-loss or down-modulation current CAR T cell therapies directed at CD19 are ineffective, an outcome which has been generalizable to other CAR-targeted antigens beyond CD19.

Thus, there is a need in the art for alternative approaches to mitigate relapse after CAR T cell therapy to improve patient outcomes by enhancing the persistence and antigen-sensitivity of CAR T cells to improve the clinical efficacy of CAR T cell therapy against a variety of antigens and malignancies. The present invention addresses the unmet needs in the art and provides bispecific antibodies which retarget and enhance the activity of CAR-T cells towards other target antigens when the original target is ineffective.

### SUMMARY

The disclosure provides a method of T cell re-targeting to a tumor cell population in a subject, the method comprising administering to the subject a bispecific antibody, wherein the subject was previously administered a population of modified T cells that bind a first target antigen on the tumor cell, wherein the bispecific antibody comprises a first antigen binding region that binds to a target antigen on the modified T cell and a second antigen binding region that binds to a second target antigen on the tumor cell.

The disclosure provides a method for stimulating a T cell mediated immune response to a tumor cell population in a subject, the method comprising administering to the subject a bispecific antibody, wherein the subject was previously administered a population of modified T cells that bind a first target antigen on the tumor cell, wherein the bispecific antibody comprises a first antigen binding region that binds to a target antigen on the modified T cell and a second antigen binding region that binds to a second target antigen on the tumor cell.

The disclosure provides a method of treating a cancer in a subject, the method comprising administering to the subject a bispecific antibody, wherein the subject was previously administered a population of modified T cells that bind a first target antigen on the tumor cell, wherein the bispecific antibody comprises a first antigen binding region that binds to a target antigen on the modified T cell and a second antigen binding region that binds to a second target antigen on the tumor cell.

The disclosure provides a method of T cell re-targeting to a tumor cell population in a subject, the method comprising administering to the subject a population of modified T cells that bind a first target antigen on the tumor cell and a bispecific antibody, wherein the bispecific antibody comprises a first antigen binding region that binds to a target antigen on the modified T cell and a second antigen binding region that binds to a second target antigen on the tumor cell.

The disclosure provides a method for stimulating a T cell mediated immune response to a tumor cell population in a subject, the method comprising administering to the subject a population of modified T cells that bind a first target antigen on the tumor cell and a bispecific antibody, wherein the bispecific antibody comprises a first antigen binding region that binds to a target antigen on the modified T cell and a second antigen binding region that binds to a second target antigen on the tumor cell.

The disclosure provides a method of treating a cancer in a subject, the method comprising administering to the subject a population of modified T cells that bind a first target antigen on the tumor cell and a bispecific antibody, wherein the bispecific antibody comprises a first antigen binding region that binds to a target antigen on the modified T cell and a second antigen binding region that binds to a second target antigen on the tumor cell.

In some embodiments, the subject is resistant, inadequately responsive to, or relapsed following the administration of a population of modified T cells that bind a first target antigen on the tumor cell.

In some embodiments, the first target antigen is no longer expressed on the tumor cell following the administration of the population of modified T cells that bind a first target on the tumor cell.

In some embodiments, the first target antigen on the tumor cell is expressed at a low level. In some embodiments, the first target antigen on the tumor cell is expressed at a lower level than the second target antigen on the tumor cell. In some embodiments, the first target antigen on the tumor cell and the second target antigen on the tumor cell is expressed at an equal level.

In some embodiments, the administration of the bispecific antibody results in higher levels of tumor cell killing in comparison to the level of tumor cell killing from administration of a population of modified T cells alone.

In some embodiments, the level of tumor cell killing is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater than the level of tumor cell killing from administration of a population of modified T cells alone.

In some embodiments, the administration of the bispecific antibody increases an immune response against the tumor cell population in comparison to an immune response from administration of the population of modified T cells alone. In some embodiments, the immune response is T cell activation and/or T cell proliferation of the modified T cell and/or a natural (NT)-T cell. In some embodiments, the immune response is modified T-cell mediated tumor killing. In some embodiments, the immune response is natural (NT)-T cell mediated tumor killing. In some embodiments, the immune response is modified T-cell mediated tumor killing and natural (NT)-T cell mediated tumor killing.

In some embodiments, the increased immune response is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater against a tumor cell in comparison to the immune response from administration of the population of modified T cells alone.

In some embodiments, the target antigen on the modified T cell is CD3.

In some embodiments, the target antigen on the modified T cell is an antigen binding region of a chimeric antigen receptor. In some embodiments, the target antigen on the modified T cell is a linker region of a chimeric antigen receptor.

In some embodiments, the first target antigen on the tumor cell and the second target antigen on the tumor cell are the same. In some embodiments, the first target antigen on the tumor cell and the second target antigen on the tumor cell is CD19, BCMA or CD22.

In some embodiments, the first target antigen on the tumor cell and the second target antigen on the tumor cell are different. In some embodiments, the first target antigen on the tumor cell is CD19, BCMA or CD22. In some embodiments, the second target antigen on the tumor cell is CD20. CD180, BCMA, CD79A, CD79B or CD22.

In some embodiments, the bispecific antibody comprises a first antigen binding region that binds to CD3 and a second antigen binding region that binds to CD19. In some embodiments, the bispecific antibody comprises a first antigen binding region that binds to CD3 and a second antigen binding region that binds to CD20. In some embodiments, the bispecific antibody comprises a first antigen binding region that binds to CD3 and a second antigen binding region that binds to CD180. In some embodiments, the bispecific antibody comprises a first antigen binding region that binds to CD3 and a second antigen binding region that binds to BCMA.

In some embodiments, the modified T cell is a CAR T cell.

In some embodiments, the bispecific antibody has the following structure: a first heavy chain polypeptide (H1) comprising a variable region (VH1), and a constant region (CH1) having a constant region 1 domain (CH1_H1), a hinge region (H1H), a constant region 2 domain (CH1_H2) and a constant region 3 domain (CH1_H3); and a first light chain polypeptide (L1) comprising a variable region (VL1) and a constant region (CL1); and a second heavy chain polypeptide (H2) comprising a variable region (VH2), and a constant region (CH2) having a constant region 1 domain (CH2_H1), a hinge region (H2H), a constant region 2 domain (CH2_H2) and a constant region 3 domain (CH2_H3); and second light chain polypeptide (L2) comprising a variable region (VL2) and a constant region (CL2), and wherein i) the amino acid at position 39 (Kabat numbering) of the VH1 is a K and the amino acid at position 38 (Kabat numbering) of the VL1 is a D; ii) the amino acid at position 147 (EU numbering) of the CH1_H1 is a K and the amino acid at position 131 (EU numbering) of the CL1 is a D; iii) the amino acid at position 173 (EU numbering) of the CH1_H1 is a C and the amino acid at position 162 (EU numbering) of the CL1 is a C; iv) the amino acid at position 220 (EU numbering) in the H1H is a S and the amino acid at position 214 (EU numbering) of the CL1 is a S; v) the amino acid at position 39 (Kabat numbering) of the VH2 is a D and the amino acid at position 38 (Kabat numbering) of the VL2 is a K; and vi) the amino acid at position 147 (EU numbering) of the CH2_H1 is a D and the amino acid at position 180 (EU numbering) of the CL2 is a R.

In some embodiments, the bispecific antibody comprises the following: i) the amino acid at position 87 (Kabat numbering) of the VH1 and/or VH2 is a G; and ii) the amino acid at position 45 (Kabat numbering) of the VL1 and/or VL2 is a W.

In some embodiments, the bispecific antibody comprises the following: i) the CH1_H3 has a C at position 349, an S at position 366, an A at position 368 and a V at position 407 (EU numbering); and the CH2_H3 has a C at position 354 and a W at position 366 (EU numbering); ii) the CH2_H3 has a C at position 349, an S at position 366, an A at position 368 and a V at position 407 (EU numbering); and the CH1_H3 has a C at position 354 and a W at position 366 (EU numbering); iii) the CH1_H3 has a C at position 354, an S at position 366, an A at position 368 and a V at position 407 (EU numbering); and the CH2_H3 has a C at position 349 and a W at position 366 (EU numbering); or iv) the CH2_H3 has a C at position 354, an S at position 366, an A at position 368 and a V at position 407 (EU numbering); and the CH1_H3 has a C at position 349 and a W at position 366 (EU numbering).

In some embodiments, the bispecific antibody comprises the following: the amino acid at position 447 (EU numbering) of the CH1_H3 and/or of the CH2_H3 is deleted.

In some embodiments, the bispecific antibody comprises the following: i) the H1H and/or the H2H has an A at positions 234 and 235 (EU numbering); ii) the H1H and/or the H2H has an A at positions 234, 235 and 237 (EU numbering); or iii) the H1H and/or the H2H has an A at positions 234 and 235 and G at position 329 (EU numbering).

In some embodiments, the bispecific antibody comprises the following: i) the CH1_H3 and/or the CH2_H3 has an A at position 297 (EU numbering); ii) the CH1_H3 and/or the CH2_H3 has a G at position 297 (EU numbering); or iii) the CH1_H3 and/or the CH2_H3 has a S at position 297 (EU numbering).

In some embodiments, the bispecific antibody comprises the following: the CH1_H3 and/or the CH2_H3 has an S at position 331 (EU numbering).

In some embodiments, a polypeptide is fused to the N-terminus or the C-terminus of the first heavy chain polypeptide or the second heavy chain polypeptide of the bispecific antibody. In some embodiments, the polypeptide is fused to the C-terminus of the first heavy chain polypeptide of the bispecific antibody. In some embodiments, the polypeptide is fused via a linker peptide. In some embodiments, the linker peptide comprises the amino acid sequence of SEQ ID NO: 53. In some embodiments, the polypeptide comprises a CD58 or a fragment thereof. In some embodiments, the CD58 comprises the amino acid sequence of SEQ ID NO: 49.

In some embodiments, the bispecific antibody is an IgG1 or an IgG4 antibody.

In some embodiments, the bispecific antibody is a monoclonal antibody, a chimeric antibody or a humanized antibody.

In some embodiments, the methods of the disclosure further comprise administering a modified T cell activation agent.

### BRIEF DESCRIPTION OF THE DRAWINGS

FIGS. 1A-1E is a series of diagrams depicting structural variants of bispecific antibodies of the present disclosure. Dark shaded domains represent a first heavy chain polypeptide (H1) having a heavy chain variable region (VH1), having a constant region 1 domain (CH1_{H1}), a hinge region (H1H), a constant region 2 domain (CH1_{H2}) and a constant region 3 domain (CH1_{H3}), and a first light chain polypeptide (L1) comprising a variable region (VL1) and a constant region (CL1). Light shaded domains represent a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2). FIG. 1A depicts an exemplary bispecific antibody fusion molecule comprising a peptide fused to the CH1_{H3} domain. FIG. 1B depicts an exemplary bispecific antibody fusion molecule comprising a peptide fused to the VL1 domain. FIG. 1C depicts an exemplary bispecific antibody fusion molecule comprising a peptide fused to the VH1 domain. FIG. 1D depicts an exemplary bispecific antibody fusion molecule comprising a peptide fused to the CH1_{H3} domain and a first antigen binding region (dark shading), and a second antigen binding region (light shading) operably linked to a third antigen binding region (light shading). FIG. 1E depicts an exemplary bispecific antibody fusion molecule comprising a peptide fused to the CH1_{H3} domain and a first antigen binding region (dark shading), a second antigen binding region (light shading) and a third antigen binding region (dark shading), wherein first antigen binding region is operably linked to the third antigen binding region.

FIGS. 2A-2D are flow cytometry analysis plots of CD19 (FIG. 2A), CD20 (FIG. 2B), BCMA (FIG. 2C), and CD180 (FIG. 2D) relative to isotype control (dotted lines) in Jeko-1 GFP-Luc CD19 knockout cells (black lines) compared to AAVS1 negative control cells (gray lines), analyzed via flow staining using the respective antibodies and controls.

FIGS. 3A-3B are two schematics depicting bispecific antibody retargeting of CAR-T cells to tumor cells that have lost CD19. Anti-CD19 FMC63 CAR-T cells efficiently kill Jeko-1 CD19+ tumor cells (FIG. 3A: top panel), but not Jeko-1 CD19-KO tumor cells (FIG. 3A; lower panel). However, if bispecific antibodies with or without the CD58 fusion are used in the co-culture), they can bridge the CAR-T cell to a different tumor antigen and the CAR-T cell can kill the tumor cell through this retargeting mechanism (FIG. 3B).

FIGS. 4A-D are a series of graphs depicting the function and phenotype of naïve, non-transfected (NT) and CAR T cells. FIG. 4A depicts the tumor killing assay with Jeko-1 GFP-Luc AAVS1-KO (left) or CD19-KO (right) cells co-cultured with CAR or non-transduced (NT) T cells at an E:T ratio of 1:3. Tumor cell killing is presented as % tumor cells remaining after 48 hours. FIGS. 4B-C shows levels of T cell receptors on CAR (black line), NT (medium gray), and naïve (light gray) T cells relative to isotype controls (dotted lines), specifically in CD4 T cells. FIG. 4B shows CD3. FIG. 4C shows CD2. FIG. 4D shows CD28.

FIGS. 5A-5B are two line graphs depicting killing of CD19-knockout (CD19-KO) or control (AAVS1-KO) Jeko-1 GFP-Luc tumor cells co-cultured with CD19-targeted antigen expressing CAR-T cells or non-transduced control T cells at an E:T ratio of 1:3 (30,000 tumor cells: 10,000 T cells) and treated with non-targeting control CD3-bispecific antibody with CD58 fusion (EIP0614), anti-CD3/anti-CD20 bispecific antibody (EIP0961), or anti-CD3/anti-CD20 bispecific antibody with CD58 fusion (EIP0929) antibody. Cells were detected via flow cytometry. FIG. 5A shows % tumor cells remaining after co-culture with CD19-targeted antigen expressing CAR-T cells. FIG. 5B shows % tumor cells remaining after co-culture with non-transduced control T cells.

FIGS. 6A-6B are two line graphs depicting killing of CD19-knockout (CD19-KO) or control (AAVS1-KO) Jeko-1 GFP-Luc tumor cells co-cultured with CD19-targeted antigen expressing CAR-T cells or non-transduced control T cells at an E:T ratio of 1:3 (30,000 tumor cells: 10,000 T cells) and treated with non-targeting control CD3-bispecific antibody with CD58 fusion (EIP0614), anti-CD3/anti-BCMA bispecific antibody (EIP0506), or anti-CD3/anti-BCMA bispecific antibody with CD58 fusion (EIP534) antibody. Cells were detected via flow cytometry. FIG. 6A shows % tumor cells remaining after co-culture with CD19-targeted antigen expressing CAR-T cells. FIG. 6B shows % tumor cells remaining after co-culture with non-transduced control T cells.

FIGS. 7A-7B are two line graphs depicting killing of CD19-knockout (CD19-KO) or control (AAVS1-KO) Jeko-1 GFP-Luc tumor cells co-cultured with CD19-targeted antigen expressing CAR-T cells or non-transduced control T cells at an E:T ratio of 1:3 (30,000 tumor cells: 10,000 T cells) and treated with non-targeting control CD3-bispecific antibody with CD58 fusion (EIP0614), anti-CD3/anti-CD180 bispecific antibody with higher CD3 affinity (EIP0696), anti-CD3/anti-CD180 bispecific antibody with higher CD3 affinity and with CD58 fusion (EIP0716) antibody, anti-CD3/anti-CD180 bispecific antibody with lower CD3 affinity (EIP0698), and anti-CD3/anti-CD180 bispecific antibody with lower CD3 affinity and with CD58 fusion (EIP0699) antibody. Cells were detected via flow cytometry. FIG. 7A shows % tumor cells remaining after co-culture with CD19-targeted antigen expressing CAR-T cells. FIG. 7B shows % tumor cells remaining after co-culture with non-transduced control T cells.

FIGS. 8A-8B are two line graphs depicting killing of control (AAVS1-KO) Jeko-1 GFP-Luc tumor cells co-cultured with CD19-targeted antigen expressing CAR-T cells at an E:T ratio of 1:5 (50,000 tumor cells: 10,000 T cells) or 1:10 (100,000 tumor cells: 10,000 T cells) and treated with non-targeting control CD3-bispecific antibody with CD58 fusion (EIP0614), anti-CD3/anti-BCMA bispecific antibody (EIP0506), or anti-CD3/anti-BCMA bispecific antibody with CD58 fusion (EIP534) antibody. Cells were detected via flow cytometry and normalized to no biologic which already constitutes about 30% of cells remaining due to the killing through the CAR. FIG. 8A shows E:T ratio of 1:5. FIG. 8B shows E:T ratio of 1:10.

FIGS. 9A-9C are three flow cytometry analysis plots of CD19 (FIG. 9A), CD20 (FIG. 9B), and CD180 (FIG. 9C) relative to isotype control (dotted lines) in Jeko-1 GFP-Luc CD19 knockout cells (black lines), CD19 and CD20 knockout cells (light gray lines), and AAVS1 knockout negative control cells (medium gray lines), analyzed via flow staining using the respective antibodies and controls.

FIG. 10 is a set of schematics depicting bispecific antibody retargeting of CAR-T cells to tumor cells that have lost either CD19 or both CD19 and CD20. Anti-CD19 FMC63 CAR-T cells cannot kill Jeko-1 CD19- and- CD20-knockout tumor cells (FIG. 10; top panel), but neither can anti-CD20 biologics (FIG. 10; middle panel). However, anti-CD180 biologics can still effectively retarget CAR-T cells to CD19- and CD20-knockout tumor cells (FIG. 10; bottom panel).

FIG. 11 is a line graph depicting killing of CD19-knockout (CD19-KO; black lines), CD19- and CD20 knockout (CD19-KO and CD20-KO; light gray lines) or control (AAVS1-KO: medium gray lines) Jeko-1 GFP-Luc tumor cells co-cultured with CD19-targeted antigen expressing CAR-T cells at an E:T ratio of 1:3 (30,000 tumor cells: 10,000 T cells) and treated with non-targeting control CD3-bispecific antibody with CD58 fusion (EIP0614; thin dotted lines), anti-CD3/anti-CD20 bispecific antibody with CD58 fusion (EIP0929; thick dashed lines) antibody, and anti-CD3/anti-CD180 bispecific antibody with CD58 fusion (EIP699: solid lines) antibody. Cells were detected via flow cytometry. Percent tumor cells remaining after co-culture with CD19-targeted antigen expressing CAR-T cells are shown.

FIG. 12 is a schematic depicting various formats of bispecific antibodies to retarget CAR-T cells. Herein, antigens such as CD20, BCMA, and CD180 are shown. Both cell targeting arms of the bispecific antibodies can be varied to target the correct combination of T cell and tumor cells. The T cell targeting arm can be altered, namely through targeting FMC63 itself or other extracellular CAR domains. The tumor targeting arms can be changed to target other tumor antigens, such as CD22.

### DETAILED DESCRIPTION

Acute lymphoblastic leukemia (ALL) is one of the most common malignancies in children and adults. Although the 5-year overall survival (OS) rate is 80-90% at present in children, relapsed and/or refractory (R/R) ALL remains one of the most important causes of cancer death in children. Chimeric antigen receptor (CAR) T-cell therapy has performed well and has promising applications as an emerging immunotherapy, among which CD19-directed CAR is a remarkable innovation in the treatment of R/R B-ALL. Numerous clinical trials have shown that 70-90% complete remission (CR) can be achieved in pediatric and adult patients treated with CD19-directed CAR T-cells. However, 30-60% of patients relapse after CAR treatment, and among those, 10-20% are CD19-negative relapse. The eligible treatment options for post-CAR relapse are limited, making it more difficult to achieve CR and improve survival rate, thus, treatments for post-CAR relapse are significant. Patient relapse after CD22 directed CAR-T cell and BCMA directed CAR-T cell therapeutics have also been observed. The present invention addresses these needs.

The sensitivity of CAR T cells for their target antigen greatly impacts patient outcomes of those who received CAR T therapy. Exemplary therapies include CD19 targeted CAR-T cells including but not limited to BREYANZI® (lisocabtagene maraleucel), TECARTUS™ (brexucabtagene autoleucel), KYMRIAH™ (tisagenlecleucel), or YESCARTA™ (axicabtagene ciloleucel), and BCMA targeted CAR-T cells including but not limited to ABECMA® (idecabtagene vicleucel), or CARVYKTI™ (ciltacabtagene autoleucel) and CD22 targeted CAR-T cells. For example while multiple anti-CD19 CAR-T therapies are approved for patients, these therapies are known to lose efficacy because patients develop CD19-loss relapses. In some embodiments, the subject is suffering from a disease associated with expression of CD19, such as a hematological cancer (e.g., a hematological cancer described herein such as CLL, MCL, or ALL) and the subject is, or is identified as, a partial responder, non-responder, or relapser to one or more therapies for the hematological cancer, e.g., an anti-CD19 adoptive CAR-T cell therapy. In some embodiments, the subject is resistant, inadequately responsive to, or relapsed.

In some embodiments, the subject has, or is identified as having, a CD19, BCMA or CD22 mutation. The mutation may be, e.g., a point mutation, an insertion, or a deletion. The mutation may be, e.g., a mutation at the binding site for CD19, BCMA or CD22 or a mutation that results in reduced or abolished expression of CD19, BCMA or CD22, thereby resulting in a decreased response (e.g., resistance) to the anti-CD19, BCMA or CD22 CAR-T cell therapy. In some embodiments, the target antigen of the CAR-T cell therapy (e.g. anti-CD19, BCMA or CD22 CAR-T) is no longer expressed on the tumor cell following the administration of the CAR-T therapy to the subject. In some instances, this occurrence could be a result of B-cell lineage switching. Due to the plasticity of the B-cells' inherent lineage, they can be induced to different phenotypic conversions, including myeloid conversion under immune pressure. In some embodiments, CD19 is not expressed. In some embodiments. BCMA is not expressed. In some embodiments, CD22 is not expressed. In some embodiments, CD19 is expressed at a low level. In some embodiments, BCMA is expressed at a low level. In some embodiments, CD22 is expressed at a low level. The inability of CAR T cells to target low-levels of antigen is of clinical importance, as this is the major mechanism for relapse in patients treated with immunotherapy.

In some embodiments, the bispecific antibodies of the invention re-target an anti-CD19 CAR-T cell to a different target antigen. Accordingly, the bispecific antibodies disclosed herein provides a novel approach to addressing the shortcomings of current CAR T therapy by effectively retargeting CAR-T cells (e.g. anti-CD19, BCMA or CD22 CAR-T). The bispecific antibodies herein also address a novel approach to treating B-lineage leukemias, lymphomas and multiple myeloma that have undergone a B-cell lineage switch, by effectively retargeting CAR-T cells (e.g. anti-CD19, BCMA or CD22 CAR-T) to the switched B cell lineage (e.g. cancers that do not express the target antigen of the CAR) but rather express a second disease associated antigen. For example, once CD19 antigen and/or a CD20 antigen is not expressed, targets from other B cell lineages such as CD180 antigen can be targeted. The bispecific antibodies herein also address a novel approach to treating secondary malignancies by effectively retargeting CAR-T cells (e.g. anti-CD19, BCMA or CD22 CAR-T) to secondary malignancies (e.g. cancers that do not express the target antigen of the CAR) but rather express a second disease associated antigen. The present invention is based, at last in part, on the finding that the bispecific antibodies of the invention can retarget and improve the ability of the T cells to recognize to CD19-negative tumor cells or tumor cells with low expression of CD19, to a different target antigen on the same tumor cell. Without being bound by theory, tumor cells retain the expression of a second tumor associated antigen (e.g. CD20 or CD180), and T-cell retargeting can thereby increase effective tumor killing and improve clinical patient outcomes.

In some embodiments, the bispecific antibodies of the invention have a first binding region that binds the antigen binding region (e.g. FMC63 scFv) of anti-CD19 CAR T cell and a second binding region that binds a second tumor cell antigen. Without being bound by theory, targeting the antigen binding region of the CAR may make the CAR T cell more durable, as signaling is going through the CAR and not an endogenous TCR.

In some embodiments, the bispecific antibodies of the invention have a first binding region that targets CD3 of a CAR-T cell, a second antigen binding region that binds to a second tumor antigen, and a CD58 fusion peptide at the N-terminus or the C-terminus of the bispecific antibody that binds CD2. The present invention is based, at least in part, on the finding that engaging CD2 (on CAR-T cells) could improve the clinical outcomes of the therapy by activating a second co-stimulatory pathway which can activation and induce T-cell proliferation to overcome anergy.

### METHODS OF THE DISCLOSURE

T cell retargeting (or T cell redirecting) bispecific antibodies is a novel class of therapeutics, capable of recruiting T cells to tumor cells and inducing tumor-specific (but MHC-independent) activation of T cell effector activities. The present disclosure provides a method of T cell retargeting to a tumor population in a subject, comprising administering a bispecific antibody comprising a first antigen binding region that targets a modified T cell (e.g. CD3 portion of the T cell receptor) for T cell recruitment, and second an antigen binding region that targets a second tumor associated antigen (e.g. CD20, CD180, BCMA). In some embodiments of any one of the methods disclosed herein, the subject has already received an adoptive cell therapy, such as a CAR-expressing cell therapy (e.g., an anti-CD19 CAR T, anti-BCMA CAR T or anti-CD22 CAR T cell therapy). In some embodiments of any of the methods disclosed herein, the method comprises administering the bispecific or multispecific antibody or a pharmaceutical composition comprising the bispecific or multispecific antibody to the subject, after administration of a CAR-expressing cell (e.g., an anti-CD19 CAR T, anti-BCMA CAR T or anti-CD22 CAR T cell therapy) to the subject.

This targeting design promotes the recruitment of T cell and positions it in close contact with a second tumor associated antigen-expressing cell, resulting in the formation of an immunological synapse, local T cell activation and the subsequent destruction of the target cell, such as, but not limited to a cancer cell, by perforin and granzyme released from T cell cytotoxic granules.

Accordingly, the present disclosure provides a method for stimulating a T cell mediated immune response to a tumor cell population in a subject, the method comprising administering to the subject a bispecific antibody comprising a first antigen binding region that targets a modified T cell (e.g. CD3 portion of the T cell receptor) for T cell recruitment, and second an antigen binding region that targets a second tumor associated antigen (e.g. CD20, CD180, BCMA). In some embodiments, the subject was previously administered a population of modified T cells that bind a first target antigen on the tumor cell (e.g., a CAR-T cell. In some embodiments, the subject is co-administered with a modified T cell that binds a first target antigen.

In some embodiments, the administration of the bispecific antibody results in higher levels of tumor cell killing in comparison to the level of tumor cell killing from administration of a population of modified T cells alone.

In some embodiments, the level of tumor cell killing is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater than the level of tumor cell killing from administration of a population of modified T cells alone. In some embodiments, the level of tumor cell killing is about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80% or any percentage in between greater than the level of tumor cell killing from administration of a population of modified T cells alone.

In some embodiments, the administration of the bispecific antibody increases an immune response against the tumor cell population in comparison to an immune response from administration of the population of modified T cells alone.

In some embodiments, the increased immune response is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater against a tumor cell in comparison to the immune response from administration of the population of modified T cells alone. In some embodiments, the increased immune response is at about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80% or any percentage in between greater against a tumor cell in comparison to the immune response from administration of the population of modified T cells alone.

As the CD3 binding affinity of the T-cell retargeting bispecific antibodies is crucial for recruitment of T cells, the present invention also relates to the generation of a panel of antibodies that display different binding affinities. The affinity of the CD3 arm of a bispecific or multispecific antibody can significantly modify the functional activity of the bispecific or multispecific antibody. Thus, it is desirable and advantageous to have bispecific antibodies with varied affinities.

Additionally, bispecific antibodies disclosed herein may have a cytokine or costimulatory molecule fusion peptide that acts as antagonist to inhibit or block deleterious interactions or as an agonist to mimic or enhance physiological responses. Physiological responses include but are not limited to T-cell activation, T-cell proliferation. T-cell persistence and prevention of T-cell exhaustion. These properties are advantageous over conventional CD3-bispecific antibodies or tumor targeted co-stimulatory receptor agonists which do not optimally activate T-cells and induce (or promote) T-cell dysfunction. In accordance, cytokine and/or costimulatory fusion peptides are advantageous for enhancing the therapeutic potential of bispecific antibodies. In some embodiments, the costimulatory molecule of the bispecific antibodies of the present disclosure is CD58, or a fragment thereof.

Bispecific or multispecific T cell retargeting agents of the disclosure share the drug-like properties of human monoclonal antibodies. Further, T-cell retargeting bispecific and multispecific antibodies are advantageous over other existing therapies (e.g. CAR-T therapies) because it provides an off-the-shelf product with a high safety profile (e.g. mitigation of cytokine release syndrome and reduced levels of tonic signaling leading to T-cell dysfunction) and the possibility of dose titration and escalation. Bispecific or multispecific T cell retargeting agents of the disclosure, for adoptive cancer immunotherapy has clinically relevant applications and benefits such as 1) increased ability to recognize tumor cells expressing low levels of antigen 2) increased cell persistence and proliferation.

### CD19 Antigen

CD19 (Cluster of Differentiation 19), also known as B-lymphocyte surface antigen B4, is a type I transmembrane glycoprotein belonging to immunoglobulin (Ig) subfamily that serves as a biomarker for normal and neoplastic B cells. CD19 is a co-receptor for the B cell receptor (BCR) signaling complex and has a critical role in regulating B cell signaling and immune response. The CD19 protein contains an extracellular N-terminus containing two C2 Ig-like domains separated by a helical non-Ig domain, a single pass transmembrane domain, and a highly conserved cytoplasmic C-terminal domain. The human CD19 protein, encoded by the CD19 gene located on chromosome 16p11.2, is 556 amino acids (aa) in length with a calculated theoretical molecular weight (MW) of 61 kDa and an observed molecular weight of 95 kDa. CD19 associates with other molecules—CD21, CD81, and CD225—to form the BCR co-complex, also called the CD19 complex, through CD21 binding to the complement C3d complex. Complement C3d bridges the BCR with the CD19 complex into lipid rafts of the plasma membrane. CD19 is capable of modulating B cell development through both BCR-dependent and -independent signaling. Upon BCR activation, the tyrosine residues of CD19's cytoplasmic tail recruits multiple kinases including Lyn, Vav, and PI3K, amplifying BCR-mediated immune signaling and B cell activation.

Considering the role of CD19 in BCR signaling and its expression in development from pre-B cells through plasma cells, it is understandable that CD19 dysfunction and abnormal expression is associated with numerous B cell malignancies and autoimmune disorders. CD19 expression is typically observed at relatively normal levels in B cell acute lymphoblastic leukemia (B-ALL) and chronic lymphoblastic leukemia (CLL) but is often reduced other types of lymphoma including diffuse large B cell lymphoma (DLBCL) and follicular lymphoma (FL). On the other hand, CD19 expression is typically increased in autoimmune disorders such as systemic sclerosis (SSc) and multiple sclerosis (MS) as modeled by experimental autoimmune encephalomyelitis (EAE). CD19 has become a therapeutic molecular target for the treatment of B cell lymphomas and autoimmune disorders using monoclonal antibodies (mAbs), bi-specific T cell engaging (BiTE) antibodies, and CD19-specific chimeric antigen receptor (CAR) T cells. Although anti-CD19 CAR T cell therapy has become the standard for the treatment of B cell malignancies, patients may experience relapse due to resistance mechanisms.

As described in Wutti-In et. al., (Wutti-In Y, Sujjitjoon J, Sawasdee N. et al. Development of a Novel Anti-CD19 CAR Containing a Fully Human scFv and Three Costimulatory Domains. Front Oncol. 2022; 11:802876. Published 2022 Jan. 18. doi:10.3389/fonc.2021.802876), cellular immunotherapy using autologous T cells expressing chimeric antigen receptor (CAR) is a promising strategy as evidenced by CD19-CAR T cell therapy for B-cell malignancies. The U.S Food and Drug Administration (FDA) has approved four CD19-CAR T products for relapsed/refractory acute lymphoblastic leukemia (r/r ALL), r/r large B-cell lymphoma and mantle cell lymphoma. Although objective clinical responses after CAR T treatment were observed and documented, adverse effects [e.g., cytokine release syndrome (CRS) and/or neurologic toxicity] and tumor relapse were reported. The mechanism of CD19+ relapse is associated with poor T cell function and early CD19-CAR T cell disappearance. The previously approved CD19-CAR T products are based on murine-derived scFv (FMC63) that is fused to second-generation CAR (CD28ζ or 4-1BBζ).

Acute lymphoblastic leukemia (ALL) is one of the most common malignancies in children and adults. Although the 5-year overall survival (OS) rate is 80-90% at present in children, relapsed and/or refractory (R,R) ALL remains one of the most important causes of cancer death in children. Chimeric antigen receptor (CAR) T-cell therapy has performed well and has promising applications as an emerging immunotherapy, among which CD19-directed CAR is a remarkable innovation in the treatment of RR B-ALL Numerous clinical trials have shown that 70-90% complete remission (CR) can be achieved in pediatric and adult patients treated with CD19-directed CAR T-cells. However, 30-60% of patients relapse after CAR treatment, and among those, 10-20% are CD19-negative relapse (See Table 1; Xu, Xinjie et al. “Mechanisms of Relapse After CD19 CAR T-Cell Therapy for Acute Lymphoblastic Leukemia and Its Prevention and Treatment Strategies.” Frontiers in immunology vol 10 2664. 12 Nov. 2019, doi:10.3389/fimmu.2019.02664).

There are two patterns of post-CAR relapse in B-ALL, including CD19-positive relapse and CD19-negative relapse. In regard to CD19-positive relapse, whose key mechanism lies in poor persistence of CAR T-cells, CD19 is still present on the surface of B-ALL cells and can be detected by flow cytometry. For CD19-negative relapse. CD19 is absent, causing tumors that evade CAR-mediated recognition and clearance in spite of CAR T-cell persistence.

### Antibody Compositions and Structures

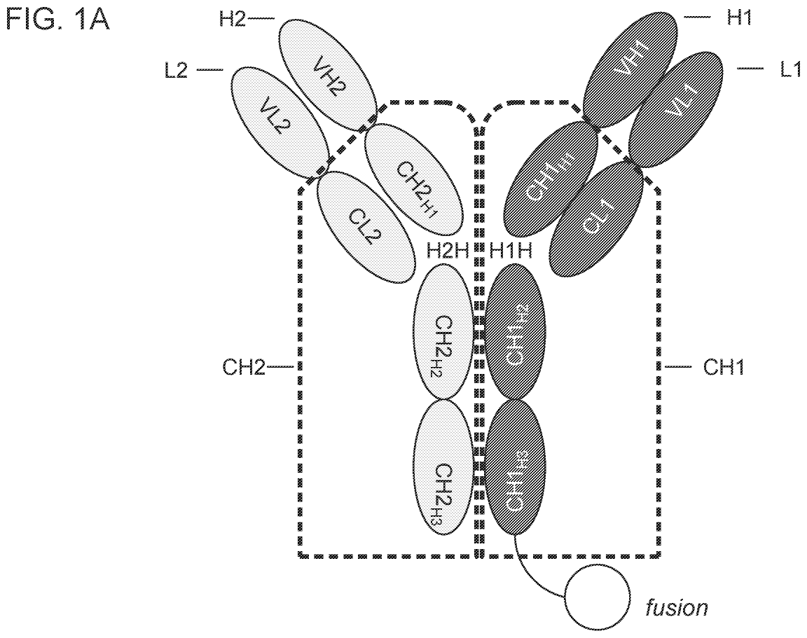

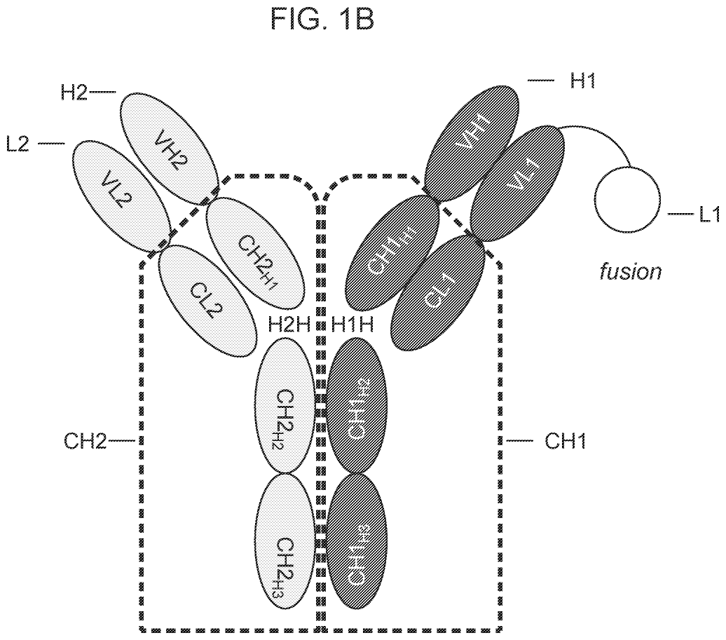

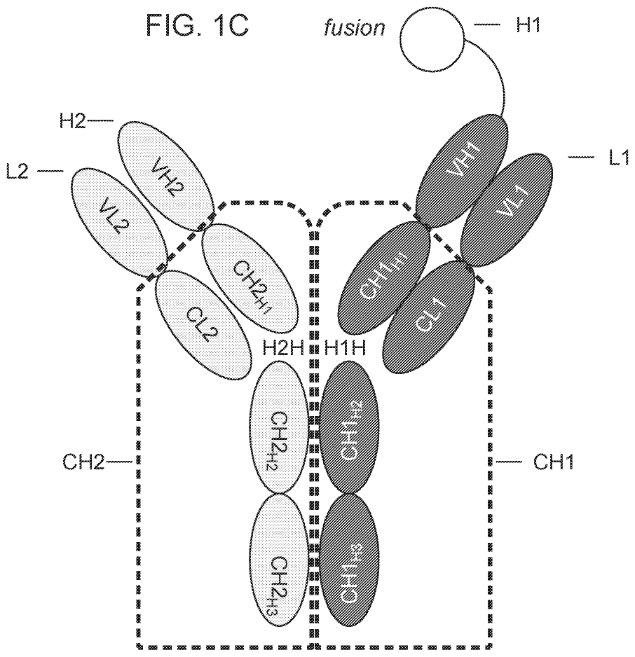

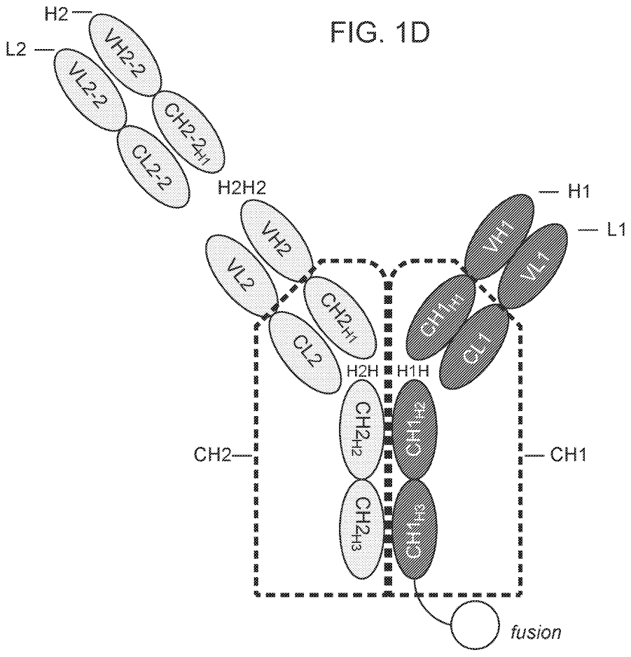

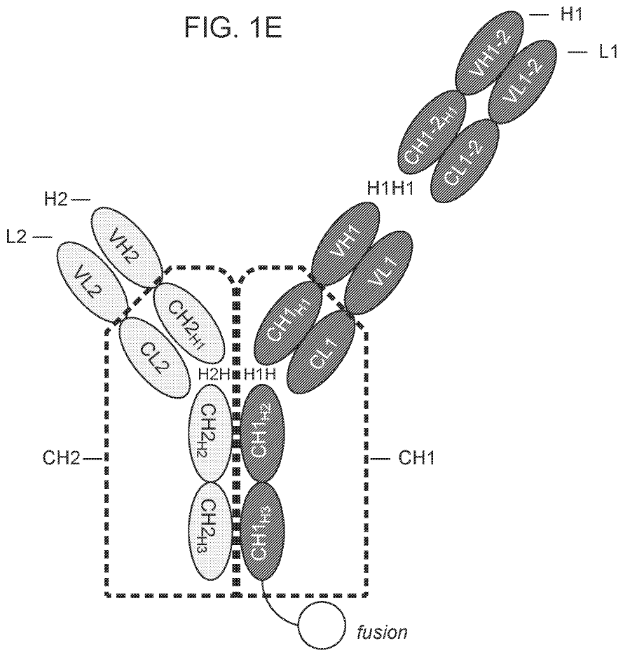

The present disclosure provides an antibody comprising the following domain structure: a) a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1H3); and a first light chain polypeptide (L1) comprising a variable region (VL1) and a constant region (CL1), and b) a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2). A schematic diagram of the antibody structure of the disclosure is shown in FIGS. 1A-1E.

As used herein, the term “antibody” refers to an immunoglobulin (Ig) molecule and immunologically active portions of an immunoglobulin molecule, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K_{d}>10⁻⁶).

Antibodies include, but are not limited to, polyclonal antibodies, monoclonal antibodies, chimeric antibodies. The antibody may be from recombinant sources and/or produced in transgenic animals.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, IgG4 and others. Furthermore, in humans, the light chain may be a kappa chain or a lambda chain. Accordingly, in one embodiment, the antibody disclosed herein is an IgG antibody.

Antibodies may be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “antibody fragment” as used herein is intended to include without limitation, Fv, Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Techniques can be adapted for the production of single-chain antibodies specific to an antigenic protein of the disclosure (see e.g., U.S. Pat. No. 4,946,778). In addition, methods can be adapted for the construction of Fab expression libraries (see e.g., Huse, et al., 1989 Science 246:1275-1281) to allow rapid and effective identification of monoclonal Fab fragments with the desired specificity for a protein or derivatives, fragments, analogs or homologs thereof.

As used herein, the term “epitope” refers to the site on an antigen that is recognized by the antibodies and fragments disclosed herein. The term “epitope” includes any protein determinant capable of specific binding to an immunoglobulin. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is <1 micromolar; e.g., <100 nM, preferably <10 nM and more preferably <1 nM.

Bispecific antibodies are antibodies that have binding specificities for at least two different antigens. The present disclosure provides a bispecific antibody having a first antigen binding region that binds to a first antigen (e.g. CD3) and a second antigen binding region that binds to a second antigen (e.g. disease associated antigen)

Antibodies with more than two valencies are also contemplated. For example, trispecific antibodies can be prepared. Tutt et al., J. Immunol. 147:60 (1991).

### Antibody Variants

In certain embodiments, amino acid sequence variants of the antibodies provided herein are contemplated. For example, it may be desirable to improve the heavy chain heterodimerization, light chain heterodimerization, binding affinity, and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics (e.g., light chain heterodimerization, heavy chain heterodimerization, antigen binding).

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic (negatively charged): Asp, Glu;
(4) basic (positively charged): His, Lys, Arg;
(5) residues that influence chain orientation: Gly, Pro;
(6) aromatic: Trp, Tyr, Phe.

Functional variants of the antibody or antigen-binding fragments described herein are also encompassed by the present disclosure. The term “functional variant” as used herein includes modifications or chemical equivalents of the amino acid and nucleic acid sequences disclosed herein that perform substantially the same function as the polypeptides or nucleic acid molecules disclosed herein in substantially the same way. For example, functional variants of polypeptides disclosed herein include, without limitation, conservative amino acid substitutions.

A “conservative amino acid substitution” as used herein, is one in which one amino acid residue is replaced with another amino acid residue that change an amino acid to a different amino acid with similar biochemical properties (e.g. charge, hydrophobicity and size). Variants of polypeptides also include additions and deletions to the polypeptide sequences disclosed herein. In addition, variant nucleotide sequences include analogs and derivatives thereof. A variant of the binding proteins disclosed herein include proteins that bind to the same antigen or epitope as the binding proteins.

In some embodiments, the charged amino acid residue is a naturally occurring amino acid or a non-naturally occurring amino acid. In some embodiments, the naturally occurring charged amino acid residue is an arginine, a lysine, a histidine, a glutamic acid or an aspartic acid.

### Light Chain and Heavy Chain Substitution Variants

To generate a substantially homogeneous population of bispecific antibodies with the correct pairing of heavy chain and light chains (i.e. cognate pairing or heterodimerization of a light chain with the heavy chain necessary to form the variable domain or antigen binding domain of the original antibody), the first heavy chain polypeptide (H1) has a strong preference for binding with the first light chain polypeptide (L1) relative to the second light chain polypeptide (L2), and the second heavy chain polypeptide (H2) has a strong preference for binding with the second light chain polypeptide (L2) relative to first light chain polypeptide (L1). In addition, the first heavy chain polypeptide (H1) and the second heavy chain polypeptide (H2) have a stronger preference for heterodimerization than homodimerization (i.e. heavy chain heterodimerization).

Antibody variants having one or more amino acid substitutions are provided herein. Exemplary substitutional mutagenesis sites include the charged substitution pairs shown in Tables 1-6.

TABLE 1
Kappa Light Chain and Heavy Chain -
Constant Domain Mutations Pairs

| Mutation | Heavy Chain | Light Chain |
|---|---|---|
| Type | Constant Domain | Constant Domain |
| Charged pair | K147D | S131K |
| Charged pair | K147D | T180R |
| Charged pair | K147(WT) | T180E |
| Charged pair | T187D | N137K/N138R |
| Charged pair | T187D | N138R |
| Charged pair | T187D | N138K |
| Charged pair | K147(WT) | S131D |
| Charged pair | L145S | S131K |

All position information is reported using the EU numbering scheme
Wild type (WT) indicates the natural amino acid at the indicated position
Charge pairs with negative and positive charge residues could be reversed between heavy and light chains, where D or E (negative charge) are replaced by K or R (positive charge) and cognate chain K or R (positive charge) are replaced by D or E (negative charge).

TABLE 2
Kappa Light Chain and Heavy Chain -
Variable Domain Mutations Pairs

| Mutation | Heavy Chain | Light Chain |
|---|---|---|
| Type | Variable Domain | Variable Domain |
| Charged pair | Q39K | Q38D |
| Charged pair | Q39K | Q38E |
| Charged pair | Q39D | Q38K |
| Charged pair | Q39E | Q38K |
| Charged pair | G100K | G44D |
| Knob-into-Hole | Y87G | L45W |

All position information is reported using the Kabat numbering scheme
Wild type (WT) indicates the natural amino acid at the indicated position
Charged pairs with negative and positive charged residues could be reversed between heavy and light chains, where D or E (negative charged) are replaced by K or R (positive charged) and cognate chain K or R (positive charged) are replaced by D or E (negative charged).

TABLE 3
Lambda Light Chain and Heavy Chain -
Constant Domain Mutations Pairs

| Mutation | Heavy Chain | Light Chain |
|---|---|---|
| Type | Constant Domain | Constant Domain |
| Charged pair | V185K | S137D |
| Charged pair | V185E | S137K |
| Charged pair | K147D | T131R |
| Charged pair | K147D | S179R |
| Charged pair | K147D | S179K |
| Charged pair | K147D | T131K |
| Charged pair | L145S | T131K |
| Charged pair | K147(WT) | T131D |
| Charged pair | K147(WT) | S179E |

All position information is reported using the EU numbering scheme
Charge pairs with negative and positive charge residues could be reversed between heavy and light chains, where D or E (negative charge) are replaced by K or R (positive charge) and cognate chain K or R (positive charge) are replaced by D or E (negative charge).

TABLE 4
Lambda Light Chain and Heavy Chain -
Variable Domain Mutations Pairs

| Mutation | Heavy Chain | Light Chain |
|---|---|---|
| Type | Variable Domain | Variable Domain |
| Charged pair | Q39K | Q38D |
| Charged pair | Q39K | Q38E |
| Charged pair | Q39D | Q38K |
| Charged pair | Q39E | Q38K |
| Charged pair | G100D | G44K |
| Knob-into-Hole | Y87G | L45W |

All position information is reported using the Kabat numbering scheme
Charge pairs with negative and positive charge residues could be reversed between heavy and light chains, where D or E (negative charge) are replaced by K or R (positive charge) and cognate chain K or R (positive charge) are replaced by D or E (negative charge).

TABLE 5
Kappa Constant Chain Cysteine Mutation Pairs

| Mutation | Heavy Chain | Light Chain |
|---|---|---|
| Type | Constant Domain | Constant Domain |
| Cys-Cys | S131C | S114C |
| Cys-Cys | S136C | S114C |
| Cys-Cys | S134C | F116C |
| Cys-Cys | F170C | S162C |
| Cys-Cys | P171C | S162C |

All position information is reported using the EU numbering scheme

TABLE 6
Lambda Constant Chain Cysteine Mutation Pairs

| Mutation | Heavy Chain | Light Chain |
|---|---|---|
| Type | Constant Domain | Constant Domain |
| Cys-Cys | V173C | T162C |
| Cys-Cys | L128C | S118C |
| Cys-Cys | S134C | T116C |

All position information is reported using the EU numbering scheme

In certain embodiments, the antibody variant comprises the “light chain pairing mutation set D” comprising the following substitutions: a) the heavy chain and light chain of the anti-CD3 arm comprise the following: i) the amino acid at position 39 (Kabat numbering) of the VH1 is a K and the amino acid at position 38 (Kabat numbering) of the VL1 is a D; ii) the amino acid at position 147 (EU numbering) of the CH1_{H1} is a K and the amino acid at position 131 (EU numbering) of the CL1 is a D; iii) the amino acid at position 173 (EU numbering) of the CH1_{H1} is a C and the amino acid at position 162 (EU numbering) of the CL1 is a C; iv) the amino acid at position 220 (EU numbering) in the H1H is a S and the amino acid at position 214 (EU numbering) of the CL1 is a S; and b) the heavy chain and light chain of the anti-CD20 arm comprise the following: i) the amino acid at position 39 (Kabat numbering) of the VH2 is a D and the amino acid at position 38 (Kabat numbering) of the VL2 is a K; and ii) the amino acid at position 147 (EU numbering) of the CH2_{H1} is a D and the amino acid at position 180 (EU numbering) of the CL2 is a R.

In certain embodiments, the antibody variant comprises the “light chain pairing mutation set D” comprising the following substitutions: a) the heavy chain and light chain of the anti-CD20 arm comprise the following: i) the amino acid at position 39 (Kabat numbering) of the VH1 is a K and the amino acid at position 38 (Kabat numbering) of the VL1 is a D; ii) the amino acid at position 147 (EU numbering) of the CH1_{H1} is a K and the amino acid at position 131 (EU numbering) of the CL1 is a D; iii) the amino acid at position 173 (EU numbering) of the CH1_{H1} is a C and the amino acid at position 162 (EU numbering) of the CL1 is a C; iv) the amino acid at position 220 (EU numbering) in the H1H is a S and the amino acid at position 214 (EU numbering) of the CL1 is a S; and b) the heavy chain and light chain of the anti-CD3 arm comprise the following: i) the amino acid at position 39 (Kabat numbering) of the VH2 is a D and the amino acid at position 38 (Kabat numbering) of the VL2 is a K; and ii) the amino acid at position 147 (EU numbering) of the CH2_{H1} is a D and the amino acid at position 180 (EU numbering) of the CL2 is a R.

It can be desirable to modify an antibody disclosed herein with respect to effector function, so as to enhance, e.g., the effectiveness of the antibody in treating diseases and disorders. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J Exp Med., 176:1191-1195 (1992) and Shopes, J. Immunol., 148:2918-2922. (1992)). Alternatively, an antibody can be engineered that has dual Fc regions and can thereby have enhanced complement lysis and ADCC capabilities. (See Stevenson et al., Anti-Cancer Drug Design, 3:219-230 (1989)).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, antibodies may comprise a substitution mutation in the Fc region that reduces effector function. In some embodiments, the substitution mutation is an aglycosylation site mutation. In some embodiments, the aglycosylation site mutation is at amino acid residue 297 and amino acid substitutions at residues 234, 235, 265 and 331 (EU numbering) to disrupt the Fc receptor binding interface. In some embodiments, the aglycosylation site mutation reduces effector function of the antibody.

In some embodiments, i) the H1H and/or the H2H has an A at positions 234 and 235 (EU numbering); or ii) the H1H and/or the H2H has an A at positions 234, 235 and 237 (EU numbering) iii) the H1H and/or the H2H has an A at positions 234 and 235 and G at position 329 (EU numbering). In some embodiments, i) the CH1_{H3} and/or the CH2_{H3} has an A at position 297 (EU numbering) ii) the CH1_{H1} and/or the CH2_{H3} has a G at position 297 (EU numbering); or iii) the CH1_{H3} and/or the CH2_{H3} has a S at position 297 (EU numbering). In some embodiments, the CH1_{H3} and/or the CH2_{H3} has an S at position 331 (EU numbering).

The use of knobs into holes as a method of producing multispecific antibodies is well known in the art. See U.S. Pat. No. 5,731,168 granted 24 Mar. 1998 assigned to Genentech, PCT Pub. No. WO2009089004 published 16 Jul. 2009 and assigned to Amgen, and US Pat. Pub. No. 20090182127 published 16 Jul. 2009 and assigned to Novo Nordisk A/S. See also Marvin and Zhu, Acta Pharmacologica Sincia (2005) 26(6):649-658 and Kontermann (2005) Acta Pharacol. Sin., 26:1-9.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimeric antibody, and thereby favor heteromultimeric antibody formation over homomultimeric antibody formation, for example. The protuberance may exist in the original interface or may be introduced synthetically (e.g. by altering nucleic acid encoding the interface). Normally, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The upper limit for the number of original residues which are replaced is the total number of residues in the interface of the first polypeptide.

The preferred import residues for the formation of a protuberance are generally naturally occurring amino acid residues and are preferably selected from arginine (R), phenylalanine (F), tyrosine (Y) and tryptophan (W). Most preferred are tryptophan and tyrosine. In one embodiment, the original residue for the formation of the protuberance has a small side chain volume, such as alanine, asparagine, aspartic acid, glycine, serine, threonine or valine. Exemplary amino acid substitutions in the CH1_{H3} or CH2_{H3} domain for forming the protuberance include without limitation the T366W substitution.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). Normally, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. The upper limit for the number of original residues which are replaced is the total number of residues in the interface of the second polypeptide. The side chain volumes of the various amino residues are shown in Table 1 above. The preferred import residues for the formation of a cavity are usually naturally occurring amino acid residues and are preferably selected from alanine (A), serine (S), threonine (T) and valine (V). Most preferred are serine, alanine or threonine. In one embodiment, the original residue for the formation of the cavity has a large side chain volume, such as tyrosine, arginine, phenylalanine or tryptophan. Exemplary amino acid substitutions in the CH1_{H3} or CH2_{H3} domain for generating the cavity include without limitation the T366S, L368A, Y407A, Y407T and Y407V substitutions. In certain embodiments, the knob half-antibody comprises T366W substitution, and the hole half-antibody comprises the T366S/L368A/Y407V substitutions.

In certain embodiments, the antibody variant comprises the following substitutions: the CH1_{H3} has a C at position 349, an S at position 366, an A at position 368 and a V at position 407 (EU numbering); and the CH2_{H3} has a C at position 354 and a W at position 366 (EU numbering).

In certain embodiments, the antibody variant comprises the following substitutions: the CH2_{H3} has a C at position 349, an S at position 366, an A at position 368 and a V at position 407 (EU numbering); and the CH1_{H3} has a C at position 354 and a W at position 366 (EU numbering).

In certain embodiments, the antibody variant comprises the following substitutions: the CH1_{H3} has a C at position 354, an S at position 366, an A at position 368 and a V at position 407 (EU numbering); and the CH2_{H3} has a C at position 349 and a W at position 366 (EU numbering).

In certain embodiments, the antibody variant comprises the following substitutions: the CH2_{H3} has a C at position 354, an S at position 366, an A at position 368 and a V at position 407 (EU numbering); and the CH1H3 has a C at position 349 and a W at position 366 (EU numbering).

### Fusion Peptides

Provided herein is an antibody (e.g. monospecific antibody or bispecific antibody) that has a fusion peptide fused to the N-terminus or the C-terminus of the first heavy chain polypeptide or the second heavy chain polypeptide.

Critical to the initial T cell response is the capacity for T cells to detect foreign and mutated proteins through their T cell receptor. This response, often referred to as signal 1 of T cell activation, occurs when the T cell receptor engages a cell that displays a foreign or mutated protein fragment or antigen in a specific protein complex called the Major Histocompatibility Complex I (MHCI). The activation of the T cell receptor is by itself both activating and auto-regulatory to T cells. Strong binding of the TCR to an MHCI complex creates chronic activation of the TCR. This form of signal is associated with T cells that are reactive to self-antigens. T cells are programed to inactivate when they experience this form activation. T cells with TCR that bind weaker, but sufficient for activation, experience acute signaling with the potential to remain active and differentiate into memory T cells. This is emerging as important consideration in the design the T cell therapeutics.

T cell cytokine activation, often referred to signal 3, is important in T cell transitions, either from non-dividing to a state of rapid cell division or from one phenotypic state to another. T cell cytokine receptors bind to cytokines that are produced by immune and non-immune cells and depending on the cytokine and the state of the T cell at the time of receiving the cytokine signal can induce cell proliferation, can sustain vitality, or can induce differentiation of T cells into a specialized cell state appropriate for sustained activation or inactivation following infection.

One example is the transition that naïve cells experience through cytokines which can induce naïve T cells to proliferate and promote T cell differentiation into memory T cells. Exemplary cytokines include but are not limited to IL-2, IL-7, IL-10, IL-12, IL-15, IL-18 and IL-21.

Costimulatory receptor activation, referred to as signal 2 provides a context specific cell-to-cell reinforcement of T activation. The most recognized form of costimulation occurs when T cells interact with activated antigen presenting cells through the T cell costimulatory receptor CD28 with CD80 and CD86 ligands found on APCs. These interactions can “prime” specific T cells armed with T cell receptors responsive to pathogen or cancer proteins.

Less appreciated is costimulation induced at the site of infection and malignancies. This includes costimulation that acts through CD2 and NKG2D receptors responsive to ligands like CD58 and UL 16 binding proteins (e.g. ULBP2/5/6) that are induced in immune cells and epithelial cells upon viral infection. These signals provide not only reinforcement of T activation, but confirmation that the T cell's lethal effector activities are targeted with single cell accuracy. While many costimulatory receptors have been discovered, the importance of each receptor's specific context and the impact of concurrent signaling of multiple costimulatory receptors remains largely unknown and an area to greatly advance our understanding of T cell biology and creating possibilities for novel tumor-targeted T cell therapeutic development.

Costimulatory ligands include but are not limited to CD48, CD58, CD86, TNFSF9, OX40L, 4-1BBL, GITL, CD70, CD80, MR1, TNFSF4, ICOSL or ICOSLG.

CD58 is advantageous over other costimulatory ligands in that it is the primary costimulatory pathway available at the tumor site as tumor infiltrating T lymphocytes often lose expression of other costimulatory receptors like CD28, or due to the low immunogenicity of tumor cells, tumor cells do not sufficiently activate T cell, thus limiting the potential of inducible costimulatory receptors like 41BB.

As described previously, the anti-CD3ε antibodies of the disclosure induce varying levels of T cell receptor activation that confer alteration in T cell vitality and cytokine production. Accordingly, a fusion of the costimulatory ligand CD58 to the anti-CD3ε bispecific antibody provides integrated costimulatory T cell activation for optimal T cell activation.

In some embodiments, the bispecific antibody has a peptide fused to the N-terminus of the first heavy chain polypeptide (H1). In some embodiments, the bispecific antibody has a peptide fused to the C-terminus of the first heavy chain polypeptide (H1). In some embodiments, the bispecific antibody has a polypeptide fused to the N-terminus of the second heavy chain polypeptide (H2). In some embodiments, the bispecific antibody has a peptide fused to the C-terminus of the second heavy chain polypeptide (H2). Exemplary peptides include but are not limited to IL-2, IL-7, IL-10, IL-12, IL-15, IL-18, IL-21 or portions thereof. Exemplary peptides include but are not limited to CD48, CD58, CD86, TNFSF9, OX40L, 4-1BBL, GITL, CD70, CD80, MR1, TNFSF4, ICOSL, ICOSLG or portions thereof. Exemplary peptide sequences that are fused to the bispecific antibodies include but are not limited to those listed in Table 7.1 and Table 7.2.

TABLE 7.1
Exemplary Fusion Peptide Sequences

| | | SEQ ID |
|---|---|---|
| Domain | Amino Acid Sequence | NO: |
| CD58-ECD | FSQQIYGVVYGNVTFHVPSNVPLKEVLWKKQKDKVAELENSEFRAFS | SEQ |
| (CD58) | SFKNRVYLDTVSGSLTIYNLTSSDEDEYEMESPNITDTMKFFLYVLE | ID |
| | SLPSPILTCALINGSIEVQCMIPEHYNSHRGLIMYSWDCPMEQCKRN | NO: |
| | STSIYFKMENDLPQKIQCTLSNPLFNTTSSIILTTCIPSSGHSRHRY | 49 |
| CD58- | FSQQIYGVVYGNVTFHVPSNVPLKEVLWKKQKDKVAELENSEFRAFS | SEQ |
| variable | SFKNRVYLDTVSGSLTIYNLTSSDEDEYEMESPNITDTMKFFLYVLE | ID |
| domain | | NO: |
| (″CD58v*″) | | 50 |

TABLE 7.2
Exemplary Fusion Peptide Sequences

| | | | SEQ |
|---|---|---|---|
| Name | Description | Sequence | ID NO: |
| CD58-1 | Full length CD58, | MVAGSDAGRALGVLSVVCLLHCFGFISCFSQQIYGVVYGNVT | 624 |
| | including signal | FHVPSNVPLKEVLWKKQKDKVAELENSEFRAFSSFKNRVYLD | |
| | sequence | TVSGSLTIYNLISSDEDEYEMESPNITDTMKFFLYVLESLPS | |
| | | PTLTCALINGSIEVQCMIPEHYNSHRGLIMYSWDCPMEQCKR | |
| | | NSTSIYFKMENDLPQKIQCTLSNPLFNTTSSIILTTCIPSSG | |
| | | HSRHRYALIPIPLAVITTCIVLYMNGILKCDRKPDRTNSN | |
| CD58-2 | Extracellular | FSQQIYGVVYGNVTFHVPSNVPLKEVLWKKQKDKVAELENSE | 625 |
| | domain of CD58, | FRAFSSFKNRVYLDTVSGSLTIYNLTSSDEDEYEMESPNITD | |
| | corresponding to | TMKFFLYVLESLPSPILTCALINGSIEVQCMIPEHYNSHRGL | |
| | amino acids 29- | IMYSWDCPMEQCKRNSTSIYFKMENDLPQKIQCTLSNPLFNT | |
| | 215 of CD58 (WT) | TSSIILTTCIPSSGHSRHR | |
| CD58-3 | Extracellular | BSQQIYGVJYGNVTFHVPSNQPLKEVLWKKQKDKVAELENSE | 626 |
| | domain of CD58, | FRAFSSFKNRVYLDTUSGSLTIYNLTSSDEDEYEMESPNITD | |
| | corresponding to | XMKFFLYVZESLPSPTLICALINGSIEVQCMIPEHYNSHRGL | |
| | amino acids 29- | IMYSWDCPMEQCKRNSTSIYFKMENDLPQKIQCTLSNPLFNT | |
| | 215 of CD58 (with | TSSIILTTCIPSSGHSRAR | |
| | permitted | B = F or S | |
| | substitutions) | J = V or K | |
| | | O = V or Q | |
| | | U = V or K | |
| | | X = T or S | |
| | | Z = L or G | |
| CD58-4 | Amino acids | SQQIYGVVYGNVTFHVPSNVPLKEVLWKKQKDKVAELENSEF | 627 |
| | 130-23 (WT) | RA | |
| CD58-5 | Amino acids | SQQIYGVJYGNVTFHVPSNQPLKEVLWKKQKDKVAELENSEF | 628 |
| | 130-23 (with | RA | |
| | permitted | J = V or K | |
| | substitutions) | O = V or Q | |

In some embodiments the polypeptide is fused directly to the bispecific antibody. In some embodiments, the polypeptide is fused indirectly through a linker. In some embodiments, the bispecific antibody fused with a peptide comprises a linker sequence. Exemplary linker sequences include but are not limited to those listed in Table 8.1 and Table 8.2.

TABLE 8.1
Exemplary Linker Sequences

| Domain | Amino Acid Sequence | SEQ ID NO: |
|---|---|---|
| Linker-1 | GGGGS | SEQ ID NO: |
| | | 52 |
| Linker-2 | GGGGSGGGGS | SEQ ID NO: |
| | | 53 |
| Linker-3 | GGGGSGGGGSGGGGS | SEQ ID NO: |
| | | 54 |

TABLE. 8.2
Exemplary Linker Sequences

| Linker | | SEQ |
|---|---|---|
| Name | Linker Sequence | ID NO: |
| L1 | ADAAP | 620 |
| L2 | ADAAPTVSIFP | 621 |
| L3 | ADAAPTVSIFPP | 623 |
| L4 | AKTTAP | 624 |
| L5 | AKTTAPSVYPLAP | 625 |
| L6 | AKTTPKLEEGEFSEARV | 626 |
| L7 | AKTTPKLGG | 627 |
| L8 | AKTTPP | 628 |
| L9 | AKTTPPSVTPLAP | 629 |
| L10 | ASTKGP | 630 |
| L11 | ASTKGPSVFPLAP | 631 |
| L12 | ASTKGPSVFPLAPASTKGPSVFPLAP | 632 |
| L13 | EGKSSGSGSESKST | 633 |
| L14 | GEGESGEGESGEGES | 634 |
| L15 | GEGESGEGESGEGESGEGES | 635 |
| L16 | GEGGSGEGGSGEGGS | 636 |
| L17 | GENKVEYAPALMALS | 637 |
| L18 | GGEGSGGEGSGGEGS | 638 |
| L19 | GGGESGGEGSGEGGS | 639 |
| L20 | GGGESGGGESGGGES | 640 |
| L21 | (GGGGS)ₙ | 641 |
| | (also referred to as (G4S)ₙ), wherein n can be | |
| | 1-10 | |
| L22 | GGGGSGGGGS | 642 |
| L23 | GGGGGGGGSGGGGS | 643 |
| L24 | GGGGSGGGGSGGGGSGGGGS | 644 |
| L25 | GGGKSGGGKSGGGKS | 645 |
| L26 | GGGKSGGKGSGKGGS | 646 |
| L27 | GGKGSGGKGSGGKGS | 647 |
| L28 | GGSGG | 648 |
| L29 | GGSGGGGSG | 649 |
| L30 | GGSGGGGGGGGGS | 650 |
| L31 | GHEAAAVMQVQYPAS | 651 |
| L32 | GKGGSGKGGSGKGGS | 652 |
| L33 | GKGKSGKGKSGKGKS | 653 |
| L34 | GKGKSGKGKSGKGKSGKGKS | 654 |
| L35 | GKPGSGKPGSGKPGS | 655 |
| L36 | GKPGSGKPGSGKPGSGKPGS | 656 |
| L37 | GPAKELTPLKEAKVS | 657 |
| L38 | GSAGSAAGSGEF | 658 |
| L39 | IRPRAIGGSKPRVA | 659 |
| L40 | KESGSVSSEQLAQFRSLD | 660 |
| L41 | KTTPKLEEGEFSEAR | 661 |
| L42 | QPKAAP | 662 |
| L43 | QPKAAPSVTLFPP | 663 |
| L44 | RADAAAA(G4S)₄ | 664 |
| L45 | RADAAAAGGPGS | 665 |
| L46 | RADAAP | 666 |
| L47 | RADAAPTVS | 667 |
| L48 | SAKTTP | 668 |
| L49 | SAKTTPKLEEGEFSEARV | 669 |
| L50 | SAKTTPKLGG | 670 |
| L51 | STAGDTHLGGEDFD | 671 |
| L52 | TVAAP | 672 |
| L53 | TVAAPSVFIFPP | 673 |
| L54 | TVAAPSVFIFPPTVAAPSVFIFPP | 674 |

### Bispecific and Multispecific Antibodies of the Disclosure

The present disclosure provides a method of T cell re-targeting to a tumor cell population in a subject, the method comprising administering to the subject a bispecific antibody or a multispecific antibody. The present disclosure provides a method for stimulating an T cell mediated immune response to a tumor cell population in a subject, the method comprising administering to the subject a bispecific antibody or a multispecific antibody. The present disclosure provides a method for stimulating an T cell mediated immune response to a tumor cell population in a subject, the method comprising administering to the subject a bispecific antibody or a multispecific antibody. In some embodiments, the subject was previously administered a population of modified T-cells that bind a first target antigen on the tumor cell.

In some embodiments, the subject is suffering from a disease associated with expression of CD19, such as a hematological cancer (e.g., a hematological cancer described herein such as CLL, MCL, or ALL) and the subject is, or is identified as, a partial responder, non-responder, or relapser to one or more therapies for the hematological cancer, e.g., an anti-CD19 adoptive CAR-T cell therapy. In some embodiments, the subject has, or is identified as having, a CD19 mutation. The mutation may be, e.g., a point mutation, an insertion, or a deletion. The mutation may be, e.g., a mutation at the binding site for CD19, or a mutation that results in reduced or abolished expression of CD19. The mutation may confer a decreased response (e.g., resistance) to the anti-CD19 CAR-T cell therapy.

In some embodiments, the subject was previously administered a population of modified T-cells binds to CD19 of a tumor cell. In some embodiments, the subject was previously administered a population of modified T-cells comprising FMC63 that binds to CD19 of a tumor cell.

In some embodiments, the bispecific antibody of the present disclosure binds to an anti-CD19 antibody. For example, the anti-CD19 antibody can include FMC63 (IgG2a) (Chemicon Int'l., Temecula, Calif.) (Nicholson et al., Mol. Immunol. 34:1157-1165 (1997). The FMC63 antibody (described in Nicholson et al., Molecular Immunology, 34(16-17): 1157-1165 (1997)) is one example of a murine anti-CD19 monoclonal antibody that can be recognized by the bispecific antibody of the present disclosure. Variable regions of the FMC63 monoclonal antibody have been utilized in CARs that have been tested in clinical trials (see, e.g., Kochenderfer et al., Nature Review Clinical Oncol., 10(5); 267-276 (2013); Porter et al., New Eng. J Med., 365(8): 725-733 (2011); Kalos et al., Science Translational Medicine, 3(95): 95ra73 (2011); Kochenderfer et al., Blood, 116(20): 4099-4102 (2010); and Kochenderfer et al., Blood, 119(12): 2709-2720 (2012)).

In some embodiments, the bispecific antibody of the present disclosure can bind to a CD19 targeting CAR T cell. In some embodiments, the bispecific antibody of the present disclosure can bind to a FMC63 scFv portion of a CAR T cell.

In some embodiments, the bispecific antibody comprises a first antigen binding region that binds to a target antigen on the modified T cell (e.g., CD3, a CAR domain such as FMC63, a CAR hinge, or a T cell antigen) and a second antigen binding region that binds to a second target antigen on the tumor cell (e.g., a cell surface antigen, or DAA, such as CD20, CD180, BMCA or CD22). In some embodiments, the bispecific antibody further comprises a polypeptide fused to the N-terminus or the C-terminus of the bispecific antibody. In some embodiments, the polypeptide fusion comprises a CD58 or a fragment thereof. In some embodiments, the bispecific antibody comprises a third antigen binding region that binds a third target antigen (e.g., a third biological molecule, e.g., a cell surface antigen, e.g., a disease associated antigen).

In some embodiments, the first target antigen is not expressed on the tumor cell. In some embodiments, the first target antigen on the tumor cell is expressed at a low level. In some embodiments, the first target antigen on the tumor cell is expressed at a lower level than the second target antigen on the tumor cell. An exemplary first target antigen on a tumor cell includes CD19. A mutation at the binding site for CD19, or a mutation that results in reduced (low level expression) or abolished expression of CD19. Accordingly, the disclosure provides methods of using bispecific antibodies to modified T cells to a second target antigen that still has expression on the tumor cell, thereby preserving the function of the modified T cell.

In some embodiments, the target antigen on the modified T cell is CD3, a CAR domain, a CAR hinge domain, or a T cell antigen. In some embodiments, the target antigen on the modified T cell is a CD3. In some embodiments, the target antigen on the modified T cell is a T cell antigen other than CD3. In some embodiments, the target antigen on the modified T cell is an FMC63 CAR domain. In some embodiments, the target antigen on the modified T cell is CD8.

In some embodiments, the second target antigen on the tumor cell is a cell surface antigen. In some embodiments, the cell surface antigen is a disease-associated antigen. In some embodiments, the disease associated antigen is a tumor antigen. In some embodiments, the tumor antigen is a hematologic tumor antigen. In some embodiments, the tumor antigen is a solid tumor antigen.

In some embodiments, the first antigen binding domain and the second antigen binding domain bind to T cell. In some embodiments, the bispecific antibody comprises two binding domains that bind to a T cell. In some embodiments, the first antigen binding domain and the second antigen binding domain bind to T cell surface antigen.

In some embodiments, the first antigen binding domain and the second antigen binding domain bind to a chimeric antigen receptor (CAR) domain. In some embodiments, the first antigen binding domain and the second antigen binding domain bind to CAR expressed on the surface of a T cell. In some embodiments, the first antigen binding domain binds to a T cell antigen and the second antigen binding domain binds to a CAR domain on a CAR T cell. In some embodiments, the first antigen binding domain binds to a T cell antigen on a CAR T cell and the second antigen binding domain binds to a CAR domain on a CAR T cell. In some embodiments, the first antibody binding domain and the second antigen binding domain bind to different antigens on the same CAR-T cell.

In some embodiments, the bispecific antibody comprises a first antigen binding region that binds to CD3 on the modified T cell and a second antigen binding region that binds to CD20 on the tumor cell. In some embodiments, a CD58 or fragment thereof is fused to the C-terminus of the first heavy chain polypeptide of the bispecific antibody. In some embodiments, the bispecific antibody comprises a first antigen binding region that binds to FMC63 on the modified T cell and a second antigen binding region that binds to CD20 on the tumor cell. In certain embodiments, the bispecific antibody comprises substitutions in the variable heavy, variable light, constant heavy or constant light chain domains of the anti-CD3 or the anti-CD20 arms or both. Exemplary anti-CD3 and anti-CD20 antibodies are described in U.S. Application No. 63/368,852, 63/432,665 and PCT Publication Nos. WO 2019/104075, WO 2023/178357, each of which are incorporated by reference herein in their entireties.

In some embodiments, the bispecific antibody comprises a first antigen binding region that binds to CD3 on the modified T cell and a second antigen binding region that binds to CD180 on the tumor cell. In some embodiments, the bispecific antibody comprises a first antigen binding region that binds to FMC63 on the modified T cell and a second antigen binding region that binds to CD180 on the tumor cell. In some embodiments, a CD58 or fragment thereof is fused to the C-terminus of the first heavy chain polypeptide of the bispecific antibody. In certain embodiments, the bispecific antibody comprises substitutions in the variable heavy, variable light, constant heavy or constant light chain domains of the anti-CD3 or the anti-CD180 arms or both. Exemplary anti-CD3 and anti-CD180 antibodies are described in U.S. Application No. 63/584,166 and PCT Publication Nos. WO 2019/104075, WO 2023/178357, each of which are incorporated by reference herein in their entireties.

In some embodiments, the bispecific antibody comprises a first antigen binding region that binds to CD3 on the modified T cell and a second antigen binding region that binds to BCMA on the tumor cell. In some embodiments, the bispecific antibody comprises a first antigen binding region that binds to FMC63 on the modified T cell and a second antigen binding region that binds to BCMA on the tumor cell. In some embodiments, a CD58 or fragment thereof is fused to the C-terminus of the first heavy chain polypeptide of the bispecific antibody. In certain embodiments, the bispecific antibody comprises substitutions in the variable heavy, variable light, constant heavy or constant light chain domains of the anti-CD3 or the anti-BCMA arms or both. Exemplary anti-CD3 and anti-BCMA antibodies are described in PCT Publication No. WO 2023/178357, which is incorporated by reference herein in its entirety.

In some embodiments, the bispecific antibody comprises a first antigen binding region that binds to FMC63 on the modified T cell and a second antigen binding region that binds to BCMA on the tumor cell. In some embodiments, a CD58 or fragment thereof is fused to the C-terminus of the first heavy chain polypeptide of the bispecific antibody. In certain embodiments, the bispecific antibody comprises substitutions in the variable heavy, variable light, constant heavy or constant light chain domains of the anti-FMC63 or the anti-BCMA arms or both. Exemplary anti-FMC63 and anti-BCMA antibodies are described in PCT Publication No. WO 2023/178357, which is incorporated by reference herein in its entirety.

In some embodiments, the bispecific antibody comprises a first antigen binding region that binds to FMC63 on the modified T cell and a second antigen binding region that binds to CD22 on the tumor cell. In some embodiments, a CD58 or fragment thereof is fused to the C-terminus of the first heavy chain polypeptide of the bispecific antibody. In certain embodiments, the bispecific antibody comprises substitutions in the variable heavy, variable light, constant heavy or constant light chain domains of the anti-FMC63 or the anti-CD22 arms or both. Exemplary anti-FMC63 and anti-CD22 antibodies are described in PCT Publication No. WO 2023/178357, which is incorporated by reference herein in its entirety.

### Cluster of Differentiation 3 (CD3)

The term “cluster of differentiation 3” or “CD3,” as used herein, refers to any native CD3 from any vertebrate source, including mammals such as primates (e.g. humans, cynomolgus monkey) and rodents (e.g., mice and rats), unless otherwise indicated, including, for example CD3ε, CD3γ, CD3α, and CD3β chains. CD3 is a cell surface complex expressed on T cells in association with the T cell receptor. The CD3 complex is required for the activation of CD8+ and CD4+ T lymphocytes. It is formed of three different but highly related chains: one CD3 gamma chain, one CD3 delta chain, and two CD3 epsilon chains, which associate with each other to form a CD3 epsilon/gamma heterodimer, and a CD3 epsilon/delta heterodimer. The two CD3 heterodimers, together with the T cell receptor (TCR) and the signal-transducing zeta chain homodimer form the T cell receptor complex.

The term encompasses “full-length” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length.

In some embodiments, the invention provides isolated antibodies that bind to CD3. In some embodiments, the invention provides antibodies that bind to CD3ε. In some instances, the anti-CD3ε antibody binds to a human CD3ε polypeptide or a cynomolgus monkey (cyno) CD3ε polypeptide. In some instances, the human CD3 polypeptide or the cyno CD3 polypeptide is a human CD3ε polypeptide of amino acid sequence: MQSGTHWRVLGLCLLSVGVWGQDGNEEMGGITQTPYKVSISGTTVILTCPQY PGSEILWQHNDKNIGGDEDDKNIGSDEDHLSLKEFSELEQSGYYVCYPRGSKP EDANFYLYLRARVCENCMEMDVMSVATIVIVDICITGGLLLLVYYWSKNRK AKAKPVTRGAGAGGRQRGQNKERPPPVPNPDYEPIRKGQRDLYSGLNQRRI (SEQ ID NO: 419) or a cyno CD3ε polypeptide of amino acid sequence MQSGTRWRVLGLCLLSIGVWGQDGNEEMGSITQTPYQVSISGTTVILTCSQHL GSEAQWQHNGKNKEDSGDRLFLPEFSEMEQSGYYVCYPRGSNPEDASHHLY LKARVCENCMEMDVMAVATIVIVDICITLGLLLLVYYWSKNRKAKAKPVTR GAGAGGRQRGQNKERPPPVPNPDYEPIRKGQQDLYSGLNQRRI (SEQ ID NO: 420), respectively. In some instances, the anti-CD3 antibody binds to an epitope within a fragment of CD3ε (e.g., human CD3ε) consisting of amino acid residues 1-26 or amino acid residues 1-27 of human CD3ε (SEQ ID NO: 419).

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

In some embodiments, alanine scanning mutagenesis was performed on the “SP34” anti-CD3ε antibody to produce affinity modulated anti-CD3ε antibodies of the invention.

In some embodiments, the first antigen binding region that binds to CD3 comprises any one of the VH and VL sequences listed in Table 9.1. In Table 9.1, the underlined sequences are CDR sequence according to Kabat and the bolded sequences are CDR sequences according to Chothia.

In some embodiments, a anti-CD3 antibody of the disclosure comprises: a) a heavy chain variable region (VH) comprising a VH complementarity determining region 1 (VH_{CDR1}), a VH complementarity determining region 2 (VH_{CDR2}) and a VH complementarity determining region 3 (VH_{CDR3}); and b) a light chain variable region (VL) comprising a VL complementarity determining region 1 (VL_{CDR1}), a VL complementarity determining region 2 (VL_{CDR2}) and a VL complementarity determining region 3 (VL_{CDR3}). Tables 9.2 and 9.3 provide exemplary of CDR sequences of the anti-CD3 antibodies provided herein.

TABLE 9.1
Anti-CD3 Variable Heavy Chain and Variable Light Chain
Domains

| Ab | Domain | Amino Acid Sequence | SEQ ID NO |
|---|---|---|---|
| CD3-01 | CD3_VL- | ELVVTQEPSLTVSPGGTVTLTCRSSTGAVTTSNYANW | SEQ ID NO: |
| (SP34) | 1 | VQDKPGQAPRGLIGGTNKRAPGTPARFSGSLLGGKAA | 22 |
| | | LTLSGVQPEDEAEYYCALWYSNLWVFGGGTKLTVL | |
| | CD3_VH- | EVQLLESGGGLVQPGGSLKLSCAASGFTFNTYAMNWV | SEQ ID NO: |
| | 1 | RKAPGKGLEWVARIRSKYNNYATYYADSVKDRFTISR | 13 |
| | | DDSKNTAYLQMNNLKTEDTAVYYCVRHGNFGNSYVSW | |
| | | FAYWGQGTLVTVSS | |
| CD3-A1 | CD3_VL- | ELVVTQEPSLTVSPGGTVTLTCRSSTGAVITSNYANW | SEQ ID NO: |
| | 4 | VQDKPGQAPRGLIGGTNKRAPGTPARFSGSLLGGKAA | 25 |
| | | LTLSGVQPEDEAEYYCALWDSNLWVFGGGTKLTVL | |
| | CD3_VH- | EVQLLESGGGLVQPGGSLKLSCAASGFTFNTYAMNWV | SEQ ID NO: |
| | 1 | RKAPGKGLEWVARIRSKYNNYATYYADSVKDRFTISR | 13 |
| | | DDSKNTAYLQMNNLKTEDTAVYYCVRHGNFGNSYVSW | |
| | | FAYWGQGTLVTVSS | |
| CD3-A2 | CD3_VL- | ELVVTQEPSLTVSPGGTVTLTCRSSTGAVTTSNYANW | SEQ ID NO: |
| | 6 | VQDKPGQAPRGLIGDTNKRAPGTPARFSGSLLGGKAA | 27 |
| | | LTLSGVQPEDEAEYYCALWYSNLWVFGGGTKLTVL | |
| | CD3_VH- | EVQLLESGGGLVQPGGSLKLSCAASGFTFNTYAMNWV | SEQ ID NO: |
| | 1 | RKAPGKGLEWVARIRSKYNNYATYYADSVKDRFTISR | 13 |
| | | DDSKNTAYLQMNNLKTEDTAVYYCVRHGNFGNSYVSW | |
| | | FAYWGQGTLVTVSS | |
| CD3-A3 | CD3_VL- | ELVVTQEPSLTVSPGGTVTLTCRSSTGAVTTSNYANW | SEQ ID NO: |
| | 2 | VQKKPGQAPRGLIGGTNKRAPGTPARFSGSLLGGKAA | 23 |
| | | LTLSGVQPEDEAEYYCALWYSNLWVFGGGTKLTVL | |
| | CD3_VH- | EVQLLESGGGLVQPGGSLKLSCAASGFTFNTYAMNWV | SEQ ID NO: |
| | 2 | RDAPGKGLEWVARIRSKYNNYATYYADSVKDRFTISR | 14 |
| | | DDSKNTAYLQMNNLKTEDTAVYYCVRHGNFGNSYVSW | |
| | | FAYWGQGTLVTVSS | |
| CD3-A4 | CD3_VL- | ELVVTQEPSLTVSPGGTVTLTCRSSTGAVTTSNYANW | SEQ ID NO: |
| | 5 | VQDKPGQAPRGLIGGTNKRAPGTPARFSGSLLGGKAA | 26 |
| | | LTLSGVQPEDEAEYYCALWYSQLWVFGGGTKLTVL | |
| | CD3_VH- | EVQLLESGGGLVQPGGSLKLSCAASGFTFNTYAMNWV | SEQ ID NO: |
| | 3 | RKAPGKGLEWVARIRSKYNNYATYYADSVKDRFTISR | 15 |
| | | DDSKNTAYLQMNNLKTEDTAVYYCVRHGNFGDSYVSW | |
| | | FAYWGQGTLVTVSS | |
| CD3-A5 | CD3_VL- | ELVVTQEPSLTVSPGGTVTLTCRSSTGAVITSNYANW | SEQ ID NO: |
| | 5 | VQDKPGQAPRGLIGGTNKRAPGTPARFSGSLLGGKAA | 26 |
| | | LTLSGVQPEDEAEYYCALWYSQLWVFGGGTKLTVL | |
| | CD3_VH- | EVQLLESGGGLVQPGGSLKLSCAASGFTFNTYAMNWV | SEQ ID NO: |
| | 4 | RKAPGKGLEWVARIRSKYNNYATYYADSVKDRFTISR | 16 |
| | | DDSKNTAYLQMNNLKTEDTAVYYCVRHGNFGDDYVSW | |
| | | FAYWGQGTLVTVSS | |
| CD3-A6 | CD3_VL- | ELVVTQEPSLTVSPGGTVTLTCRSSTGAVTTSNYANW | SEQ ID NO: |
| | 1 | VQDKPGQAPRGLIGGTNKRAPGTPARFSGSLLGGKAA | 22 |
| | | LTLSGVQPEDEAEYYCALWYSNLWVFGGGTKLTVL | |
| | CD3_VH- | EVQLLESGGGLVQPGGSLKLSCAASGFTFNDYAMNWV | SEQ ID NO: |
| | 5 | RKAPGKGLEWVARIRSKYNNYATYYADSVKDRFTISR | 17 |
| | | DDSKNTAYLQMNNLKTEDTAVYYCVRHGNFGNSYVSW | |
| | | FAYWGQGTLVTVSS | |
| CD3-A7 | CD3_VL- | ELVVTQEPSLTVSPGGTVTLTCRSSTGAVTTSNYANW | SEQ ID NO: |
| | 1 | VQDKPGQAPRGLIGGTNKRAPGTPARFSGSLLGGKAA | 22 |
| | | LTLSGVQPEDEAEYYCALWYSNLWVFGGGTKLTVL | |
| | CD3_VH- | EVQLLESGGGLVQPGGSLKLSCAASGFTFNTYAMNWV | SEQ ID NO: |
| | 6 | RKAPGKGLEWVAKIRSKYNNYATYYADSVKDRFTISR | 18 |
| | | DDSKNTAYLQMNNLKTEDTAVYYCVRHGNFGDSYVSW | |
| | | FAYWGQGTLVTVSS | |
| CD3-A8 | CD3_VL- | ELVVTQEPSLTVSPGGTVTLTCRSSTGAVTTSNYANW | SEQ ID NO: |
| | 5 | VQDKPGQAPRGLIGGTNKRAPGTPARFSGSLLGGKAA | 26 |
| | | LTLSGVQPEDEAEYYCALWYSQLWVFGGGTKLTVL | |
| | CD3_VH- | EVQLLESGGGLVQPGGSLKLSCAASGFTFNTYAMNWV | SEQ ID NO: |
| | 6 | RKAPGKGLEWVAKIRSKYNNYATYYADSVKDRFTISR | 18 |
| | | DDSKNTAYLQMNNLKTEDTAVYYCVRHGNFGDSYVSW | |
| | | FAYWGQGTLVTVSS | |
| CD3-A9 | CD3_VL- | ELVVTQEPSLTVSPGGTVTLTCRSSTGAVTTSNYANW | SEQ ID NO: |
| | 5 | VQDKPGQAPRGLIGGTNKRAPGTPARFSGSLLGGKAA | 26 |
| | | LTLSGVQPEDEAEYYCALWYSQLWVFGGGTKLTVL | |
| | CD3_VH- | EVQLLESGGGLVQPGGSLKLSCAASGFTFNTYAMNWV | SEQ ID NO: |
| | 7 | RKAPGKGLEWVARIRSKYNNYATYYADSVKDRFTISR | 19 |
| | | DDSKNTAYLQMNNLKTEDTAVYYCVRHGSFGNSYVSW | |
| | | FAYWGQGTLVTVSS | |
| CD3- | CD3_VL | ELVVTQEPSLTVSPGGTVTLTCRSSTGAVITSNYANW | SEQ ID NO: |
| A10 | 5 | VQDKPGQAPRGLIGGTNKRAPGTPARFSGSLLGGKAA | 26 |
| | | LTLSGVQPEDEAEYYCALWYSQLWVFGGGTKLTVL | |
| | CD3_VH- | EVQLLESGGGLVQPGGSLKLSCAASGFTFNTYAMNWV | SEQ ID NO: |
| | 8 | RKAPGKGLEWVARIRSKYNNYATYYADSVKDRFTISR | 20 |
| | | DDSKNTAYLQMNNLKTEDTAVYYCVRHGTFGNSYVSW | |
| | | FAYWGQGTLVTVSS | |
| CD3- | CD3_VL- | ELVVTQEPSLTVSPGGTVTLTCRSSTGAVTTSNYANW | SEQ ID NO: |
| A11 | 1 | VQDKPGQAPRGLIGGTNKRAPGTPARFSGSLLGGKAA | 22 |
| | | LTLSGVQPEDEAEYYCALWYSNLWVFGGGTKLTVL | |
| | CD3_VH- | EVQLLESGGGLVQPGGSLKLSCAASGFTFNTYAMDWV | SEQ ID NO: |
| | 9 | RKAPGKGLEWVARIRSKYNNYATYYADSVKDRFTISR | 21 |
| | | DDSKNTAYLQMNNLKTEDTAVYYCVRHGNFGNSYVSW | |
| | | FAYWGQGTLVTVSS | |
| CD3- | CD3_VL- | ELVVTQEPSLTVSPGGTVTLTCRSSTGAVTTSNYANW | SEQ ID NO: |
| A12 | 3 | VQEKPGQAPRGLIGGTNKRAPGTPARFSGSLLGGKAA | 24 |
| | | LTLSGVQPEDEAEYYCALWYSNLWVFGGGTKLTVL | |
| | CD3_VH- | EVQLLESGGGLVQPGGSLKLSCAASGFTFNTYAMNWV | SEQ ID NO: |
| | 1 | RKAPGKGLEWVARIRSKYNNYATYYADSVKDRFTISR | 13 |
| | | DDSKNTAYLQMNNLKTEDTAVYYCVRHGNFGNSYVSW | |
| | | FAYWGQGTLVTVSS | |
| CD3- | CD3_VL- | ELVVTQEPSLTVSPGGTVTLTCRSSTGAVTTSNYANW | SEQ ID NO: |
| A13 | 7 | VQDKPGQAPRGLIGGTNKRAPGTPARFSGSLLGGKAA | 28 |
| | | LTLSGVQPEDEAEYYCALWYSDLWVFGGGTKLTVL | |
| | CD3_VH- | EVQLLESGGGLVQPGGSLKLSCAASGFTFNTYAMNWV | SEQ ID NO: |
| | 4 | RKAPGKGLEWVARIRSKYNNYATYYADSVKDRFTISR | 16 |
| | | DDSKNTAYLQMNNLKTEDTAVYYCVRHGNFGDDYVSW | |
| | | FAYWGQGTLVTVSS | |

TABLE 9.2
Anti-CD3 Heavy Chain CDRs

| | CDRH1 | CDRH2 | CDRH3 |
|---|---|---|---|
| CD3-01 | TYAMN (SEQ ID NO: | RIRSKYNNYATYYADSVKD | HGNFGNSYVSWFAY |
| (SP34) | 29) (Kabat) | (SEQ ID NO: 34) | (SEQ ID NO: |
| | GFTFNTY (SEQ ID | (Kabat) | 37) |
| | NO: 32) (Chothia) | RSKYNNYA (SEQ ID NO: | |
| | | 36) (Chothia) | |
| CD3-A1 | TYAMN (SEQ ID NO: | RIRSKYNNYATYYADSVKD | HGNFGNSYVSWFAY |
| | 29) (Kabat) | (SEQ ID NO: 34) | (SEQ ID NO: |
| | GFTENTY (SEQ ID | (Kabat) | 37) |
| | NO: 32) (Chothia) | RSKYNNYA (SEQ ID NO: | |
| | | 36) (Chothia) | |
| CD3-A2 | TYAMN (SEQ ID NO: | RIRSKYNNYATYYADSVKD | HGNFGNSYVSWFAY |
| | 29) (Kabat) | (SEQ ID NO: 34) | (SEQ ID NO: |
| | GFTENTY (SEQ ID | (Kabat) | 37) |
| | NO: 32) (Chothia) | RSKYNNYA (SEQ ID NO: | |
| | | 36) (Chothia) | |
| CD3-A3 | TYAMN (SEQ ID NO: | RIRSKYNNYATYYADSVKD | HGNFGNSYVSWFAY |
| | 29) (Kabat) | (SEQ ID NO: 34) | (SEQ ID NO: |
| | GFTFNTY (SEQ ID | (Kabat) | 37) |
| | NO: 32) (Chothia) | RSKYNNYA (SEQ ID NO: | |
| | | 36) (Chothia) | |
| CD3-A4 | TYAMN (SEQ ID NO: | RIRSKYNNYATYYADSVKD | HGNFGDSYVSWFAY |
| | 29) (Kabat) | (SEQ ID NO: 34) | (SEQ ID NO: |
| | GFTFNTY (SEQ ID | (Kabat) | 38) |
| | NO: 32) (Chothia) | RSKYNNYA (SEQ ID NO: | |
| | | 36) (Chothia) | |
| CD3-A5 | TYAMN (SEQ ID NO: | RIRSKYNNYATYYADSVKD | HGNFGDDYVSWFAY |
| | 29) (Kabat) | (SEQ ID NO: 34) | (SEQ ID NO: |
| | GFTFNTY (SEQ ID | (Kabat) | 39) |
| | NO: 32) (Chothia) | RSKYNNYA (SEQ ID NO: | |
| | | 36) (Chothia) | |
| CD3-A6 | DYAMN (SEQ ID NO: | RIRSKYNNYATYYADSVKD | HGNFGNSYVSWFAY |
| | 30) (Kabat) | (SEQ ID NO: 34) | (SEQ ID NO: |
| | GFTFNDY (SEQ ID | (Kabat) | 37) |
| | NO: 33) (Chothia) | RSKYNNYA (SEQ ID NO: | |
| | | 36) (Chothia) | |
| CD3-A7 | TYAMN (SEQ ID NO: | KIRSKYNNYATYYADSVKD | HGNFGDSYVSWFAY |
| | 29) (Kabat) | (SEQ ID NO: 35) | (SEQ ID NO: |
| | GFTFNTY (SEQ ID | (Kabat) | 38) |
| | NO: 32) (Chothia) | RSKYNNYA (SEQ ID NO: | |
| | | 36) (Chothia) | |
| CD3-A8 | TYAMN (SEQ ID NO: | KIRSKYNNYATYYADSVKD | HGNFGDSYVSWFAY |
| | 29) (Kabat) | (SEQ ID NO: 35) | (SEQ ID NO: |
| | GFTFNTY (SEQ ID | (Kabat) | 38) |
| | NO: 32) (Chothia) | RSKYNNYA (SEQ ID NO: | |
| | | 36) (Chothia) | |
| CD3-A9 | TYAMN (SEQ ID NO: | RIRSKYNNYATYYADSVKD | HGSFGNSYVSWFAY |
| | 29) (Kabat) | (SEQ ID NO: 34) | (SEQ ID NO: |
| | GFTFNTY (SEQ ID | (Kabat) | 40) |
| | NO: 32) (Chothia) | RSKYNNYA (SEQ ID NO: | |
| | | 36) (Chothia) | |
| CD3- | TYAMN (SEQ ID NO: | RIRSKYNNYATYYADSVKD | HGTFGNSYVSWFAY |
| A10 | 29) (Kabat) | (SEQ ID NO: 34) | (SEQ ID NO: |
| | GFTFNTY (SEQ ID | (Kabat) | 41) |
| | NO: 32) (Chothia) | RSKYNNYA (SEQ ID NO: | |
| | | 36) (Chothia) | |
| CD3- | TYAMD (SEQ ID NO: | RIRSKYNNYATYYADSVKD | HGNFGNSYVSWFAY |
| A11 | 31) (Kabat) | (SEQ ID NO: 34) | (SEQ ID NO: |
| | GFTFNTY (SEQ ID | (Kabat) | 37) |
| | NO: 32) (Chothia) | RSKYNNYA (SEQ ID NO: | |
| | | 36) (Chothia) | |
| CD3- | TYAMN (SEQ ID NO: | RIRSKYNNYATYYADSVKD | HGNFGNSYVSWFAY |
| A12 | 29) (Kabat) | (SEQ ID NO: 34) | (SEQ ID NO: |
| | GFTFNTY (SEQ ID | (Kabat) | 37) |
| | NO: 32) (Chothia) | RSKYNNYA (SEQ ID NO: | |
| | | 36) (Chothia) | |
| CD3- | TYAMN (SEQ ID NO: | RIRSKYNNYATYYADSVKD | HGNFGDDYVSWFAY |
| A13 | 29) (Kabat) | (SEQ ID NO: 34) | (SEQ ID NO: |
| | GFTFNTY (SEQ ID | (Kabat) | 39) |
| | NO: 32) (Chothia) | RSKYNNYA (SEQ ID NO: | |
| | | 36) (Chothia) | |

TABLE 9.3
Anti-CD3 Light Chain CDRs

| | CDRL1 | CDRL2 | CDRL3 |
|---|---|---|---|
| CD3-01 | RSSTGAVTTSNYAN | GTNKRAP (SEQ ID | ALWYSNLWV (SEQ ID |
| (SP34) | (SEQ ID NO: 42) | NO: 43) | NO: 45) |
| CD3-A1 | RSSTGAVTTSNYAN | GTNKRAP (SEQ ID | ALWDSNLWV (SEQ ID |
| | (SEQ ID NO: 42) | NO: 43) | NO: 46) |
| CD3-A2 | RSSTGAVTTSNYAN | DTNKRAP (SEQ ID | ALWYSNLWV (SEQ ID |
| | (SEQ ID NO: 42) | NO: 44) | NO: 45) |
| CD3-A3 | RSSTGAVTTSNYAN | GTNKRAP (SEQ ID | ALWYSNLWV (SEQ ID |
| | (SEQ ID NO: 42) | NO: 43) | NO: 45) |
| CD3-A4 | RSSTGAVTTSNYAN | GTNKRAP (SEQ ID | ALWYSQLWV (SEQ ID |
| | (SEQ ID NO: 42) | NO: 43) | NO: 47) |
| CD3-A5 | RSSTGAVTTSNYAN | GTNKRAP (SEQ ID | ALWYSQLWV (SEQ ID |
| | (SEQ ID NO: 42) | NO: 43) | NO: 47) |
| CD3-A6 | RSSTGAVTTSNYAN | GTNKRAP (SEQ ID | ALWYSNLWV (SEQ ID |
| | (SEQ ID NO: 42) | NO: 43) | NO: 45) |
| CD3-A7 | RSSTGAVTTSNYAN | GTNKRAP (SEQ ID | ALWYSNLWV (SEQ ID |
| | (SEQ ID NO: 42) | NO: 43) | NO: 45) |
| CD3-A8 | RSSTGAVTTSNYAN | GTNKRAP (SEQ ID | ALWYSQLWV (SEQ ID |
| | (SEQ ID NO: 42) | NO: 43) | NO: 47) |
| CD3-A9 | RSSTGAVTTSNYAN | GTNKRAP (SEQ ID | ALWYSQLWV (SEQ ID |
| | (SEQ ID NO: 42) | NO: 43) | NO: 47) |
| CD3-A10 | RSSTGAVTTSNYAN | GTNKRAP (SEQ ID | ALWYSQLWV (SEQ ID |
| | (SEQ ID NO: 42) | NO: 43) | NO: 47) |
| CD3-A11 | RSSTGAVTTSNYAN | GTNKRAP (SEQ ID | ALWYSNLWV (SEQ ID |
| | (SEQ ID NO: 42) | NO: 43) | NO: 45) |
| CD3-A12 | RSSTGAVTTSNYAN | GTNKRAP (SEQ ID | ALWYSNLWV (SEQ ID |
| | (SEQ ID NO: 42) | NO: 43) | NO: 45) |
| CD3-A13 | RSSTGAVTTSNYAN | GTNKRAP (SEQ ID | ALWYSDLWV (SEQ ID |
| | (SEQ ID NO: 42) | NO: 43) | NO: 48) |

Provided herein are bispecific antibodies comprising a first antigen binding domain that binds a first antigen (e.g. CD3) and a second antigen binding domain that binds to a second antigen (e.g. CD20). In some embodiments the bispecific antibody has the following structure: a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_{H1}), a hinge region (H21H), a constant region 2 domain (CH2_{H2}) and a constant region 3 domain (CH2_{H3}) and second light chain polypeptide (L2) comprising a variable region (VL2) and a constant region (CL2).

In some embodiments, the bispecific antibody of the disclosure comprises a first antigen binding domain (e.g. binding to CD3) comprising any one of the VH1 and VL1 sequences listed in Table 9.1. In Table 9.1, the underlined sequences are CDR sequence according to Kabat and the bolded sequences are CDR sequences according to Chothia.

In some embodiments, the bispecific antibody of the disclosure comprises a first antigen binding domain (e.g. binding to CD3ε) comprising: a) a heavy chain variable region (VH1) comprising a VH complementarity determining region 1 (VH1_{CDR1}), a VH complementarity determining region 2 (VH1_{CDR2}) and a VH complementarity determining region 3 (VH1_{CDR3}); and b) a light chain variable region (VL) comprising a VL complementarity determining region 1 (VL1_{CDR1}), a VL complementarity determining region 2 (VL1_{CDR2}) and a VL complementarity determining region 3 (VL1_{CDR3}). Tables 9.2 and 9.3 provide exemplary of CDR sequences of the anti-CD3 antibodies provided herein.

In some embodiments, the bispecific antibody comprises any one of the anti-CD3 antibodies of the disclosure. Exemplary anti-CD3 antibodies of the invention include CD3-A1, CD3-A2, CD3-A3, CD3-A4, CD3-A5, CD3-A6, CD3-A7, CD3-A8, CD3-A9, CD3-A10, CD3-A11, CD3-A12 and CD3-A13.

In some embodiments, the binding affinity (Kr) of the first antigen binding region of the bispecific antibody that binds to CD3 (e.g., human CD3ε (e.g., (SEQ ID NO: 419) or cynomolgus CD3ε (e.g., (SEQ ID NO: 422)) is about 0.001 nM to about 5000 nM. In some embodiments, the binding affinity to CD3 is about 0.001 nM to about 0.01 nM, about 0.01 to about 0.1 nM or about 0.1 to about 1 nM. In some embodiments, the binding affinity is about 1 nM to about 1000 nM, about 10 nM to about 1000 nM or about 100 nM to about 1000 nM.

In some embodiments, the binding affinity is about 50 nM to about 5000 nM, about 50 nM to about 4000 nM, about 50 nM to about 3000 nM, about 50 nM to about 2000 nM, about 50 nM to about 1000 nM, about 50 nM to about 900 nM, about 50 nM to about 800 nM, about 50 nM to about 700 nM, about 50 nM to about 600 nM, about 50 nM to about 500 nM, about 50 nM to about 400 nM, about 50 nM to about 300 nM, about 50 nM to about 200 nM, about 50 nM to about 100 nM or about 50 nM to about 500 nM. In some embodiments, the binding affinity is about 50 nM to about 200 nM.

In some embodiments, the binding affinity is about 10 nM to about 20 nM, about 20 nM to about 30 nM, about 30 nM to about 40 nM, about 50 nM to about 60 nM, about 60 nM to about 70 nM, about 70 nM to about 80 nM, about 80 nM to about 90 nM, about 90 nM to about 100 nM, about 100 nM to about 110 nM, about 10 nM to about 120 nM, about 120 nM to about 130 nM, about 130 nM to about 140 nM, about 150 nM to about 160 nM, about 160 nM to about 170 nM, about 170 nM to about 180 nM, about 180 nM to about 190 nM, or about 190 nM to about 200 nM.

In some embodiments, the binding affinity is less than about 5000 nM, 4000 nM, 3000 nM, 2000 nM, 1000 nM, 900 nM, 800 nM, 700 nM, 600 nM, 500 nM, 400 nM, 300 nM, 275 nM, 250 nM, 225 nM, 200 nM, 175 nM, 150 nM, 125 nM, 100 nM, 90 nM, 80 nM, 70 nM, 60 nM, 50 nM, 40 nM, 30 nM, 20 nM, 10 nM, 9.5 nM, 9 nM, 8.5 nM, 8 nM, 7.5 nM, 7 nM, 6.5 nM, 6 nM, 5 nM, 4.5 nM, 4 nM, 3.5 nM, 3 nM, 2.5 nM, 2 nM, 1.5 nM, 1 nM, or 0.5 nM.

### Disease Associated Antigens

Provided herein is a bispecific antibody comprises a first antigen binding region that binds to a target antigen on the modified T cell (e.g., CD3, a CAR domain such as FMC63, a CAR hinge, or a T cell antigen) and a second antigen binding region that binds to a second target antigen on the tumor cell (e.g., a cell surface antigen, or DAA, such as CD20, CD180, BMCA or CD22). In some embodiments, the bispecific antibody further comprises a polypeptide fused to the N-terminus or the C-terminus of the bispecific antibody. In some embodiments, the polypeptide fusion comprises a CD58 or a fragment thereof. In some embodiments, the bispecific antibody comprises a third antigen binding region that binds a third target antigen (e.g., a third biological molecule, e.g., a cell surface antigen, e.g., a disease associated antigen).

In some embodiments, the third target antigen is the same as the second target antigen. In some embodiments, the third antigen is different than the first target antigen or the second target antigen.

In some embodiments, the second target antigen is a cell surface antigen, and the third target antigen is a cell surface antigen. In some embodiments, the second target antigen is a disease associated antigen. In some embodiments, the third target antigen is a disease associated antigen. In some embodiments, the second target antigen and the third antigen are a disease associated antigens.

Disease associated antigens include but are not limited to those described in PCT Publication No. WO 2016/204966, WO 2019/104075 and WO 2023/178357 each of which are incorporated by reference herein in their entirety. Disease associated antigens include but are not limited to ACVR1, ADAM21, AGL10, ALPPL2, APCDD1, ASPRV1, BCMA, BMPR1B, CD151, CD19, CD20, CD22, CD274, CD276, CD33, CD38, CD47, CD6, CD70, CD74, CD79A, CD79B, CD84, CD180, CDCP1, CDH17, CDH3, CDHR2, CDHR5, CEACAM5, CEACAM6, CEACAM7, CELSR1, CLCA2, CLDN1, CLDN18, CLDN6, CNGB1, CNGB3, COL11A1, COL17A1, CRB1, CPSG4, CTAG2, CTAGE4, CXADR, CXCR4, DCBLD2, DCST1, DLL3, DLL4, DPCR1, DSG3, DSG4, DUOX2, EBI3, EFNA4, EGFR, ENTPD1, ENTPD2, EPCAM, EPHA10, EPHA6, EPHA8, EPHB3, EPS8L1, ERBB2, ERMP1, F11R, FAP, FAT1, FCER2, FCRL3, FER1L6, FGFR2, FLT3, FLVCR1, FN1, FXYD3, GABRA3, GGT2, GGT3P, GJB3, GLG1, GPC1, GPC2, GPNMB, GPC5A, GRIND2D, GUCY2C, HAVCR2, HEPHL1, HHLA1, IGSF3, IGSF9, IL2RB, IL3RA, ITGA2, ITGA6, ITGAV, ITGB4, ITGB6, LCN15, LILRB4, LNPEP, LRFN4, LRRC15, LY6D, LY75, MAL2, MET, MFI2, MICA, MICB, MMP13, MMP14, MPZL2, MS4A1, MSLN, MST1R, MTDH, MUC1, MUC13, MUC16, MUC17, NAALADL2, NCSTN, NIPAL4, NLGN1, NOTCH3, NOX1, OC90, OR10Q1, OR511, PAEP, PANX3, PCDH15, PDCHA9, PCDHB12, PCDHB2, PKD1L1, PODXL, POLR2J2, PROM1, PSMA, PTK7, PVR, PVRL1, PVRL4, RAET1E, RAET1G, RAET1L, ROR1, ROR2, SDC1, SDC4, SDK2, SHISA8, SIGLEC7, SIT1, SLAMF1, SLAMF6, SLAMF7, SLC11A2, SLC12A2, SLC15A1, SLC1A5, SLC22A25, SLC2A9, SLC34A2, SLC38A2, SLC39A4, SLC6A14, SLC7A11, SLC7A3, SLC7A5, SYT8, TAS2R5, TMEM132A, TMPRSS3, TMPRSS4, TMX1, TNFRSF17, TNFRSF21, TNFRSF9, TNFRSF11, TNFRSF15, TNFRSF4, TNFRSF9, TNMD, TP53111, TPBG, TRPC5, TRPV2, TSPAN10, TSPAN8, UGT2A1, UGT3A2, ULBP1, ULBP2, ULBP3, UMODL1, UPK1B, VANGL1, VANGL2, VASN, VMP1, VSIG4, VTCN1, WNT16, YIF1B, and ZNRF4. In some embodiments, the disease associated antigen is CD19, CD20, CD180, BCMA, CD79A, CD79B or CD22. In some embodiments, the disease associated antigen is CD19. In some embodiments, the disease associated antigen is CD20. In some embodiments, the disease associated antigen is CD180. In some embodiments, the disease associated antigen is BCMA. In some embodiments, the disease associated antigen is CD79A. In some embodiments, the disease associated antigen is CD79B. In some embodiments, the disease associated antigen is CD22.

In some embodiments, wherein the bispecific antibodies comprise a first antigen binding domain that binds a target antigen on a modified T cell, a second antigen binding domain that binds to a target antigen on a tumor cell, and a third antigen binding domain that binds to a target antigen on a tumor cell, wherein the second antigen binding domain and the third antigen binding domain bind to a cell surface antigen on the same cell.

In some embodiments, wherein the bispecific antibodies comprise a first antigen binding domain that binds a target antigen on a modified T cell, a second antigen binding domain that binds to a target antigen on a tumor cell, and a third antigen binding domain that binds to a target antigen on a tumor cell, wherein the second antigen binding domain and the third antigen binding domain bind to a cell surface antigen on different cells.

Multispecific antibodies are antibodies (e.g., monoclonal antibodies) that have binding specificities for at least two different sites. In some embodiments, the anti-CD3 CD3ε antibody provided herein is a multispecific antibody (e.g., a bispecific antibody).

In certain embodiments, bispecific antibodies may bind to two different epitopes of CD3 (e.g., CD3ε or CD3γ). In certain embodiments, one of the binding specificities is for CD3 (e.g., CD3ε or CD3γ) and the other is for any other antigen (e.g., a second biological molecule, e.g., a cell surface antigen, e.g., a disease associated antigen). In some embodiments, the cell surface antigen is CD19. In some embodiments, the cell surface antigen is CD20. In some embodiments, the cell surface antigen is CD180. In some embodiments, the cell surface antigen is BCMA. In some embodiments, the cell surface antigen is CD79A. In some embodiments, the cell surface antigen is CD79B. In some embodiments, the cell surface antigen is CD22.

In some embodiments, the bispecific antibodies of the present disclosure have a first binding epitope that is for CD3, and a second binding epitope that is for any other antigen (e.g., a second biological molecule, e.g., a cell surface antigen, e.g., a disease associated antigen), and a third binding epitope that is for any other antigen (e.g., a third biological molecule, e.g., a cell surface antigen, e.g., a disease associated antigen).

In some embodiments, the bispecific antibody comprises a first antigen binding region that binds CD3, and a second antigen binding region that binds CD20. In some embodiments, the bispecific antibody comprises a first antigen binding region that binds CD3, and a second antigen binding region that binds CD19. In some embodiments, the bispecific antibody comprises a first antigen binding region that binds CD3, and a second antigen binding region that binds CD180. In some embodiments, the bispecific antibody comprises a first antigen binding region that binds CD3, and a second antigen binding region that binds BCMA. In some embodiments, the bispecific antibody comprises a first antigen binding region that binds CD3, and a second antigen binding region that binds CD79A. In some embodiments, the bispecific antibody comprises a first antigen binding region that binds CD3, and a second antigen binding region that binds CD79B. In some embodiments, the bispecific antibody comprises a first antigen binding region that binds CD3, and a second antigen binding region that binds CD22.

In some embodiments, the bispecific antibody comprises a first antigen binding region that binds a CAR domain expressed on a CAR T cell, and a second antigen binding region that binds CD20. In some embodiments, the bispecific antibody comprises a first antigen binding region that binds a CAR domain expressed on a CAR T cell, and a second antigen binding region that binds CD19. In some embodiments, the bispecific antibody comprises a first antigen binding region that binds a CAR domain expressed on a CAR T cell, and a second antigen binding region that binds CD180. In some embodiments, the bispecific antibody comprises a first antigen binding region that binds a CAR domain expressed on a CAR T cell, and a second antigen binding region that binds BCMA. In some embodiments, the bispecific antibody comprises a first antigen binding region that binds a CAR domain expressed on a CAR T cell, and a second antigen binding region that binds CD79A. In some embodiments, the bispecific antibody comprises a first antigen binding region that binds a CAR domain expressed on a CAR T cell, and a second antigen binding region that binds CD79B. In some embodiments, the bispecific antibody comprises a first antigen binding region that binds a CAR domain expressed on a CAR T cell, and a second antigen binding region that binds CD22.

In some embodiments, the bispecific antibody comprises a first antigen binding region that binds a scFv region of a CAR domain expressed on a CAR T cell, and a second antigen binding region that binds CD20. In some embodiments, the bispecific antibody comprises a first antigen binding region that binds a scFv region of a CAR domain expressed on a CAR T cell, and a second antigen binding region that binds CD180. In some embodiments, the bispecific antibody comprises a first antigen binding region that binds a scFv region of a CAR domain expressed on a CAR T cell, and a second antigen binding region that binds BCMA. In some embodiments, the bispecific antibody comprises a first antigen binding region that binds a scFv region of a CAR domain expressed on a CAR T cell, and a second antigen binding region that binds CD79A. In some embodiments, the bispecific antibody comprises a first antigen binding region that binds a scFv region of a CAR domain expressed on a CAR T cell, and a second antigen binding region that binds CD79B. In some embodiments, the bispecific antibody comprises a first antigen binding region that binds a scFv region of a CAR domain expressed on a CAR T cell, and a second antigen binding region that binds CD22. In some embodiments, the scFv region of a CAR domain expressed on a CAR T cell is FMC63.

In some embodiments, the bispecific antibody comprises a first antigen binding region that binds a linker region of a CAR domain expressed on a CAR T cell, and a second antigen binding region that binds CD20. In some embodiments, the bispecific antibody comprises a first antigen binding region that binds a linker region of a CAR domain expressed on a CAR T cell, and a second antigen binding region that binds CD180. In some embodiments, the bispecific antibody comprises a first antigen binding region that binds a linker region of a CAR domain expressed on a CAR T cell, and a second antigen binding region that binds BCMA. In some embodiments, the bispecific antibody comprises a first antigen binding region that binds a linker region of a CAR domain expressed on a CAR T cell, and a second antigen binding region that binds CD79A. In some embodiments, the bispecific antibody comprises a first antigen binding region that binds a linker region of a CAR domain expressed on a CAR T cell, and a second antigen binding region that binds CD79B. In some embodiments, the bispecific antibody comprises a first antigen binding region that binds a linker region of a CAR domain expressed on a CAR T cell, and a second antigen binding region that binds CD22.

In some embodiments, the bispecific antibody comprises a first antigen binding region that binds CD3, and a second antigen binding region that binds CD20 and a third antigen binding region that binds a T cell surface antigen. In some embodiments, the bispecific antibody comprises a first antigen binding region that binds CD3, and a second antigen binding region that binds CD19 and a third antigen binding region that binds a T cell surface antigen. In some embodiments, the bispecific antibody comprises a first antigen binding region that binds CD3, and a second antigen binding region that binds CD180 and a third antigen binding region that binds a T cell surface antigen. In some embodiments, the bispecific antibody comprises a first antigen binding region that binds CD3, and a second antigen binding region that binds BCMA and a third antigen binding region that binds a T cell surface antigen. In some embodiments, the bispecific antibody comprises a first antigen binding region that binds CD3, and a second antigen binding region that binds CD79A and a third antigen binding region that binds a T cell surface antigen. In some embodiments, the bispecific antibody comprises a first antigen binding region that binds CD3, and a second antigen binding region that binds CD79B and a third antigen binding region that binds a T cell surface antigen. In some embodiments, the bispecific antibody comprises a first antigen binding region that binds CD3, and a second antigen binding region that binds CD22 and a third antigen binding region that binds a T cell surface antigen.

In some embodiments, the bispecific antibody comprises a first antigen binding region that binds CD3, and a second antigen binding region that binds CD20 and a third antigen binding region that binds a CAR domain expressed on a CAR T cell. In some embodiments, the bispecific antibody comprises a first antigen binding region that binds CD3, and a second antigen binding region that binds CD19 and a third antigen binding region that binds a CAR domain expressed on a CAR T cell. In some embodiments, the bispecific antibody comprises a first antigen binding region that binds CD3, and a second antigen binding region that binds CD180 and a third antigen binding region that binds a CAR domain expressed on a CAR T cell. In some embodiments, the bispecific antibody comprises a first antigen binding region that binds CD3, and a second antigen binding region that binds BCMA and a third antigen binding region that binds a CAR domain expressed on a CAR T cell. In some embodiments, the bispecific antibody comprises a first antigen binding region that binds CD3, and a second antigen binding region that binds CD79A and a third antigen binding region that binds a CAR domain expressed on a CAR T cell. In some embodiments, the bispecific antibody comprises a first antigen binding region that binds CD3, and a second antigen binding region that binds CD79B and a third antigen binding region that binds a CAR domain expressed on a CAR T cell. In some embodiments, the bispecific antibody comprises a first antigen binding region that binds CD3, and a second antigen binding region that binds CD22 and a third antigen binding region that binds a CAR domain expressed on a CAR T cell.

In some embodiments, the bispecific antibody comprises a first antigen binding region that binds CD3, and a second antigen binding region that binds CD20 and a third antigen binding region that binds a disease associated antigen. In some embodiments, the bispecific antibody comprises a first antigen binding region that binds CD3, and a second antigen binding region that binds CD19 and a third antigen binding region that binds a disease associated antigen. In some embodiments, the bispecific antibody comprises a first antigen binding region that binds CD3, and a second antigen binding region that binds CD180 and a third antigen binding region that binds a disease associated antigen. In some embodiments, the bispecific antibody comprises a first antigen binding region that binds CD3, and a second antigen binding region that binds BCMA and a third antigen binding region that binds a disease associated antigen. In some embodiments, the bispecific antibody comprises a first antigen binding region that binds CD3, and a second antigen binding region that binds CD79A and a third antigen binding region that binds a disease associated antigen. In some embodiments, the bispecific antibody comprises a first antigen binding region that binds CD3, and a second antigen binding region that binds CD79B and a third antigen binding region that binds a disease associated antigen. In some embodiments, the bispecific antibody comprises a first antigen binding region that binds CD3, and a second antigen binding region that binds CD22 and a third antigen binding region that binds a disease associated antigen.

In some embodiments, the bispecific antibody comprises a first antigen binding region that binds CD3, and a second antigen binding region that binds CD20 and a third antigen binding region that binds a tumor associated antigen. In some embodiments, the bispecific antibody comprises a first antigen binding region that binds CD3, and a second antigen binding region that binds CD19 and a third antigen binding region that binds a tumor associated antigen. In some embodiments, the bispecific antibody comprises a first antigen binding region that binds CD3, and a second antigen binding region that binds CD180 and a third antigen binding region that binds a tumor associated antigen. In some embodiments, the bispecific antibody comprises a first antigen binding region that binds CD3, and a second antigen binding region that binds BCMA and a third antigen binding region that binds a tumor associated antigen. In some embodiments, the bispecific antibody comprises a first antigen binding region that binds CD3, and a second antigen binding region that binds CD79A and a third antigen binding region that binds a tumor associated antigen. In some embodiments, the bispecific antibody comprises a first antigen binding region that binds CD3, and a second antigen binding region that binds CD79B and a third antigen binding region that binds a tumor associated antigen. In some embodiments, the bispecific antibody comprises a first antigen binding region that binds CD3, and a second antigen binding region that binds CD22 and a third antigen binding region that binds a tumor associated antigen.

In some embodiments, the bispecific antibody comprises a first antigen binding region that binds CD3, and a second antigen binding region that binds CD20 and a third antigen binding region that binds a hematologic tumor antigen. In some embodiments, the bispecific antibody comprises a first antigen binding region that binds CD3, and a second antigen binding region that binds CD19 and a third antigen binding region that binds a hematologic tumor antigen. In some embodiments, the bispecific antibody comprises a first antigen binding region that binds CD3, and a second antigen binding region that binds CD180 and a third antigen binding region that binds a hematologic tumor antigen. In some embodiments, the bispecific antibody comprises a first antigen binding region that binds CD3, and a second antigen binding region that binds BCMA and a third antigen binding region that binds a hematologic tumor antigen. In some embodiments, the bispecific antibody comprises a first antigen binding region that binds CD3, and a second antigen binding region that binds CD79A and a third antigen binding region that binds a hematologic tumor antigen. In some embodiments, the bispecific antibody comprises a first antigen binding region that binds CD3, and a second antigen binding region that binds CD79B and a third antigen binding region that binds a hematologic tumor antigen. In some embodiments, the bispecific antibody comprises a first antigen binding region that binds CD3, and a second antigen binding region that binds CD22 and a third antigen binding region that binds a hematologic tumor antigen.

In some embodiments, the bispecific antibody comprises a first antigen binding region that binds CD3, and a second antigen binding region that binds CD20 and a third antigen binding region that binds a solid tumor antigen. In some embodiments, the bispecific antibody comprises a first antigen binding region that binds CD3, and a second antigen binding region that binds CD19 and a third antigen binding region that binds a solid tumor antigen. In some embodiments, the bispecific antibody comprises a first antigen binding region that binds CD3, and a second antigen binding region that binds CD180 and a third antigen binding region that binds a solid tumor antigen. In some embodiments, the bispecific antibody comprises a first antigen binding region that binds CD3, and a second antigen binding region that binds BCMA and a third antigen binding region that binds a solid tumor antigen. In some embodiments, the bispecific antibody comprises a first antigen binding region that binds CD3, and a second antigen binding region that binds CD79A and a third antigen binding region that binds a solid tumor antigen. In some embodiments, the bispecific antibody comprises a first antigen binding region that binds CD3, and a second antigen binding region that binds CD79B and a third antigen binding region that binds a solid tumor antigen. In some embodiments, the bispecific antibody comprises a first antigen binding region that binds CD3, and a second antigen binding region that binds CD22 and a third antigen binding region that binds a solid tumor antigen.

In some embodiments, the bispecific antibodies of the present disclosure have a first binding epitope that is for a CAR domain expressed on a CAR-T cell, and a second binding epitope that is for any other antigen (e.g., a second biological molecule, e.g., a cell surface antigen, e.g., a disease associated antigen), and a third binding epitope that is for any other antigen (e.g., a third biological molecule, e.g., a cell surface antigen, e.g., a disease associated antigen).

In some embodiments, the bispecific antibodies of the present disclosure have a first binding epitope that is for a CAR binding domain, and a second binding epitope that is for any other antigen (e.g., a second biological molecule, e.g., a cell surface antigen, e.g., a disease associated antigen), and a third binding epitope that is for any other antigen (e.g., a third biological molecule, e.g., a cell surface antigen, e.g., a disease associated antigen).

In some embodiments, the bispecific antibodies of the present disclosure have a first binding epitope that is for a CAR hinge domain, and a second binding epitope that is for any other antigen (e.g., a second biological molecule, e.g., a cell surface antigen, e.g., a disease associated antigen), and a third binding epitope that is for any other antigen (e.g., a third biological molecule, e.g., a cell surface antigen, e.g., a disease associated antigen).

In some instances, the cell surface antigen (e.g. disease associated antigen) may be expressed in low copy number on the target cell (e.g. tumor cell). For example, in some instances, the cell surface antigen is expressed or present at less than 35,000 copies per target cell. In some embodiments, the low copy number cell surface antigen is present between 100 and 35,000 copies per target cell; between 100 and 30,000 copies per target cell; between 100 and 25,000 copies per target cell; between 100 and 20,000 copies per target cell; between 100 and 15,000 copies per target cell; between 100 and 10,000 copies per target cell; between 100 and 5,000 copies per target cell; between 100 and 2,000 copies per target cell; between 100 and 1,000 copies per target cell; or between 100 and 500 copies per target cell. Copy number of the cell surface antigen can be determined, for example, using a standard Scratchcard plot.

### CD20 Antigen

“CD20” as used herein refers to the human B-lymphocyte antigen CD20 (also known as CD20, human B-lymphocyte-restricted differentiation antigen, B-lymphocyte surface antigen B1, Leu-16, Bp35, BMS, and LFS; the sequence is characterized by the SwissProt database entry P11836), which is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder. T. F., et al, Proc. Natl. Acad. Sci. U.S.A. 85 (1988) 208-12; Stamenkovic, I., et al., J. Exp. Med. 167 (1988) 1975-80: Einfeld, D. A., et al., EMBO J. 7 (1988) 711-7; Tedder, T. F., et al., J. Immunol. 142 (1989) 2560-8). CD20 is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin's lymphomas (NHL) (Anderson, K. C., et al., Blood 63(6) (1984) 1424-1433) but is not found on hematopoietic stem cells, pro-B cells, normal plasma cells, or other normal tissues (Tedder, T. F., et al., J. Immunol. 135(2)(1985) 973-979).

The 85 amino acid carboxyl-terminal region of the CD20 protein is located within the cytoplasm. The length of this region contrasts with that of other B cell-specific surface structures such as IgM, IgD, and IgG heavy chains or histocompatibility antigens class II a or B chains, which have relatively short intracytoplasmic regions of 3, 3, 28, 15, and 16 amino acids, respectively (Komaromy. M., et al., NAR 11 (1983) 6775-6785). Of the last 61 carboxyl-terminal amino acids, 21 are acidic residues, whereas only 2 are basic, indicating that this region has a strong net negative charge. The GenBank Accession No. is NP-690605. It is thought that CD20 might be involved in regulating an early step(s) in the activation and differentiation process of B cells (Tedder, T. F., et al., Eur. J. Immunol. 16 (1986) 881-887) and could function as a calcium ion channel (Tedder. T. F., et al., J. Cell. Biochem. 14D (1990) 195).

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction. ADCC and CDC.

Synonyms of CD20, as recognized in the art, include B-lymphocyte antigen CD20, B-lymphocyte surface antigen B1, Leu-16, Bp35, BMS, and LF5.

The term “anti-CD20 antibody” according to the invention is an antibody that binds specifically to CD20 antigen. Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052.

The two different types of anti-CD20 antibodies differ significantly in their mode of CD20 binding and biological activities (Cragg, M. S., et al., Blood 103 (2004) 2738-2743; and Cragg, M. S., et al., Blood 101 (2003) 1045-1052). Type I antibodies, as e.g. rituximab, are potent in complement mediated cytotoxicity, whereas type II antibodies, as e.g. Tositumomab (B1), 11B8, AT80 or humanized B-Ly1 antibodies, effectively initiate target cell death via caspase-independent apoptosis with concomitant phosphatidylserine exposure.

In some instances, CD20 may be expressed in low copy number on the target cell (e.g. tumor cell). For example, in some instances. CD20 is expressed or present at less than 35,000 copies per target cell. In some embodiments, the low copy number cell surface CD20 is present between 100 and 35,000 copies per target cell; between 100 and 30,000 copies per target cell; between 100 and 25,000 copies per target cell; between 100 and 20,000 copies per target cell, between 100 and 15,000 copies per target cell; between 100 and 10,000 copies per target cell; between 100 and 5,000 copies per target cell: between 100 and 2,000 copies per target cell; between 100 and 1,000 copies per target cell; or between 100 and 500 copies per target cell. Copy number of the cell surface CD20 can be determined, for example, using a standard Scratchcard plot.

This disclosure provides an bispecific antibody comprising a first antigen binding region that binds CD3 and a second binding region that binds CD20.

In son embodiments the bispecific antibody has the following structure: a first heavy chain polypeptide (H1) comprising a variable region (VH1), and a constant region (CH1) having a constant region 1 domain (CH1_H1), a hinge region (H1H), a constant region 2 domain (CH1_H2) and a constant region 3 domain (CH1_H3); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_H1), a hinge region (H2H), a constant region 2 domain (CH2_H2) and a constant region 3 domain (CH2_H3); and second light chain polypeptide (L2) comprising a variable region (VL2) and a constant region (CL2).

For example, the second binding region that binds to CD20 can be derived from the binding regions of an anti-CD20 antibody. Exemplary anti-CD20 antibodies include but are not limited to obinutuzumab (GA101; GAZYVA® or GAZYVARO®), tositumomab, rituximab, ofatumumab, veltuzumab, ocaratuzumab, ocrelizumab, PRO131921, ublituximab, H147 IgG3 (ECACC, hybridoma), 2C6 IgG1 (as disclosed in WO 2005/103081), 2F2 IgG1 (as disclosed in WO 2004/035607 and WO 2005/103081) and 2H7 IgG1 (as disclosed in WO 2004/056312).

In some embodiments, a second antigen binding region that binds CD20 comprises any one of the VH and VL sequences listed in Table 10.1. In Table 10.1, the underlined sequences are CDR sequence according to Kabat and the bolded sequences are CDR sequences according to Chothia.

In some embodiments, the second binding region that binds to CD20 comprises: a) a heavy chain variable region (VH) comprising a VH complementarity determining region 1 (VH_{CDR1}), a VH complementarity determining region 2 (VH_{CDR2}) and a VH complementarity determining region 3 (VH_{CDR3}); and b) a light chain variable region (VL) comprising a VL complementarity determining region 1 (VL_{CDR1}), a VL complementarity determining region 2 (VL_{CDR2}) and a VL complementarity determining region 3 (VL_{CDR3}). Tables 10.2 and 10.3 provide exemplary of CDR sequences of the anti-CD20 antibodies.

TABLE 10.1
Anti-CD20 Variable Heavy Chain and Variable Light Chain
Domains

| Ab | Amino Acid Sequence | |
|---|---|---|
| CD20_VL_ | DIVMTQTPLSLPVTPGEPASISCRSSKSLLHSNGITYLYWYLKKPGQSPQLLIYQMSN | SEQ ID |
| obinutuzumab | LVSGVPDRFSGSGSGTDFTLKISRVEAEDVGVYYCAQNLELPYTFGGGTKVEIKRTV | NO: 450 |
| CD20_VH_ | EVQLVQSGAEVKKPGSSVKVSCKASGYAFSYSWINWVRDAPGQGLEWMGRIFPGDGDT | SEQ ID |
| obinutuzumab | DYNGKFKGRVTITADKSTSTAYMELSSLRSEDTAVYYCARNVFDGYWLVYWGQGTLVT | NO: 451 |
| | VSS | |
| CD20_VL_ | EIVLTQSPATLSLSPGERATLSCRASQSVSSYLAWYQKKPGQAPRLLIYDASNRATGI | SEQ ID |
| ofatumumab | PARFSGSGSGTDFTLTISSLEPEDFAVYYCQQRSNWPITFGQGTRLEIKRTV | NO: 452 |
| CD20_VH_ | EVQLVESGGGLVQPGRSLRLSCAASGFTFNDYAMHWVRDAPGKGLEWVSTISWNSGSI | SEQ ID |
| ofatumumab | GYADSVKGRFTISRDNAKKSLYLQMNSLRAEDTALYYCAKDIQYGNYYYGMDVWGQGT | NO: 453 |
| | TVTVSS | |

TABLE 10.2
Anti-CD20 Heavy Chain CDRs

| | CDRH1 | CDRH2 | CDRH3 |
|---|---|---|---|
| CD20- | YSWIN (SEQ ID NO: | RIFPGDGDTDYNGKFKGG | NVFDGYWLVY |
| obinutuzumab | 454) (Kabat) | (SEQ ID NO: 456) | (SEQ ID NO: |
| | GYAFSYS (SEQ ID | (Kabat) | 458) |
| | NO: 455) (Chothia) | FPGDGD (SEQ ID | |
| | | NO: 457) (Chothia) | |
| CD20- | DYAMH (SEQ ID NO: | TISWNSGSIGYADSVKG | DIQYGNYYYGMDV |
| ofatumumab | 459) (Kabat) | (SEQ ID NO: 460) | (SEQ ID NO: |
| | GFTFNDY (SEQ ID | (Kabat) | 462) |
| | NO: 33) (Chothia) | SWNSGS (SEQ ID NO: | |
| | | 461) (Chothia) | |

TABLE 10.3
Anti-CD20 Light Chain CDRs

| | CDRL1 | CDRL2 | CDRL3 |
|---|---|---|---|
| CD20- | RSSKSLLHSNGITYLY | QMSNLVS (SEQ ID | AQNLELPYT |
| obinutuzumab | (SEQ ID NO: 463) | NO: 464) | (SEQ ID NO: 465) |
| CD20- | RASQSVSSYLA | DASNRAT | QQRSNWPIT |
| ofatumumab | (SEQ ID NO: 466) | (SEQ ID NO: 467) | (SEQ ID NO: 468) |

In some embodiments, the second antigen binding region comprises a) a heavy chain variable region (VH) comprising a i) a VH complementarity determining region 1 (VH_{CDR1}) comprising the amino acid sequence of SEQ ID NO: 459 or 33, ii) a VH complementarity determining region 2 (VH_{CDR2}) comprising the amino acid sequence of SEQ ID NO: 460 or 461, iii) a VH complementarity determining region 3 (VH_{CDR3}) comprising the amino acid sequence of SEQ ID NO: 462; and b) a light chain variable region (VL) comprising a i) a VL complementarity determining region 1 (VL_{CDR1}) comprising the amino acid sequence of SEQ ID NO: 466, ii) a VL complementarity determining region 2 (VL_{CDR2}) comprising the amino acid sequence of SEQ ID NO: 467, iii) a VL complementarity determining region 3 (VL_{CDR3}) comprising the amino acid sequence of SEQ ID NO: 468.

In some embodiments, the second antigen binding region comprises a VH region comprising a VH_{CDR1} comprising the amino acid sequence of SEQ ID NO: 455, a VH_{CDR2} comprising the amino acid sequence of SEQ ID NO: 457, and a VH_{CDR3} comprising the amino acid sequence of SEQ ID NO: 458; and a VL region comprising a VL_{CDR1} comprising the amino acid sequence of SEQ ID NO: 463, a VL_{CDR2} comprising the amino acid sequence of SEQ ID NO: 464, and a VL_{CDR3} comprising the amino acid sequence of SEQ ID NO: 465.

In some embodiments, the second antigen binding region comprises a VH region comprising a VH_{CDR1} comprising the amino acid sequence of SEQ ID NO: 454, a VH_{CDR2} comprising the amino acid sequence of SEQ ID NO: 456, and a VH_{CDR3} comprising the amino acid sequence of SEQ ID NO: 458; and a VL region comprising a VL_{CDR1} comprising the amino acid sequence of SEQ ID NO: 463, a VL_{CDR2} comprising the amino acid sequence of SEQ ID NO: 464, and a VL_{CDR3} comprising the amino acid sequence of SEQ ID NO: 465.

In some embodiments, the second antigen binding region comprises a VH region comprising the amino acid sequence shown in SEQ ID NO: 451 and a VL region comprising the amino acid sequence shown in SEQ ID NO: 450.

In some embodiments, the second antigen binding region comprises a VH region comprising a VH_{CDR1} comprising the amino acid sequence of SEQ ID NO: 459, a VH_{CDR2} comprising the amino acid sequence of SEQ ID NO: 460, and a VH_{CDR3} comprising the amino acid sequence of SEQ ID NO: 462; and a VL region comprising a VL_{CDR1} comprising the amino acid sequence of SEQ ID NO: 466, a VL_{CDR2} comprising the amino acid sequence of SEQ ID NO: 467, and a VL_{CDR3} comprising the amino acid sequence of SEQ ID NO: 468.

In some embodiments, the second antigen binding region comprises a VH region comprising a VH_{CDR1} comprising the amino acid sequence of SEQ ID NO: 33, a VH_{CDR2} comprising the amino acid sequence of SEQ ID NO: 461, and a VH_{CDR3} comprising the amino acid sequence of SEQ ID NO: 462; and a VL region comprising a VL_{CDR1} comprising the amino acid sequence of SEQ ID NO: 466, a VL_{CDR2} comprising the amino acid sequence of SEQ ID NO: 467, and a VL_{CDR3} comprising the amino acid sequence of SEQ ID NO: 468.

In some embodiments, the second antigen binding region comprises a VH region comprising the amino acid sequence shown in SEQ ID NO: 453 and a VL region comprising the amino acid sequence shown in SEQ ID NO: 452.

Exemplary Bispecific Antibodies that Bind to Cd3 and Cd20

Provided herein are bispecific antibodies comprising a first antigen binding domain that binds a first antigen (e.g. CD3ε) and a second antigen binding domain that binds to a CD20 In some embodiments the bispecific antibody has the following structure: a first heavy chain polypeptide (H1) comprising a variable region (VH1), and a constant region (CH1) having a constant region 1 domain (CH1_H1), a hinge region (H1H), a constant region 2 domain (CH1_H2) and a constant region 3 domain (CH1_H3); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_H1), a hinge region (H2H), a constant region 2 domain (CH2_H2) and a constant region 3 domain (CH2_H3); and second light chain polypeptide (L2) comprising a variable region (VL2) and a constant region (CL2).

In some embodiments, the bispecific antibody of the disclosure comprises a first antigen binding domain (e.g. binding to CD3ε) comprising: a) a heavy chain variable region (VH1) comprising a VH complementarity determining region 1 (VH1_CDR1), a VH complementarity determining region 2 (VH1_CDR2) and a VH complementarity determining region 3 (VH1_CDR3); and b) a light chain variable region (VL) comprising a VL complementarity determining region 1 (VL1_CDR1), a VL complementarity determining region 2 (VL1_CDR2) and a VL complementarity determining region 3 (VL1_CDR3); and a second antigen binding domain (e.g. binding to CD20) comprising: a) a heavy chain variable region (VH2) comprising a VH complementarity determining region 1 (VH2_CDR1), a VH complementarity determining region 2 (VH2_CDR2) and a VH2 complementarity determining region 3 (VH2_CDR3); and b) a light chain variable region (VL2) comprising a VL complementarity determining region 1 (VL2_CDR1), a VL complementarity determining region 2 (VL2_CDR2) and a VL complementarity determining region 3 (VL2_CDR3). Tables 9.2 and 9.3 provide exemplary of CDR sequences of the anti-CD3ε antibodies provided herein. Tables 10.2 and 10.3 provide exemplary of CDR sequences of the anti-CD20 antibodies provided herein.

In some embodiments, the bispecific antibody of the disclosure comprises a first antigen binding domain (e.g. binding to CD3ε) comprising any one of the VH1 and VL1 sequences listed in Table 9.1 and a second antigen binding domain (e.g. binding to CD20) comprising any one of the VH2 and VL2 sequences listed in Table 10.1.

In some embodiments, the bispecific antibody of the disclosure comprises a first heavy chain polypeptide (H1) and a first light chain polypeptide (L1); and a second heavy chain polypeptide (H2) and a second light chain polypeptide (L2) comprising any one of the sequence listed in Table 10.4 and Table 10.5. The italicized sequences are the heavy chain variable regions and the light chain variable regions. The underlined sequences are CDRs according to Kabat and the bolded sequences are CDRs according to Chothia.

In some embodiments, the bispecific antibody of the disclosure provided herein comprises a H1 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 16.

In some embodiments, the bispecific antibody of the disclosure provided herein comprises a L1 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 10.4 and Table 10.5.

In some embodiments, the bispecific antibody of the disclosure provided herein comprises a 112 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 16.

In some embodiments, the bispecific antibody of the disclosure provided herein comprises a L2 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 16.

In some embodiments, the H1 amino acid sequence is numbered in accordance with SEQ ID NO: 473. In some embodiments the L1 amino acid sequence is numbered in accordance with SEQ ID NO: 149. In some embodiments, the H12 amino acid sequence is numbered in accordance with SEQ ID NO: 472. In some embodiments, the L2 amino acid sequence is numbered in accordance with SEQ ID NO: 471.

TABLE 10.4
Exemplary Bispecific Antibodies that bind CD38 and CD20

| | Heavy/ | | |
|---|---|---|---|
| Antibody | Light | | SEQ ID |
| Name | Chain | Amino Acid Sequence | NO: |
| EIP0960 | CD20 | EIVLTQSPATLSLSPGERATLSCRASQSVSSYLAWYQKKPGQAPRLLIYDA | SEQ ID NO: |
| | light | SNRATGIPARFSGSGSGTDFTLTISSLEPEDFAVYYCQQRSNWPITFGQGT | 471 |
| | chain | RLEIKRTVAAPSVFIFPPSDEQLKSGTASVVCLLNNFYPREAKVQWKVDNA | |
| | (L2) | LQSGNSQESVTEQDSKQSTYSLSSTLRLSKADYEKAKVYACEVTHQGLSSP | |
| | | VTKSFNRGEC | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 472 |
| | chain | GNYYYGMDVWGQGTTVTVSSASTKGPSVFPLAPSSKSTSGGTAALGCLVDD | |
| | (H2) | YFPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYR | |
| | | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPG | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 554 |
| | chain | GNYYYGMDVWGQGTTVTVSSASTKGPSVFPLAPSSKSTSGGTAALGCLVDD | |
| | with a C- | YFPEPVTVSWNSGALTSGVHTFPAVLQSSGLYSLSSVVTVPSSSLGTQTYI | |
| | terminal | CNVNHKPSNTKVDKKVEPKSCDKTHTCPPCPAPEAAGAPSVFLFPPKPKDT | |
| | K residue | LMISRTPEVTCVVVDVSHEDPEVKFNWYVDGVEVHNAKTKPREEQYNSTYR | |
| | (H2) | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSQLWVFGG | 149 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 473 |
| | (H1) | SFGNSYVSWFAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KQTLMISRTPEVTQVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 583 |
| | with a C- | SFGNSYVSWFAYWGQGTLVTVSSASTKGPSVFPLAPSSKSTSGGTAALGCL | |
| | terminal | VKDYPPEPVTVSWNSGALTSGVHTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0963 | CD20 | EIVLTQSPATLSLSPGERATLSCRASQSVSSYLAWYQKKPGQAPRLLIYDA | SEQ ID NO: |
| | light | SNRATGIPARFSGSGSGTDFTLTISSLEPEDFAVYYCQQRSNWPITFGQGT | 471 |
| | chain | RLEIKRTVAAPSVFIFPPSDEQLKSGTASVVCLLNNFYPREAKVQWKVDNA | |
| | (L2) | LQSGNSQESVTEQDSKDSTYSLSSTLRLSKADYEKHKVYACEVTHQGLSSP | |
| | | VTKSFNRGEC | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 475 |
| | chain | GNYYYGMDVWGQGTTVTVSSASTKGPSVFPLAPSSKSTSGGTAALGCLVDD | |
| | (H2) | YFPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYR | |
| | | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPG | |
| | CD20 | EVQLVESGGGLVQPGRSARLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 555 |
| | chain | GNYYYGMDVWGQGTTVTVSSASTKGPSVFPLAPSSKSTSGGTAALGCLVDD | |
| | with a C- | YFPEPVTVSWNSGALTSGVHTFPAVLQSSGLYSLSSVVTVPSSSLGTQTYI | |
| | terminal | CNVNHKPSNTKVDKKVEPKSCDKTHTCPPCPAPEAAGAPSVFLFPPKPKDT | |
| | K residue | LMISRTPEVTCVVVDVSHEDPEVKFNWYVDGVEVHNAKTKPREEQYNSTYR | |
| | (H2) | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSQLWVFGG | 149 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVAKI | SEQ ID NO: |
| | chain | RSKYNNYATYYADSVKDRFTISRDDSKNTAYLQMNNLKTEDTAVYYCVRHG | 476 |
| | (H1) | NFGDSYVSWFAYWGQGTLVTVSSASTKGPSVFPLAPSSKSTSGGTAALGCL | |
| | | VKDYFPEPVTVSWNSGALTSGVHTSPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKESCAASGFTFNTYAMNWVRKAPGKGLEWVAKI | SEQ ID NO: |
| | chain | RSKYNNYATYYADSVKDRFTISRDDSKNTAYLQMNNLKTEDTAVYYCVRHG | 584 |
| | with a C- | NFGDSYVSWFAYWGQGTLVTVSSASTKGPSVFPLAPSSKSTSGGTAALGCL | |
| | terminal | VKDYFPEPVTVSANSGALTSGVHTSPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0961 | CD20 | EIVLTQSPATLSLSPGERATLSCRASQSVSSYLAWYQKKPGQAPRLLIYDA | SEQ ID NO: |
| | light | SNRATGIPARFSGSGSGTDFTLTISSLEPEDFAVYYCQQRSNWFITFGQGT | 471 |
| | chain | RLEIKRTVAAPSVFIFPPSDEQLKSGTASVVCLLNNFYPREAKVQWKVDNA | |
| | (L2) | LQSGNSQESVTEQDSKDSTYSLSSTLRLSKADYEKHKVYACEVTHQGLSSP | |
| | | VTKSFNRGEC | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 478 |
| | chain | GNYYYGMDVWGQGTTVTVSSASTKGPSVFPLAPSSKSTSGGTAALGQLVDD | |
| | (H2) | YFPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYR | |
| | | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPG | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 556 |
| | chain | GNYYYGMDVWGQGTTVTVSSASTKGPSVFPLAPSSKSTSGGTAALGCLVDD | |
| | with a C- | YFPEPVTVSQNSGALTSGVHTFPAVLQSSGLYSLSSVVTVPSSSLGTQTYI | |
| | terminal | CNVNHKPSNTKVDKKVEPKSCDKTHTCPPCPAPEAAGAPSVFLFPPKPKDT | |
| | K residue | LMISRTPEVTCVVVDVSHEDPEVKFNWYVDGVEVHNAKTKPREEQYNSTYR | |
| | (H2) | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLQSDGS | |
| | | FFLYSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSNLWVFGG | 141 |
| | (L1) | GTKLTVLGQPKANPTVLLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNDYAMNWVRKAPGKGLEWVARI | SEQ ID NO: |
| | chain | RSKYNNYATYYADSVKDRFTISRDDSKNTAYLQMNNLKTEDTAVYYCVRHG | 479 |
| | (H1) | NFGNSYVSWFAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNDYAMNWVRKAPGKGLEWVARI | SEQ ID NO: |
| | chain | RSKYNNYATYYADSVKDRFTISRDDSKNTAYLQMNNLKTEDTAVYYCVRHG | 585 |
| | with a C- | NFGNSYVSWFAYWGQGTLVTVSSASTKGPSVFPLAPSSKSTSGGTAALGCL | |
| | terminal | VKDYFPEPVTVSWNSGALTSGVHTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0964 | CD20 | EIVLTQSPATLSLSPGERATLSCRASQSVSSYLAWYQKKPGQAPRLLIYDA | SEQ ID NO: |
| | light | SNRATGIPARFSGSGSGTDFTLTISSLEPEDFAVYYCQQRSNWPITFGQGT | 471 |
| | chain | RLEIKRTVAAPSVFIFPPSDEQLKSGTASVVCLLNNFYPREAKVQWKVDNA | |
| | (L2) | LQSGNSQESVTEQDSKDSTYSLSSTLRLSKADYEKHKVYACEVTHQGLSSP | |
| | | VTKSFNRGEC | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 481 |
| | chain | GNYYYGMDVWGQGTTVTVSSASTKGPSVFPLAPSSKSTSGGTAALGCLVDD | |
| | (H2) | YFPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYR | |
| | | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPG | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 557 |
| | chain | GNYYYGMDVWGQGTTVTVSSASTKGPSVFPLAPSSKSTSGGTAALGCLVDD | |
| | with a C- | YFPEPVTVSWNSGALTSGVHTFPAVLQSSGLYSLSSVVTVPSSSLGTQTYI | |
| | terminal | CNVNHKPSNTKVDKKVEPKSCDKTHTCPPCPAPEAAGAPSVFLFPPKPKDT | |
| | K residue | LMISRTPEVTCVVVDVSHEDPEVKFNWYVDGVEVHNAKTKPREEQYNSTYR | |
| | (H2) | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLQSDGS | |
| | | FFLYSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSQLWVFGG | 149 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 482 |
| | (H1) | NFGDDYVSWFAYWGQGTLVTVSSASTKGPSVFPLAPSSKSTSGGTAALGCL | |
| | | VKDYFPEPVTVSWNSGALTSGVHTSPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 586 |
| | with a C- | NFGDDYVSWFAYWGQGTLVTVSSASTKGPSVFPLAPSSKSTSGGTAALGCL | |
| | terminal | VKDYFPEPVTVSWNSGALTSGVHTSPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0962 | CD20 | EIVLTQSPATLSLSPGERATLSCRASQSVSSYLAWYQKKPGQAPRLLIYDA | SEQ ID NO: |
| | light | SNRATGIPARFSGSGSGTDFTLTISSLEPEDFAVYYCQQRSNWPITFGQGT | 471 |
| | chain | RLEIKRTVAAPSVEIFPPSDEQLKSGTASVVCLLNNFYPREAKVQWKVDNA | |
| | (L2) | LQSGNSQESVTEQDSKDSTYSLSSTLRLSKADYEKHKVYACEVTHQGLSSP | |
| | | VTKSFNRGEC | |
| | CD20 | EVQLVESGGGLVQPGRSARLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 484 |
| | chain | GNYYYGMDVWGQGTTVTVSSASTKGPSVFPLAPSSKSTSGGTAALGCLVDD | |
| | (H2) | YFPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYR | |
| | | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPG | |
| | CD20 | EVQLVESGGGLVQPGRSARLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 558 |
| | chain | GNYYYGMDVWGQGTTVTVSSASTKGPSVFPLAPSSKSTSGGTAALGCLVDD | |
| | with a C- | YFPEPVTVSANSGALTSGVHTFPAVLQSSGLYSLSSVVTVPSSSLGTQTYI | |
| | terminal | CNVNHKPSNTKVDKKVEPKSCDKTHTCPPCPAPEAAGAPSVFLFPPKPKDT | |
| | K residue | LMISRTPEVTCVVVDVSHEDPEVKFNWYVDGVEVHNAKTKPREEQYNSTYR | |
| | (H2) | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | DTNKRAPGTPARFSGSLLGGKAALTLSGVQPEDEAEYYCALWYSNLWVFGG | 153 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 485 |
| | (H1) | NFGNSYVSWFAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKETVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 587 |
| | with a C- | NFGNSYVSWFAYWGQGTLVTVSSASTKGPSVFPLAPSSKSTSGGTAALGCL | |
| | terminal | VKDYPPEPVTVSWNSGALTSGVHTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0865 | CD20 | EIVLTQSPATLSLSPGERATLSCRASQSVSSYLAWYQKKPGQAPRLLIYDA | SEQ ID NO: |
| | light | SNRATGIPARFSGSGSGTDFTLTISSLEPEDFAVYYCQQRSNWPITFGQGT | 471 |
| | chain | RLEIKRTVAAPSVFIFPPSDEQLKSGTASVVCLLNNFYPREAKVQWKVDNA | |
| | (L2) | LQSGNSQESVTEQDSKDSTYSLSSTLRLSKADYEKHKVYACEVTHQGLSSP | |
| | | VTKSFNRGEC | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 487 |
| | chain | GNYYYGMDVWGQGTTVTVSSASTKGPSVFPLAPSSKSTSGGTAALGQLVDD | |
| | (H2) | YFPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYR | |
| | | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPG | |
| | CD20 | EVQLVESGGGLVQPGRSLRLSCAASGFTFNDYAMHWVRDAPGKGLEWVSTI | SEQ ID NO: |
| | heavy | SWNSGSIGYADSVKGRFTISRDNAKKSLYLQMNSLRAEDTALYYCAKDIQY | 559 |
| | chain | GNYYYGMDVWGQGTTVTVSSASTKGPSVFPLAPSSKSTSGGTAALGCLVDD | |
| | with a C- | YFPEPVTVSWNSGALTSGVHTFPAVLQSSGLYSLSSVVTVPSSSLGTQTYI | |
| | terminal | CNVNHKPSNTKVDKKVEPKSCDKTHTCPPCPAPEAAGAPSVFLFPPKPKDT | |
| | K residue | LMISRTPEVTCVVVDVSHEDPEVKFNWYVDGVEVHNAKTKPREEQYNSTYR | |
| | (H2) | VVSVLTVLHQDWLNGKEYKCKVSNKALPAPIEKTISKAKGQPREPQVYTLP | |
| | | PCREEMTKNQVSLWCLVKGFYPSDIAVEWESNGQPENNYKTTPPVLDSDGS | |
| | | FFLYSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSNLWVFGG | 141 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 488 |
| | (H1) | NFGNSYVSWFAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKIKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 588 |
| | with a C- | NFGNSYVSWFAYWGQGTLVTVSSASTKGPSVFPLAPSSKSTSGGTAALGCL | |
| | terminal | VKDYFPEPVTVSWNSGALTSGVHTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVQVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0826 | CD20 | DIVMTQTPLSLPVTPGEPASISCRSSKSLLHSNGITYLYWYLKKPGQSPQL | SEQ ID NO: |
| | light | LIYQMSNLVSGVPDRFSGSGSGTDFTLKISRVEAEDVGVYYCAQNLELPYT | 507 |
| | chain | FGGGTKVEIKRTVAAPSVFIFPPSDEQLKSGTASVVCLLNNFYPREAKVQW | |
| | (L2-1) | KVDNALQSGNSQESVTEQDSKDSTYSLSSTLRLSKADYEKAKVYACEVTHQ | |
| | | GLSSPVTKSFNRGEC | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 508 |
| | chain | GYWLVYWGQGTLVTVSSASTKGPSVFPLAPSSKSTSGGTAALGCLVDDYFP | |
| | (H2-1) | EPVTVSWNSGALTSGVHTFPAVLQSSGLYSLSSVVTVPSSSLGTQTYICNV | |
| | | NHKPSNTKVDKKVEPKSCDKTHTCPPCPAPEAAGAPSVFLFPPKPKDTLMI | |
| | | SRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPG | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 560 |
| | chain | GYWLVYWGQGTLVTVSSASTKGPSVFPLAPSSKSTSGGTAALGCLVDDYFP | |
| | with a C- | EPVTVSWNSGALTSGVHTFPAVLQSSGLYSLSSVVTVPSSSLGTQTYICNV | |
| | terminal | NHKPSNTKVDKKVEPKSCDKTHTCPPCPAPEAAGAPSVFLFPPKPKDTLMI | |
| | K residue | SRTPEVTCVVVDVSHEDPEVKFNWYVDGVEVHNAKTKPREEQYNSTYRVVS | |
| | (H2) | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSQLWVFGG | 149 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 509 |
| | (H1) | SFGNSYVSWFAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSIGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 589 |
| | with a C- | SFGNSYVSWFAYWGQGTLVTVSSASTKGPSVFPLAPSSKSTSGGTAALGCL | |
| | terminal | VKDYFPEPVTVSWNSGALTSGVHTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0825 | CD20 | DIVMTQTPLSLPVTPGEPASISCRSSKSLLKSNGITYLYWYLKKPGQSPQL | SEQ ID NO: |
| | light | LIYQMSNLVSGVPDRFSGSGSGTDFTLKISRVEAEDVGVYYCAQNLELPYT | 510 |
| | chain | FGGGTKVEIKRTVAAPSVFIFPPSDEQLKSGTASVVCLLNNFYPREAKVQW | |
| | (L2-1) | KVDNALQSGNSQESVTEQDSKDSTYSLSSTLRLSKADYEKAKVYACEVTHQ | |
| | | GLSSPVTKSFNRGEC | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 511 |
| | chain | GYWLVYWGQGTLVTVSSASTKGPSVFPLAPSSKSTSGGTAALGCLVDDYFP | |
| | (H2-1) | EPVTVSWNSGALTSGVHTFPAVLQSSGLYSLSSVVTVPSSSLGTQTYICNV | |
| | | NHKPSNTKVDKKVEPKSCDKTHTCPPCPAPEAAGAPSVFLFPPKPKDTLMI | |
| | | SRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPG | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 561 |
| | chain | GYWLVYWGQGTLVTVSSASTKGPSVFPLAPSSKSTSGGTAALGCLVDDYFP | |
| | with a C- | EPVTVSWNSGALTSGVHTFPAVLQSSGLYSLSSVVTVPSSSLGTQTYICNV | |
| | terminal | NHKPSNTKVDKKVEPKSCDKTHTCPPCPAPEAAGAPSVFLFPPKPKDTLMI | |
| | K residue | SRTPEVTCVVVDVSHEDPEVKFNWYVDGVEVHNAKTKPREEQYNSTYRVVS | |
| | (H2) | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPGK | |
| | CD3 light | ELVVTQEPSLTVSPGGTVLLTCRSSIGAVTTSNYANWVQDKPGQAPRGLIG | SEQ ID NO: |
| | chain | GTNKRAPGTPARFSGSLLGGKAALTLSGVQPEDEAEYYCALWYSQLWVFGG | 149 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVAKI | SEQ ID NO: |
| | chain | RSKYNNYATYYADSVKDRFTISRDDSKNTAYLQMNNLKTEDTAVYYCVRHG | 512 |
| | (H1) | NFGDSYVSWFAYWGQGTLVTVSSASTKGPSVFPLAPSSKSTSGGTAALGCL | |
| | | VKDYFPEPVTVSWNSGALTSGVHTSPACLQSSGLYSLSSVVTVPSSSLGTQ | |
| | | TYICNVNHKPSNTKVDKKVEPKSSDKTHTCPPCPAPEAAGAPSVFLFPPKP | |
| | | KQTLMISRTPEVTQVVVDVSHEDPEVKFNWYVQGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVAKI | SEQ ID NO: |
| | chain | RSKYNNYATYYADSVKDRFTISRDDSKNTAYLQMNNLKTEDTAVYYCVRHG | 590 |
| | with a C- | NFGDSYVSWFAYWGQGTLVTVSSASTKGPSVFPLAPSSKSTSGGTAALGCL | |
| | terminal | VKDYFPEPVTVSWNSGALTSGVHTSPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KQTLMISRTPEVTCVVVDVSREDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0824 | CD20 | DIVMTQTPLSLPVTPGEPASISCRSSKSLLHSNGITYLYWYLKKPGQSPQD | SEQ ID NO: |
| | light | LIYQMSNLVSGVPDRFSGSGSGTDFTLKISRVEAEDVGVYYCAQNLELPYT | 513 |
| | chain | FGGGTKVEIKRTVAAPSVFIFPPSDEQLKSGTASVVCLLNNFYPREAKVQW | |
| | (L2-1) | KVDNALQSGNSQESVTEQDSKDSTYSLSSTLRLSKADYEKHKVYACEVTHQ | |
| | | GLSSPVTKSFNRGEC | |
| | CD20 | EVQLVQSGAEVKKPGSSVKVSCKASGYAPSYSWINWVRDAPGQGLEWMGRI | SEQ ID NO: |
| | heavy | FPGDGDTDYNGKFKGRVTITADKSTSTAYMELSSLRSEDTAVYYCARNVFD | 514 |
| | chain | GYWLVYWGQGTLVTVSSASTKGPSVFPLAPSSKSTSGGTAALGCLVQDYFP | |
| | (H2-1) | EPVTVSWNSGALTSGVHTFPAVLQSSGLYSLSSVVTVPSSSLGTQTYICNV | |
| | | NHKPSNTKVDKKVEPKSCDKTHTCPPCPAPEAAGAPSVFLFPPKPKDTLMI | |
| | | SRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPG | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 562 |
| | chain | GYWLVYWGQGTLVTVSSASTKGPSVEPLAPSSKSTSGGTAALGCLVDDYFP | |
| | with a C- | EPVTVSWNSGALTSGVHTFPAVLQSSGLYSLSSVVTVPSSSLGTQTYICNV | |
| | terminal | NHKPSNTKVDKKVEPKSCDKTHTCPPCPAPEAAGAPSVFLFPPKPKDTLMI | |
| | K residue | SRTPEVTCVVVDVSHEDPEVKFNWYVDGVEVHNAKTKPREEQYNSTYRVVS | |
| | (H2) | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSNLWVFGG | 141 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNDYAMNWVRKAPGKGLEWVARI | SEQ ID NO: |
| | chain | RSKYNNYATYYADSVKDRFTISRDDSKNTAYLQMNNLKTEDTAVYYCVRHG | 515 |
| | (H1) | NFGNSYVSWFAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KQTLMISRTPEVTQVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNDYAMNWVRKAPGKGLEWVARI | SEQ ID NO: |
| | chain | RSKYNNYATYYADSVKDRFTISRDDSKNTAYLQMNNLKTEDTAVYYCVRHG | 591 |
| | with a C- | NFGNSYVSWFAYWGQGTLVTVSSASTKGPSVFPLAPSSKSTSGGTAALGCL | |
| | terminal | VKDYFPEPVTVSWNSGALTSGVHTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KQTLMISRTPEVTQVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0846 | CD20 | DIVMTQTPLSLPVTPGEPASISCRSSKSLLHSNGITYLYWYLKKPGQSPQL | SEQ ID NO: |
| | light | LIYQMSNLVSGVPDRFSGSGSGTDFTLKISRVEAEDVGVYYCAQNLELPYT | 516 |
| | chain | FGGGTKVEIKRTVAAPSVFIFPPSDEQLKSGTASVVCLLNNFYPREAKVQW | |
| | (L2-1) | KVDNALQSGNSQESVTEQDSKDSTYSLSSTLRLSKADYEKAKVYACEVTHQ | |
| | | GLSSPVTKSFNRGEC | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 517 |
| | chain | GYWLVYWGQGTLVTVSSASTKGPSVFPLAPSSKSTSGGTAALGCLVDDYFP | |
| | (H2-1) | EPVTVSWNSGALTSGVHTFPAVLQSSGLYSLSSVVTVPSSSLGTQTYICNV | |
| | | NHKPSNTKVDKKVEPKSCDKTHTCPPCPAPEAAGAPSVFLFPPKPKDTLMI | |
| | | SRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPG | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 563 |
| | chain | GYWLVYWGQGTLVTVSSASTKGPSVFPLAPSSKSTSGGTAALGCLVDDYFP | |
| | with a C- | EPVTVSWNSGALTSGVHTFPAVLQSSGLYSLSSVVTVPSSSLGTQTYICNV | |
| | terminal | NHKPSNTKVDKKVEPKSCDKTHTCPPCPAPEAAGAPSVFLFPPKPKDTLMI | |
| | K residue | SRTPEVTCVVVDVSHEDPEVKFNWYVDGVEVHNAKTKPREEQYNSTYRVVS | |
| | (H2) | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVQKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSQLWVFGG | 149 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 518 |
| | (H1) | NFGDDYVSWFAYWGQGTLVTVSSASTKGPSVFPLAPSSKSTSGGTAALGCL | |
| | (N96S) | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 592 |
| | with a C- | NFGDDYVSWFAYWGQGTLVTVSSASTKGPSVFPLAPSSKSTSGGTAALGCL | |
| | terminal | VKDYFPEPVTVSWNSGALTSGVHTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) (N96S) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0965 | CD20 | DIVMTQTPLSLPVTPGEPASISCRSSKSLLHSNGITYLYWYLKKPGQSPQL | SEQ ID NO: |
| | light | LIYQMSNLVSGVPDRFSGSGSGTDFTLKISRVEAEDVGVYYCAQNLELPYT | 519 |
| | chain | FGGGTKVEIKRTVAAPSVFIFPPSDEQLKSGTASVVCLLNNFYPREAKVQW | |
| | (L2-1) | KVDNALQSGNSQESVTEQDSKDSTYSLSSTLRLSKADYEKHKVYACEVTHQ | |
| | | GLSSPVTKSFNRGEC | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 520 |
| | chain | GYWLVYWGQGTLVTVSSASTKGPSVFPLAPSSKSTSGGTAALGCLVDDYFP | |
| | (H2-1) | EPVTVSWNSGALTSGVHTFPAVLQSSGLYSLSSVVTVPSSSLGTQTYICNV | |
| | | NHKPSNTKVDKKVEPKSCDKTHTCPPCPAPEAAGAPSVFLFPPKPKDTLMI | |
| | | SRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVQKSRWQQGNVFSCSVMHEALHNHYTQKSLSLSPG | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 564 |
| | chain | GYWLVYWGQGTLVTVSSASTKGPSVFPLAPSSKSTSGGTAALGCLVDDYFP | |
| | with a C- | EPVTVSWNSGALTSGVHTFPAVLQSSGLYSLSSVVTVPSSSLGTQTYICNV | |
| | terminal | NHKPSNTKVDKKVEPKSCDKTHTCPPCPAPEAAGAPSVFLFPPKPKDTLMI | |
| | K residue | SRTPEVTCVVVDVSHEDPEVKFNWYVDGVEVHNAKTKPREEQYNSTYRVVS | |
| | (H2) | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVQKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | DTNKRAPGTPARFSGSLLGGKAALTLSGVQPEDEAEYYCALWYSNLWVFGG | 153 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 521 |
| | (H1) | NFGNSYVSWEAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KQTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 593 |
| | with a C- | NFGNSYVSWFAYWGQGTLVTVSSASTKGPSVFPLAPSSKSTSGGTAALGCL | |
| | terminal | VKDYFPEPVTVSWNSGALTSGVRTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTQVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |
| EIP0867 | CD20 | DIVMTQTPLSLPVTPGEPASISCRSSKSLLHSNGITYLYWYLKKPGQSPQL | SEQ ID NO: |
| | light | LIYQMSNLVSGVPDRFSGSGSGIDFTLKISRVEAEDVGVYYCAQNLELPYT | 522 |
| | chain | FGGGTKVEIKRTVAAPSVFIFPPSDEQLKSGTASVVCLLNNFYPREAKVQW | |
| | (L2-1) | KVDNALQSGNSQESVTEQDSKDSTYSLSSTERLSKADYEKHKVYACEVTHQ | |
| | | GLSSPVTKSFNRGEC | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 523 |
| | chain | GYWLVYWGQGTLVTVSSASTKGPSVFPLAPSSKSTSGGTAALGCLVDDYFP | |
| | (H2-1) | EPVTVSWNSGALTSGVHTFPAVLQSSGLYSLSSVVTVPSSSLGTQTYICNV | |
| | | NHKPSNTKVDKKVEPKSCDKTHTCPPCPAPEAAGAPSVFLFPPKPKDTLMI | |
| | | SRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPG | |
| | CD20 | EVQLVQSGAEVKKPGSSVKVSCKASGYAFSYSWINWVRDAPGQGLEWMGRI | SEQ ID NO: |
| | heavy | FPGDGDTDYNGKFKGRVTITADKSTSTAYMELSSLRSEDTAVYYCARNVFD | 565 |
| | chain | GYWLVYWGQGTLVTVSSASTKGPSVFPLAPSSKSTSGGTAALGCLVDDYFP | |
| | with a C- | EPVTVSWNSGALTSGVHTFPAVLQSSGLYSLSSVVTVPSSSLGTQTYICNV | |
| | terminal | NHKPSNTKVDKKVEPKSCDKTHTCPPCPAPEAAGAPSVFLFPPKPKDTLMI | |
| | K residue | SRTPEVTCVVVDVSHEDPEVKFNWYVDGVEVHNAKTKPREEQYNSTYRVVS | |
| | (H2) | VLTVLHQDWLNGKEYKCKVSNKALPAPIEKTISKAKGQPREPQVYTLPPCR | |
| | | EEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIG | SEQ ID NO: |
| | chain | GTNKRAPGTPARFSGSLLGGKAALTLSGVQPEDEAEYYCALWYSNLWVFGG | 141 |
| | (L1) | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 524 |
| | (H1) | NFGNSYVSWFAYWGQGTLVTVSSASTKGPSVFPLAPSSKSTSGGTAALGCL | |
| | | VKDYFPEPVTVSWNSGALTSGVHTFPACLQSSGLYSLSSVVTVPSSSLGTQ | |
| | | TYICNVNHKPSNTKVDKKVEPKSSDKTHTCPPCPAPEAAGAPSVFLFPPKP | |
| | | KQTLMISRTPEVTCVVVDVSHEDPEVKFNWYVDGVEVENAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARI | SEQ ID NO: |
| | chain | RSKYNNYATYYADSVKDRFTISRDDSKNTAYLQMNNLKTEDTAVYYCVRHG | 594 |
| | with a C- | NFGNSYVSWFAYWGQGTLVTVSSASTKGPSVFPLAPSSKSTSGGTAALGCL | |
| | terminal | VKDYFPEPVTVSWNSGALTSGVRTFPACLQSSGLYSLSSVVTVPSSSLGTQ | |
| | K | TYICNVNHKPSNTKVDKKVEPKSSDKTHTCPPCPAPEAAGAPSVFLFPPKP | |
| | residue | KDTLMISRTPEVTCVVVDVSHEDPEVKFNWYVDGVEVHNAKTKPREEQYNS | |
| | (H1) | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPGK | |

Exemplary, anti-CD3ε×anti-CD20 bispecific antibodies of the invention include EIP0960, EIP0960-2, EIP0960-3, EIP0960-4, EIP0963, EIP0963-2, EIP0963-3, EIP0963-4, EIP0961, EIP0961-2, EIP0961-3, EIP0961-4, EIP0964, EIP0964-2, EIP0964-3, EIP0964-4, EIP0962, EIP0962-2, EIP0962-3, EIP0962-4, EIP0865, EIP0865-2, EIP0865-3, EIP0865-4, EIP0826, EIP0826-2, EIP0826-3, EIP0826-4, EIP0825, EIP0825-2, EIP0825-3, EIP0825-4, EIP0824, EIP0824-2, EIP0824-3, EIP0824-4, EIP0846, EIP0846-2, EIP0846-3, EIP0846-4, EIP0965, EIP0965-2, EIP0965-3, EIP0965-4, EIP0867, EIP0867-2, EIP0867-3, and EIP0867-4.

In some embodiments, the bispecific antibody EIP0960-1, EIP0960-2, EIP0960-3, EIP0960-4, EIP0826-1, EIP0826-2, EIP0826-3, and EIP0826-4, comprises a first antigen binding domain that binds CD3ε, comprising a VH1_CDR1 comprising the amino acid sequence of SEQ ID NO: 29; a VH1_CDR2 comprising the amino acid sequence of SEQ ID NO: 34; a VH1_CDR3 comprising the amino acid sequence of SEQ ID NO: 40: a VL1_CDR1 comprising the amino acid sequence of SEQ ID NO: 42: a VL1_CDR2 comprising the amino acid sequence of SEQ ID NO: 43; and a VL1_CDR3 comprising the amino acid sequence of SEQ ID NO: 47.

In some embodiments, the bispecific antibody EIP0963-1, EIP0963-2, EIP0963-3, EIP0963-4, EIP0825-1, EIP0825-2, EIP0825-3, and EIP0825-4, comprises a first antigen binding domain that binds CD3ε, comprising a VH1_CDR1 comprising the amino acid sequence of SEQ ID NO: 29; a VH1_CDR2 comprising the amino acid sequence of SEQ ID NO: 35: a VH1_CDR3 comprising the amino acid sequence of SEQ ID NO: 38; a VL1_CDR1 comprising the amino acid sequence of SEQ ID NO: 42; a VL1_CDR2 comprising the amino acid sequence of SEQ ID NO: 43; and a VL1_CDR3 comprising the amino acid sequence of SEQ ID NO: 47.

In some embodiments, the bispecific antibody EIP0961-1, EIP0961-2, EIP0961-3, EIP0961-4, EIP0824-1, EIP0824-2, EIP0824-3, and EIP0824-4, comprises a first antigen binding domain that binds CD3ε, comprising a VH1_CDR1 comprising the amino acid sequence of SEQ ID NO: 30; a VH1_CDR2 comprising the amino acid sequence of SEQ ID NO: 34: a VH1_CDR3 comprising the amino acid sequence of SEQ ID NO: 37; a VL1_CDR1 comprising the amino acid sequence of SEQ ID NO: 42; a VL1_CDR2 comprising the amino acid sequence of SEQ ID NO: 43; and a VL1_CDR3 comprising the amino acid sequence of SEQ ID NO: 45.

In some embodiments, the bispecific antibody EIP0964-1, EIP0964-2, EIP0964-3, EIP0964-4, EIP0846-1, EIP0846-2, EIP0846-3, and EIP0846-4, comprises a first antigen binding domain that binds CD3ε, comprising a VH1_CDR1 comprising the amino acid sequence of SEQ ID NO: 29; a VH1_CDR2 comprising the amino acid sequence of SEQ ID NO: 34; a VH1_CDR3 comprising the amino acid sequence of SEQ ID NO: 39; a VL1_CDR1 comprising the amino acid sequence of SEQ ID NO: 42; a VL1_CDR2 comprising the amino acid sequence of SEQ ID NO: 43; and a VL1 CDR3 comprising the amino acid sequence of SEQ ID NO: 47.

In some embodiments, the bispecific antibody EIP0962-1, EIP0962-2, EIP0962-3, EIP0962-4, EIP0%5-1, EIP0965-2, EIP0965-3, and EIP0965-4, comprises a first antigen binding domain that binds CD3ε, comprising a VH1_CDR1 comprising the amino acid sequence of SEQ ID NO: 29: a VH1_CDR2 comprising the amino acid sequence of SEQ ID NO: 34; a VH1_CDR3 comprising the amino acid sequence of SEQ ID NO: 37; a VL1_CDR1 comprising the amino acid sequence of SEQ ID NO: 42: a VL1_CDR2 comprising the amino acid sequence of SEQ ID NO: 44; and a VL1_CDR3 comprising the amino acid sequence of SEQ ID NO: 45.

In some embodiments, the bispecific antibody EIP0865-1. EIP0865-2, EIP0865-3, and EIP0865-4, EIP0867-1, EIP0867-2, EIP0867-3, and EIP0867-4, comprises a first antigen binding domain that binds CD3ε, comprising a VH1_CDR1 comprising the amino acid sequence of SEQ ID NO: 29; a VH1_CDR2 comprising the amino acid sequence of SEQ ID NO: 34: a VH1_CDR3 comprising the amino acid sequence of SEQ ID NO: 37: a VL1_CDR1 comprising the amino acid sequence of SEQ ID NO: 42: a VL1_CDR2 comprising the amino acid sequence of SEQ ID NO: 43; and a VL1_CDR3 comprising the amino acid sequence of SEQ ID NO: 45.

In some embodiments, the bispecific antibody EIP0960, EIP0960-2, EIP0960-3, EIP0960-4, EIP0963, EIP0963-2, EIP0963-3, EIP0963-4, EIP0961, EIP0961-2, EIP0961-3, EIP0961-4, EIP0964, EIP0964-2, EIP0964-3, EIP0964-4, EIP0962, EIP0962-2, EIP0962-3, EIP0962-4, EIP0865, EIP0865-2, EIP0865-3, and EIP0865-4, comprises a second antigen binding domain that binds CD20, comprising a VH2_CDR1 comprising the amino acid sequence of SEQ ID NO: 459; a VH2_CDR2 comprising the amino acid sequence of SEQ ID NO: 460; a VH2_CDR3 comprising the amino acid sequence of SEQ ID NO: 462; a VL2_CDR1 comprising the amino acid sequence of SEQ ID NO: 466: a VL2_CDR2 comprising the amino acid sequence of SEQ ID NO: 467; and a VL2_CDR3 comprising the amino acid sequence of SEQ ID NO: 468.

In some embodiments, the bispecific antibody EIP0826, EIP0826-2, EIP0826-3, EIP0826-4, EIP0825, EIP0825-2, EIP0825-3, EIP0825-4, EIP0824, EIP0824-2, EIP0824-3, EIP0824-4, EIP0846, EIP0846-2, EIP0846-3, EIP0846-4, EIP0965, EIP0965-2, EIP0965-3, EIP0965-4, EIP0867, EIP0867-2, EIP0867-3, and EIP0867-4, comprises a second antigen binding domain that binds CD20, comprising a VH2_CDR1 comprising the amino acid sequence of SEQ ID NO: 455; a VH2_CDR2 comprising the amino acid sequence of SEQ ID NO: 457: a VH2_CDR3 comprising the amino acid sequence of SEQ ID NO: 458; a VL2_CDR1 comprising the amino acid sequence of SEQ ID NO: 463: a VL2_CDR2 comprising the amino acid sequence of SEQ ID NO: 464; and a VL2_CDR3 comprising the amino acid sequence of SEQ ID NO: 465. In some embodiments, the bispecific antibody EIP0960 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0960-2 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0960-3 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0960-4 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0963 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0963-2 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0963-3 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0963-4 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0961 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0961-2 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0961-3 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0961-4 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0964 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0964-2 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0964-3 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0964-4 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0962 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0962-2 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0962-3 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0962-4 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0865 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0865-2 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0865-3 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0865-4 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0826 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0826-2 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0826-3 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0826-4 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0825 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0825-2 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0825-3 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0825-4 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0824 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0824-2 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0824-3 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0824-4 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0846 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0846-2 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0846-3 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0846-4 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0965 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0965-2 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0965-3 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0965-4 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0867 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0867-2 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0867-3 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0867-4 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0960-1 comprises a H1 comprising the amino acid sequence of SEQ ID NO: 473, a L1 comprising the amino acid sequence of SEQ ID NO: 149, a H2 comprising the amino acid sequence of SEQ ID NO: 472, and a L2 comprising the amino acid sequence of SEQ ID NO: 471.

In some embodiments, the bispecific antibody EIP0960-2 comprises a H1 comprising the amino acid sequence of SEQ ID NO: 473, a L1 comprising the amino acid sequence of SEQ ID NO: 149, a H2 comprising the amino acid sequence of SEQ ID NO: 554, and a L2 comprising the amino acid sequence of SEQ ID NO: 471.

In some embodiments, the bispecific antibody EIP0960-3 comprises a H1 comprising the amino acid sequence of SEQ ID NO: 583, a L1 comprising the amino acid sequence of SEQ ID NO: 149, a H2 comprising the amino acid sequence of SEQ ID NO: 472, and a L2 comprising the amino acid sequence of SEQ ID NO: 471.

In some embodiments, the bispecific antibody EIP0960-4 comprises a H1 comprising the amino acid sequence of SEQ ID NO: 583, a L1 comprising the amino acid sequence of SEQ ID NO: 149, a H2 comprising the amino acid sequence of SEQ ID NO: 554, and a L2 comprising the amino acid sequence of SEQ ID NO: 471.

In some embodiments, the bispecific antibody EIP0963-1 comprises a H1 comprising the amino acid sequence of SEQ ID NO: 476, a L1 comprising the amino acid sequence of SEQ ID NO: 149, a H2 comprising the amino acid sequence of SEQ ID NO: 475, and a L2 comprising the amino acid sequence of SEQ ID NO: 471.

In some embodiments, the bispecific antibody EIP0963-2 comprises a H1 comprising the amino acid sequence of SEQ ID NO: 476, a L1 comprising the amino acid sequence of SEQ ID NO: 149, a H2 comprising the amino acid sequence of SEQ ID NO: 555, and a L2 comprising the amino acid sequence of SEQ ID NO: 471.

In some embodiments, the bispecific antibody EIP0963-3 comprises a H1 comprising the amino acid sequence of SEQ ID NO: 584, a L1 comprising the amino acid sequence of SEQ ID NO: 149, a H2 comprising the amino acid sequence of SEQ ID NO: 475, and a L2 comprising the amino acid sequence of SEQ ID NO: 471.

In some embodiments, the bispecific antibody EIP0963-4 comprises a H1 comprising the amino acid sequence of SEQ ID NO: 584, a L1 comprising the amino acid sequence of SEQ ID NO: 149, a H2 comprising the amino acid sequence of SEQ ID NO: 555, and a L2 comprising the amino acid sequence of SEQ ID NO: 471.

In some embodiments, the bispecific antibody EIP0961-1 comprises a H1 comprising the amino acid sequence of SEQ ID NO: 479, a L1 comprising the amino acid sequence of SEQ ID NO: 141, a H2 comprising the amino acid sequence of SEQ ID NO: 478, and a L2 comprising the amino acid sequence of SEQ ID NO: 471.

In some embodiments, the bispecific antibody EIP0961-2 comprises a H1 comprising the amino acid sequence of SEQ ID NO: 479, a L1 comprising the amino acid sequence of SEQ ID NO: 141, a H2 comprising the amino acid sequence of SEQ ID NO: 556, and a L2 comprising the amino acid sequence of SEQ ID NO: 471.

In some embodiments, the bispecific antibody EIP0961-3 comprises a H1 comprising the amino acid sequence of SEQ ID NO: 585, a L1 comprising the amino acid sequence of SEQ ID NO: 141, a H2 comprising the amino acid sequence of SEQ ID NO: 478, and a L2 comprising the amino acid sequence of SEQ ID NO: 471.

In some embodiments, the bispecific antibody EIP0961-4 comprises a H1 comprising the amino acid sequence of SEQ ID NO: 585, a L1 comprising the amino acid sequence of SEQ ID NO: 141, a H2 comprising the amino acid sequence of SEQ ID NO: 556, and a L2 comprising the amino acid sequence of SEQ ID NO: 471.

In some embodiments, the bispecific antibody EIP0964-1 comprises a H1 comprising the amino acid sequence of SEQ ID NO: 482, a L1 comprising the amino acid sequence of SEQ ID NO: 149, a H2 comprising the amino acid sequence of SEQ ID NO: 481, and a L2 comprising the amino acid sequence of SEQ ID NO: 471.

In some embodiments, the bispecific antibody EIP0964-2 comprises a H1 comprising the amino acid sequence of SEQ ID NO: 482, a L1 comprising the amino acid sequence of SEQ ID NO: 149, a H2 comprising the amino acid sequence of SEQ ID NO: 557, and a L2 comprising the amino acid sequence of SEQ ID NO: 471.

In some embodiments, the bispecific antibody EIP0964-3 comprises a H1 comprising the amino acid sequence of SEQ ID NO: 586, a L1 comprising the amino acid sequence of SEQ ID NO: 149, a H2 comprising the amino acid sequence of SEQ ID NO: 481, and a L2 comprising the amino acid sequence of SEQ ID NO: 471.

In some embodiments, the bispecific antibody EIP0964-4 comprises a H1 comprising the amino acid sequence of SEQ ID NO: 586, a L1 comprising the amino acid sequence of SEQ ID NO: 149, a H2 comprising the amino acid sequence of SEQ ID NO: 557, and a L2 comprising the amino acid sequence of SEQ ID NO: 471.

In some embodiments, the bispecific antibody EIP0962-1 comprises a H1 comprising the amino acid sequence of SEQ ID NO: 485, a L1 comprising the amino acid sequence of SEQ ID NO: 153, a H2 comprising the amino acid sequence of SEQ ID NO: 484, and a L2 comprising the amino acid sequence of SEQ ID NO: 471.

In some embodiments, the bispecific antibody EIP0962-2 comprises a H1 comprising the amino acid sequence of SEQ ID NO: 485, a L1 comprising the amino acid sequence of SEQ ID NO: 153, a H2 comprising the amino acid sequence of SEQ ID NO: 558, and a L2 comprising the amino acid sequence of SEQ ID NO: 471.

In some embodiments, the bispecific antibody EIP0962-3 comprises a H1 comprising the amino acid sequence of SEQ ID NO: 587, a L1 comprising the amino acid sequence of SEQ ID NO: 153, a H2 comprising the amino acid sequence of SEQ ID NO: 484, and a L2 comprising the amino acid sequence of SEQ ID NO: 471.

In some embodiments, the bispecific antibody EIP0962-4 comprises a H1 comprising the amino acid sequence of SEQ ID NO: 587, a L1 comprising the amino acid sequence of SEQ ID NO: 153, a H2 comprising the amino acid sequence of SEQ ID NO: 558, and a L2 comprising the amino acid sequence of SEQ ID NO: 471.

In some embodiments, the bispecific antibody EIP0865-1 comprises a H1 comprising the amino acid sequence of SEQ ID NO: 488, a L1 comprising the amino acid sequence of SEQ ID NO: 141, a H2 comprising the amino acid sequence of SEQ ID NO: 487, and a L2 comprising the amino acid sequence of SEQ ID NO: 471.

In some embodiments, the bispecific antibody EIP0865-2 comprises a H1 comprising the amino acid sequence of SEQ ID NO: 488, a L1 comprising the amino acid sequence of SEQ ID NO: 141, a H2 comprising the amino acid sequence of SEQ ID NO: 559, and a L2 comprising the amino acid sequence of SEQ ID NO: 471.

In some embodiments, the bispecific antibody EIP0865-3 comprises a H1 comprising the amino acid sequence of SEQ ID NO: 588, a L1 comprising the amino acid sequence of SEQ ID NO: 141, a H2 comprising the amino acid sequence of SEQ ID NO: 487, and a L2 comprising the amino acid sequence of SEQ ID NO: 471.

In some embodiments, the bispecific antibody EIP0865-4 comprises a H1 comprising the amino acid sequence of SEQ ID NO: 588, a L1 comprising the amino acid sequence of SEQ ID NO: 141, a H2 comprising the amino acid sequence of SEQ ID NO: 559, and a L2 comprising the amino acid sequence of SEQ ID NO: 471.

In some embodiments, the bispecific antibody EIP0826-1 comprises a H1 comprising the amino acid sequence of SEQ ID NO: 509, a L1 comprising the amino acid sequence of SEQ ID NO: 149, a H2 comprising the amino acid sequence of SEQ ID NO: 508, and a L2 comprising the amino acid sequence of SEQ ID NO: 507.

In some embodiments, the bispecific antibody EIP0826-2 comprises a H1 comprising the amino acid sequence of SEQ ID NO: 509, a L1 comprising the amino acid sequence of SEQ ID NO: 149, a H2 comprising the amino acid sequence of SEQ ID NO: 560, and a L2 comprising the amino acid sequence of SEQ ID NO: 507.

In some embodiments, the bispecific antibody EIP0826-3 comprises a H1 comprising the amino acid sequence of SEQ ID NO: 589, a L1 comprising the amino acid sequence of SEQ ID NO: 149, a H2 comprising the amino acid sequence of SEQ ID NO: 508, and a L2 comprising the amino acid sequence of SEQ ID NO: 507.

In some embodiments, the bispecific antibody EIP0826-4 comprises a H1 comprising the amino acid sequence of SEQ ID NO: 589, a L1 comprising the amino acid sequence of SEQ ID NO: 149, a H2 comprising the amino acid sequence of SEQ ID NO: 560, and a L2 comprising the amino acid sequence of SEQ ID NO: 507.

In some embodiments, the bispecific antibody EIP0825-1 comprises a H1 comprising the amino acid sequence of SEQ ID NO: 512, a L1 comprising the amino acid sequence of SEQ ID NO: 149, a H2 comprising the amino acid sequence of SEQ ID NO: 511, and a L2 comprising the amino acid sequence of SEQ ID NO: 510.

In some embodiments, the bispecific antibody EIP0825-2 comprises a H1 comprising the amino acid sequence of SEQ ID NO: 512, a L1 comprising the amino acid sequence of SEQ ID NO: 149, a H2 comprising the amino acid sequence of SEQ ID NO: 561, and a L2 comprising the amino acid sequence of SEQ ID NO: 510.

In some embodiments, the bispecific antibody EIP0825-3 comprises a H1 comprising the amino acid sequence of SEQ ID NO: 590, a L1 comprising the amino acid sequence of SEQ ID NO: 149, a H2 comprising the amino acid sequence of SEQ ID NO: 511, and a L2 comprising the amino acid sequence of SEQ ID NO: 510.

In some embodiments, the bispecific antibody EIP0825-4 comprises a H1 comprising the amino acid sequence of SEQ ID NO: 590, a L1 comprising the amino acid sequence of SEQ ID NO: 149, a H2 comprising the amino acid sequence of SEQ ID NO: 561, and a L2 comprising the amino acid sequence of SEQ ID NO: 510.

In some embodiments, the bispecific antibody EIP0824-1 comprises a H1 comprising the amino acid sequence of SEQ ID NO: 515, a L1 comprising the amino acid sequence of SEQ ID NO: 141, a H2 comprising the amino acid sequence of SEQ ID NO: 514, and a L2 comprising the amino acid sequence of SEQ ID NO: 513.

In some embodiments, the bispecific antibody EIP0824-2 comprises a H1 comprising the amino acid sequence of SEQ ID NO: 515, a L1 comprising the amino acid sequence of SEQ ID NO: 141, a H2 comprising the amino acid sequence of SEQ ID NO: 562, and a L2 comprising the amino acid sequence of SEQ ID NO: 513.

In some embodiments, the bispecific antibody EIP0824-3 comprises a H1 comprising the amino acid sequence of SEQ ID NO: 591, a L1 comprising the amino acid sequence of SEQ ID NO: 141, a H2 comprising the amino acid sequence of SEQ ID NO: 514, and a L2 comprising the amino acid sequence of SEQ ID NO: 513.

In some embodiments, the bispecific antibody EIP0824-4 comprises a H1 comprising the amino acid sequence of SEQ ID NO: 591, a L1 comprising the amino acid sequence of SEQ ID NO: 141, a H2 comprising the amino acid sequence of SEQ ID NO: 562, and a L2 comprising the amino acid sequence of SEQ ID NO: 513.

In some embodiments, the bispecific antibody EIP0846-1 comprises a H1 comprising the amino acid sequence of SEQ ID NO: 518, a L1 comprising the amino acid sequence of SEQ ID NO: 149, a H2 comprising the amino acid sequence of SEQ ID NO: 517, and a L2 comprising the amino acid sequence of SEQ ID NO: 516.

In some embodiments, the bispecific antibody EIP0846-2 comprises a H1 comprising the amino acid sequence of SEQ ID NO: 518, a L1 comprising the amino acid sequence of SEQ ID NO: 149, a H2 comprising the amino acid sequence of SEQ ID NO: 563, and a L2 comprising the amino acid sequence of SEQ ID NO: 516.

In some embodiments, the bispecific antibody EIP0846-3 comprises a H1 comprising the amino acid sequence of SEQ ID NO: 592, a L1 comprising the amino acid sequence of SEQ ID NO: 149, a H2 comprising the amino acid sequence of SEQ ID NO: 517, and a L2 comprising the amino acid sequence of SEQ ID NO: 516.

In some embodiments, the bispecific antibody EIP0846-4 comprises a H1 comprising the amino acid sequence of SEQ ID NO: 592, a L1 comprising the amino acid sequence of SEQ ID NO: 149, a H2 comprising the amino acid sequence of SEQ ID NO: 563, and a L2 comprising the amino acid sequence of SEQ ID NO: 516.

In some embodiments, the bispecific antibody EIP0965-1 comprises a H1 comprising the amino acid sequence of SEQ ID NO: 521, a L1 comprising the amino acid sequence of SEQ ID NO: 153, a H2 comprising the amino acid sequence of SEQ ID NO: 520, and a L2 comprising the amino acid sequence of SEQ ID NO: 519.

In some embodiments, the bispecific antibody EIP0965-2 comprises a H1 comprising the amino acid sequence of SEQ ID NO: 521, a L1 comprising the amino acid sequence of SEQ ID NO: 153, a H2 comprising the amino acid sequence of SEQ ID NO: 564, and a L2 comprising the amino acid sequence of SEQ ID NO: 519.

In some embodiments, the bispecific antibody EIP0965-3 comprises a H1 comprising the amino acid sequence of SEQ ID NO: 593, a L1 comprising the amino acid sequence of SEQ ID NO: 153, a H2 comprising the amino acid sequence of SEQ ID NO: 520, and a L2 comprising the amino acid sequence of SEQ ID NO: 519.

In some embodiments, the bispecific antibody EIP0965-4 comprises a H1 comprising the amino acid sequence of SEQ ID NO: 593, a L1 comprising the amino acid sequence of SEQ ID NO: 153, a H2 comprising the amino acid sequence of SEQ ID NO: 564, and a L2 comprising the amino acid sequence of SEQ ID NO: 519.

In some embodiments, the bispecific antibody EIP0867-1 comprises a H1 comprising the amino acid sequence of SEQ ID NO: 524, a L1 comprising the amino acid sequence of SEQ ID NO: 141, a H2 comprising the amino acid sequence of SEQ ID NO: 523, and a L2 comprising the amino acid sequence of SEQ ID NO: 522.

In some embodiments, the bispecific antibody EIP0867-2 comprises a H1 comprising the amino acid sequence of SEQ ID NO: 524, a L1 comprising the amino acid sequence of SEQ ID NO: 141, a H2 comprising the amino acid sequence of SEQ ID NO: 565, and a L2 comprising the amino acid sequence of SEQ ID NO: 522.

In some embodiments, the bispecific antibody EIP0867-3 comprises a H1 comprising the amino acid sequence of SEQ ID NO: 594, a L1 comprising the amino acid sequence of SEQ ID NO: 141, a H2 comprising the amino acid sequence of SEQ ID NO: 523, and a L2 comprising the amino acid sequence of SEQ ID NO: 522.

In some embodiments, the bispecific antibody EIP0867-4 comprises a H1 comprising the amino acid sequence of SEQ ID NO: 594, a L1 comprising the amino acid sequence of SEQ ID NO: 141, a H2 comprising the amino acid sequence of SEQ ID NO: 565, and a L2 comprising the amino acid sequence of SEQ ID NO: 522.

Any one of the bispecific antibodies shown above in Table 10.4 can be further modified by substituting any one of the anti-CD3ε antigen binding regions with any one of the anti-CD3ε binding regions shown in Tables 9.1, 9.2 and 9.3. For example, the anti-CD3ε antigen binding regions of bispecific antibody “EIP0960” can be substituted with any one of the anti-CD3ε binding regions shown in Tables 9.1, 9.2 and 9.3 to produce the bispecific antibodies of the invention. Exemplary antibodies are shown in Table 10.4.

In some embodiments, the CD3×CD20 bispecific antibodies of the invention have a CD58 fusion peptide (SEQ ID NO: 49) fused indirectly at the C-terminus of the first heavy chain polypeptide (H1) using linker-1 (SEQ ID NO: 52). In some embodiments, the CD3×CD20 bispecific antibodies of the invention have a CD58v* fusion peptide (SEQ ID NO: 50) fused indirectly at the C-terminus of the first heavy chain polypeptide (H1) using linker-1 (SEQ ID NO: 52).

In some embodiments, the CD3×CD20 bispecific antibodies of the invention have a CD58 fusion peptide (SEQ ID NO: 49) fused indirectly at the C-terminus of the first heavy chain polypeptide (H1) using linker-2 (SEQ ID NO: 53). In some embodiments, the CD3×CD20 bispecific antibodies of the invention have a CD58v* fusion peptide (SEQ ID NO: 50) fused indirectly at the C-terminus of the first heavy chain polypeptide (H1) using linker-2 (SEQ ID NO: 53).

In some embodiments, the CD3×CD20 bispecific antibodies of the invention have a CD58 fusion peptide (SEQ ID NO: 49) fused indirectly at the C-terminus of the first heavy chain polypeptide (H1) using linker-3 (SEQ ID NO: 54). In some embodiments, the CD3×CD20 bispecific antibodies of the invention have a CD58v* fusion peptide (SEQ ID NO: 50) fused indirectly at the C-terminus of the first heavy chain polypeptide (H1) using linker-3 (SEQ ID NO: 54).

Exemplary CD3×CD20 bispecific antibodies having a CD58 fusion peptide are shown in Table 10.5.

Table 10.5
Exemplary Bispecific Antibodies that bind to CD3s and CD20
with a C-terminus CD58 fusion peptide

| Antibody | Domain | Amino Acid Sequence | SEQ ID NO |
|---|---|---|---|
| EIP0969 | CD20 light | EIVLTQSPATLSLSPGERATLSCRASQSVSSYLAWYQKKPGQAPRLLIYDAS | SEQ ID NO: |
| | chain (L2) | NRATGIPARFSGSGSGTDFTLTISSLEPEDFAVYYCQQRSNWPITFGQGTRL | 471 |
| | | EIKRTVAAPSVFIFPPSDEQLKSGTASVVCLLNNFYPREAKVQWKVDNALQS | |
| | | GNSQESVTEQDSKDSTYSLSSTLRLSKADYEKHKVYACEVTHQGLSSPVTKS | |
| | | FNRGEC | |
| | CD20 heavy | EVQLVESGGGLVQPGRSLRLSCAASGFTFNDYAMHWVRDAPGKGLEWVSTIS | SEQ ID NO: |
| | chain (H2) | WNSGSIGYADSVKGRFTISRDNAKKSLYLQMNSLRAEDTALYYCARDIQYGN | 490 |
| | | YYYGMDVWGQGTTVTVSSASTKGPSVFPLAPSSKSTSGGTAALGCLVDDYFP | |
| | | EPVTVSWNSGALTSGVRTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSDSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 566 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSQLWVFGGGT | 149 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGSF | 491 |
| | | GNSYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLTSSDEDEYEMESPNITDTMKFFLYVLESLPSPILTCALTNGS | |
| | | IEVQCMIPEHYNSHRGLIMYSWDCPMEQCKANSTSIYFKMENDLPQKIQCTL | |
| | | SNPLFNTISSIILTTCIPSSGHSRHRY | |
| | CD3 heavy | EVQLLESGGGLVQPGGSLKLSCAASGFTFNTYAMNWVRKAPGKGLEWVARIR | SEQ ID |
| | chain with | SKYNNYATYYADSVKDRFTISRDDSKNTAYLQMNNLKTEDTAVYYCVRHGSF | NO: 595 |
| | a K residue | GNSYVSWFAYWGQGTLVTVSSASTKGPSVFPLAPSSKSTSGGTAALGCLVKD | |
| | prior to | YEPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVENAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPILTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0892 | CD20 light | EIVLTQSPATLSLSPGERATLSCRASQSVSSYLAWYQKKPGQAPRLLIYDAS | SEQ ID NO: |
| | chain (L2) | NRATGIPARFSGSGSGTDFTLTISSLEPEDFAVYYCQQRSNWPITFGQGTRL | 471 |
| | | EIKRTVAAPSVFIFPPSDEQLKSGTASVVCLLNNFYPREAKVQWKVDNALQS | |
| | | GNSQESVTEQDSKDSTYSLSSTLRLSKADYEKHKVYACEVTHQGLSSPVTKS | |
| | | FNRGEC | |
| | CD20 light | EVQLVESGGGLVQPGRSLRLSCAASGFTFNDYAMHWVRDAPGKLGEWVSTIS | SEQ ID NO: |
| | chain (H2) | WNSGSIGYADSVKGRFTISRDNAKKSLYLQMNSLRAEDTALYYCAKDIQYGN | 493 |
| | | YYYGMDVWGQGTTVTVSSASTKGPSVFPLAPSSKSTSGGTAALGCLVDDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 567 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSQLWVFGGGT | 149 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSERSYSCQVTHEGSTVEKT | |
| | | VAPTESS | |
| | CD3 heavy | EVQLLESGGGLVQPGGSLKLSCAASGFTFNTYAMNWVRKAPGKGLEWVAKIR | SEQ ID NO: |
| | chain (H1) | SKYNNYATYYADSVKDRFTISRDDSKNTAYLQMNNLKTEDTAVYYCVRHGNF | 494 |
| | | GDSYVSWFAYWGQGTLVTVSSASTKGPSVFPLAPSSKSTSGGTAALGQLVKD | |
| | | YPPEPVTVSWNSGALISGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVENAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKFFLYVLESLPSPTLTCALTNGS | |
| | | IEVQCMIFERYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVAKIR | SEQ ID NO: |
| | chain with | SKYNNYATYYADSVKDRFTISRDDSKNTAYLQMNNLKTEDTAVYYCVRHGNF | 596 |
| | a K residue | GDSYVSWFAYWGQGTLVTVSSASTKGPSVFPLAPSSKSTSGGTAALGCLVKD | |
| | prior to | YFPEPVTVSWNSGALTSGVHTSPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVQVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPILTCALIN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0929 | CD20 light | EIVLTQSPATLSLSPGERATLSCRASQSVSSYLAWYQKKPGQAPRLLIYDAS | SEQ ID NO: |
| | chain (L2) | NRATGIPARFSGSGSGTDFTLTISSLEPEDFAVYYCQQRSNWPITFGQGTRL | 471 |
| | | EIKRTVAAPSVFIFPPSDEQLKSGTASVVCLLNNFYPREAKVQWKVDNALQS | |
| | | GNSQESVTEQDSKDSTYSLSSTLRLSKADYEKHKVYACEVTHQGLSSPVTKS | |
| | | FNRGEC | |
| | CD20 heavy | EVQLVESGGGLVQPGRSLRLSCAASGFTFNDYAMHWVRDAPGKGLEWVSTIS | SEQ ID NO: |
| | chain (H2) | WNSGSIGYADSVKGRFTISRDNAKKSLYLQMNSLRAEDTALYYCAKDIQYGN | 496 |
| | | YYYGMDVWGQGTTVTVSSASTKGPSVFPLAPSSKSTSGGTAALGCLVDDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVDVSHEDPEVKFNWYVDGVEVHNAKTKPREEQYNSTYRVVSVLT | |
| | | VLRQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 568 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSNLWVFGGGT | 141 |
| | | KLTVLGQPKANPTVTLFPPSSEELCANKADLVCLISDFYPGAVTVAWKADGS | |
| | | PVKAGVETCKPSKQSNNKYAASSYLSLTPEQWKSERSYSCQVTHEGSTVEKT | |
| | | VAPTESS | |
| | CD3 heavy | EVQLLESGGGLVQPGGSLKLSCAASGFTFNDYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 497 |
| | | GNSYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKEFLYVLESLPSPTLTCALTNGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNDYAMNWVRKAPGKGLEWVARIR | SEQ ID NO: |
| | chain with | SKYNNYATYYADSVKDRFTISRDDSKNTAYLQMNNLKTEDTAVYYCVRHGNF | 597 |
| | a K residue | GNSYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLQSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILITCIPSSGHSRHRY | |
| EIP0893 | CD20 light | EIVLTQSPATLSLSPGERATLSCRASQSVSSYLAWYQKKPGQAPRLLLYDAS | SEQ ID NO: |
| | chain (L2) | NRATGIPARFSGSGSGTDFTLTISSLEPEDFAVYYCQQRSNWPITFGQGTRL | 471 |
| | | EIKRTVAAPSVFIFPPSDEQLKSGTASVVCLLNNFYPREAKVQWKVDNALQS | |
| | | GNSQESVTEQDSKDSTYSLSSTLRLSKADYEKHKVYACEVTHQGLSSPVTKS | |
| | | FNRGEC | |
| | CD20 heavy | EVQLVESGGGLVQPGRSLRLSCAASGFTFNDYAMHWVRDAPGKGLEWVSTIS | SEQ ID NO: |
| | chain (H2) | WNSGSIGYADSVKGRFTISRDNAKKSLYLQMNSLRAEDTALYYCAKDIQYGN | 499 |
| | | YYYGMDVWGQGTTVTVSSASTKGPSVFPLAPSSKSTSGGTAALGCLVQDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVQVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 569 |
| | K residue | YYYGMQVWGQGTTVTVSSASTKGPSVEPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSQLWVFGGGT | 149 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 500 |
| | | GDDYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLTSSDEDEYEMESPNITDTMKFFLYVLESLPSPTLTCALTNGS | |
| | | IEVQCMIPEHYNSHRGLIMYSWDCPMEQCKENSTSIYEKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKDRFTISRDDSKNTAYLQMNNLKTEDTAVYYCVRHGNF | 598 |
| | a K residue | GDDYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLISSDEDEYEMESPNITDLNKFFLYVLESLPSPTLTCALTN | |
| | | GSIEVQCMIPEHYNSHRGLIMYSWDCPMEQCKANSTSIYFKMENDLPQKIQC | |
| | | TLSNPLENTTSSIILTTCIPSSGHSRHRY | |
| EIP0886 | CD20 light | EIVLTQSPATLSLSPGERATLSCRASQSVSSYLAWYQKKPGQAPRLLIYDAS | SEQ ID NO: |
| | chain (L2) | NRATGIPARFSGSGSGTDFTLTISSLEPEDFAVYYCQQRSNWPITFGQGTRL | 471 |
| | | EIKRTVAAPSVFIFPPSDEQLKSGTASVVCLLNNFYPREAKVQWKVDNALQS | |
| | | GNSQESVTEQDSKDSTYSLSSTLRLSKADYEKHKVYACEVTHQGLSSPVTKS | |
| | | FNRGEC | |
| | CD20 heavy | EVQLVESGGGLVQPGRSLRLSCAASGFTFNDYAMHWVRDAPGKGLEWVSTIS | SEQ ID NO: |
| | chain (H2) | WNSGSIGYADSVKGRFTISRDNAKKSLYLQMNSLRAEDTALYYCAKDIQYGN | 502 |
| | | YYYGMDVWGQGTTVTVSSASTKGPSVFPLAPSSKSTSGGTAALGCLVDDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 570 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGD | SEQ ID NO: |
| | chain (L1) | TNKRAPGTPARFSGSLLGGKAALTLSGVQPEDEAEYYCALWYSNLWVFGGGT | 153 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 503 |
| | | GNSYVSWFAYWGQGTLVTVSSASTKGPSVFPLAPSSKSTSGGTAALGQLVKD | |
| | | YEPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVENAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVIFHVPSNVPLKEVLWKKQKDKVAELENSEFRAFSSFKNRVYLDT | |
| | | VSGSLTIYNLISSDEDEYEMESPNITDIMKFFLYVLESLPSPTLTCALTNGS | |
| | | IEVQCMIFERYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKDRFTISRDDSKNTAYLQMNNLKTEDTAVYYCVRHGNF | 599 |
| | a K residue | GNSYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVQVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKEFLYVLESLPSPTLTCALTN | |
| | | GSIEVQCMIPERYNSHRGLIMYSWDCPMEQCKRNSTSIYFKMENDLPQKIQC | |
| | | TLSNPLENTTSSIILTTCIPSSGHSRHRY | |
| EIP0866 | CD20 light | EIVLTQSPATLSLSPGERATLSCRASQSVSSYLAWYQKKPGQAPRLLIYDAS | SEQ ID NO: |
| | chain (L2) | NRATGIPARFSGSGSGTDFTLTISSLEPEDFAVYYCQQRSNWPITFGQGTRL | 471 |
| | | EIKRTVAAPSVFIFPPSDEQLKSGTASVVCLLNNFYPREAKVQWKVDNALQS | |
| | | GNSQESVTEQDSKDSTYSLSSTLRLSKADYEKEKVYACEVTHQGLSSPVTKS | |
| | | FNRGEC | |
| | CD20 heavy | EVQLVESGGGLVQPGRSLRLSCAASGFTFNDYAMHWVRDAPGKGLEWVSTIS | SEQ ID NO: |
| | chain (H2) | WNSGSIGYADSVKGRFTISRDNAKKSLYLQMNSLRAEDTALYYCAKDIQYGN | 505 |
| | | YYYGMDVWGQGTTVTVSSASTKGPSVFPLAPSSKSTSGGTAALGCLVDDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVQVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 571 |
| | K residue | YYYGMDVWGQGTTVTVSSASTKGPSVFPLAPSSKSTSGGTAALGCLVDDYEP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVITSNYANWVQDKPGQAPRGLIGG | SEQ ID NO: |
| | chain (L1) | TNKRAPGTPARFSGSLLGGKAALTLSGVQPEDEAEYYCALWYSNLWVFGGGT | 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 506 |
| | | GNSYVSWFAYWGQGTLVTVSSASTKGPSVFPLAPSSKSTSGGTAALGCLVKQ | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKFFLYVLESLPSPTLTCALTNGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKGRFTISRQDSKNTAYLQMNNLKTEDTAVYYCVRHGNF | 600 |
| | a K residue | GNSYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVIFHVPSNVPLKEVLWKKQKDKVAELENSEFRAESSEKNRVYL | |
| | | DTVSGSLTIYNLISSDEDEYEMESPNITDIKKEFLYVLESLPSPTLTCALTN | |
| | | GSIEVQCMIPERYNSHRGLIMYSWDCPMEQCKRNSTSIYFKMENDLPQKIQC | |
| | | ILSNPLENTTSSIILTTCIPSSGHSRHRY | |
| EIP0966 | CD20 light | DIVMTQTPLSLPVTPGEPASISCRSSKSLLHSNGITYLYWYLKKPGQSPQLL | SEQ ID NO: |
| | chain (L2- | IYQMSNLVSGVPDRFSGSGSGTDFTLKISRVEAEDVGVYYCAQNLELPYTFG | 525 |
| | 1) | GGTKVEIKRTVAAPSVFIFPPSDEQLKSGTASVVCLLNNFYPREAKVQWKVD | |
| | | NALQSGNSQESVTEQDSKDSTYSLSSTERLSKADYEKHKVYACEVTHQGLSS | |
| | | PVTKSFNRGEC | |
| | CD20 heavy | EVQLVQSGAEVKKPGSSVKVSCKASGYAFSYSWINWVRDAPGQGLEWMGRIF | SEQ ID NO: |
| | chain (H2- | PGDGDTDYNGKFKGRVTITADKSTSTAYMELSSLRSEDTAVYYCARNVFDGY | 526 |
| | 1) | WLVYWGQGTLVTVSSASTKGPSVFPLAPSSKSTSGGTAALGCLVDDYFPEPV | |
| | | TVSWNSGALTSGVHTFPAVLQSSGLYSLSSVVTVPSSSLGTQTYICNVNHKP | |
| | | SNTKVDKKVEPKSCDKTHTCPPCPAPEAAGAPSVFLFPPKPKDTLMISRTPE | |
| | | VTCVVVDVSHEDPEVKFNWYVDGVEVHNAKTKPREEQYNSTYRVVSVLTVLH | |
| | | QDWLNGKEYKCKVSNKALPAPIEKTISKAKGQPREPQVYTLPPCREEMTKNQ | |
| | | VSLWCLVKGFYPSDIAVEWESNGQPENNYKTTPPVLDSDGSFFLYSKLTVDK | |
| | | SRWQQGNVFSCSVMHEALANHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 572 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKQTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSQLWVFGGGT | 149 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGSF | 527 |
| | | GNSYVSWFAYWGQGTLVTVSSASTKGPSVFPLAPSSKSTSGGTAALGCLVKD | |
| | | YPPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVENAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVIFHVPSNVPLKEVLWKKQKDKVAELENSEFRAFSSEKNRVYLDT | |
| | | VSGSLTIYNLISSDEDEYEMESPNITDTMKEFLYVLESLPSPTLTCALTNGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKDRFTISRDDSKNTAYLQMNNLKTEDTAVYYCVRHGSF | 601 |
| | a K residue | GNSYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIFEHYNSHRGLIMYSWDCPMEQCKRNSTSIYFKMENDLPQKIQC | |
| | | TLSNPLENTISSIILITCIPSSGHSRHRY | |
| EIP0967 | CD20 light | DIVMTQTPLSLPVTPGEPASISCRSSKSLLHSNGITYLYWYLKKPGQSPQLL | SEQ ID NO: |
| | chain (L2- | IYQMSNLVSGVPDRFSGSGSGTDFTLKISRVEAEDVGVYYCAQNLELPYTFG | 528 |
| | 1) | GGTKVEIKRTVAAPSVFIFPPSDEQLKSGTASVVCLLNNFYPREAKVQWKVD | |
| | | NALQSGNSQESVTEQDSKDSTYSLSSTLRLSKADYEKHKVYACEVTHQGLSS | |
| | | PVTKSFNRGEC | |
| | CD20 heavy | EVQLVQSGAEVKKPGSSVKVSCKASGYAFSYSWINWVRDAPGQGLEWMGRIF | SEQ ID NO: |
| | chain (H2- | PGDGDTDYNGKFKGRVTITADKSTSTAYMELSSLRSEDTAVYYCARNVFDGY | 529 |
| | 1) | WLVYWGQGTLVTVSSASTKGPSVFPLAPSSKSTSGGTAALGCLVQDYFPEPV | |
| | | TVSWNSGALTSGVHTFPAVLQSSGLYSLSSVVTVPSSSLGTQTYICNVNHKP | |
| | | SNTKVDKKVEPKSCDKTHTCPPCPAPEAAGAPSVFLFPPKPKDTLMISRTPE | |
| | | VTCVVVDVSHEDPEVKFNWYVDGVEVHNAKTKPREEQYNSTYRVVSVLTVLH | |
| | | QDWLNGKEYKCKVSNKALPAPIEKTISKAKGQPREPQVYTLPPCREEMTKNQ | |
| | | VSLWCLVKGFYPSDIAVEWESNGQPENNYKTTPPVLDSDGSFFLYSKLTVDK | |
| | | SRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 573 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSQLWVFGGGT | 149 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVAKIR | SEQ ID NO: |
| | chain (H1) | SKYNNYATYYADSVKDRFTISRDDSKNTAYLQMNNLKTEDTAVYYCVRHGNF | 530 |
| | | GDSYVSWFAYWGQGTLVTVSSASTKGPSVFPLAPSSKSTSGGTAALGCLVKD | |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVQVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSEKNRVYLDT | |
| | | VSGSLTIYNLTSSDEDEYEMESPNITDTMKFFLYVLESLPSPTLTCALTNGS | |
| | | IEVQCMIPEHYNSHRGLIMYSWDCPMEQCKANSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVAKIR | SEQ ID NO: |
| | chain with | SKYNNYATYYADSVKDRFTISRDDSKNTAYLQMNNLKTEDTAVYYCVRHGNF | 602 |
| | a K residue | GDSYVSWFAYWGQGTLVTVSSASTKGPSVFPLAPSSKSTSGGTAALGQLVKD | |
| | prior to | YFPEPVTVSWNSGALTSGVHTSPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DIVSGSLTIYNLISSDEDEYEMESPNITDIMKFFLYVLESLPSPTLTCALTN | |
| | | GSIEVQCMIPEHYNSHRGLIMYSWDCPMEQCKANSTSIYFKMENDLPQKIQC | |
| | | TLSNPLENTTSSIILTTCIPSSGHSRHRY | |
| EIP0930 | CD20 light | DIVMTQTPLSLPVTPGEPASISCRSSKSLLHSNGITYLYWYLKKPGQSPQLD | SEQ ID NO: |
| | chain (L2- | IYQMSNLVSGVPDRFSGSGSGTDFTLKISRVEAEDVGVYYCAQNLELPYTFG | 531 |
| | 1) | GGTKVEIKRTVAAPSVFIFPPSDEQLKSGTASVVCLLNNFYPREAKVQWKVD | |
| | | NALQSGNSQESVTEQDSKDSTYSLSSTLRLSKADYEKHKVYACEVTHQGLSS | |
| | | PVTKSFNRGEC | |
| | CD20 heavy | EVQLVQSGAEVKKPGSSVKVSCKASGYAFSYSWINWVRDAPGQGLEWMGRIF | SEQ ID NO: |
| | chain (H2- | PGDGDTDYNGKFKGRVTITADKSTSTAYMELSSLRSEDTAVYYCARNVFDGY | 532 |
| | | WLVYWGQGTLVTVSSASTKGPSVFPLAPSSKSTSGGTAALGCLVDDYFPEPV | |
| | | TVSWNSGALTSGVHTFPAVLQSSGLYSLSSVVTVPSSSLGTQTYICNVNHKP | |
| | | SNTKVDKKVEPKSCDKTHTCPPCPAPEAAGAPSVFLFPPKPKDTLMISRTPE | |
| | | VTCVVVDVSHEDPEVKFNWYVDGVEVHNAKTKPREEQYNSTYRVVSVLTVLH | |
| | | QDWLNGKEYKCKVSNKALPAPIEKTISKAKGQPREPQVYTLPPCREEMTKNQ | |
| | | VSLWCLVKGFYPSDIAVEWESNGQPENNYKTTPPVLDSDGSFFLYSKLTVDK | |
| | | SRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 574 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGIQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSNLWVFGGGT | 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNDYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 533 |
| | | GNSYVSWFAYWGQGTLVTVSSASTKGPSVFPLAPSSKSTSGGTAALGQLVKD | |
| | | YPPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKFFLYVLESLPSPTLTCALTNGS | |
| | | IEVQCMIFEHYNSHRGLIMYSWDCEMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNDYAMNWVRKAPGKGLEWVARIR | SEQ ID NO: |
| | chain with | SKYNNYATYYADSVKDRFTISRDDSKNTAYLQMNNLKTEDTAVYYCVRHGNF | 603 |
| | a K residue | GNSYVSWFAYWGQGTLVTVSSASTKGPSVPPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVQVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDIMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0968 | CD20 light | DIVMTQTPLSLPVTPGEPASISCRSSKSLLHSNGITYLYWYLKKPGQSPQLL | SEQ ID NO: |
| | chain (L2- | IYQMSNLVSGVPDRFSGSGSGTDFTLKISRVEAEDVGVYYCAQNLELPYTFG | 534 |
| | 1) | GGTKVEIKRTVAAPSVFIFPPSDEQLKSGTASVVCLLNNFYPREAKVQWKVD | |
| | | NALQSGNSQESVTEQDSKDSTYSLSSTLRLSKADYEKHKVYACEVTHQGLSS | |
| | | PVTKSFNRGEC | |
| | CD20 heavy | EVQLVQSGAEVKKPGSSVKVSCKASGYAFSYSWINWVRDAPGQGLEWMGRIF | SEQ ID NO: |
| | chain (H2- | PGDGDTDYNGKFKGRVTITADKSTSTAYMELSSLRSEDTAVYYCARNVFDGY | 535 |
| | 1) | WLVYWGQGTLVTVSSASTKGPSVFPLAPSSKSTSGGTAALGCLVDDYFPEPV | |
| | | TVSWNSGALTSGVHTFPAVLQSSGLYSLSSVVTVPSSSLGTQTYICNVNHKP | |
| | | SNTKVDKKVEPKSCDKTHTCPPCPAPEAAGAPSVFLFPPKPKDTLMISRTPE | |
| | | VTCVVVDVSHEDPEVKFNWYVDGVEVHNAKTKPREEQYNSTYRVVSVLTVLH | |
| | | QDWLNGKEYKCKVSNKALPAPIEKTISKAKGQPREPQVYTLPPCREEMTKNQ | |
| | | VSLWCLVKGFYPSDIAVEWESNGQPENNYKTTPPVLDSDGSFFLYSKLTVDK | |
| | | SRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 575 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSIGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSQLWVFGGGT | 149 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSERSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 536 |
| | (N96S) | GDDYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVELFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKEFLYVLESLPSPTLTCALTNGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO : |
| | chain with | SKYNNYATYYADSVKDRFTISRDDSKNTAYLQMNNLKTEDTAVYYCVRHGNF | 604 |
| | a K residue | GDDYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | (N96S) | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | ILSNPLENTISSIILITCIPSSGHSRHRY | |
| EIP0891 | CD20 light | DIVMTQTPLSLPVTPGEPASISCRSSKSLLHSNGITYLYWYLKKPGQSPQLL | SEQ ID NO: |
| | chain (L2- | IYQMSNLVSGVPDRFSGSGSGTDFTLKISRVEAEDVGVYYCAQNLELPYTFG | 537 |
| | 1) | GGTKVEIKRTVAAPSVFIFPPSDEQLKSGTASVVCLLNNFYPREAKVQWKVD | |
| | | NALQSGNSQESVTEQDSKDSTYSLSSTERLSKADYEKRKVYACEVTHQGLSS | |
| | | PVTKSFNRGEC | |
| | CD20 heavy | EVQLVQSGAEVKKPGSSVKVSCKASGYAFSYSWINWVRDAPGQGLEWMGRIF | SEQ ID NO: |
| | chain (H2- | PGDGDTDYNGKFKGRVTITADKSTSTAYMELSSLRSEDTAVYYCARNVFDGY | 538 |
| | 1) | WLVYWGQGTLVTVSSASTKGPSVFPLAPSSKSTSGGTAALGCLVQDYFPEPV | |
| | | TVSWNSGALTSGVHTFPAVLQSSGLYSLSSVVTVPSSSLGTQTYICNVNHKP | |
| | | SNTKVDKKVEPKSCDKTHTCPPCPAPEAAGAPSVFLFPPKPKDTLMISRTPE | |
| | | VTCVVVDVSHEDPEVKFNWYVDGVEVHNAKTKPREEQYNSTYRVVSVLTVLH | |
| | | QDWLNGKEYKCKVSNKALPAPIEKTISKAKGQPREPQVYTLPPCREEMTKNQ | |
| | | VSLWCLVKGFYPSDIAVEWESNGQPENNYKTTPPVLDSDGSFFLYSKLTVDK | |
| | | SRWQQGNVFSCSVMREALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 576 |
| | residue | YYYGMDVWGQGTTVTVSSASTKGPSVFPLAPSSKSTSGGTAALGCLVDDYFP | |
| | at last | EPVTVSWNSGALTSGVHIFPAVLQSSGLYSLSSVVTVPSSSLGTQTYICNVN | |
| | position | HKPSNTKVDKKVEPKSCDKTHTCPPCPAPEAAGAPSVFLFPPKPKDELMISR | |
| | (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGD | SEQ ID NO: |
| | chain (L1) | TNKRAPGTPARFSGSLLGGKAALTLSGVQPEDEAEYYCALWYSNLWVFGGGT | 153 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 539 |
| | | GNSYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVQVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLTSSDEDEYEMESPNITDTMKFFLYVLESLPSPTLTCALTNGS | |
| | | IEVQCMIPEHYNSHRGLIMYSWDCPMEQCKRNSTSIYEKMENDLPQKIQCTL | |
| | | SNPLFNTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKDRFTISRDDSKNTAYLQMNNLKTEDTAVYYCVRHGNF | 605 |
| | a K residue | GNSYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVELFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DIVSGSLTIYNLISSDEDEYEMESPNITDIMKFFLYVLESLPSPTLTCALTN | |
| | | GSIEVQCMIPHYNSHRGLIMYSWDCPMEQCKERNSTSIYFKMENDLPQKIQC | |
| | | TLSNPLENTTSSIILTTCIPSSGHSRHRY | |
| EIP0868 | CD20 light | DIVMTQTPLSLPVTPGEPASISCRSSKSLLHSNGITYLYWYLKKPGQSPQLL | SEQ ID NO: |
| | chain (L2- | IYQMSNLVSGVPDRFSGSGSGTDFTLKISRVEAEDVGVYYCAQNLELPYTFG | 540 |
| | 1) | GGTKVEIKRTVAAPSVFIFPPSDEQLKSGTASVVCLLNNFYPREAKVQWKVD | |
| | | NALQSGNSQESVTEQDSKDSTYSLSSTLRLSKADYEKHKVYACEVTHQGLSS | |
| | | PVTKSFNRGEC | |
| | CD20 heavy | EVQLVQSGAEVKKPGSSVKVSCKASGYAFSYSWINWVRDAPGQGLEWMGRIF | SEQ ID NO: |
| | chain (H2- | PGDGDTDYNGKFKGRVTITADKSTSTAYMELSSLRSEDTAVYYCARNVFDGY | 541 |
| | 1) | WLVYWGQGTLVTVSSASTKGPSVFPLAPSSKSTSGGTAALGCLVDDYFPEPV | |
| | | TVSWNSGALTSGVHTFPAVLQSSGLYSLSSVVTVPSSSLGTQTYICNVNHKP | |
| | | SNTKVDKKVEPKSCDKTHTCPPCPAPEAAGAPSVFLFPPKPKDTLMISRTPE | |
| | | VTCVVVDVSHEDPEVKFNWYVDGVEVHNAKTKPREEQYNSTYRVVSVLTVLH | |
| | | QDWLNGKEYKCKVSNKALPAPIEKTISKAKGQPREPQVYTLPPCREEMTKNQ | |
| | | VSLWCLVKGFYPSDIAVEWESNGQPENNYKTTPPVLDSDGSFFLYSKLTVDK | |
| | | SRWQQGNVFSCSVMHEALHNHYTQKSLSLSPG | |
| | CD20 heavy | EVQLVESGGGLVQPGRSLRLSCAASGFTFNDYAMHWVRDAPGKGLEWVSTIS | SEQ ID NO: |
| | chain with | WNSGSIGYADSVKGRFTISRDNAKKSLYLQMNSLRAEDTALYYCAKDIQYGN | 577 |
| | K residue | YYYGMDVWGQGTTVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTQVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSNLWVFGGGT | 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSERSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 542 |
| | | GNSYVSWFAYWGQGTLVTVSSASTKGPSVFPLAPSSKSTSGGTAALGQLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVENAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKFFLYVLESLPSPTLTCALTNGS | |
| | | IEVQCMIPERYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKDRFTISRDDSKNTAYLQMNNLKTEDTAVYYCVRHGNF | 606 |
| | a K residue | GNSYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVQVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLISSDEDEYEMESPNITDTMKEFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0607 | Target | EIVLTQSPGTLSLSPGERATFSCRSSHSIRSRRVAWYQKKPGQAPRLVIHGV | SEQ ID NO: |
| (Control) | light chain | SNRASGISDRFSGSGSGTQFTLTITRVEPEDFALYYCQVYGASSYTFGQGTK | 543 |
| | (L2) | LERKRTVAAPSVFIFPPSDEQLKSGTASVVCLLNNFYPREAKVQWKVDNALQ | |
| | | SGNSQESVTEQDSKQSTYSLSSTLRLSKADYEKHKVYACEVTHQGLSSPVTK | |
| | | SFNRGEC | |
| | Target | QVQLVQSGARVKKPGASVKVSCQASGYRFSNFVIHWVRDAPGQRQEWMGWIN | SEQ ID NO: |
| | heavy chain | PYNGNKEFSAKFQDRVTFTADTSANTAYMELRSLRSADTAVYYCARVGPYSW | 544 |
| | (H2) | DDSPQDNYYMDVWGKGTTVIVSSASTKGPSVFPLAPSSKSTSGGTAALGCLV | |
| | | DDYFPEPVTVSWNSGALTSGVHTFPAVLQSSGLYSLSSVVTVPSSSLGTQTY | |
| | | ICNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTLPPC | |
| | | REEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPG | |
| | Target | QVQLVQSGAEVKKPGASVKVSCQASGYRFSNFVIHWVRDAPGQRQEWMGWIN | SEQ ID NO: |
| | heavy chain | PYNGNKEFSAKFQDRVTFTADTSANTAYMELRSLRSADTAVYYCARVGPYSW | 611 |
| | with K | DDSPQDNYYMDVWGKGTTVIVSSASTKGPSVFPLAPSSKSTSGGTAALGCLV | |
| | residue at | DDYFPEPVTVSWNSGALTSGVHTFPAVLQSSGLYSLSSVVTVPSSSLGTQTY | |
| | last | ICNVNHKPSNTKVDKKVEPKSCDKTHTCPPCPAPEAAGAPSVFLFPPKPKDT | |
| | position | LMISRTPEVTCVVVDVSHEDPEVKFNWYVDGVEVHNAKTKPREEQYNSTYRV | |
| | (H2) | VSVLTVLHQDWLNGKEYKCKVSNKALPAPIEKTISKAKGQPREPQVYTLPPC | |
| | | REEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSQLWVFGGGT | 149 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 545 |
| | | GDDYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKFFLYVLESLPSPTLTCALTNGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKDRFTISRQDSKNTAYLQMNNLKTEDTAVYYCVRHGNF | 612 |
| | a K residue | GDDYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVIFHVPSNVPLKEVLWKKQKDKVAELENSEFRAFSSFKNRVYL | |
| | | DTVSGSLTIYNLISSDEDEYEMESPNITDIMKFFLYVLESLPSPTLTCALTN | |
| | | GSIEVQCMIPEHYNSHRGLIMYSWDCPMEQCKRNSTSIYFKMENDLPQKIQC | |
| | | ILSNPLENTTSSIILTTCIPSSGHSRHRY | |
| EIP0614 | Target | EIVETQSPGTLSLSPGERATFSCRSSHSIRSRRVAWYQKKPGQAPRLVIHGV | SEQ ID NO: |
| (Control) | light chain | SNRASGISDRFSGSGSGTDFTLTITRVEPEDFALYYCQVYGASSYTFGQGTK | 543 |
| | (L2) | LERKRTVAAPSVFIFPPSDEQLKSGTASVVCLLNNFYPREAKVQWKVDNALQ | |
| | | SGNSQESVTEQDSKDSTYSLSSTLRLSKADYEKHKVYACEVTHQGLSSPVTK | |
| | | SFNRGEC | |
| | Target | QVQLVQSGAEVKKPGASVKVSCQASGYRFSNFVIHWVRDAPGQRQEWMGWIN | SEQ ID NO: |
| | heavy chain | PYNGNKEFSAKFQDRVTFTADTSANTAYMELRSLRSADTAVYYCARVGPYSW | 544 |
| | (H2) | DDSPQDNYYMDVWGKGTTVIVSSASTKGPSVFPLAPSSKSTSGGTAALGCLV | |
| | | DDYFPEPVTVSWNSGALTSGVHTFPAVLQSSGLYSLSSVVTVPSSSLGTQTY | |
| | | ICNVNHKPSNTKVDKKVEPKSCDKTHTCPPCPAPEAAGAPSVFLFPPKPKDT | |
| | | LMISRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTLPPC | |
| | | REEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPG | |
| | Target | QVQLVQSGAEVKKPGASVKVSCQASGYRFSNFVIHWVRDAPGQRQEWMGWIN | SEQ ID NO: |
| | heavy chain | PYNGNKEFSAKFQDRVTFTADTSANTAYMELRSLRSADTAVYYCARVGPYSW | 613 |
| | with K | DDSPQDNYYMDVWGKGTTVIVSSASTKGPSVFPLAPSSKSTSGGTAALGCLV | |
| | residue at | DDYFPEPVTVSWNSGALTSGVHTFPAVLQSSGLYSLSSVVTVPSSSLGTQTY | |
| | last | ICNVNHKPSNTKVDKKVEPKSCDKTHTCPPCPAPEAAGAPSVFLFPPKPKDT | |
| | position | LMISRTPEVTCVVVQVSHEDPEVKFNWYVDGVEVHNAKTKPREEQYNSTYRV | |
| | (H2) | VSVLTVLHQDWLNGKEYKCKVSNKALPAPIEKTISKAKGQPREPQVYTLPPC | |
| | | REEMTKNQVSLWCLVKGFYPSDIAVEWESNGQPENNYKTTPPVLDSDGSFFL | |
| | | YSKLTVDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSNLWVFGGGT | 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSERSYSCQVTHEGSTVEKT | |
| | | VAPTESS | |
| | CD3 heavy | EVQLLESGGGLVQPGGSLKLSCAASGFTFNDYAMNWVRKAPGKGLEWVARIR | SEQ ID NO: |
| | chain (H1) | SKYNNYATYYADSVKDRFTISRDDSKNTAYLQMNNIKTEDTAVYYCVRHGNF | 545 |
| | | GNSYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVENAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKEFLYVLESLPSPTLTCALTNGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNDYAMNWVRKAPGKGLEWVARIR | SEQ ID NO: |
| | chain with | SKYNNYATYYADSVKDRFTISRDDSKNTAYLQMNNLKTEDTAVYYCVRHGNF | 614 |
| | a K residue | GNSYVSWPAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIFEHYNSHRGLIMYSWDCPMEQCKRNSTSIYFKMENDLPQKIQC | |
| | | ILSNPLENTISSIILITCIFSSGHSRHRY | |
| EIP0863 | CD20 light | DIQMTQSPSSLSASVGDRVTITCRASSSVSYMHWYQKKPGKAPKPLIYAPSN | SEQ ID NO: |
| (Mosun- | chain (L2) | LASGVPSRFSGSGSGTDFTLTISSLQPEDFATYYCQQWSENPPTFGQGTKVE | 546 |
| etuzumab | | IKRTVAAPSVFIFPPSDEQLKSGTASVVCLLNNFYPREAKVQWKVDNALQSG | |
| surro- | | NSQESVTEQDSKDSTYSLSSTLRLSKADYEKHKVYACEVTHQGLSSPVTKSF | |
| gate) | | NRGEC | |
| | CD20 heavy | EVQLVESGGGLVQPGGSLRLSCAASGYTFTSYNMHWVRDAPGKGLEWVGAIY | SEQ ID NO: |
| | chain (H2) | PGNGDTSYNQKEKGRFTISVDKSKNTLYLQMNSLRAEDTAVYYCARVVYYSN | 547 |
| | | SYWYFDVWGQGTLVTVSSASTKGPSVFPLAPSSKSTSGGTAALGCLVQDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVQVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPG | |
| | CD20 heavy | EVQLVESGGGLVQPGGSLRLSCAASGYTFTSYNMAWVRDAPGKGLEWVGAIY | SEQ ID NO: |
| | chain with | PGNGDTSYNQKEKGRFTISVDKSKNTLYLQMNSLRAEDTAVYYCARVVYYSN | 615 |
| | K residue | SYWYFDVWGQGTLVTVSSASTKGPSVFPLAPSSKSTSGGTAALGCLVDDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) (H2) | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSNLWVFGGGT | 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNLKTEDTAVYYCVRHGNF | 548 |
| | | GNSYVSWFAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVQVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPG | |
| EIP0864 | CD20 light | DIQMTQSPSSLSASVGDRVTITCRASSSVSYMHWYQKKPGKAPKPLIYAPSN | SEQ ID NO: |
| (Mosun- | chain (L2) | LASGVPSRFSGSGSGTDFTLTISSLQPEDFATYYCQQWSENPPTFGQGTKVE | 546 |
| etuzumab | | IKRTVAAPSVFIFPPSDEQLKSGTASVVCLLNNFYPREAKVQWKVDNALQSG | |
| surro- | | NSQESVTEQDSKDSTYSLSSTLRLSKADYEKHKVYACEVTHQGLSSPVTKSF | |
| gate_ | | NRGEC | |
| CD58) | CD20 heavy | EVQLVESGGGLVQPGGSLRLSCAASGYTFTSYNMHWVRDAPGKGLEWVGAIY | SEQ ID NO: |
| | chain (H2) | PGNGDTSYNQKEKGRFTISVDKSKNTLYLQMNSLRAEDTAVYYCARVVYYSN | 547 |
| | | SYWYFDVWGQGTLVTVSSASTKGPSVFPLAPSSKSTSGGTAALGCLVQDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVDVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPG | |
| | CD20 heavy | EVQLVESGGGLVQPGGSLRLSCAASGYTFTSYNMHWVRDAPGKGLEWVGAIY | SEQ ID NO: |
| | chain with | PGNGDTSYNQKEKGRFTISVDKSKNTLYLQMNSLRAEDTAVYYCARVVYYSN | 616 |
| | residue | SYWYFDVWGQGTLVTVSSASTKGPSVFPLAPSSKSTSGGTAALGCLVQDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) (H2) | TPEVTCVVVQVSHEDPEVKFNWYVDGVEVHNAKTKPREEQYNSTYRVVSVLT | |
| | | VLHQDWLNGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID NO: |
| | chain (L1) | TNKRAPGTPARFSGSLLGGKAALTLSGVQPEDEAEYYCALWYSNLWVFGGGT | 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSERSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID NO: |
| | chain (H1) | SKYNNYATYYADSVKDRFTISRDDSKNTAYLQMNNTKTEDTAVYYCVRHGNF | 549 |
| | | GNSYVSWEAYWGQGTLVTVSSASTKGPSVFPLAPSSKSTSGGTAALGCLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLISSDEDEYEMESPNITDTMKEFLYVLESLPSPTLTCALTNGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| | CD3 heavy | EVQLLESGGGLVQPGGSLKLSCAASGFTFNTYAMNWVRKAPGKGLEWVARIR | SEQ ID NO: |
| | chain with | SKYNNYATYYADSVKDRFTISRDDSKNTAYLQMNNLKTEDTAVYYCVRHGNF | 617 |
| | a K residue | GNSYVSWFAYWGQGTLVTVSSASTKGPSVFPLAPSSKSTSGGTAALGCLVKD | |
| | prior to | YFPEPVTVSWNSGALTSGVHTFPACLQSSGLYSLSSVVTVPSSSLGTQTYIC | |
| | linker (H1) | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIFEHYNSHRGLIMYSWDCPMEQCKRNSTSIYFKMENDLPQKIQC | |
| | | TLSNPLENTISSIILITCIESSGHSRHRY | |
| EIP0887- | CD20 light | DIVMTQTPLSLPVTPGEPASISCRSSKSLLHSNGITYLYWYLQKPGQSPQLL | SEQ ID NO: |
| Glo- | chain (L2) | IYQMSNLVSGVPDRFSGSGSGTDFTLKISRVEAEDVGVYYCAQNLELPYTFG | 550 |
| fitamab | | GGTKVEIKRTVAAPSVFIFPPSDRKLKSGTASVVCLLNNFYPREAKVQWKVD | |
| | | NALQSGNSQESVTEQDSKDSTYSLSSTLTLSKADYEKRKVYACEVTHQGLSS | |
| | | PVTKSFNRGEC | |
| | CD20 heavy | QVQLVQSGAEVKKPGSSVKVSCKASGYAFSYSWINWVRQAPGQGLEWMGRIF | SEQ ID NO: |
| | chain (H2) | PGDGDTDYNGKFKGRVTITADKSTSTAYMELSSLRSEDTAVYYCARNVFDGY | 551 |
| | | WLVYWGQGTLVTVSSASTKGPSVFPLAPSSKSTSGGTAALGCLVEDYFPEPV | |
| | | TVSWNSGALTSGVHTFPAVLQSSGLYSLSSVVTVPSSSLGTQTYICNVNHKP | |
| | | SNTKVDEKVEPKSCDKTHTCPPCPAPEAAGGPSVFLFPPKPKDTLMISRTPE | |
| | | VTCVVVDVSHEDPEVKFNWYVDGVEVHNAKTKPREEQYNSTYRVVSVLTVLH | |
| | | QDWLNGKEYKCKVSNKALGAPIEKTISKAKGQPREPQVCTLPPSRDELTKNQ | |
| | | VSLSCAVKGFYPSDIAVEWESNGQPENNYKTTPPVLDSDGSFFLVSKLTVDK | |
| | | SRWQQGNVFSCSVMHEALHNHYTQKSLLSPG | |
| | CD20 heavy | QVQLVQSGARVKKPGSSVKVSCKASGYARSYSWINWVRQAPGQGLEWMGRIF | SEQ ID NO: |
| | chain with | PGDGDTDYNGKFKGRVTITADKSTSTAYMELSSLRSEDTAVYYCARNVFDGY | 618 |
| | K residue | WLVYWGQGTLVTVSSASTKGPSVFPLAPSSKSTSGGTAALGCLVEDYFPEPV | |
| | at last | TVSWNSGALTSGVHTFPAVLQSSGLYSLSSVVTVPSSSLGTQTYICNVNHKP | |
| | position | SNTKVDEKVEPKSCDKTHTCPPCPAPEAAGGPSVFLFPPKPKDTLMISRTPE | |
| | (H2) (H2) | VTCVVVDVSHEDPEVKFNWYVDGVEVHNAKTKPREEQYNSTYRVVSVLTVLH | |
| | | QDWLNGKEYKCKVSNKALGAPIEKTISKAKGQPREPQVCTLPPSRDELTKNQ | |
| | | VSLSCAVKGFYPSDIAVEWESNGQPENNYKTTPPVLDSDGSFFLVSKLTVDK | |
| | | SRWQQGNVFSCSVMREALHNHYTQKSLLSPGK | |
| | CD3 light | EVQLLESGGGLVQPGGSLRLSCAASGFTFSTYAMNWVRQAPGKGLEWVSRIR | SEQ ID NO: |
| | chain (L1) | SKYNNYATYYADSVKGRFTISRDDSKNTLYLQMNSLRAEDTAVYYCVRHGNF | 552 |
| | | GNSYVSWFAYWGQGTLVTVSSASVAAPSVFIFPPSDEQLKSGTASVVCLLNN | |
| | | FYPREAKVQWKVDNALQSGNSQESVTEQDSKDSTYSLSSTLTLSKADYEKHK | |
| | | VYACEVTHQGLSSPVTKSFNRGEC | |
| | CD20 and | QVQLVQSGAEVKKPGSSVKVSCKASGYAFSYSWINWVRQAPGQGLEWMGRIF | SEQ ID NO: |
| | CD3 heavy | PGDGDTDYNGKFKGRVTITADKSTSTAYMELSSLRSEDTAVYYCARNVFDGY | 619 |
| | chain with | WLVYWGQGTLVTVSSASTKGPSVFPLAPSSKSTSGGTAALGCLVEDYFPEPV | |
| | K residue | TVSWNSGALTSGVHTFPAVLQSSGLYSLSSVVTVPSSSLGTQTYICNVNHKP | |
| | at last | SNTKVDEKVEPKSCDGGGGSGGGGSQAVVTQEPSLTVSPGGTVTLTCGSSTG | |
| | position | AVTTSNYANWVQEKPGQAFRGLIGGTNKRAPGTPARFSGSLEGGKAALTLSG | |
| | (H1) | AQPEDEAEYYCALWYSNLWVFGGGTKLTVLSSASTKGPSVFPLAPSSKSTSG | |
| | | GTAALGCLVKDYFPEPVTVSWNSGALTSGVHTFPAVLQSSGLYSLSSVVTVP | |
| | | SSSLGTQTYICNVNRKPSNTKVDKKVEPKSCDKTHTCPPCPAPEAAGGPSVF | |
| | | LFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALGAPIEKTISKAKGQPRE | |
| | | PQVYTLPPCRDELTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | CD20 and | QVQLVQSGAEVKKPGSSVKVSCKASGYAFSYSWINWVRQAPGQGLEWMGRIF | SEQ ID NO: |
| | CD3 heavy | PGDGDTDYNGKFKGRVTITADKSTSTAYMELSSLRSEDTAVYYCARNVFDGY | 553 |
| | chain (H1) | WLVYWGQGTLVTVSSASTKGPSVFPLAPSSKSTSGGTAALGCLVEDYFPEPV | |
| | | TVSWNSGALTSGVHTFPAVLQSSGLYSLSSVVTVPSSSLGTQTYICNVNHKP | |
| | | SNTKVDEKVEPKSCDGGGGSGGGGSQAVVTQEPSLTVSPGGTVTLTCGSSTG | |
| | | AVTTSNYANWVQEKPGQAFRGLIGGTNKRAPGTPARFSGSLLGGKAALTLSG | |
| | | AQPEDEAEYYCALWYSNLWVFGGGTKLTVLSSASTKGPSVFPLAPSSKSTSG | |
| | | GTAALGCLVKDYFPEPVTVSWNSGALTSGVHTFPAVLQSSGLYSLSSVVTVP | |
| | | SSSLGTQTYICNVNHKPSNTKVDKKVEPKSCDKTHTCPPCPAPEAAGGPSVF | |
| | | LFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALGAPIEKTISKAKGQPRE | |
| | | PQVYTLPPCRDELTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPG | |

Exemplary, anti-CD3ε×anti-CD20 bispecific antibodies with a C-terminus fusion peptide of the invention include EIP0969, EIP0969-2, EIP0969-3, EIP0969-4, EIP0892. EIP0892-2, EIP0892-3, EIP0892-4, EIP0929. EIP0929-2, EIP0929-3, EIP0929-4, EIP0893, EIP0893-2, EIP0893-3, EIP0893-4, EIP0886, EIP0886-2, EIP0886-3, EIP0886-4, EIP0866, EIP0866-2, EIP0866-3, EIP0866-4, EIP0966, EIP0966-2, EIP0966-3, EIP0966-4, EIP0967, EIP0967-2, EIP0967-3, EIP0967-4, EIP0930, EIP0930-2, EIP0930-3, EIP0930-4, EIP0968, EIP0968-2, EIP0968-3, EIP0968-4, EIP0891, EIP0891-2, EIP0891-3, EIP0891-4, EIP0868, EIP0868-2, EIP0868-3, and EIP0868-4.

In some embodiments, the bispecific antibody EIP0969, EIP0969-2, EIP0969-3, EIP0969-4, EIP0966, EIP0966-2, EIP0966-3, and EIP0966-4, comprises a first antigen binding domain that binds CD3ε, comprising a VH1_CDR1 comprising the amino acid sequence of SEQ ID NO: 29: a VH1_CDR2 comprising the amino acid sequence of SEQ ID NO: 34; a VH1_CDR3 comprising the amino acid sequence of SEQ ID NO: 40; a VL1_CDR1 comprising the amino acid sequence of SEQ ID NO: 42: a VL1_CDR2 comprising the amino acid sequence of SEQ ID NO: 43; and a VL1_CDR3 comprising the amino acid sequence of SEQ ID NO: 47.

In some embodiments, the bispecific antibody EIP0892, EIP0892-2, EIP0892-3, EIP0892-4, EIP0967, EIP0967-2, EIP0967-3, and EIP0967-4, comprises a first antigen binding domain that binds CD3ε, comprising a VH1_CDR1 comprising the amino acid sequence of SEQ ID NO: 29: a VH1_CDR2 comprising the amino acid sequence of SEQ ID NO: 35; a VH1_CDR3 comprising the amino acid sequence of SEQ ID NO: 38: a VL1_CDR1 comprising the amino acid sequence of SEQ ID NO: 42; a VL1_CDR2 comprising the amino acid sequence of SEQ ID NO: 43; and a VL1_CDR3 comprising the amino acid sequence of SEQ ID NO: 47.

In some embodiments, the bispecific antibody EIP0929, EIP0929-2, EIP0929-3, EIP0929-4, EIP0930, EIP0930-2, EIP0930-3, and EIP0930-4, comprises a first antigen binding domain that binds CD3ε, comprising a VH1_CDR1 comprising the amino acid sequence of SEQ ID NO: 30: a VH1_CDR2 comprising the amino acid sequence of SEQ ID NO: 34: a VH1_CDR3 comprising the amino acid sequence of SEQ ID NO: 37: a VL1_CDR1 comprising the amino acid sequence of SEQ ID NO: 42; a VL1_CDR2 comprising the amino acid sequence of SEQ ID NO: 43; and a VL1_CDR3 comprising the amino acid sequence of SEQ ID NO: 45.

In some embodiments, the bispecific antibody EIP0893, EIP0893-2, EIP0893-3, EIP0893-4, EIP0968, EIP0968-2, EIP0968-3, and EIP0968-4, comprises a first antigen binding domain that binds CD3ε, comprising a VH1_CDR1 comprising the amino acid sequence of SEQ ID NO: 29; a VH1_CDR2 comprising the amino acid sequence of SEQ ID NO: 34: a VH1_CDR3 comprising the amino acid sequence of SEQ ID NO: 39; a VL1_CDR1 comprising the amino acid sequence of SEQ ID NO: 42; a VL1_CDR2 comprising the amino acid sequence of SEQ ID NO: 43; and a VL1 CDR3 comprising the amino acid sequence of SEQ ID NO: 47.

In some embodiments, the bispecific antibody EIP0886, EIP0886-2, EIP0886-3, EIP0886-4, EIP0891, EIP0891-2, EIP0891-3, and EIP0891-4, comprises a first antigen binding domain that binds CD3ε, comprising a VH1_CDR1 comprising the amino acid sequence of SEQ ID NO: 29; a VH1_CDR2 comprising the amino acid sequence of SEQ ID NO: 34; a VH1_CDR3 comprising the amino acid sequence of SEQ ID NO: 37; a VL1_CDR1 comprising the amino acid sequence of SEQ ID NO: 42; a VL1_CDR2 comprising the amino acid sequence of SEQ ID NO: 44; and a VL1 CDR3 comprising the amino acid sequence of SEQ ID NO: 45.

In some embodiments, the bispecific antibody EIP0866, EIP0866-2, EIP0866-3, EIP0866-4, EIP0868, EIP0868-2, EIP0868-3, and EIP0868-4, comprises a first antigen binding domain that binds CD3ε, comprising a VH1_CDR1 comprising the amino acid sequence of SEQ ID NO: 29; a VH1_CDR2 comprising the amino acid sequence of SEQ ID NO: 34; a VH1_CDR3 comprising the amino acid sequence of SEQ ID NO: 37; a VL1_CDR1 comprising the amino acid sequence of SEQ ID NO: 42; a VL1_CDR2 comprising the amino acid sequence of SEQ ID NO: 43; and a VL1_CDR3 comprising the amino acid sequence of SEQ ID NO: 45.

In some embodiments, the bispecific antibody EIP0969, EIP0969-2, EIP0969-3, EIP0969-4, EIP0892, EIP0892-2, EIP0892-3, EIP0892-4, EIP0929, EIP0929-2, EIP0929-3, EIP0929-4, EIP0893, EIP0893-2, EIP0893-3, EIP0893-4, EIP0886, EIP0886-2, EIP0886-3, EIP0886-4, EIP0866, EIP0866-2, EIP0866-3 and EIP0866-4, comprises a second antigen binding domain that binds CD20, comprising a VH2_CDR1 comprising the amino acid sequence of SEQ ID NO: 459; a VH2_CDR2 comprising the amino acid sequence of SEQ ID NO: 460; a VH2_CDR3 comprising the amino acid sequence of SEQ ID NO: 462: a VL2_CDR1 comprising the amino acid sequence of SEQ ID NO: 466: a VL2_CDR2 comprising the amino acid sequence of SEQ ID NO: 467; and a VL2_CDR3 comprising the amino acid sequence of SEQ ID NO: 468.

In some embodiments, the bispecific antibody EIP0966, EIP0966-2, EIP0966-3, EIP0966-4, EIP0967, EIP0967-2, EIP0967-3, EIP0967-4, EIP0930, EIP0930-2, EIP0930-3, EIP0930-4, EIP0968, EIP0968-2, EIP0968-3, EIP0968-4, EIP0891, EIP0891-2, EIP0891-3, EIP0891-4, EIP0868, EIP0868-2, EIP0868-3, and EIP0868-4, comprises a second antigen binding domain that binds CD20, comprising a VH2_CDR1 comprising the amino acid sequence of SEQ ID NO: 455; a VH2_CDR2 comprising the amino acid sequence of SEQ ID NO: 457; a VH2_CDR3 comprising the amino acid sequence of SEQ ID NO: 458; a VL2_CDR1 comprising the amino acid sequence of SEQ ID NO: 463; a VL2_CDR2 comprising the amino acid sequence of SEQ ID NO: 464; and a VL2_CDR3 comprising the amino acid sequence of SEQ ID NO: 465.

In some embodiments, the bispecific antibody EIP0969 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0969-2 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0969-3 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0969-4 comprises a VH1 comprising the amino acid sequence of SEQ ID NO: 19, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0892 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0892-2 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0892-3 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0892-4 comprises a VH1 comprising the amino acid sequence of SEQ ID NO: 18, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0929 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0929-2 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0929-3 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0929-4 comprises a VH1 comprising the amino acid sequence of SEQ ID NO: 17, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0893 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0893-2 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0893-3 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0893-4 comprises a VH1 comprising the amino acid sequence of SEQ ID NO: 16, a VL1 comprising the amino acid sequence of SEQ ID NO: 26, a VH2 comprising the amino acid sequence of SEQ ID NO: 453, and a VL2 comprising the amino acid sequence of SEQ ID NO: 452.

In some embodiments, the bispecific antibody EIP0886 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0886-2 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0886-3 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0886-4 comprises a VH1 comprising the amino acid sequence of SEQ ID NO: 13, a VL1 comprising the amino acid sequence of SEQ ID NO: 27, a VH2 comprising the amino acid sequence of SEQ ID NO: 453, and a VL2 comprising the amino acid sequence of SEQ ID NO: 452.

In some embodiments, the bispecific antibody EIP0866 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0866-2 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0866-3 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0866-4 comprises a VH1 comprising the amino acid sequence of SEQ ID NO: 13, a VL1 comprising the amino acid sequence of SEQ ID NO: 22, a VH2 comprising the amino acid sequence of SEQ ID NO: 453, and a VL2 comprising the amino acid sequence of SEQ ID NO: 452.

In some embodiments, the bispecific antibody EIP0966 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0966-2 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0966-3 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0966-4 comprises a VH1 comprising the amino acid sequence of SEQ ID NO: 19, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0967 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0967-2 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0967-3 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0967-4 comprises a VH1 comprising the amino acid sequence of SEQ ID NO: 18, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0930 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0930-2 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0930-3 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0930-4 comprises a VH1 comprising the amino acid sequence of SEQ ID NO: 17, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0968 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0968-2 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0968-3 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0968-4 comprises a VH1 comprising the amino acid sequence of SEQ ID NO: 16, a VL1 comprising the amino acid sequence of SEQ ID NO: 26, a VH2 comprising the amino acid sequence of SEQ ID NO: 451, and a VL2 comprising the amino acid sequence of SEQ ID NO: 450.

In some embodiments, the bispecific antibody EIP0891 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0891-2 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0891-3 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0891-4 comprises a VH1 comprising the amino acid sequence of SEQ ID NO: 13, a VL1 comprising the amino acid sequence of SEQ ID NO: 27, a VH2 comprising the amino acid sequence of SEQ ID NO: 451, and a VL2 comprising the amino acid sequence of SEQ ID NO: 450.

In some embodiments, the bispecific antibody EIP0868 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0868-2 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0868-3 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0868-4 comprises a VH1 comprising the amino acid sequence of SEQ ID NO: 13, a VL1 comprising the amino acid sequence of SEQ ID NO: 22, a VH2 comprising the amino acid sequence of SEQ ID NO: 451, and a VL2 comprising the amino acid sequence of SEQ ID NO: 450.

In some embodiments, the bispecific antibody EIP0960 comprises a H1 comprising the amino acid sequence of SEQ ID NO: 473, a L1 comprising the amino acid sequence of SEQ ID NO: 149, a H2 comprising the amino acid sequence of SEQ ID NO: 472, and a L2 comprising the amino acid sequence of SEQ ID NO: 471.

In some embodiments, the bispecific antibody EIP0960-2 comprises a H1 comprising the amino acid sequence of SEQ ID NO: 473, a L1 comprising the amino acid sequence of SEQ ID NO: 149, a H2 comprising the amino acid sequence of SEQ ID NO: 554, and a L2 comprising the amino acid sequence of SEQ ID NO: 471.

In some embodiments, the bispecific antibody EIP0960-3 comprises a H1 comprising the amino acid sequence of SEQ ID NO: 583, a L1 comprising the amino acid sequence of SEQ ID NO: 149, a H2 comprising the amino acid sequence of SEQ ID NO: 472, and a L2 comprising the amino acid sequence of SEQ ID NO: 471.

In some embodiments, the bispecific antibody EIP0960-4 comprises a H1 comprising the amino acid sequence of SEQ ID NO: 583, a L1 comprising the amino acid sequence of SEQ ID NO: 149, a H2 comprising the amino acid sequence of SEQ ID NO: 554, and a L2 comprising the amino acid sequence of SEQ ID NO: 471.

In some embodiments, the bispecific antibody EIP0963 comprises a H1 comprising the amino acid sequence of SEQ ID NO: 476, a L1 comprising the amino acid sequence of SEQ ID NO: 149, a H2 comprising the amino acid sequence of SEQ ID NO: 475, and a L2 comprising the amino acid sequence of SEQ ID NO: 471.

In some embodiments, the bispecific antibody EIP0963-2 comprises a H1 comprising the amino acid sequence of SEQ ID NO: 476, a L1 comprising the amino acid sequence of SEQ ID NO: 149, a H2 comprising the amino acid sequence of SEQ ID NO: 555, and a L2 comprising the amino acid sequence of SEQ ID NO: 471.

In some embodiments, the bispecific antibody EIP0963-3 comprises a H1 comprising the amino acid sequence of SEQ ID NO: 584, a L1 comprising the amino acid sequence of SEQ ID NO: 149, a H2 comprising the amino acid sequence of SEQ ID NO: 475, and a L2 comprising the amino acid sequence of SEQ ID NO: 471.

In some embodiments, the bispecific antibody EIP0963-4 comprises a H1 comprising the amino acid sequence of SEQ ID NO: 584, a L1 comprising the amino acid sequence of SEQ ID NO: 149, a H2 comprising the amino acid sequence of SEQ ID NO: 555, and a L2 comprising the amino acid sequence of SEQ ID NO: 471.

In some embodiments, the bispecific antibody EIP0961 comprises a H1 comprising the amino acid sequence of SEQ ID NO: 479, a L1 comprising the amino acid sequence of SEQ ID NO: 141, a H2 comprising the amino acid sequence of SEQ ID NO: 478, and a L2 comprising the amino acid sequence of SEQ ID NO: 471.

In some embodiments, the bispecific antibody EIP0961-2 comprises a H1 comprising the amino acid sequence of SEQ ID NO: 479, a L1 comprising the amino acid sequence of SEQ ID NO: 141, a H2 comprising the amino acid sequence of SEQ ID NO: 556, and a L2 comprising the amino acid sequence of SEQ ID NO: 471.

In some embodiments, the bispecific antibody EIP0961-3 comprises a H1 comprising the amino acid sequence of SEQ ID NO: 585, a L1 comprising the amino acid sequence of SEQ ID NO: 141, a H2 comprising the amino acid sequence of SEQ ID NO: 478, and a L2 comprising the amino acid sequence of SEQ ID NO: 471.

In some embodiments, the bispecific antibody EIP0961-4 comprises a H1 comprising the amino acid sequence of SEQ ID NO: 585, a L1 comprising the amino acid sequence of SEQ ID NO: 141, a H2 comprising the amino acid sequence of SEQ ID NO: 556, and a L2 comprising the amino acid sequence of SEQ ID NO: 471.

In some embodiments, the bispecific antibody EIP0964 comprises a H1 comprising the amino acid sequence of SEQ ID NO: 482, a L1 comprising the amino acid sequence of SEQ ID NO: 149, a H2 comprising the amino acid sequence of SEQ ID NO: 481, and a L2 comprising the amino acid sequence of SEQ ID NO: 471.

In some embodiments, the bispecific antibody EIP0964-2 comprises a H1 comprising the amino acid sequence of SEQ ID NO: 482, a L1 comprising the amino acid sequence of SEQ ID NO: 149, a H2 comprising the amino acid sequence of SEQ ID NO: 557, and a L2 comprising the amino acid sequence of SEQ ID NO: 471.

In some embodiments, the bispecific antibody EIP0964-3 comprises a H1 comprising the amino acid sequence of SEQ ID NO: 586, a L1 comprising the amino acid sequence of SEQ ID NO: 149, a H2 comprising the amino acid sequence of SEQ ID NO: 481, and a L2 comprising the amino acid sequence of SEQ ID NO: 471.

In some embodiments, the bispecific antibody EIP0964-4 comprises a H1 comprising the amino acid sequence of SEQ ID NO: 586, a L1 comprising the amino acid sequence of SEQ ID NO: 149, a H2 comprising the amino acid sequence of SEQ ID NO: 557, and a L2 comprising the amino acid sequence of SEQ ID NO: 471.

In some embodiments, the bispecific antibody EIP0962 comprises a H1 comprising the amino acid sequence of SEQ ID NO: 485, a L1 comprising the amino acid sequence of SEQ ID NO: 153, a H2 comprising the amino acid sequence of SEQ ID NO: 484, and a L2 comprising the amino acid sequence of SEQ ID NO: 471.

In some embodiments, the bispecific antibody EIP0962-2 comprises a H1 comprising the amino acid sequence of SEQ ID NO: 485, a L1 comprising the amino acid sequence of SEQ ID NO: 153, a H2 comprising the amino acid sequence of SEQ ID NO: 558, and a L2 comprising the amino acid sequence of SEQ ID NO: 471.

In some embodiments, the bispecific antibody EIP0962-3 comprises a H1 comprising the amino acid sequence of SEQ ID NO: 587, a L1 comprising the amino acid sequence of SEQ ID NO: 153, a H2 comprising the amino acid sequence of SEQ ID NO: 484, and a L2 comprising the amino acid sequence of SEQ ID NO: 471.

In some embodiments, the bispecific antibody EIP0962-4 comprises a H1 comprising the amino acid sequence of SEQ ID NO: 587, a L1 comprising the amino acid sequence of SEQ ID NO: 153, a H2 comprising the amino acid sequence of SEQ ID NO: 558, and a L2 comprising the amino acid sequence of SEQ ID NO: 471.

In some embodiments, the bispecific antibody EIP0865 comprises a H1 comprising the amino acid sequence of SEQ ID NO: 488, a L1 comprising the amino acid sequence of SEQ ID NO: 141, a H2 comprising the amino acid sequence of SEQ ID NO: 487, and a L2 comprising the amino acid sequence of SEQ ID NO: 471.

In some embodiments, the bispecific antibody EIP0865-2 comprises a H1 comprising the amino acid sequence of SEQ ID NO: 488, a L1 comprising the amino acid sequence of SEQ ID NO: 141, a H2 comprising the amino acid sequence of SEQ ID NO: 559, and a L2 comprising the amino acid sequence of SEQ ID NO: 471.

In some embodiments, the bispecific antibody EIP0865-3 comprises a H1 comprising the amino acid sequence of SEQ ID NO: 588, a L1 comprising the amino acid sequence of SEQ ID NO: 141, a H2 comprising the amino acid sequence of SEQ ID NO: 487, and a L2 comprising the amino acid sequence of SEQ ID NO: 471.

In some embodiments, the bispecific antibody EIP0865-4 comprises a H1 comprising the amino acid sequence of SEQ ID NO: 588, a L1 comprising the amino acid sequence of SEQ ID NO: 141, a H2 comprising the amino acid sequence of SEQ ID NO: 559, and a L2 comprising the amino acid sequence of SEQ ID NO: 471.

In some embodiments, the bispecific antibody EIP0826 comprises a H1 comprising the amino acid sequence of SEQ ID NO: 509, a L1 comprising the amino acid sequence of SEQ ID NO: 149, a H2 comprising the amino acid sequence of SEQ ID NO: 508, and a L2 comprising the amino acid sequence of SEQ ID NO: 507.

In some embodiments, the bispecific antibody EIP0826-2 comprises a H1 comprising the amino acid sequence of SEQ ID NO: 509, a L1 comprising the amino acid sequence of SEQ ID NO: 149, a H2 comprising the amino acid sequence of SEQ ID NO: 560, and a L2 comprising the amino acid sequence of SEQ ID NO: 507.

In some embodiments, the bispecific antibody EIP0826-3 comprises a H1 comprising the amino acid sequence of SEQ ID NO: 589, a L1 comprising the amino acid sequence of SEQ ID NO: 149, a H2 comprising the amino acid sequence of SEQ ID NO: 508, and a L2 comprising the amino acid sequence of SEQ ID NO: 507.

In some embodiments, the bispecific antibody EIP0826-4 comprises a H1 comprising the amino acid sequence of SEQ ID NO: 589, a L1 comprising the amino acid sequence of SEQ ID NO: 149, a H2 comprising the amino acid sequence of SEQ ID NO: 560, and a L2 comprising the amino acid sequence of SEQ ID NO: 507.

In some embodiments, the bispecific antibody EIP0825 comprises a H1 comprising the amino acid sequence of SEQ ID NO: 512, a L1 comprising the amino acid sequence of SEQ ID NO: 149, a H2 comprising the amino acid sequence of SEQ ID NO: 511, and a L2 comprising the amino acid sequence of SEQ ID NO: 510.

In some embodiments, the bispecific antibody EIP0825-2 comprises a H1 comprising the amino acid sequence of SEQ ID NO: 512, a L1 comprising the amino acid sequence of SEQ ID NO: 149, a H2 comprising the amino acid sequence of SEQ ID NO: 561, and a L2 comprising the amino acid sequence of SEQ ID NO: 510.

In some embodiments, the bispecific antibody EIP0825-3 comprises a H1 comprising the amino acid sequence of SEQ ID NO: 590, a L1 comprising the amino acid sequence of SEQ ID NO: 149, a H2 comprising the amino acid sequence of SEQ ID NO: 511, and a L2 comprising the amino acid sequence of SEQ ID NO: 510.

In some embodiments, the bispecific antibody EIP0825-4 comprises a H1 comprising the amino acid sequence of SEQ ID NO: 590, a L1 comprising the amino acid sequence of SEQ ID NO: 149, a H2 comprising the amino acid sequence of SEQ ID NO: 561, and a L2 comprising the amino acid sequence of SEQ ID NO: 510.

In some embodiments, the bispecific antibody EIP0824 comprises a H1 comprising the amino acid sequence of SEQ ID NO: 515, a L1 comprising the amino acid sequence of SEQ ID NO: 141, a H2 comprising the amino acid sequence of SEQ ID NO: 514, and a L2 comprising the amino acid sequence of SEQ ID NO: 513.

In some embodiments, the bispecific antibody EIP0824-2 comprises a H1 comprising the amino acid sequence of SEQ ID NO: 515, a L1 comprising the amino acid sequence of SEQ ID NO: 141, a H2 comprising the amino acid sequence of SEQ ID NO: 562, and a L2 comprising the amino acid sequence of SEQ ID NO: 513.

In some embodiments, the bispecific antibody EIP0824-3 comprises a H1 comprising the amino acid sequence of SEQ ID NO: 591, a L1 comprising the amino acid sequence of SEQ ID NO: 141, a H2 comprising the amino acid sequence of SEQ ID NO: 514, and a L2 comprising the amino acid sequence of SEQ ID NO: 513.

In some embodiments, the bispecific antibody EIP0824-4 comprises a H1 comprising the amino acid sequence of SEQ ID NO: 591, a L1 comprising the amino acid sequence of SEQ ID NO: 141, a H2 comprising the amino acid sequence of SEQ ID NO: 562, and a L2 comprising the amino acid sequence of SEQ ID NO: 513.

In some embodiments, the bispecific antibody EIP0846 comprises a H1 comprising the amino acid sequence of SEQ ID NO: 518, a L1 comprising the amino acid sequence of SEQ ID NO: 149, a H2 comprising the amino acid sequence of SEQ ID NO: 517, and a L2 comprising the amino acid sequence of SEQ ID NO: 516.

In some embodiments, the bispecific antibody EIP0846-2 comprises a H1 comprising the amino acid sequence of SEQ ID NO: 518, a L1 comprising the amino acid sequence of SEQ ID NO: 149, a H2 comprising the amino acid sequence of SEQ ID NO: 563, and a L2 comprising the amino acid sequence of SEQ ID NO: 516.

In some embodiments, the bispecific antibody EIP0846-3 comprises a H1 comprising the amino acid sequence of SEQ ID NO: 592, a L1 comprising the amino acid sequence of SEQ ID NO: 149, a H2 comprising the amino acid sequence of SEQ ID NO: 517, and a L2 comprising the amino acid sequence of SEQ ID NO: 516.

In some embodiments, the bispecific antibody EIP0846-4 comprises a H1 comprising the amino acid sequence of SEQ ID NO: 592, a L1 comprising the amino acid sequence of SEQ ID NO: 149, a H2 comprising the amino acid sequence of SEQ ID NO: 563, and a L2 comprising the amino acid sequence of SEQ ID NO: 516.

In some embodiments, the bispecific antibody EIP0965 comprises a H1 comprising the amino acid sequence of SEQ ID NO: 521, a L1 comprising the amino acid sequence of SEQ ID NO: 153, a H2 comprising the amino acid sequence of SEQ ID NO: 520, and a L2 comprising the amino acid sequence of SEQ ID NO: 519.

In some embodiments, the bispecific antibody EIP0965-2 comprises a H1 comprising the amino acid sequence of SEQ ID NO: 521, a L1 comprising the amino acid sequence of SEQ ID NO: 153, a H2 comprising the amino acid sequence of SEQ ID NO: 564, and a L2 comprising the amino acid sequence of SEQ ID NO: 519.

In some embodiments, the bispecific antibody EIP0965-3 comprises a H1 comprising the amino acid sequence of SEQ ID NO: 593, a L1 comprising the amino acid sequence of SEQ ID NO: 153, a H2 comprising the amino acid sequence of SEQ ID NO: 520, and a L2 comprising the amino acid sequence of SEQ ID NO: 519.

In some embodiments, the bispecific antibody EIP0965-4 comprises a H1 comprising the amino acid sequence of SEQ ID NO: 593, a L1 comprising the amino acid sequence of SEQ ID NO: 153, a H2 comprising the amino acid sequence of SEQ ID NO: 564, and a L2 comprising the amino acid sequence of SEQ ID NO: 519.

In some embodiments, the bispecific antibody EIP0867 comprises a H1 comprising the amino acid sequence of SEQ ID NO: 524, a L1 comprising the amino acid sequence of SEQ ID NO: 141, a H2 comprising the amino acid sequence of SEQ ID NO: 523, and a L2 comprising the amino acid sequence of SEQ ID NO: 522.

In some embodiments, the bispecific antibody EIP0867-2 comprises a H1 comprising the amino acid sequence of SEQ ID NO: 524, a L1 comprising the amino acid sequence of SEQ ID NO: 141, a H2 comprising the amino acid sequence of SEQ ID NO: 565, and a L2 comprising the amino acid sequence of SEQ ID NO: 522.

In some embodiments, the bispecific antibody EIP0867-3 comprises a H1 comprising the amino acid sequence of SEQ ID NO: 594, a L1 comprising the amino acid sequence of SEQ ID NO: 141, a H2 comprising the amino acid sequence of SEQ ID NO: 523, and a L2 comprising the amino acid sequence of SEQ ID NO: 522.

In some embodiments, the bispecific antibody EIP0867-4 comprises a H1 comprising the amino acid sequence of SEQ ID NO: 594, a L1 comprising the amino acid sequence of SEQ ID NO: 141, a H2 comprising the amino acid sequence of SEQ ID NO: 565, and a L2 comprising the amino acid sequence of SEQ ID NO: 522.

In some embodiments, the bispecific antibody EIP0969 comprises a H1 comprising the amino acid sequence of SEQ ID NO: 491, a L1 comprising the amino acid sequence of SEQ ID NO: 149, a H2 comprising the amino acid sequence of SEQ ID NO: 490, and a L2 comprising the amino acid sequence of SEQ ID NO: 471.

In some embodiments, the bispecific antibody EIP0969-2 comprises a H1 comprising the amino acid sequence of SEQ ID NO: 491, a L1 comprising the amino acid sequence of SEQ ID NO: 149, a H2 comprising the amino acid sequence of SEQ ID NO: 566, and a L2 comprising the amino acid sequence of SEQ ID NO: 471.

In some embodiments, the bispecific antibody EIP0969-3 comprises a H1 comprising the amino acid sequence of SEQ ID NO: 595, a L1 comprising the amino acid sequence of SEQ ID NO: 149, a H2 comprising the amino acid sequence of SEQ ID NO: 490, and a L2 comprising the amino acid sequence of SEQ ID NO: 471.

In some embodiments, the bispecific antibody EIP0969-4 comprises a H1 comprising the amino acid sequence of SEQ ID NO: 595, a L1 comprising the amino acid sequence of SEQ ID NO: 149, a H2 comprising the amino acid sequence of SEQ ID NO: 566, and a L2 comprising the amino acid sequence of SEQ ID NO: 471.

In some embodiments, the bispecific antibody EIP0892 comprises a H1 comprising the amino acid sequence of SEQ ID NO: 494, a L1 comprising the amino acid sequence of SEQ ID NO: 149, a H2 comprising the amino acid sequence of SEQ ID NO: 493, and a L2 comprising the amino acid sequence of SEQ ID NO: 471.

In some embodiments, the bispecific antibody EIP0892-2 comprises a H1 comprising the amino acid sequence of SEQ ID NO: 494, a L1 comprising the amino acid sequence of SEQ ID NO: 149, a H2 comprising the amino acid sequence of SEQ ID NO: 567, and a L2 comprising the amino acid sequence of SEQ ID NO: 471.

In some embodiments, the bispecific antibody EIP0892-3 comprises a H1 comprising the amino acid sequence of SEQ ID NO: 596, a L1 comprising the amino acid sequence of SEQ ID NO: 149, a H2 comprising the amino acid sequence of SEQ ID NO: 493, and a L2 comprising the amino acid sequence of SEQ ID NO: 471.

In some embodiments, the bispecific antibody EIP0892-4 comprises a H1 comprising the amino acid sequence of SEQ ID NO: 596, a L1 comprising the amino acid sequence of SEQ ID NO: 149, a H2 comprising the amino acid sequence of SEQ ID NO: 567, and a L2 comprising the amino acid sequence of SEQ ID NO: 471.

In some embodiments, the bispecific antibody EIP0929 comprises a H1 comprising the amino acid sequence of SEQ ID NO: 497, a L1 comprising the amino acid sequence of SEQ ID NO: 141, a H2 comprising the amino acid sequence of SEQ ID NO: 496, and a L2 comprising the amino acid sequence of SEQ ID NO: 471.

In some embodiments, the bispecific antibody EIP0929-2 comprises a H1 comprising the amino acid sequence of SEQ ID NO: 497, a L1 comprising the amino acid sequence of SEQ ID NO: 141, a H2 comprising the amino acid sequence of SEQ ID NO: 568, and a L2 comprising the amino acid sequence of SEQ ID NO: 471.

In some embodiments, the bispecific antibody EIP0929-3 comprises a H1 comprising the amino acid sequence of SEQ ID NO: 597, a L1 comprising the amino acid sequence of SEQ ID NO: 141, a H2 comprising the amino acid sequence of SEQ ID NO: 496, and a L2 comprising the amino acid sequence of SEQ ID NO: 471.

In some embodiments, the bispecific antibody EIP0929-4 comprises a H1 comprising the amino acid sequence of SEQ ID NO: 597, a L1 comprising the amino acid sequence of SEQ ID NO: 141, a H2 comprising the amino acid sequence of SEQ ID NO: 568, and a L2 comprising the amino acid sequence of SEQ ID NO: 471.

In some embodiments, the bispecific antibody EIP0893 comprises a H1 comprising the amino acid sequence of SEQ ID NO: 500, a L1 comprising the amino acid sequence of SEQ ID NO: 149, a H2 comprising the amino acid sequence of SEQ ID NO: 499, and a L2 comprising the amino acid sequence of SEQ ID NO: 471.

In some embodiments, the bispecific antibody EIP0893-2 comprises a H1 comprising the amino acid sequence of SEQ ID NO: 500, a L1 comprising the amino acid sequence of SEQ ID NO: 149, a H2 comprising the amino acid sequence of SEQ ID NO: 569, and a L2 comprising the amino acid sequence of SEQ ID NO: 471.

In some embodiments, the bispecific antibody EIP0893-3 comprises a H1 comprising the amino acid sequence of SEQ ID NO: 598, a L1 comprising the amino acid sequence of SEQ ID NO: 149, a H2 comprising the amino acid sequence of SEQ ID NO: 499, and a L2 comprising the amino acid sequence of SEQ ID NO: 471.

In some embodiments, the bispecific antibody EIP0893-4 comprises a H1 comprising the amino acid sequence of SEQ ID NO: 598, a L1 comprising the amino acid sequence of SEQ ID NO: 149, a H2 comprising the amino acid sequence of SEQ ID NO: 569, and a L2 comprising the amino acid sequence of SEQ ID NO: 471.

In some embodiments, the bispecific antibody EIP0886 comprises a H1 comprising the amino acid sequence of SEQ ID NO: 503, a L1 comprising the amino acid sequence of SEQ ID NO: 153, a H2 comprising the amino acid sequence of SEQ ID NO: 502, and a L2 comprising the amino acid sequence of SEQ ID NO: 471.

In some embodiments, the bispecific antibody EIP0886-2 comprises a H1 comprising the amino acid sequence of SEQ ID NO: 503, a L1 comprising the amino acid sequence of SEQ ID NO: 153, a H2 comprising the amino acid sequence of SEQ ID NO: 570, and a L2 comprising the amino acid sequence of SEQ ID NO: 471.

In some embodiments, the bispecific antibody EIP0886-3 comprises a H1 comprising the amino acid sequence of SEQ ID NO: 599, a L1 comprising the amino acid sequence of SEQ ID NO: 153, a H2 comprising the amino acid sequence of SEQ ID NO: 502, and a L2 comprising the amino acid sequence of SEQ ID NO: 471.

In some embodiments, the bispecific antibody EIP0886-4 comprises a H1 comprising the amino acid sequence of SEQ ID NO: 599, a L1 comprising the amino acid sequence of SEQ ID NO: 153, a H2 comprising the amino acid sequence of SEQ ID NO: 570, and a L2 comprising the amino acid sequence of SEQ ID NO: 471.

In some embodiments, the bispecific antibody EIP0866 comprises a H1 comprising the amino acid sequence of SEQ ID NO: 506, a L1 comprising the amino acid sequence of SEQ ID NO: 141, a H2 comprising the amino acid sequence of SEQ ID NO: 505, and a L2 comprising the amino acid sequence of SEQ ID NO: 471.

In some embodiments, the bispecific antibody EIP0866-2 comprises a H1 comprising the amino acid sequence of SEQ ID NO: 506, a L1 comprising the amino acid sequence of SEQ ID NO: 141, a H2 comprising the amino acid sequence of SEQ ID NO: 571, and a L2 comprising the amino acid sequence of SEQ ID NO: 471.

In some embodiments, the bispecific antibody EIP0866-3 comprises a H1 comprising the amino acid sequence of SEQ ID NO: 600, a L1 comprising the amino acid sequence of SEQ ID NO: 141, a H2 comprising the amino acid sequence of SEQ ID NO: 505, and a L2 comprising the amino acid sequence of SEQ ID NO: 471.

In some embodiments, the bispecific antibody EIP0866-4 comprises a H1 comprising the amino acid sequence of SEQ ID NO: 600, a L1 comprising the amino acid sequence of SEQ ID NO: 141, a H2 comprising the amino acid sequence of SEQ ID NO: 571, and a L2 comprising the amino acid sequence of SEQ ID NO: 471.

In some embodiments, the bispecific antibody EIP0966 comprises a H1 comprising the amino acid sequence of SEQ ID NO: 527, a L1 comprising the amino acid sequence of SEQ ID NO: 149, a H2 comprising the amino acid sequence of SEQ ID NO: 526, and a L2 comprising the amino acid sequence of SEQ ID NO: 525.

In some embodiments, the bispecific antibody EIP0966-2 comprises a H1 comprising the amino acid sequence of SEQ ID NO: 527, a L1 comprising the amino acid sequence of SEQ ID NO: 149, a H2 comprising the amino acid sequence of SEQ ID NO: 572, and a L2 comprising the amino acid sequence of SEQ ID NO: 525.

In some embodiments, the bispecific antibody EIP0966-3 comprises a H1 comprising the amino acid sequence of SEQ ID NO: 601, a L1 comprising the amino acid sequence of SEQ ID NO: 149, a H2 comprising the amino acid sequence of SEQ ID NO: 526, and a L2 comprising the amino acid sequence of SEQ ID NO: 525.

In some embodiments, the bispecific antibody EIP0966-4 comprises a H1 comprising the amino acid sequence of SEQ ID NO: 601, a L1 comprising the amino acid sequence of SEQ ID NO: 149, a H2 comprising the amino acid sequence of SEQ ID NO: 572, and a L2 comprising the amino acid sequence of SEQ ID NO: 525.

In some embodiments, the bispecific antibody EIP0967 comprises a H1 comprising the amino acid sequence of SEQ ID NO: 530, a L1 comprising the amino acid sequence of SEQ ID NO: 149, a H2 comprising the amino acid sequence of SEQ ID NO: 529, and a L2 comprising the amino acid sequence of SEQ ID NO: 528.

In some embodiments, the bispecific antibody EIP0967-2 comprises a H1 comprising the amino acid sequence of SEQ ID NO: 530, a L1 comprising the amino acid sequence of SEQ ID NO: 149, a H2 comprising the amino acid sequence of SEQ ID NO: 573, and a L2 comprising the amino acid sequence of SEQ ID NO: 528.

In some embodiments, the bispecific antibody EIP0967-3 comprises a H1 comprising the amino acid sequence of SEQ ID NO: 602, a L1 comprising the amino acid sequence of SEQ ID NO: 149, a H2 comprising the amino acid sequence of SEQ ID NO: 529, and a L2 comprising the amino acid sequence of SEQ ID NO: 528.

In some embodiments, the bispecific antibody EIP0967-4 comprises a H1 comprising the amino acid sequence of SEQ ID NO: 602, a L1 comprising the amino acid sequence of SEQ ID NO: 149, a H2 comprising the amino acid sequence of SEQ ID NO: 573, and a L2 comprising the amino acid sequence of SEQ ID NO: 528.

In some embodiments, the bispecific antibody EIP0930 comprises a H1 comprising the amino acid sequence of SEQ ID NO: 533, a L1 comprising the amino acid sequence of SEQ ID NO: 141, a H2 comprising the amino acid sequence of SEQ ID NO: 532, and a L2 comprising the amino acid sequence of SEQ ID NO: 531.

In some embodiments, the bispecific antibody EIP0930-2 comprises a H1 comprising the amino acid sequence of SEQ ID NO: 533, a L1 comprising the amino acid sequence of SEQ ID NO: 141, a H2 comprising the amino acid sequence of SEQ ID NO: 574, and a L2 comprising the amino acid sequence of SEQ ID NO: 531.

In some embodiments, the bispecific antibody EIP0930-3 comprises a H1 comprising the amino acid sequence of SEQ ID NO: 603, a L1 comprising the amino acid sequence of SEQ ID NO: 141, a H2 comprising the amino acid sequence of SEQ ID NO: 532, and a L2 comprising the amino acid sequence of SEQ ID NO: 531.

In some embodiments, the bispecific antibody EIP0930-4 comprises a H1 comprising the amino acid sequence of SEQ ID NO: 603, a L1 comprising the amino acid sequence of SEQ ID NO: 141, a H2 comprising the amino acid sequence of SEQ ID NO: 574, and a L2 comprising the amino acid sequence of SEQ ID NO: 531.

In some embodiments, the bispecific antibody EIP0968 comprises a H1 comprising the amino acid sequence of SEQ ID NO: 536, a L1 comprising the amino acid sequence of SEQ ID NO: 149, a H2 comprising the amino acid sequence of SEQ ID NO: 535, and a L2 comprising the amino acid sequence of SEQ ID NO: 534.

In some embodiments, the bispecific antibody EIP0968-2 comprises a H1 comprising the amino acid sequence of SEQ ID NO: 536, a L1 comprising the amino acid sequence of SEQ ID NO: 149, a H2 comprising the amino acid sequence of SEQ ID NO: 575, and a L2 comprising the amino acid sequence of SEQ ID NO: 534.

In some embodiments, the bispecific antibody EIP0968-3 comprises a H1 comprising the amino acid sequence of SEQ ID NO: 604, a L1 comprising the amino acid sequence of SEQ ID NO: 149, a H2 comprising the amino acid sequence of SEQ ID NO: 535, and a L2 comprising the amino acid sequence of SEQ ID NO: 534.

In some embodiments, the bispecific antibody EIP0968-4 comprises a H1 comprising the amino acid sequence of SEQ ID NO: 604, a L1 comprising the amino acid sequence of SEQ ID NO: 149, a H2 comprising the amino acid sequence of SEQ ID NO: 575, and a L2 comprising the amino acid sequence of SEQ ID NO: 534.

In some embodiments, the bispecific antibody EIP0891 comprises a H1 comprising the amino acid sequence of SEQ ID NO: 539, a L1 comprising the amino acid sequence of SEQ ID NO: 153, a H2 comprising the amino acid sequence of SEQ ID NO: 538, and a L2 comprising the amino acid sequence of SEQ ID NO: 537.

In some embodiments, the bispecific antibody EIP0891-2 comprises a H1 comprising the amino acid sequence of SEQ ID NO: 539, a L1 comprising the amino acid sequence of SEQ ID NO: 153, a H2 comprising the amino acid sequence of SEQ ID NO: 576, and a L2 comprising the amino acid sequence of SEQ ID NO: 537.

In some embodiments, the bispecific antibody EIP0891-3 comprises a H1 comprising the amino acid sequence of SEQ ID NO: 605, a L1 comprising the amino acid sequence of SEQ ID NO: 153, a H2 comprising the amino acid sequence of SEQ ID NO: 538, and a L2 comprising the amino acid sequence of SEQ ID NO: 537.

In some embodiments, the bispecific antibody EIP0891-4 comprises a H1 comprising the amino acid sequence of SEQ ID NO: 605, a L1 comprising the amino acid sequence of SEQ ID NO: 153, a H2 comprising the amino acid sequence of SEQ ID NO: 576, and a L2 comprising the amino acid sequence of SEQ ID NO: 537.

In some embodiments, the bispecific antibody EIP0868 comprises a H1 comprising the amino acid sequence of SEQ ID NO: 542, a L1 comprising the amino acid sequence of SEQ ID NO: 141, a H2 comprising the amino acid sequence of SEQ ID NO: 541, and a L2 comprising the amino acid sequence of SEQ ID NO: 540.

In some embodiments, the bispecific antibody EIP0868-2 comprises a H1 comprising the amino acid sequence of SEQ ID NO: 542, a L1 comprising the amino acid sequence of SEQ ID NO: 141, a H2 comprising the amino acid sequence of SEQ ID NO: 577, and a L2 comprising the amino acid sequence of SEQ ID NO: 540.

In some embodiments, the bispecific antibody EIP0868-3 comprises a H1 comprising the amino acid sequence of SEQ ID NO: 606, a L1 comprising the amino acid sequence of SEQ ID NO: 141, a H2 comprising the amino acid sequence of SEQ ID NO: 541, and a L2 comprising the amino acid sequence of SEQ ID NO: 540.

In some embodiments, the bispecific antibody EIP0868-4 comprises a H1 comprising the amino acid sequence of SEQ ID NO: 606, a L1 comprising the amino acid sequence of SEQ ID NO: 141, a H2 comprising the amino acid sequence of SEQ ID NO: 577, and a L2 comprising the amino acid sequence of SEQ ID NO: 540.

Any one of the anti-CD3/anti-20 bispecific antibodies shown above in Table 10.5 can be further modified by substituting any one of the anti-CD3ε antigen binding regions with any one of the anti-CD3ε binding regions shown in Tables 9.1, 9.2 and 9.3. For example, the anti-CD3ε antigen binding regions of bispecific antibody “EIP0969” can be substituted with any one of the anti-CD3ε binding regions shown in Tables 9.1, 9.2 and 9.3 to produce the bispecific antibodies of the invention. Exemplary antibodies are shown in Table 10.4 and Table 10.5.

### CD180 Antigen

CD180 is a type I transmembrane toll-like receptor (TLR) with the highest sequence similarity to TLR4 and was initially discovered as a mouse B lymphocyte activation receptor that protects B cells from irradiation and dexamethasone-mediated cell death (Miyake et al., 1995, J. Immunol. 154: 3333-3340; Divanovic et al., 2005, Nat. Immunol. 6: 571-578). CD180 and MD-1, an interacting protein required for CD180 surface expression and function, are found on the surface of B, but not T lymphocytes and monocytes, but not other myeloid cells with similar expression in humans and mice (Ohto et al., 2011, J. Mol. Biol. 413: 815-825; Maycur-Rousse et al., 2016, Cytometry B. Clin. Cytom. 90: 462-466). CD180/MD1 is also found on hematological malignancies such as B cell lymphoma (Mayeur-Rousse et al., 2016, Cytometry B. Clin. Cytom. 90: 462-466; Miguet et al., 2013, Leukemia 27: 1748-1750; Mansour et al., 2020, J. Hematopathol. 13: 205-211; Fidyt et al., 2022, Blood 140; 10273-10274), chronic lymphocytic leukemia (CLL)(Porakishvili et al., 2005, Br. J. Haematol. 131: 313-319; Edwards et al., 2021, Br. J. Haematol. 195), and acute myeloid leukemia (AML) (Kramer et al., 2022, Blood 140: 1533-1548; Saito et al., 2010, Sci. Transl. Med. 2) implicating CD180/MD1 as a novel target for cancer immunotherapy with differentiated opportunities in relapse and/or refractory B cell malignancies as an alternative B cell lineage antigen, where CD19 and CD20 loss emerge from CD19/CD20 targeted treatments, and in myeloid malignancies of the monocytic lineage (Marshalek et al., 2022. JCO 40: e19537-e19537).

The terms “CD180” and “CD180 antigen” are used interchangeably herein, and include any variants, isoforms and species homologs of human CD180 which are naturally expressed by cells or are expressed on cells transfected with the CD180 gene. Binding of an antibody of the invention to the CD180 antigen mediate the killing of cells expressing CD180 (e.g., a tumor cell) by inactivating CD180. The killing of the cells expressing CD180 may occur by one or more of the following mechanisms: Cell death/apoptosis induction, ADCC and CDC.

The term “anti-CD180 antibody” according to the invention is an antibody that binds specifically to CD180 antigen. Depending on binding properties and biological activities of anti-CD180 antibodies to the CD180 antigen, two types of anti-CD180 antibodies (type I and type II anti-CD180 antibodies) can be distinguished according to Cragg, M. S., et al., Blood 103 (2004) 2738-2743; and Cragg, M. S., et al., Blood 101 (2003) 1045-1052.

In some instances, CD180 may be expressed in low copy number on the target cell (e.g. tumor cell). For example, in some instances, CD180 is expressed or present at less than 35,000 copies per target cell. In some embodiments, the low copy number cell surface CD180 is present between 100 and 35.000 copies per target cell; between 100 and 30,000 copies per target cell; between 100 and 25,000 copies per target cell; between 100 and 20,000 copies per target cell; between 100 and 15,000 copies per target cell: between 100 and 10,000 copies per target cell; between 100 and 5,000 copies per target cell: between 100 and 2,000 copies per target cell; between 100 and 1,000 copies per target cell; or between 100 and 500 copies per target cell. Copy number of the cell surface CD180 can be determined, for example, using a standard Scratchcard plot.

This disclosure provides a bispecific antibody comprising a first antigen binding region that binds CD3 and a second binding region that binds CD180.

For example, the second binding region that binds to CD180 can be derived from the binding regions of an anti-CD180 antibody. Exemplary anti-CD180 antibodies include but are not limited to MHR73-11 and G28-8 monoclonal antibody (BD Biosciences, Thermo Scientific Sigma Aldrich).

In some embodiments, a second antigen binding region that binds CD180 comprises any one of the VH and VL sequences listed in Table 11.1. In Table 11.1, the underlined sequences are CDR sequence according to Kabat and the bolded sequences are CDR sequences according to Chothia.

In some embodiments, the second binding region that binds to CD180 comprises: a) a heavy chain variable region (VH) comprising a VH complementarity determining region 1 (VH_{CDR1}), a VH complementarity determining region 2 (VH_{CDR2}) and a VH complementarity determining region 3 (VH_{CDR3}); and b) alight chain variable region (VL) comprising a VL complementarity determining region 1 (VL_{CDR1}), a VL complementarity determining region 2 (VL_{CDR2}) and a VL complementarity determining region 3 (VL_{CDR3}). Tables 11.2 and 11.3 provide exemplary of CDR sequences of the anti-CD180 antibodies.

TABLE 11.1
CD180 Variable Heavy Chain and Variable Light Chain Domains

| | | SEQ ID |
|---|---|---|
| Ab | Amino Acid Sequence | NO: |
| (VL) EIP464 | DIQMTQSPASLSASVGETVTITCRASENIYSYLAWYQQKQGKSP | 160 |
| | QLLVNNAKTLAEGVPSRFSGSGSGTQFSLKINSLQPEDFGSYYC | |
| | QHHYGSPWTFGGGTELEIK | |
| (VH) EIP464 | EVQLQQSGPELVKPGASMKISCKASGYSFTDYTMNWVKQSHGKN | 161 |
| | LEWIGLINPYNGVTSYNPNFQGKATLTVDKSSSTAYMELLSLTS | |
| | EDSAVYYCARSGDYDRNYFDYWGQGTTLTVSS | |
| (VL) | DIQMTQSPASLSASVGETVTITCRASEKIYSYLAWYQQKQGKSP | 162 |
| EIP0051 | QLLVYNAKTLAEGVPSRFSVSGSGTQFSLRINSLQPEDFGTYYC | |
| | QHHFGSPRTFGGGTKLEIK | |
| (VH) | EVQLQQSGPELVKPGASMKISCKASGYSFTGYTMNWVKQSHGKT | 163 |
| EIP0051 | LEWIGLINPYNGVTSYNQKFKDKATLTVDKSSSTAYMELLSLTS | |
| | EDSAIYYCARDYNYDYFDYWGQGTTLTVSS | |
| (VL) EIP435 | DIQMTQSPSTLSASVGDRVTITCRASENIYSYLAWYQQKPGKAP | 164 |
| | KLLVNNAKTLAEGVPSRFSGSGSGTEFTLTISSLQPDDFATYYC | |
| | QHHYGSPWTFGQGTKVEIK | |
| (VH) EIP435 | EVQLVQSGAEVKKPGSSVKVSCKASGYSFTDYTMNWVRQAPGQG | 165 |
| | LEWIGLINPYNGVTSYNPNFQGRATLTVDKSTSTAYMELSSLRS | |
| | EDTAVYYCARSGDYDRNYFDYWGQGTLVTVSS | |
| (VL) EIP463 | DIQMTQSPSTLSASVGDRVTITCRASENIYSYLAWYQQKPGKAP | 166 |
| | KLLVNNAKTLAEGVPSRFSGSGSGTEFTLTISSLQPDDFATYYC | |
| | QHHYGSPWTFGQGTKVEIK | |
| (VH) EIP463 | EVQLVQSGAEVKKPGSSVKVSCKASGYSFTDYTMNWVRQAPGQG | 167 |
| | LEWIGLINPYNGVTSYNPNFQGRATLTVDKSTSTAYMELSSLRS | |
| | EDTAVYYCARSGDYDRNYFDYWGQGTLVTVSS | |
| (VL) EIP436 | DIQMTQSPSTLSASVGDRVTITCRASENIYSYLAWYQQKPGKAP | 168 |
| | KLLVNNAKTLAEGVPSRFSGSGSGTEFTLTISSLQPDDFATYYC | |
| | QHHYGSPWTFGQGTKVEIK | |
| (VH) EIP436 | QVQLVQSGAEVKKPGASVKVSCKASGYTFTDYTMNWVRQAPGQG | 169 |
| | LEWIGLINPYNGVTSYNPNFQGRVTLTVDKSTSTAYMELSSLRS | |
| | EDTAVYYCARSGDYDRNYFDYWGQGTLVTVSS | |
| (VL) EIP437 | DIQMTQSPSSLSASVGDRVTITCRASENIYSYLAWYQQKPGKAP | 170 |
| | KLLVNNAKTLAEGVPSRFSGSGSGTDFTLTISSLQPEDFATYYC | |
| | QHHYGSPWTFGGGTELEIK | |
| (VH) EIP437 | QVQLVQSGAEVKKPGSSVKVSCKASGYSFTDYTMNWVRQAPGQG | 171 |
| | LEWIGLINPYNGVTSYNPNFQGRATLTVDKSTSTAYMELSSLRS | |
| | EDTAVYYCARSGDYDRNYFDYWGQGTTLTVSS | |
| (VL) VVV2.1 | DIQMTQSPSSLSASVGDRVTITCRASENIYSYLAWYQQKPGKAP | 172 |
| | KLLINNAKTLQSGVPSRFSGSGSGTDFTLTISSLQPEDFATYYC | |
| | QHRYGSPWTFGGGTELEIK | |
| (VH) VVV2.1 | QVQLVQSGAEVKKPGASVKVSCKASGYTFTDYTMNWVRQAPGQG | 173 |
| | LEWIGIINPYNGVTSYNPNFQGRVTLTVDKSTSTVYMELSSLRS | |
| | EDTAVYYCARSGDYDRNYFDYWGQGTLVTVSS | |
| (VL) EIP459 | DIQMTQSPSTLSASVGDRVTITCRASENIYSYLAWYQQKPGKAP | 174 |
| | KLLVNNAKTLAEGVPSRFSGSGSGTEFTLTISSLQPDDFATYYC | |
| | QHHYGSPWTFGQGTKVEIK | |
| (VH) EIP459 | QVQLVQSGAEVKKPGASVKVSCKASGYTFTDYTMNWVRQAPGQG | 175 |
| | LEWIGIINPYNGVTSYNPNFQGRVTLTVDKSTSTVYMELSSLRS | |
| | EDTAVYYCARSGDYDRNYFDYWGQGTLVTVSS | |
| (VL) EIP460 | DIQMTQSPSSLSASVGDRVTITCRASENIYSYLAWYQQKPGKAP | 176 |
| | KLLVNNAKTLAEGVPSRFSGSGSGTDFTLTISSLQPEDFATYYC | |
| | QHHYGSPWTFGGGTELEIK | |
| (VH) EIP460 | QVQLVQSGAEVKKPGASVKVSCKASGYTFTDYTMNWVRQAPGQG | 177 |
| | LEWIGIINPYNGVTSYNPNFQGRVTLTVDKSTSTVYMELSSLRS | |
| | EDTAVYYCARSGDYDRNYEDYWGQGTLVTVSS | |
| (VL) EIP461 | DIQMTQSPSSLSASVGDRVTITCRASENIYSYLAWYQQKPGKAP | 178 |
| | KLLVNNAKTLAEGVPSRFSGSGSGTEFTLTISSLQPEDFATYYC | |
| | QHHYGSPWTFGGGTELEIK | |
| (VH) EIP461 | QVQLVQSGAEVKKPGASVKVSCKASGYTFTDYTMNWVRQAPGQG | 179 |
| | LEWIGIINPYNGVTSYNPNFQGRVTLTVDKSTSTVYMELSSLRS | |
| | EDTAVYYCARSGDYDRNYFDYWGQGTLVTVSS | |
| (VL) EIP438 | DIQMTQSPSSLSASVGDRVTITCRASENIYSYLAWYQQKPGKAP | 180 |
| | KLLVNNAKTLAEGVPSRFSGSGSGTEFTLTISSLQPEDFATYYC | |
| | QHHYGSPWTFGGGTELEIK | |
| (VH) EIP438 | EVQLVQSGAEVKKPGESLKISCKASGYSFTDYTMNWVRQMPGKG | 181 |
| | LEWIGLINPYNGVTSYNPNFQGQATLSVDKSISTAYLQWSSLKA | |
| | SDTAMYYCARSGDYDRNYFDYWGQGTTLTVSS | |
| (VL) EIP462 | DIQMTQSPSTLSASVGDRVTITCRASENIYSYLAWYQQKPGKAP | 182 |
| | KLLVNNAKTLAEGVPSRFSGSGSGTEFTLTISSLQPDDFATYYC | |
| | QHHYGSPWTFGQGTKVEIK | |
| (VH) EIP462 | EVQLVQSGAEVKKPGESLKISCKASGYSFTDYTMNWVRQMPGKG | 183 |
| | LEWIGLINPYNGVTSYNPNFQGQATLSVDKSISTAYLQWSSLKA | |
| | SDTAMYYCARSGDYDRNYFDYWGQGTTLTVSS | |
| (VL) VVV3.0 | DIQMTQSPSSLSASVGDRVTITCRASENIYSYLAWYQQKPGKAP | 184 |
| | KLLVNNAKTLAEGVPSRFSGSGSGTDFTFTISSLQPEDIATYYC | |
| | QHHYGSPWTFGGGTELEIK | |
| (VH) VVV3.0 | EVQLVESGGGLVQPGGSLRLSCAASGYSFTDYTMNWVRQAPGKG | 185 |
| | LEWIGLINPYNGVTSYNPNFQGRATLSVDKSKNSAYLQMNSLKT | |
| | EDTAVYYCARSGDYDRNYFDYWGQGTTLTVSS | |
| (VL) VVV3.1 | DIQMTQSPSSLSASVGDRVTITCRASENIYSYLAWYQQKPGKAP | 186 |
| | KLLINNAKTLAEGVPSRFSGSGSGTDFTLTISSLQPEDFATYYC | |
| | QHRYGSPWTFGGGTELEIK | |
| (VH) VVV3.1 | EVQLVESGGGLVQPGGSLRISCAASGYSETDYTMNWVRQAPGKG | 187 |
| | LEWIGLINPYNGVTSYNPNFQGRATLSVDKSKNSAYLQMNSLKT | |
| | EDTAVYYCARSGDYDRNYFDYWGQGTTLTVSS | |
| (VL) VVV3.2 | DIQMTQSPSSLSASVGDRVTITCRASENIYSYLAWYQQKPGKAP | 188 |
| | KLLINNAKTLAEGVPSRFSGSGSGTDFTLTISSLQPEDFATYYC | |
| | QHHYGSPWTFGGGTKVEIK | |
| (VH) VVV3.2 | EVQLVESGGGLVQPGGSLRLSCAASGYSFTDYTMNWVRQAPGKG | 189 |
| | LEWIGLINPYNGVTSYNPNFQGRATLSVDKSKNSAYLQMNSLKT | |
| | EDTAVYYCARSGDYDRNYFDYWGQGTTLTVSS | |
| (VL) VVV3.3 | DIQMTQSPSSLSASVGDRVTITCRASENIYSYLAWYQQKPGKAP | 190 |
| | KLLVNNAKTLAEGVPSRFSGSGSGTDFTFTISSLQPEDIATYYC | |
| | QHHYGSPWTFGGGTELEIK | |
| (VH) VVV3.3 | EVQLVESGGGLVQPGGSLRLSCAASGFTFTDYTMNWVRQAPGKG | 191 |
| | LEWVGLINPYNGVTSYNPNFQGRFTISVDKSKNSAYLQMNSLKT | |
| | EDTAVYYCARSGDYDRNYFDYWGQGTLVTVSS | |
| (VL) VVV3.4 | DIQMTQSPSSLSASVGDRVTITCRASENIYSYLAWYQQKPGKAP | 192 |
| | KLLINNAKTLAEGVPSRFSGSGSGTDFTLTISSLQPEDFATYYC | |
| | QHRYGSPWTFGGGTELEIK | |
| (VH) VVV3.4 | EVQLVESGGGLVQPGGSLRISCAASGFTFTDYTMNWVRQAPGKG | 193 |
| | LEWVGLINPYNGVTSYNPNFQGRFTISVDKSKNSAYLQMNSLKT | |
| | EDTAVYYCARSGDYDRNYFDYWGQGTLVTVSS | |
| (VL) VVV3.5 | DIQMTQSPSSLSASVGDRVTITCRASENIYSYLAWYQQKPGKAP | 194 |
| | KLLINNAKTLAEGVPSRFSGSGSGTDFTLTISSLQPEDFATYYC | |
| | QHHYGSPWTFGGGTKVEIK | |
| (VH) VVV3.5 | EVQLVESGGGLVQPGGSLRLSCAASGFTFTDYTMNWVRQAPGKG | 195 |
| | LEWVGLINPYNGVTSYNPNFQGRFTISVDKSKNSAYLQMNSLKT | |
| | EDTAVYYCARSGDYDRNYFDYWGQGTLVTVSS | |
| (VL) | DIQMTQSPSTLSASVGDRVTITCRASENIYSYLAWYQKKPGKAP | 196 |
| EIP1042 | KLLVNNAKTLAEGVPSRFSGSGSGTEFTLTISSLQPDDFATYYC | |
| | QHEYGSPWTFGQGTKVEIK | |
| (VH) | QVQLVQSGAEVKKPGASVKVSCKASGYTFTDYTMNWVRDAPGQG | 197 |
| EIP1042 | LEWIGLINPYNGVTSYNPNFQGRVTLTVDKSTSTAYMELSSLRS | |
| | EDTAVYYCARSGDYDRNYFDYWGQGTLVTVSS | |
| (VL) | DIQMTQSPSSLSASVGDRVTITCRASENIYSYLAWYQKKPGKAP | 198 |
| EIP1043 | KLLVNNAKTLAEGVPSRFSGSGSGTDFTLTISSLQPEDFATYYC | |
| | QHHYGSPWTFGGGTELEIK | |
| (VH) | QVQLVQSGAEVKKPGSSVKVSCKASGYSFTDYTMNWVRDAPGQG | 199 |
| EIP1043 | LEWIGLINPYNGVTSYNPNFQGRATLTVQKSTSTAYMELSSLRS | |
| | EDTAVYYCARSGDYDRNYFDYWGQGTTLTVSS | |
| (VL) | DIQMTQSPSSLSASVGDRVTITCRASENIYSYLAWYQKKPGKAP | 200 |
| EIP1044 | KLLVNNAKTLAEGVPSRFSGSGSGTEFTLTISSLQPEDFATYYC | |
| | QHHYGSPWTFGGGTELEIK | |
| (VH) | EVQLVQSGAEVKKPGESLKISCKASGYSFTDYTMNWVRDMPGKG | 201 |
| EIP1044 | LEWIGLINPYNGVTSYNPNFQGQATLSVDKSISTAYLQWSSLKA | |
| | SDTAMYYCARSGDYDRNYFDYWGQGTTLTVSS | |
| (VL) | DIQMTQSPSTLSASVGDRVTITCRASENIYSYLAWYQKKPGKAP | 202 |
| EIP1056 | KLLVNNAKTLAEGVPSRFSGSGSGTEFTLTISSLQPDDFATYYC | |
| | QHHYGSPWTFGQGTKVEIK | |
| (VH) | EVQLVQSGAEVKKPGESLKISCKASGYSFTDYTMNWVRDMPGKG | 203 |
| EIP1056 | LEWIGLINPYNGVTSYNPNFQGQATLSVDKSISTAYLQWSSLKA | |
| | SDTAMYYCARSGDYDRNYFDYWGQGTTLTVSS | |
| (VL) | DIQMTQSPSTLSASVGDRVTITCRASENIYSYLAWYQKKPGKAP | 204 |
| EIP1057 | KLLVNNAKTLAEGVPSRFSGSGSGTEFTLTISSLQPDDFATYYC | |
| | QHHYGSPWTFGQGTKVEIK | |
| (VH) | EVQLVQSGAEVKKPGSSVKVSCKASGYSFTDYTMNWVRDAPGQG | 205 |
| EIP1057 | LEWIGLINPYNGVTSYNPNFQGRATLTVDKSTSTAYMELSSLRS | |
| | EDTAVYYCARSGDYDRNYFDYWGQGTLVTVSS | |

Table 11.2
Anti-CD180 Heavy Chain CDRs

| | CDRH1 | CDRH2 | CDRH3 |
|---|---|---|---|
| EIP1042, | DYTMN (SEQ IL | LINPYNGVTSYNPNFQG (SEQ | SGDYDRNYFDY (SEQ ID |
| EIP 436 | NO: 210) (Kabat) | ID NO: 212) (Kabat) | NO: 214) |
| | GYTFTDY (SEQ ID | NPYNGV (SEQ ID NO: 213) | |
| | NO: 211) | (Chothia) | |
| | (Chothia) | | |
| EIP1043, | DYTMN (SEQ ID | LINPYNGVTSYNPNFQG (SEQ | SGDYDRNYEDY (SEQ ID |
| EIP1044, | NO: 210) (Kabat) | ID NO: 212) (Kabat) | NO: 214) |
| EIP1056, | GYSFTDY (SEQ ID | NPYNGV (SEQ ID NO: 213) | |
| EIP1057, | NO: 215) | (Chothia) | |
| VVV3.0, | (Chothia) | | |
| VVV3.1, | | | |
| VVV3.2, | | | |
| VVV3.4, | | | |
| EIP435, | | | |
| BIP437, | | | |
| EIP438, | | | |
| EIP462, | | | |
| EIP463, | | | |
| EIP464, | | | |
| EIP0696, | | | |
| EIP0716, | | | |
| EIP0706, | | | |
| EIP0707, | | | |
| EIP0710, | | | |
| EIP0717, | | | |
| EIP0698, | | | |
| EIP0699, | | | |
| EIP0708, | | | |
| EIP0709, | | | |
| EIP0745, | | | |
| EIP0746, | | | |
| EIP0751, | | | |
| EIP0752, | | | |
| EIP0133, | | | |
| EIP0553 | | | |
| BIP0554, | | | |
| VVV2.1, | DYTMN (SEQ ID | IINPYNGVTSYNPNFQG (SEQ | SGDYDRNYPDY (SEQ ID |
| EIP459, | NO: 210) (Kabat) | ID NO: 216) (Kabat) | NO: 214) |
| EIP460, | GYTFTDY (SEQ ID | NPYNGV (SEQ ID NO: 213) | |
| EIP461 | NO: 211) | (Chothia) | |
| | (Chothia) | | |
| VVV3.3, | DYTMN (SEQ ID | LINPYNGVTSYNPNEQG (SEQ | SGDYDRNYFDY (SEQ ID |
| VVV3.5 | NO: 210) ( Kabat) | ID NO: 212) (Kabat) | NO: 214) |
| | GFTFTDY (SEQ ID | NPYNGV (SEQ ID NO: 213) | |
| | NO: 217) | (Chothia) | |
| | (Chothia) | | |
| G28.8, | GYTMN (SEQ ID | LINPYNGVTSYNQKFKD (SEQ | DYNYDYFDY (SEQ ID NO: |
| EIP0051, | NO: 218) (Kabat) | ID NO: 220) (Kabat) | 221) |
| EIP0100 | GYSFIGY (SEQ ID | NPYNGV (SEQ ID NO: 213) | |
| | NO: 219) | (Chothia) | |
| | (Chothia) | | |

TABLE 11.3
Anti-CD180 Light Chain CDRs

| | CDRL1 | CDRL2 | CDRL3 |
|---|---|---|---|
| EIP1042, | RASENIYSYLA (SEQ | NAKTLAE (SEQ ID | QHHYGSPWI (SEQ |
| EIP436, | ID NO: 222) | NO: 223) | ID NO: 224) |
| EIP1043, | | | |
| EIP1044, | | | |
| EIP1056, | | | |
| EIP1057, | | | |
| VVV3.0, | | | |
| VVV3.1, | | | |
| VVV3.2, | | | |
| VVV3.4, | | | |
| EIP435, | | | |
| BIP437, | | | |
| EIP438, | | | |
| EIP462, | | | |
| EIP463, | | | |
| EIP464, | | | |
| EIP0696, | | | |
| EIP0716, | | | |
| BIP0706, | | | |
| EIP0707, | | | |
| EIP0710, | | | |
| EIP0717, | | | |
| EIP0698, | | | |

EIP0708,

| | | | |
|---|---|---|---|
| EIP0699, | | | |
| EIP0708, | | | |
| EIP0709, | | | |
| BIP0745, | | | |
| EIP0746, | | | |
| EIP0751, | | | |
| EIP0752, | | | |
| EIP0133, | | | |
| EIP0553, | | | |
| EIP0554, | | | |
| VVV2.1 | RASENIYSYLA (SEQ | NAKTLQS (SEQ ID | QHHYGSPWT (SEQ ID |
| | ID NO: 222) | NO: 225) | NO: 224) |
| G28.8, | RASEKIYSYLA (SEQ | NAKTLAE { SEQ ID | QHHFGSPRT (SEQ ID |
| EIP0051, | ID NO: 430) | NO: 431) | NO: 432) |
| EIP0100, | | | |
| EIP0546, | RSSHSIRSRRVA (SEQ | GVSNRAS (SEQ ID | QVYGASSYT (SEQ ID |
| EIP0209 | ID NO: 433) | NO: 434) | NO: 435) |

In some embodiments, the second antigen binding region comprises a) a heavy chain variable region (VH) comprising a i) a VH complementarity determining region 1 (VH_{CDR1}) comprising the amino acid sequence of SEQ ID NO: 210, ii) a VH complementarity determining region 2 (VH_{CDR2}) comprising the amino acid sequence of SEQ ID NO: 212, iii) a VH complementarity determining region 3 (VH_{CDR3}) comprising the amino acid sequence of SEQ ID NO: 214; and b) a light chain variable region (VL) comprising a i) a VL complementarity determining region 1 (VL_{CDR1}) comprising the amino acid sequence of SEQ ID NO: 222, ii) a VL complementarity determining region 2 (VL_{CDR2}) comprising the amino acid sequence of SEQ ID NO: 223, iii) a VL complementarity determining region 3 (VL_{CDR3}) comprising the amino acid sequence of SEQ ID NO: 224.

In some embodiments, the second antigen binding region comprises a) a heavy chain variable region (VH) comprising a i) a VH complementarity determining region 1 (VH_{CDR1}) comprising the amino acid sequence of SEQ ID NO: 210, ii) a VH complementarity determining region 2 (VH_{CDR2}) comprising the amino acid sequence of SEQ ID NO: 216, iii) a VH complementarity determining region 3 (VH_{CDR3}) comprising the amino acid sequence of SEQ ID NO: 214; and b) a light chain variable region (VL) comprising a i) a VL complementarity determining region 1 (VL_{CDR1}) comprising the amino acid sequence of SEQ ID NO: 222, ii) a VL complementarity determining region 2 (VL_{CDR2}) comprising the amino acid sequence of SEQ ID NO: 225, iii) a VL complementarity determining region 3 (VL_{CDR3}) comprising the amino acid sequence of SEQ ID NO: 224.

In some embodiments, the second antigen binding region comprises a) a heavy chain variable region (VH) comprising a i) a VH complementarity determining region 1 (VH_{CDR1}) comprising the amino acid sequence of SEQ ID NO: 210, ii) a VH complementarity determining region 2 (VH_{CDR2}) comprising the amino acid sequence of SEQ ID NO: 216, iii) a VH complementarity determining region 3 (VH_{CDR3}) comprising the amino acid sequence of SEQ ID NO: 214; and b) a light chain variable region (VL) comprising a i) a VL complementarity determining region 1 (VL_{CDR1}) comprising the amino acid sequence of SEQ ID NO: 222, ii) a VL complementarity determining region 2 (VL_{CDR2}) comprising the amino acid sequence of SEQ ID NO: 223, iii) a VL complementarity determining region 3 (VL_{CDR3}) comprising the amino acid sequence of SEQ ID NO: 224.

In some embodiments, the second antigen binding region comprises a VH region comprising the amino acid sequence shown in SEQ ID NO: 197 and a VL region comprising the amino acid sequence shown in SEQ ID NO: 196.

In some embodiments, the second antigen binding region comprises a VH region comprising the amino acid sequence shown in SEQ ID NO: 199 and a VL region comprising the amino acid sequence shown in SEQ ID NO: 198.

In some embodiments, the second antigen binding region comprises a VH region comprising the amino acid sequence shown in SEQ ID NO: 201 and a VL region comprising the amino acid sequence shown in SEQ ID NO: 200.

In some embodiments, the second antigen binding region comprises a VH region comprising the amino acid sequence shown in SEQ ID NO: 203 and a VL region comprising the amino acid sequence shown in SEQ ID NO: 202.

In some embodiments, the second antigen binding region comprises a VH region comprising the amino acid sequence shown in SEQ ID NO: 205 and a VL region comprising the amino acid sequence shown in SEQ ID NO: 204.

In some embodiments, the second antigen binding region comprises a VH region comprising the amino acid sequence shown in SEQ ID NO: 161 and a VL region comprising the amino acid sequence shown in SEQ ID NO: 160.

In some embodiments, the second antigen binding region comprises a VH region comprising the amino acid sequence shown in SEQ ID NO: 165 and a VL region comprising the amino acid sequence shown in SEQ ID NO: 164.

In some embodiments, the second antigen binding region comprises a VH region comprising the amino acid sequence shown in SEQ ID NO: 167 and a VL region comprising the amino acid sequence shown in SEQ ID NO: 166.

In some embodiments, the second antigen binding region comprises a VH region comprising the amino acid sequence shown in SEQ ID NO: 169 and a VL region comprising the amino acid sequence shown in SEQ ID NO: 168.

In some embodiments, the second antigen binding region comprises a VH region comprising the amino acid sequence shown in SEQ ID NO: 171 and a VL region comprising the amino acid sequence shown in SEQ ID NO: 170.

In some embodiments, the second antigen binding region comprises a VH region comprising the amino acid sequence shown in SEQ ID NO: 173 and a VL region comprising the amino acid sequence shown in SEQ ID NO: 172.

In some embodiments, the second antigen binding region comprises a VH region comprising the amino acid sequence shown in SEQ ID NO: 175 and a VL region comprising the amino acid sequence shown in SEQ ID NO: 174.

In some embodiments, the second antigen binding region comprises a VH region comprising the amino acid sequence shown in SEQ ID NO: 177 and a VL region comprising the amino acid sequence shown in SEQ ID NO: 176.

In some embodiments, the second antigen binding region comprises a VH region comprising the amino acid sequence shown in SEQ ID NO: 179 and a VL region comprising the amino acid sequence shown in SEQ ID NO: 178.

In some embodiments, the second antigen binding region comprises a VH region comprising the amino acid sequence shown in SEQ ID NO: 181 and a VL region comprising the amino acid sequence shown in SEQ ID NO: 180.

In some embodiments, the second antigen binding region comprises a VH region comprising the amino acid sequence shown in SEQ ID NO: 183 and a VL region comprising the amino acid sequence shown in SEQ ID NO: 182.

In some embodiments, the second antigen binding region comprises a VH region comprising the amino acid sequence shown in SEQ ID NO: 185 and a VL region comprising the amino acid sequence shown in SEQ ID NO: 184.

In some embodiments, the second antigen binding region comprises a VH region comprising the amino acid sequence shown in SEQ ID NO: 187 and a VL region comprising the amino acid sequence shown in SEQ ID NO: 186.

In some embodiments, the second antigen binding region comprises a VH region comprising the amino acid sequence shown in SEQ ID NO: 189 and a VL region comprising the amino acid sequence shown in SEQ ID NO: 188.

In some embodiments, the second antigen binding region comprises a VH region comprising the amino acid sequence shown in SEQ ID NO: 191 and a VL region comprising the amino acid sequence shown in SEQ ID NO: 190.

In some embodiments, the second antigen binding region comprises a VH region comprising the amino acid sequence shown in SEQ ID NO: 193 and a VL region comprising the amino acid sequence shown in SEQ ID NO: 192.

In some embodiments, the second antigen binding region comprises a VH region comprising the amino acid sequence shown in SEQ ID NO: 195 and a VL region comprising the amino acid sequence shown in SEQ ID NO: 194.

Exemplary Bispecific Antibodies that Bind to Cd3 and Cd180

Provided herein are bispecific antibodies comprising a first antigen binding domain that binds a first antigen (e.g. CD3ε) and a second antigen binding domain that binds to a CD180. In some embodiments the bispecific antibody has the following structure: a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2).

In some embodiments, the bispecific antibody of the disclosure comprises a first antigen binding domain (e.g. binding to CD3ε) comprising: a) a heavy chain variable region (VH1) comprising a VH complementarity determining region 1 (VH1_{CDR1}), a VH complementarity determining region 2 (VH1_{CDR2}) and a VH complementarity determining region 3 (VH1_{CDR3}); and b) a light chain variable region (VL) comprising a VL complementarity determining region 1 (VL1_{CDR1}), a VL complementarity determining region 2 (VL1_{CDR2}) and a VL complementarity determining region 3 (VL1_{CDR3}); and a second antigen binding domain (e.g. binding to CD180) comprising: a) a heavy chain variable region (VH2) comprising a VH complementarity determining region 1 (VH2_{CDR1}), a VH complementarity determining region 2 (VH2_{CDR2}) and a VH2 complementarity determining region 3 (VH2_{CDR3}); and b) a light chain variable region (VL2) comprising a VL complementarity determining region 1 (VL2_{CDR1}), a VL complementarity determining region 2 (VL2_{CDR2}) and a VL complementarity determining region 3 (VL2_{CDR3}). Tables 9.2 and 9.3 provide exemplary of CDR sequences of the anti-CD3ε antibodies provided herein. Tables 11.2 and 11.3 provide exemplary of CDR sequences of the anti-CD180 antibodies provided herein.

In some embodiments, the bispecific antibody of the disclosure comprises a first antigen binding domain (e.g. binding to CD3ε) comprising any one of the VH1 and VL1 sequences listed in Table 9.1 and a second antigen binding domain (e.g. binding to CD180) comprising any one of the VH2 and VL2 sequences listed in Table 11.1.

In some embodiments, the bispecific antibody of the disclosure comprises a first heavy chain polypeptide (H1) and a first light chain polypeptide (L1), and a second heavy chain polypeptide (H2) and a second light chain polypeptide (L2) comprising any one of the sequence listed in Table 11.4 and Table 11.5. The italicized sequences are the heavy chain variable regions and the light chain variable regions. The underlined sequences are CDRs according to Kabat and the bolded sequences are CDRs according to Chothia.

In some embodiments, the bispecific antibody of the disclosure provided herein comprises a H1 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 11.4 and Table 11.5. In some embodiments, the H1 comprises a C-terminal lysine (K) residue.

In some embodiments, the bispecific antibody of the disclosure provided herein comprises a L1 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 11.4 and Table 11.5.

In some embodiments, the bispecific antibody of the disclosure provided herein comprises a H2 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 11.4 and Table 11.5. In some embodiments, the H1 comprises a C-terminal lysine (K) residue.

In some embodiments, the bispecific antibody of the disclosure provided herein comprises a L2 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 16.

In some embodiments, the H1 amino acid sequence is numbered in accordance with SEQ ID NO. 229. In some embodiments the L1 amino acid sequence is numbered in accordance with SEQ ID NO: 228. In some embodiments, the H2 amino acid sequence is numbered in accordance with SEQ ID NO: 227. In some embodiments, the L2 amino acid sequence is numbered in accordance with SEQ ID NO: 226.

TABLE 11.4
Exemplary Bispecific Antibodies that bind CD3s and CD180

| | Light/ | | SEQ |
|---|---|---|---|
| Antibody | Heavy | | ID |
| Name | Chain | Amino acid Sequence | NO: |
| NNN1042 | huMHR | DIQMTQSPSTLSASVGDRVTITCRASENIYSYLAWYQKKPGKAP | 226 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | GeneID: | STLRLSKADYEKHKVYACEVTHQGLSSPVTKSFNRGEC | |
| | 507531 | | |
| | huMHR | QVQLVQSGAEVKKPGASVKVSCKASGYTFTDYTMNWVRDAPGQG | 227 |
| | heavy | LEWIGLINPYNGVTSYNPNFQGRVTLTVDKSTSTAYMELSSLRS | |
| | chain | EDTAVYYCARSGDYDRNYEDYWGQGTLVTVSSASTKGPSVFPLA | |
| | (H2) | PSSKSTSGGTAALGCLVDDYFPEPVTVSWNSGALTSGVHTFPAV | |
| | GeneID: | LQSSGLYSLSSVVTVPSSSLGTQTYICNVNHKPSNTKVDKKVEP | |
| | 507528 | KSCDKTHTCPPCPAPEAAGAPSVFLFPPKPKDTLMISRTPEVTC | |
| | | VVVDVSHEDPEVKFNWYVDGVEVHNAKIKPREEQYNSTYRVVSV | |
| | | LTVLHQDWINGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 228 |
| | light | APRGLIGGTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNDYAMNWVRKAPGKG | 229 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| NNN1043 | huMHR | DIQMTQSPSSLSASVGDRVTITCRASENIYSYLAWYQKKPGKAP | 230 |
| | light | KLLVNNAKTLAEGVPSRFSGSGSGTDFTLTISSLQPEDFATYYC | |
| | chain | QHHYGSPWTFGGGTELEIKRTVAAPSVFIFPPSDEQLKSGTASV | |
| | (L2) | VCLLNNFYPREAKVQWKVDNALQSGNSQESVTEQDSKDSTYSLS | |
| | GeneID: | STLRLSKADYEKHKVYACEVTHQGLSSPVTKSFNRGEC | |
| | 507532 | | |
| | huMHR | QVQLVQSGAEVKKPGSSVKVSCKASGYSFTDYTMNWVRDAPGQG | 231 |
| | heavy | LEWIGLINPYNGVTSYNPNFQGRATLTVDKSTSTAYMELSSLRS | |
| | chain | EDTAVYYCARSGDYDRNYEDYWGQGTTLTVSSASTKGPSVFPLA | |
| | (H2) | PSSKSTSGGTAALGCLVDDYFPEPVTVSWNSGALTSGVHTFPAV | |
| | GeneID : | LQSSGLYSLSSVVTVPSSSLGTQTYICNVNHKPSNTKVDKKVEP | |
| | 507529 | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWINGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 232 |
| | light | APRGLIGGTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNDYAMNWVRKAPGKG | 233 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| NNN1044 | huMHR | DIQMTQSPSSLSASVGDRVTITCRASENIYSYLAWYQKKPGKAP | 234 |
| | light | KLLVNNAKTLAEGVPSRFSGSGSGTEFTLTISSLQPEDFATYYC | |
| | chain | QHHYGSPWTFGGGTELEIKRTVAAPSVFIFPPSDEQLKSGTASV | |
| | (L2) | VCLLNNFYPREAKVQWKVDNALQSGNSQESVTEQDSKDSTYSLS | |
| | GeneID: | STLRLSKADYEKHKVYACEVTHQGLSSPVTKSFNRGEC | |
| | 507533 | | |
| | huMHR | EVQLVQSGAEVKKPGESLKISCKASGYSFTDYTMNWVRDMPGKG | 235 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | GeneID: | LQSSGLYSLSSVVTVPSSSLGTQTYICNVNHKPSNTKVDKKVEP | |
| | 507530 | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 236 |
| | light | APRGLIGGTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNDYAMNWVRKAPGKG | 237 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| NNN1056 | huMHR | DIQMTQSPSTLSASVGDRVTITCRASENIYSYLAWYQKKPGKAP | 238 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | GeneID: | STLRLSKADYEKHKVYACEVTHQGLSSPVTKSFNRGEC | |
| | 507531 | | |
| | huMHR | EVQLVQSGAEVKKPGESLKISCKASGYSFTDYTMNWVRDMPGKG | 239 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | GeneID: | LQSSGLYSLSSVVTVPSSSLGTQTYICNVNHKPSNTKVDKKVEP | |
| | 507530 | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 240 |
| | light | APRGLIGGTNKRAPGTPARFSGSLLGGKAALTLSGVQPEDEABY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNDYAMNWVRKAPGKG | 241 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| NNN1057 | huMHR | DIQMTQSPSTLSASVGDRVTITCRASENIYSYLAWYQKKPGKAP | 242 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | GeneID: | STERLSKADYEKHKVYACEVTHQGLSSPVTKSFNRGEC | |
| | 507531 | | |
| | huMHR | EVQLVQSGAEVKKPGSSVKVSCKASGYSFTDYTMNWVRDAPGQG | 243 |
| | heavy | LEWIGLINPYNGVTSYNPNFQGRATLTVDKSTSTAYMELSSLRS | |
| | chain | EDTAVYYCARSGDYDRNYFDYWGQGTLVTVSSASTKGPSVFPLA | |
| | (H2) | PSSKSTSGGTAALGCLVDDYFPEPVTVSWNSGALTSGVHTFPAV | |
| | GeneID: | LQSSGLYSLSSVVTVPSSSLGTQTYICNVNHKPSNTKVDKKVEP | |
| | 520067 | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 244 |
| | light | APRGLIGGTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNDYAMNWVRKAPGKG | 245 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0696 | huMHR | DIQMTQSPSTLSASVGDRVTITCRASENIYSYLAWYQKKPGKAP | 246 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | | STLRLSKADYEKHKVYACEVTHQGLSSPVTKSFNRGEC | |
| | huMHR | EVQLVQSGAEVKKPGESLKISCKASGYSFTDYTMNWVRDMPGKG | 247 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSIGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD180 | EVQLVQSGAEVKKPGESLKISCKASGYSFTDYTMNWVRDMPGKG | 690 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | with K | PSSKSTSGGTAALGCLVDDYFPEPVTVSWNSGALTSGVHTFPAV | |
| | residue | LQSSGLYSLSSVVTVPSSSLGTQTYICNVNHKPSNTKVDKKVEP | |
| | at last | KSCDKTHTCPPCPAPEAAGAPSVFLFPPKPKDTLMISRTPEVTC | |
| | position | VVVDVSHEDPEVKFNWYVDGVEVHNAKTKPREEQYNSTYRVVSV | |
| | (H2) | LTVLHQDWINGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPGK | |
| | CD3 | ELVVTQEPSLTVSPGGTVTLTCRSSTGAVTTSNYANWVQDKPGQ | 248 |
| | light | APRGLIGGTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 249 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| | CD3 | EVQLLESGGGLVQPGGSLKLSCAASGFTFNTYAMNWVRKAPGKG | 691 |
| | heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | with K | VFPLAPSSKSTSGGTAALGCLVKDYFPEPVTVSWNSGALTSGVH | |
| | residue | TFPACLQSSGLYSLSSVVTVPSSSLGTQTYICNVNHKPSNTKVD | |
| | at last | KKVEPKSSDKTHTCPPCPAPEAAGAPSVFLFPPKPKDTLMISRT | |
| | position | PEVTCVVVDVSHEDPEVKFNWYVDGVEVHNAKTKPREEQYNSTY | |
| | (H1 | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPGK | |
| EIP0706 | huMHR | DIQMTQSPSTLSASVGDRVTITCRASENIYSYLAWYQKKPGKAP | 254 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | | STLRLSKADYEKHKVYACEVTHQGLSSPVTKSFNRGEC | |
| | huMHR | EVQLVQSGAEVKKPGESLKISCKASGYSFTDYTMNWVRDMPGKG | 255 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 256 |
| | light | APRGLIGDTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 257 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSIGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0710 | huMHR | DIQMTQSPSTLSASVGDRVTITCRASENIYSYLAWYQKKPGKAP | 262 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | | STLRLSKADYEKHKVYACEVTHQGLSSPVTKSFNRGEC | |
| | huMHR | EVQLVQSGAEVKKPGESLKISCKASGYSFTDYTMNWVRDMPGKG | 263 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 264 |
| | light | APRGLIGGTNKRAPGTPARFSGSLLGGKAALTLSGVQPEDEAEY | |
| | chain | YCALWYSQLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 265 |
| | Heavy | LEWVARIRSKYNNYATYYADSVKDRFTISRDDSKNTAYLQMNNL | |
| | chain | KTEDTAVYYCVRHGNFGDDYVSWFAYWGQGTLVTVSSASTKGPS | |
| | (H1) | VFPLAPSSKSTSGGTAALGCLVKDYFPEPVTVSWNSGALTSGVH | |
| | | TSPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0698 | huMHR | DIQMTQSPSTLSASVGDRVTITCRASENIYSYLAWYQKKPGKAP | 270 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (02) | VCLLNNFYPREAKVQWKVDNALQSGNSQESVTEQDSKDSTYSLS | |
| | | STLRLSKADYEKHKVYACEVTHQGLSSPVTKSFNRGEC | |
| | huMHR | EVQLVQSGAEVKKPGESLKISCKASGYSFTDYTMNWVRDMPGKG | 271 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSIGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKENWYVDGVEVHNAKTKPREEQYNSTYRVVSV | |
| | | LTVLHQDWLNGKEYKCKVSNKALPAPIEKTISKAKGQPREPQVY | |
| | | TIPPCREEMTKNQVSLWCLVKGFYPSDIAVEWESNGQPENNYKT | |
| | | TPPVLDSDGSFFLYSKETVDKSRWQQGNVFSCSVMHEALHNHYT | |
| | | QKSLSLSPG | |
| | CD180 | EVQLVQSGAEVKKPGESLKISCKASGYSFTDYTMNWVRDMPGKG | 692 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | with K | PSSKSTSGGTAALGCLVDDYFPEPVTVSWNSGALTSGVHTFPAV | |
| | residue | LQSSGLYSLSSVVTVPSSSLGTQTYICNVNHKPSNTKVDKKVEP | |
| | at last | KSCDKTHTCPPCPAPEAAGAPSVFLFPPKPKDTLMISRTPEVTC | |
| | position | VVVDVSHEDPEVKFNWYVDGVEVHNAKTKPREEQYNSTYRVVSV | |
| | (H2) | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPGK | |
| | CD3 | ELVVTQEPSLTVSPGGTVTLTCRSSTGAVTTSNYANWVQDKPGQ | 272 |
| | light | APRGLIGGTNKRAPGTPARFSGSLLGGKAALTLSGVQPEDEABY | |
| | chain | YCALWYSNLWVFGGGTKLTVLGQPKANPTVTLFPPSSEELQANK | |
| | (1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNDYAMNWVRKAPGKG | 273 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| | CD3 | EVQLLESGGGLVQPGGSLKLSCAASGFTFNDYAMNWVRKAPGKG | 693 |
| | heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | with K | VFPLAPSSKSTSGGTAALGCLVKDYFPEPVTVSWNSGALTSGVH | |
| | residue | TFPACLQSSGLYSLSSVVTVPSSSLGTQTYICNVNHKPSNTKVD | |
| | at last | KKVEPKSSDKTHTCPPCPAPEAAGAPSVFLFPPKPKDTLMISRT | |
| | position | PEVTCVVVDVSHEDPEVKFNWYVDGVEVHNAKTKPREEQYNSTY | |
| | (H1) | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPGK | |
| EIP0708 | huMHR | DIQMTQSPSTLSASVGDRVTITCRASENIYSYLAWYQKKPGKAP | 278 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | | STLRLSKADYEKHKVYACEVTHQGLSSPVTKSFNRGEC | |
| | huMHR | EVQLVQSGAEVKKPGESLKISCKASGYSFTDYTMNWVRDMPGKG | 279 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 280 |
| | light | APRGLIGGTNKRAPGTPARFSGSLLGGKAALTLSGVQPEDEAEY | |
| | chain | YCALWYSQLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 281 |
| | Heavy | LEWVAKIRSKYNNYATYYADSVKDRFTISRDDSKNTAYLQMNNL | |
| | chain | KTEDTAVYYCVRHGNFGDSYVSWFAYWGQGTLVTVSSASTKGPS | |
| | (H1) | VFPLAPSSKSTSGGTAALGCLVKDYFPEPVTVSWNSGALTSGVH | |
| | | TSPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0745 | huMHR | DIQMTQSPSTLSASVGDRVTITCRASENIYSYLAWYQKKPGKAP | 286 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | | STLRLSKADYEKHKVYACEVTHQGLSSPVTKSFNRGEC | |
| | huMHR | EVQLVQSGAEVKKPGESLKISCKASGYSFTDYTMNWVRDMPGKG | 287 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSIGAVTTSNYANWVQDKPGQ | 288 |
| | light | APRGLIGGTNKRAPGTPARFSGSLLGGKAALTLSGVQPEDEAEY | |
| | chain | YCALWYSQLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 289 |
| | Heavy | LEWVARIRSKYNNYATYYADSVKDRFTISRDDSKNTAYLQMNNL | |
| | chain | KTEDTAVYYCVRHGSFGNSYVSWFAYWGQGTLVTVSSASTKGPS | |
| | (H1) | VFPLAPSSKSTSGGTAALGCLVKDYFPEPVTVSWNSGALTSGVH | |
| | | TSPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0751 | huMHR | DIQMTQSPSTLSASVGDRVTITCRASENIYSYLAWYQKKPGKAP | 294 |
| | light | KLLVNNAKTLAEGVPSRFSGSGSGTEFTLTISSLQPDDFATYYC | |
| | chain | QHHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | | STLRLSKADYEKHKVYACEVTHQGLSSPVTKSFNRGEC | |
| | huMHR | EVQLVQSGAEVKKPGESLKISCKASGYSFTDYTMNWVRDMPGKG | 295 |
| | heavy | LEWIGLINPYNGVTSYNPNFQGQATLSVDKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKFNWYVDGVEVHNAKTKPREEQYNSTYRVVSV | |
| | | LTVLHQDWINGKEYKCKVSNKALPAPIEKTISKAKGQPREPQVY | |
| | | TLPPCREEMTKNQVSLWCLVKGFYPSDIAVEWESNGQPENNYKT | |
| | | TPPVLDSDGSFFLYSKLTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 296 |
| | light | APRGLIGGTNKRAPGTPARFSGSLLGGKAALTLSGVQPEDEAEY | |
| | chain | YCALWYSQLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 297 |
| | Heavy | LEWVARIRSKYNNYATYYADSVKDRFTISRDDSKNTAYLQMNNL | |
| | chain | KTEDTAVYYCVRHGTFGNSYVSWFAYWGQGTLVTVSSASTKGPS | |
| | (H1) | VFPLAPSSKSTSGGTAALGCLVKDYFPEPVTVSWNSGALTSGVH | |
| | | TSPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWINGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0553 | huMHR | DIQMTQSPSTLSASVGDRVTITCRASENIYSYLAWYQKKPGKAP | 391 |
| | light | KLLVNNAKTLAEGVPSRFSGSGSGTEFTLTISSLQPDDFATYYC | |
| | chain | QKHYGSPWTFGQGTKVEIKRTVAAPSVFIFPPSDEQLKSGTASV | |
| | (L2) | VCLLNNFYPREAKVQWKVDNALQSGNSQESVTEQDSKDSTYSLS | |
| | | STLRLSKADYEKHKVYACEVTHQGLSSPVTKSENRGEC | |
| | huMHR | EVQLVQSGAEVKKPGESLKISCKASGYSFTDYTMNWVRDMPGKG | 392 |
| | heavy | LEWIGLINPYNGVTSYNPNFQGQATLSVQKSISTAYLQWSSLKA | |
| | chain | SDTAMYYCARSGDYDRNYFDYWGQGTTLTVSSASTKGPSVFPLA | |
| | (H2) | PSSKSTSGGTAALGCLVDDYFPEPVTVSWNSGALTSGVHTFPAV | |
| | | LQSSGLYSLSSVVTVPSSSIGTQTYICNVNHKPSNTKVDKKVEP | |
| | | KSCDKTHTCPPCPAPEAAGAPSVFLFPPKPKDTLMISRTPEVTC | |
| | | VVVDVSHEDPEVKENWYVDGVEVHNAKTKPREEQYNSTYRVVSV | |
| | | LTVLHQDWLNGKEYKCKVSNKALPAPIEKTISKAKGQPREPQVY | |
| | | TLPPSREEMTKNQVSLTCLVKGFYPSDIAVEWESNGQPENNYKT | |
| | | TPPVLDSDGSFFLYSKUTVDKSRWQQGNVFSCSVMHEALHNHYT | |
| | | QKSLSLSPG | |
| | CD3 | ELVVTQEPSLTVSPGGTVTLTCRSSTGAVTTSNYANWVQDKPGQ | 393 |
| | light | APRGLIGGTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 394 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0546 | B12 | EIVLTQSPGTLSLSPGERATFSCRSSHSIRSRRVAWYQKKPGQA | 399 |
| | light | PRLVIHGVSNRASGISDRFSGSGSGTDFTLTITRVEPEDFALYY | |
| | chain | CQVYGASSYTFGQGTKLERKRTVAAPSVFIFPPSDEQLKSGTAS | |
| | (L2) | VVCLINNFYPREAKVQWKVDNALQSGNSQESVTEQDSKDSTYSL | |
| | | SSTLRLSKADYEKHKVYACEVTHQGLSSPVTKSENRGEC | |
| | B12 | QVQLVQSGAEVKKPGASVKVSCQASGYRFSNFVIHWVRDAPGQR | 400 |
| | heavy | QEWMGWINPYNGNKEFSAKFQDRVTFTADTSANTAYMELRSLRS | |
| | chain | ADTAVYYCARVGPYSWDDSPQDNYYMDVWGKGTTVIVSSASTKG | |
| | (H2) | PSVFPLAPSSKSTSGGTAALGCLVDDYFPEPVTVSWNSGALTSG | |
| | | VHTFPAVLQSSGLYSLSSVVTVPSSSLGTQTYICNVNHKPSNTK | |
| | | VDKKVEPKSCDKTHTCPPCPAPEAAGAPSVFLFPPKPKDTLMIS | |
| | | RTPEVTCVVVDVSHEDPEVKENWYVDGVEVHNAKTKPREEQYNS | |
| | | TYRVVSVLTVLHQDWLNGKEYKCKVSNKALPAPIEKTISKAKGQ | |
| | | PREPQVYTLPPCREEMTKNQVSLWCLVKGFYPSDIAVEWESNGQ | |
| | | PENNYKTTPPVLDSDGSFFLYSKLTVDKSRWQQGNVFSCSVMHE | |
| | | ALHNHYTQKSLSLSPG | |
| | CD3 | ELVVTQEPSLTVSPGGTVTLTCRSSTGAVTTSNYANWVQDKPGQ | 401 |
| | light | APRGLIGGTNKRAPGTPARFSGSLLGGKAALTLSGVQPEDEAEY | |
| | chain | YCALWYSNLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 402 |
| | Heavy | LEWVARIRSKYNNYATYYADSVKDRFTISRDDSKNTAYLQMNNL | |
| | chain | KTEDTAVYYCVRHGNFGNSYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP0209 | B12 | QVQLVQSGAEVKKPGASVKVSCQASGYRFSNFVIHWVRQAPGQR | 403 |
| | light | FEWMGWINPYNGNKEFSAKFQDRVTFTADTSANTAYMELRSLRS | |
| | chain | ADTAVYYCARVGPYSWDDSPQDNYYMDVWGKGTTVIVSSASTKG | |
| | (L2) | PSVFPLAPSSKSTSGGTAALGCLVKDYFPEPVTVSWNSGALTSG | |
| | | VHTFPAVLQSSGLYSLSSVVTVPSSSIGTQTYICNVNHKPSNTK | |
| | | VDKKVEPKSCDKTHTCPPCPAPEAAGAPSVFLFPPKPKDTLMIS | |
| | | RTPEVTCVVVDVSHEDPEVKENWYVDGVEVHNAKTKPREEQYNS | |
| | | TYRVVSVLTVLHQDWLNGKEYKCKVSNKALPAPIEKTISKAKGQ | |
| | | PREPQVYTLPPCREEMTKNQVSLWCLVKGFYPSDIAVEWESNGQ | |
| | | PENNYKTTPPVLDSDGSFFLYSKLTVDKSRWQQGNVFSCSVMHE | |
| | | ALHNHYTQKSLSLSPG | |
| | B12 | EIVLTQSPGTLSLSPGERATFSCRSSHSIRSRRVAWYQHKPGQA | 404 |
| | heavy | PRLVIHGVSNRASGISDRFSGSGSGTDFTLTITRVEPEDFALYY | |
| | chain | CQVYGASSYTFGQGTKLERKRTVAAPSVFIFPPSDEQLKSGTAS | |
| | (H2) | VVCLLNNFYPREAKVQWKVDNALQSGNSQESVTEQDSKDSTYSL | |
| | | SSTLTLSKADYEKHKVYACEVTHQGLSSPVTKSFNRGEC | |
| | CD3 | DIQMTQSPSSLSASVGDRVTITCTSSQSLENVRSQKNYLAWYQQ | 405 |
| | light | KPGKAPKLLIYWASTRESGVPSRFSGSGSGTDFTLTISSLQPED | |
| | chain | FATYYCKQSYDLFTFGGGTKVEIKSSASTKGPSVFPLAPSSKST | |
| | (L1) | SGGTAALGCLVKDYFPEPVTVSWNSGALTSGVHTFPAVLQSSGL | |
| | | YSLSSVVTVPSSSLGTQTYICNVNHKPSNTKVDKKVEPKSC | |
| | CD3 | EVQLVESGGGLVQPGGSLRLSCAASGFTFSDYYMTWVRQAPGKG | 406 |
| | Heavy | LEWVAFIRNQARGYTSDHNPSVKGRFTISRDNAKNSLYLQMNSL | |
| | chain | RAEDTAVYYCARDRPSYYVLDYWGQGTTVTVSSASVAAPSVFIF | |
| | (H1) | PPSDEQLKSGTASVVCLLNNFYPREAKVQWKVDNALQSGNSQES | |
| | | VTEQDSKDSTYSLSSTLTLSKADYEKHKVYACEVTHQGLSSPVT | |
| | | KSENRGECDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPG | |
| EIP1053 | b12 | EIVLTQSPGTLSLSPGERATFSCRSSHSIRSRRVAWYQKKPGQA | 407 |
| | light | PRLVIHGVSNRASGISDRFSGSGSGTDFTLTITRVEPEDFALYY | |
| | chain | CQVYGASSYTFGQGTKLERKRTVAAPSVFIFPPSDEQLKSGTAS | |
| | (L2) | VVCLLNNFYPREAKVQWKVDNALQSGNSQESVTEQDSKDSTYSL | |
| | | SSTLRLSKADYEKHKVYACEVTHQGLSSPVTKSFNRGEC* | |
| | b12 | QVQLVQSGAEVKKPGASVKVSCQASGYRFSNFVIHWVRDAPGQR | 408 |
| | heavy | QEWMGWINPYNGNKEFSAKFQDRVTFTADTSANTAYMELRSLRS | |
| | chain | ADTAVYYCARVGPYSWDDSPQDNYYMDVWGKGTTVIVSSASTKG | |
| | (H2) | PSVFPLAPSSKSTSGGTAALGCLVDDYFPEPVTVSWNSGALTSG | |
| | | VHTFPAVLQSSGLYSLSSVVTVPSSSLGTQTYICNVNHKPSNTK | |
| | | VDKKVEPKSCDKTHTCPPCPAPEAAGAPSVFLFPPKPKDTLMIS | |
| | | RTPEVTCVVVDVSHEDPEVKENWYVDGVEVHNAKTKPREEQYNS | |
| | | TYRVVSVLTVLHQDWLNGKEYKCKVSNKALPAPIEKTISKAKGQ | |
| | | PREPQVYTLPPCREEMTKNQVSLWCLVKGFYPSDIAVEWESNGQ | |
| | | PENNYKTTPPVLDSDGSFFLYSKLTVDKSRWQQGNVFSCSVMHE | |
| | | ALHNHYTQKSLSLSPG* | |
| | CD3 | ELVVTQEPSLTVSPGGTVTLTCRSSTGAVTTSNYANWVQDKPGQ | 409 |
| | light | APRGLIGGTNKRAPGTPARFSGSLLGGKAALTLSGVQPEDEAEY | |
| | chain | YCALWYSQLWVFGGGTKLTVLGQPKANPTVTLFPPSSEELQANK | |
| | (L1) | ADLVCLISDFYPGAVTVAWKADGSPVKAGVETCKPSKQSNNKYA | |
| | | ASSYLSLTPEQWKSHRSYSCQVTHEGSTVEKTVAPTESS | |
| | CD3 | EVQLLESGGGLVQPGGSLKLSCAASGFTFNTYAMNWVRKAPGKG | 410 |
| | Heavy | LEWVARIRSKYNNYATYYADSVKDRFTISRDDSKNTAYLQMNNL | |
| | chain | KTEDTAVYYCVRHGNFGDDYVSWFAYWGQGTLVTVSSASTKGPS | |
| | (H1) | VFPLAPSSKSTSGGTAALGCLVKDYFPEPVTVSWNSGALTSGVH | |
| | | TFPACLQSSGLYSLSSVVTVPSSSLGTQTYICNVNHKPSNTKVD | |
| | | KKVEPKSSDKTHTCPPCPAPEAAGAPSVFLFPPKPKDTLMISRT | |
| | | PEVTCVVVDVSHEDPEVKFNWYVDGVEVHNAKTKPREEQYNSTY | |
| | | RVVSVLTVLHQDWLNGKEYKCKVSNKALPAPIEKTISKAKGQPR | |
| | | EPQVCTLPPSREEMTKNQVSLSCAVKGFYPSDIAVEWESNGQPE | |
| | | NNYKTTPPVLDSDGSFFLVSKLTVDKSRWQQGNVFSCSVMHEAL | |
| | | HNHYTQKSLSLSPGGGGSGGGGSFSQQIYGVVYGNVTFHVPSNV | |
| | | PLKEVLWKKQKDKVAELENSEFRAFSSFKNRVYLDTVSGSLTIY | |
| | | NLTSSDEDEYEMESPNITDTMKFFLYVLESLPSPILTCALINGS | |
| | | IEVQCMIPEHYNSHRGLIMYSWDCPMEQCKRNSTSIYFKMENDL | |
| | | PQKIQCTLSNPLFNTTSSIILTTCIPSSGHSRHRY | |

Exemplary CD3ε×CD180 bispecific antibodies of the invention include NNN1042, NNN1043, NNN1044, NNN1056, NNN1057, EIP0696, EIP0706, EIP0710, EIP0698, EIP0708, EIP0745 and EIP0751.

Bispecific antibodies NNN1042, NNN1043, NNN1044, NNN1056 and EIP0698, comprise a first antigen binding domain that binds CD3ε which comprising a VH1, comprising a VH1_{CDR1} having the amino acid sequence of SEQ ID NO: 30; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL_{CDR2} having the amino acid sequence of SEQ ID NO: 43; and a VL1_{CDR3} having the amino acid sequence of SEQ ID NO: 45.

Bispecific antibody EIP0696 comprises a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

Bispecific antibody EIP0706 comprises a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 44; and a VL1_{CDR3} having the amino acid sequence of SEQ ID NO: 45.

Bispecific antibody EIP0710 comprises a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 39; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

Bispecific antibody EIP0708 comprises a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 35; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

Bispecific antibody EIP0745 comprises a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 36; and a VH1_{CDR3} having the amino acid sequence of SEQ ID NO: 40; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

Bispecific antibody EIP0751 comprises a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 41; and a VL1, comprising a VL1_{CDR1} having the amino acid sequence of SEQ ID NO: 42: a VL_{CDR2} having the amino acid sequence of SEQ ID NO: 43; and a VL1_{CDR3} having the amino acid sequence of SEQ ID NO: 47.

Bispecific antibodies NNN1042, NNN1043, NNN1044, NNN1056, NNN1057, EIP06%, EIP0706, EIP0710, EIP0698, EIP0708, EIP0745 and EIP0751 comprise a second antigen binding domain that binds CD180 comprising a VH2, comprising VH2_{CDR1} having the amino acid sequence of SEQ ID NO: 210; a VH2_{CDR2} having the amino acid sequence of SEQ ID NO: 212; and a VH2_{CDR3} having the amino acid sequence of SEQ ID NO: 214; and a VL2, having VL2_{CDR1} having the amino acid sequence of SEQ ID NO: 222, a VL2_{CDR2} having the amino acid sequence of SEQ ID NO: 223; and a VL2_{CDR3} having the amino acid sequence of SEQ ID NO: 224.

In some embodiments, the bispecific antibody NNN1042 comprises a VH1 comprising the amino acid sequence of SEQ ID NO: 17, a VL1 comprising the amino acid sequence of SEQ ID NO: 22, a VH2 comprising the amino acid sequence of SEQ ID NO: 197, and a VL2 comprising the amino acid sequence of SEQ ID NO: 196.

In some embodiments, the bispecific antibody NNN1042 comprises a H1 comprising the amino acid sequence of SEQ ID NO: 229, a L1 comprising the amino acid sequence of SEQ ID NO: 228, a H2 comprising the amino acid sequence of SEQ ID NO: 227, and a L2 comprising the amino acid sequence of SEQ ID NO: 226.

In some embodiments, the bispecific antibody NNN1043 comprises a VH1 comprising the amino acid sequence of SEQ ID NO: 17, a VL1 comprising the amino acid sequence of SEQ ID NO: 22, a VH2 comprising the amino acid sequence of SEQ ID NO: 199, and a VL2 comprising the amino acid sequence of SEQ ID NO: 198.

In some embodiments, the bispecific antibody NNN1043 comprises a H1 comprising the amino acid sequence of SEQ ID NO: 233, a L1 comprising the amino acid sequence of SEQ ID NO: 232, a H2 comprising the amino acid sequence of SEQ ID NO: 231, and a L2 comprising the amino acid sequence of SEQ ID NO: 230.

In some embodiments, the bispecific antibody NNN1044 comprises a VH1 comprising the amino acid sequence of SEQ ID NO: 17, a VL1 comprising the amino acid sequence of SEQ ID NO: 22, a VH2 comprising the amino acid sequence of SEQ ID NO: 201, and a VL2 comprising the amino acid sequence of SEQ ID NO: 200.

In some embodiments, the bispecific antibody NNN1044 comprises a H1 comprising the amino acid sequence of SEQ ID NO: 237, a L1 comprising the amino acid sequence of SEQ ID NO: 236, a H2 comprising the amino acid sequence of SEQ ID NO: 235, and a L2 comprising the amino acid sequence of SEQ ID NO: 234.

In some embodiments, the bispecific antibody NNN1056 comprises a VH1 comprising the amino acid sequence of SEQ ID NO: 17, a VL1 comprising the amino acid sequence of SEQ ID NO: 22, a VH2 comprising the amino acid sequence of SEQ ID NO: 203, and a VL2 comprising the amino acid sequence of SEQ ID NO: 202.

In some embodiments, the bispecific antibody NNN1056 comprises a H1 comprising the amino acid sequence of SEQ ID NO: 241, a L1 comprising the amino acid sequence of SEQ ID NO: 240, a H2 comprising the amino acid sequence of SEQ ID NO: 239, and a L2 comprising the amino acid sequence of SEQ ID NO: 238.

In some embodiments, the bispecific antibody NNN1057 comprises a VH1 comprising the amino acid sequence of SEQ ID NO: 17, a VL1 comprising the amino acid sequence of SEQ ID NO: 22, a VH2 comprising the amino acid sequence of SEQ ID NO: 205, and a VL2 comprising the amino acid sequence of SEQ ID NO: 204.

In some embodiments, the bispecific antibody NNN1057 comprises a H1 comprising the amino acid sequence of SEQ ID NO: 245, a L1 comprising the amino acid sequence of SEQ ID NO: 244, a H2 comprising the amino acid sequence of SEQ ID NO: 243, and a L2 comprising the amino acid sequence of SEQ ID NO: 242.

In some embodiments, the bispecific antibody EIP06% comprises a VH1 comprising the amino acid sequence of SEQ ID NO: 13, a VL1 comprising the amino acid sequence of SEQ ID NO: 22, a VH2 comprising the amino acid sequence of SEQ ID NO: 203, and a VL2 comprising the amino acid sequence of SEQ ID NO: 202.

In some embodiments, the bispecific antibody EIP0696 comprises a H1 comprising the amino acid sequence of SEQ ID NO: 249, a L1 comprising the amino acid sequence of SEQ ID NO: 248, a H2 comprising the amino acid sequence of SEQ ID NO: 247, and a L2 comprising the amino acid sequence of SEQ ID NO: 246.

In some embodiments, the bispecific antibody EIP0706 comprises a VH1 comprising the amino acid sequence of SEQ ID NO: 13, a VL1 comprising the amino acid sequence of SEQ ID NO: 27, a VH2 comprising the amino acid sequence of SEQ ID NO: 203, and a VL2 comprising the amino acid sequence of SEQ ID NO: 202.

In some embodiments, the bispecific antibody EIP0706 comprises a H1 comprising the amino acid sequence of SEQ ID NO: 257, a L1 comprising the amino acid sequence of SEQ ID NO: 256, a H2 comprising the amino acid sequence of SEQ ID NO: 255, and a L2 comprising the amino acid sequence of SEQ ID NO: 254.

In some embodiments, the bispecific antibody EIP0710 comprises a VH1 comprising the amino acid sequence of SEQ ID NO: 16, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0710 comprises a H1 comprising the amino acid sequence of SEQ ID NO: 265, a L1 comprising the amino acid sequence of SEQ ID NO: 264, a H2 comprising the amino acid sequence of SEQ ID NO: 263, and a L2 comprising the amino acid sequence of SEQ ID NO: 262.

In some embodiments, the bispecific antibody EIP0698 comprises a VH1 comprising the amino acid sequence of SEQ ID NO: 17, a VL1 comprising the amino acid sequence of SEQ ID NO: 22, a VH2 comprising the amino acid sequence of SEQ ID NO: 203, and a VL2 comprising the amino acid sequence of SEQ ID NO: 202.

In some embodiments, the bispecific antibody EIP0698 comprises a H1 comprising the amino acid sequence of SEQ ID NO: 273, a L1 comprising the amino acid sequence of SEQ ID NO: 272, a H2 comprising the amino acid sequence of SEQ ID NO: 271, and a L2 comprising the amino acid sequence of SEQ ID NO: 270.

In some embodiments, the bispecific antibody EIP0708 comprises a VH1 comprising the amino acid sequence of SEQ ID NO: 18, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0708 comprises a H1 comprising the amino acid sequence of SEQ ID NO: 281, a L1 comprising the amino acid sequence of SEQ ID NO: 280, a H2 comprising the amino acid sequence of SEQ ID NO: 279, and a L2 comprising the amino acid sequence of SEQ ID NO: 278.

In some embodiments, the bispecific antibody EIP0745 comprises a VH1 comprising the amino acid sequence of SEQ ID NO: 19, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0745 comprises a H1 comprising the amino acid sequence of SEQ ID NO: 289, a L1 comprising the amino acid sequence of SEQ ID NO: 288, a H2 comprising the amino acid sequence of SEQ ID NO: 287, and a L2 comprising the amino acid sequence of SEQ ID NO: 286.

In some embodiments, the bispecific antibody EIP0751 comprises a VH1 comprising the amino acid sequence of SEQ ID NO: 20, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0751 comprises a H1 comprising the amino acid sequence of SEQ ID NO: 297, a L1 comprising the amino acid sequence of SEQ ID NO: 296, a H2 comprising the amino acid sequence of SEQ ID NO: 295, and a L2 comprising the amino acid sequence of SEQ ID NO: 294.

Any one of the bispecific antibodies shown above in Table 11.4 can be further modified by substituting any one of the anti-CD3ε antigen binding regions with any one of the anti-CD3ε binding regions shown in Tables 9.1, 9.2 and 9.3. For example, the anti-CD3ε antigen binding regions of bispecific antibody “EIP1224” can be substituted with any one of the anti-CD3ε binding regions shown in Tables 9.1, 9.2 and 9.3 to produce the bispecific antibodies of the invention. Exemplary antibodies are shown in Table 11.4.

In some embodiments, the CD3×CD180 bispecific antibodies of the invention have a CD58 fusion peptide (SEQ ID NO: 49) fused indirectly at the C-terminus of the first heavy chain polypeptide (H1) using linker-1 (SEQ ID NO: 52). In some embodiments, the CD3×CD180 bispecific antibodies of the invention have a CD58v* fusion peptide (SEQ ID NO: 50) fused indirectly at the C-terminus of the first heavy chain polypeptide (H1) using linker-1 (SEQ ID NO: 52).

In some embodiments, the CD3×CD180 bispecific antibodies of the invention have a CD58 fusion peptide (SEQ ID NO: 49) fused indirectly at the C-terminus of the first heavy chain polypeptide (H1) using linker-2 (SEQ ID NO: 53). In some embodiments, the CD3×CD180 bispecific antibodies of the invention have a CD58v* fusion peptide (SEQ ID NO: 50) fused indirectly at the C-terminus of the first heavy chain polypeptide (H1) using linker-2 (SEQ ID NO: 53).

In some embodiments, the CD3×CD180 bispecific antibodies of the invention have a CD58 fusion peptide (SEQ ID NO: 49) fused indirectly at the C-terminus of the first heavy chain polypeptide (H1) using linker-3 (SEQ ID NO: 54). In some embodiments, the CD3×CD180 bispecific antibodies of the invention have a CD58v* fusion peptide (SEQ ID NO: 50) fused indirectly at the C-terminus of the first heavy chain polypeptide (H1) using linker-3 (SEQ ID NO: 54).

Exemplary CD3×CD180 bispecific antibodies having a CD58 fusion are shown in Table 11.5.

TABLE 11.5
Exemplary Bispecific Antibodies that bind to CD3s and
CD180 with a C-terminus CDS8 fusion peptide

| | Light/ | | SEQ |
|---|---|---|---|
| Antibody | Heavy | | ID |
| Name | Chain | Amino acid Sequence | NO |
| CIP1042 | huMHR | DIQMTQSPSTLSASVGDRVTITCRASENIYSYLAWYQKKPGKAPKLLVNNAKTLAEGVPS | 302 |
| | light | RFSGSGSGTEFTLTISSLQPDDFATYYCQHHYGSPWTFGQGTKVEIKRTVAAPSVFIFPP | |
| | chain | SDEQUKSGTASVVCLENNFYPREAKVQWKVDNALQSGNSQESVTEQDSKQSTYSLSSTER | |
| | (L2) | LSKADYEKHKVYACEVTHQGLSSPVTKSFNRGEC | |
| | GeneID: | | |
| | 507531 | | |
| | huMHR | QVQLVQSGAEVKKPGASVKVSCKASGYTFTDYTMNWVRDAPGQGLEWIGLINPYNGVTSY | 303 |
| | heavy | NPNFQGRVTLTVDKSTSTAYMELSSLRSEDTAVYYCARSGDYDRNYFDYWGQGTLVTVSS | |
| | chain | ASTKGPSVFPLAPSSKSTSGGTAALGCLVDDYFPEPVTVSWNSGALTSGVHTEPAVLQSS | |
| | (H2) | GLYSLSSVVTVPSSSLGTQTYICNVNHKPSNTKVDKKVEPKSCDKTHTCPPCPAPEAAGA | |
| | GeneID: | PSVFLFPPKPKDTLMISRTPEVTCVVVDVSHEDPEVKFNWYVDGVEVHNAKTKPREEQYN | |
| | 507528 | STYRVVSVLTVLHQDWINGKEYKCKVSNKALPAPIEKTISKAKGQPREPQVYTLPPCREE | |
| | | MIKNQVSLWCLVKGFYPSDIAVEWESNGQPENNYKTIPPVLDSDGSFFLYSKLTVDKSRW | |
| | | QQGNVFSCSVMHEALHNHYTQKSLSLSPG | |
| | CD3 | ELVVTQEPSLTVSPGGTVTLTCRSSTGAVTTSNYANWVQDKPGQAPRGLIGGTNKRAPGT | 304 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNDYAMNWVRKAPGKGLEWVARIRSKYNNYAT | 305 |
| | Heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGCLVKDYFPEPVTVSWNSGALTSGVHTEPA | |
| | (H1) | CLQSSGLYSLSSVVTVPSSSLGTQTYICNVNHKPSNTKVDKKVEPKSSDKTHTCPPCPAP | |
| | | EAAGAPSVFLEPPKPKDTIMISRTPEVTCVVVDVSHEDPEVKENWYVDGVEVHNAKTKPR | |
| | | EEQYNSTYRVVSVLTVLHQDWINGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLISSDEDEYEM | |
| | | ESPNITDTMKFFLYVLESLPSPTLTCALINGSIEVQCMIPEHYNSHRGLIMYSWDCPMEQ | |
| | | CKRNSTSIYFKMENDLPQKIQCTLSNPLENTISSIILTTCIPSSGHSRHRY | |
| EIP1043 | huMHR | DIQMTQSPSSLSASVGDRVTITCRASENIYSYLAWYQKKPGKAPKLLVNNAKTLAEGVPS | 306 |
| | light | RFSGSGSGTDFTLTISSLQPEDFATYYCQHHYGSPWTFGGGTELEIKRTVAAPSVFIFPP | |
| | chain | SDEQLKSGTASVVCLLNNFYPREAKVQWKVDNALQSGNSQESVTEQDSKDSTYSLSSTLR | |
| | (L2) | LSKADYEKHKVYACEVTHQGLSSPVIKSENRGEC | |
| | GeneID: | | |
| | 507532 | | |
| | huMHR | QVQLVQSGAEVKKPGSSVKVSCKASGYSFTDYTMNWVRDAPGQGLEWIGLINPYNGVTSY | 307 |
| | heavy | NPNFQGRATLTVDKSTSTAYMELSSERSEDTAVYYCARSGDYDRNYFDYWGQGTILTVSS | |
| | chain | ASTKGPSVFPLAPSSKSTSGGTAALGCLVDDYFPEPVTVSWNSGALTSGVHTFPAVLQSS | |
| | (H2) | GLYSLSSVVTVPSSSLGTQTYICNVNHKPSNTKVDKKVEPKSCDKTHTCPPCPAPEAAGA | |
| | GeneID: | PSVELFPPKPKDTUMISRTPEVTQVVVDVSHEDPEVKFNWYVDGVEVANAKTKPREEQYN | |
| | 507529 | STYRVVSVLTVLHQDWINGKEYKCKVSNKALPAPIEKTISKAKGQPREPQVYTLPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSLSPG | |
| | CD3 | ELVVTQEPSLTVSPGGTVTLTCRSSTGAVTTSNYANWVQDKPGQAPRGLIGGTNKRAPGT | 308 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD 3 | EVQLLESGGGLVQPGGSLKLSCAASGFTFNDYAMNWVRKAPGKGLEWVARIRSKYNNYAT | 309 |
| | Heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGQLVKDYFPEPVTVSWNSGALTSGVHTFPA | |
| | (H1) | CLQSSGLYSLSSVVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVDGVEVHNAKTKPR | |
| | | EEQYNSTYRVVSVLTVLHQDWINGKEYKCKVSNKALPAPIERTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNUTSSDEDEYEM | |
| | | ESPNITDTMKFFLYVLESLPSPTLICALINGSIEVQCMIPEHYNSHRGLIMYSWDCPMEQ | |
| | | CKRNSTSIYFKMENDLPQKIQCTLSNPLENTTSSIILTTCIPSSGHSRHRY | |
| EIP1044 | huMHR | DIQMTQSPSSLSASVGDRVTITCRASENIYSYLAWYQKKPGKAPKLLVNNAKTLAEGVPS | 310 |
| | light | RFSGSGSGTEFTETISSLQPEDFATYYCQRHYGSPWIFGGGTELEIKRTVAAPSVFIFPP | |
| | chain | SDEQLKSGTASVVCLLNNFYPREAKVQWKVDNALQSGNSQESVTEQDSKDSTYSLSSTLR | |
| | (L2) | LSKADYEKHKVYACEVTHQGLSSPVTKSFNRGEC | |
| | GeneID: | | |
| | 507533 | | |
| | huMHR | EVQLVQSGAEVKKPGESLKISCKASGYSFTDYTMNWVRDMPGKGLEWIGLINPYNGVTSY | 311 |
| | heavy | NPNFQGQATLSVDKSISTAYLQWSSLKASDTAMYYCARSGDYDRNYFDYWGQGTTLTVSS | |
| | chain | ASTKGPSVPPLAPSSKSTSGGTAALGCLVDDYFPEPVTVSWNSGALTSGVHTFPAVLQSS | |
| | (H2) | GLYSLSSVVTVPSSSLGTQTYICNVNBKPSNTKVDKKVEPKSCDKTETCPPCPAPEAAGA | |
| | GeneID: | PSVFLFPPKPKDTLMISRTPEVTCVVVDVSHEDPEVKFNWYVDGVEVHNAKTKPREEQYN | |
| | 507530 | STYRVVSVETVLHQDWLNGKEYKCKVSNKALPAPIEKTISKAKGQPREPQVYTIPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSLSPG | |
| | CD3 | ELVVTQEPSLTVSPGGTVTLTCRSSTGAVTTSNYANWVQDKPGQAPRGLIGGTNKRAPGT | 312 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNDYAMNWVRKAPGKGLEWVARIRSKYNNYAT | 313 |
| | Heavy | YYADSVKDRFTISRDDSKNTAYLQMNNLKTEDTAVYYCVRHGNHGNSYVSWFAYWGQGTL | |
| | chain | VTVSSASTKGPSVFPLAPSSKSTSGGTAALGCLVKDYFPEPVIVSWNSGALTSGVHTFPA | |
| | (H1) | CLQSSGLYSLSSVVTVPSSSLGTQTYICNVNHKPSNTKVDKKVEPKSSDKTHTCPPCPAP | |
| | | EAAGAPSVFLFPPKPKDTIMISRTPEVTCVVVDVSHEDPEVKFNWYVDGVEVHNAKTKPR. | |
| | | EEQYNSTYRVVSVLTVLHQDWLNGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLTSSDEDEYEM | |
| | | ESPNITDTMKFFLYVLESLPSPILTCALINGSIEVQCKIPEHYNSHRGLIMYSWDCPMEQ | |
| | | CKENSTSIYEKMENDLPQKIQCILSNPLFNITSSIILTTCIPSSGHSRARY | |
| EIP1056 | huMHR | DIQMTQSPSTLSASVGDRVTITCRASENIYSYLAWYQKKPGKAPKLLVNNAKTLAEGVPS | 314 |
| | light | RFSGSGSGTEFTLTISSLQPDDFATYYCQHHYGSPWTFGQGTKVEIKRTVAAPSVFIFPP | |
| | chain | SDEQLKSGTASVVCLLNNFYPREAKVQWKVDNALQSGNSQESVTEQDSKDSTYSLSSTLR | |
| | (L2) | LSKADYEKHKVYACEVTHQGLSSPVTKSFNRGEC | |
| | GeneID: | | |
| | 507531 | | |
| | huMHR | EVQLVQSGAEVKKPGESLKISCKASGYSFTDYTMNWVRDMPGKGLEWIGLINPYNGVTSY | 315 |
| | heavy | NPNFQGQATLSVQKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTFPAVLQSS | |
| | (H2) | GLYSLSSVVTVPSSSLGTQTYICNVNHKPSNTKVDKKVEPKSCDKTHTCPPCPAPEAAGA | |
| | GeneID: | PSVFLFPPKPKDTLMISRTPEVICVVVDVSHEDPEVKENWYVDGVEVHNAKIKPREEQYN | |
| | 507530 | STYRVVSVLTVLHQDWLNGKEYKCKVSNKALPAPIEKTISKAKGQPREPQVYTLPPCREE | |
| | | MTKNQVSLWCLVKGFYPSDIAVEWESNGQPENNYKITPPVLDSDGSFFLYSKLTVDKSRW | |
| | | QQGNVFSCSVMHEALHNHYTQKSLSLSPG | |
| | CD3 | ELVVTQEPSLTVSPGGTVTLTCRSSTGAVITSNYANWVQDKPGQAPRGLIGGTNKRAPGT | 316 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSUTPEQWKSHRSYSCQVTHEGSTVEKTVAPTESS | |
| | CD3 | EVQLLESGGGLVQPGGSLKLSCAASGFTFNDYAMNWVRKAPGKGLEWVARIRSKYNNYAT | 317 |
| | Heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGCLVKDYFPEPVIVSWNSGALTSGVHIFPA | |
| | (H1) | CLQSSGLYSLSSVVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKENWYVDGVEVHNAKTKPR | |
| | | EEQYNSTYRVVSVITVLHQDWLNGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLTSSDEDEYEM | |
| | | ESPNITDIMKFFLYVLESLPSPILTCALINGSIEVQCMIPEHYNSHRGLIMYSWDCPMEQ | |
| | | CKENSTSIYFKMENDLPQKIQCTLSNPLENTTSSIILTTCIPSSGHSRHRY | |
| EIP1057 | huMHR | DIQMTQSPSTLSASVGDRVTITCRASENIYSYLAWYQKKPGKAPKLLVNNAKTLAEGVPS | 318 |
| | light | RFSGSGSGTEFTLTISSLQPDDEATYYCQHHYGSPWTFGQGTKVEIKRTVAAPSVFIFPP | |
| | chain | SDEQLKSGTASVVCLLNNFYPREAKVQWKVDNALQSGNSQESVTEQDSKDSTYSLSSTLR | |
| | (L2) | LSKADYEKHKVYACEVTHQGLSSPVTKSFNRGEC | |
| | GeneID: | | |
| | 507531 | | |
| | huMHR | EVQLVQSGAEVKKPGSSVKVSCKASGYSFTDYTMNWVRDAPGQGLEWIGLINPYNGVTSY | 319 |
| | heavy | NPNFQGRATLTVDKSTSTAYMELSSLRSEDTAVYYCARSGDYDRNYFDYWGQGTLVTVSS | |
| | chain | ASTKGPSVFPLAPSSKSTSGGTAALGCLVDDYFPEPVTVSWNSGALTSGVHTFPAVLQSS | |
| | (H2) | GLYSLSSVVTVPSSSLGTQTYICNVNHKPSNTKVDKKVEPKSCDKTHTCPPCPAPEAAGA | |
| | GeneID: | PSVFLEPPKPKDTLMISRTPEVTCVVVDVSHEDPEVKFNWYVDGVEVHNAKTKPREEQYN | |
| | 520067 | STYRVVSVLTVLHQQWINGKEYKCKVSNKALPAPIEKTISKAKGQPREPQVYTLPPCREE | |
| | | MTKNQVSLWCLVKGFYPSDIAVEWESNGQPENNYKTTPPVLDSDGSFFLYSKLTVDKSRW | |
| | | QQGNVESCSVMHEALHNHYTQKSLSLSPG | |
| | CD3 | ELVVTQEPSLTVSPGGTVTLTCRSSTGAVTTSNYANWVQDKPGQAPRGLIGGTNKRAPGT | 320 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNDYAMNWVRKAPGKGLEWVARIRSKYNNYAT | 321 |
| | Heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGCLVKDYFPEPVTVSWNSGALTSGVHTFPA | |
| | (H1) | CLQSSGLYSLSSVVTVPSSSIGTQTYICNVNHKPSNTKVDKKVEPKSSDKTHTCPPCPAP | |
| | | EAAGAPSVFLFPPKPKDILMISRTPEVTCVVVDVSHEDPEVKENWYVDGVEVHNAKIKPR | |
| | | EEQYNSTYRVVSVLTVLHQDWLNGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLTSSDEDEYEM | |
| | | ESPNITDTMKFFLYVLESLPSETLICALINGSIEVQCMIPEHYNSHRGLIMYSWDCPMEQ | |
| | | CKRNSTSIYFKMENDLPQKIQCTLSNPLENTTSSIILTTCIPSSGHSRHRY | |
| EIP0716 | huMHR | DIQMTQSPSTLSASVGDRVTITCRASENIYSYLAWYQKKPGKAPKLLVNNAKTLAEGVPS | 326 |
| | light | RFSGSGSGTEFTLTISSLQPDDFATYYCQHHYGSPWTFGQGTKVEIKRTVAAPSVFIFPP | |
| | chain | SDEQLKSGTASVVCLENNFYPREAKVQWKVQNALQSGNSQESVTEQDSKDSTYSLSSTLR | |
| | (L2) | LSKADYEKHKVYACEVTHQGLSSPVTKSFNRGEC | |
| | huMHR | EVQLVQSGAEVKKPGESLKISCKASGYSFTDYTMNWVRDMPGKGLEWIGLINPYNGVTSY | 327 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTEPAVLQSS | |
| | (H2) | GLYSLSSVVTVPSSSLGTQTYICNVNHKPSNTKVDKKVEPKSCDKTHTCPPCPAPEAAGA | |
| | | PSVFLEPPKPKDTLMISRTPEVTCVVVDVSHEDPEVKENWYVDGVEVHNAKTKPREEQYN | |
| | | STYRVVSVLTVLHQDWINGKEYKCKVSNKALPAPIEKTISKAKGQPREPQVYTLPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSLSPG | |
| | CD180 | EVQLVQSGAEVKKPGESLKISCKASGYSFTDYTMNWVRDMPGKGLEWIGLINPYNGVTSY | 695 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTFPAVLQSS | |
| | with K. | GLYSLSSVVTVPSSSLGTQTYICNVNHKPSNTKVDKKVEPKSCDKTHTCPPCPAPEAAGA | |
| | residue | PSVFLFPPKPKDTLMISRTPEVICVVVDVSHEDPEVKENWYVDGVEVHNAKTKPREEQYN | |
| | at last | STYRVVSVLTVLHQDWINGKEYKCKVSNKALPAPIEKTISKAKGQPREPQVYTLPPCREE | |
| | position | MTKNQVSLWCLVKGFYPSDIAVEWESNGQPENNYKTTPPVLDSDGSFFLYSKLTVDKSRW | |
| | (H2) | QQGNVFSCSVMHEALHNHYTQKSLSLSPGK | |
| | CD3 | ELVVTQEPSLTVSPGGTVTLTCRSSTGAVITSNYANWVQDKPGQAPRGLIGGTNKRAPGT | 328 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNTYAMNWVRKAPGKGLEWVARIRSKYNNYAT | 329 |
| | Heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGCLVKDYFPEPVTVSWNSGALTSGVHTFPA | |
| | (H1) | CLQSSGLYSLSSVVTVPSSSLGTQTYICNVNHKPSNTKVDKKVEPKSSDKTHTCPPCPAP | |
| | | EAAGAPSVFLFPPKPKDTIMISRTPEVTCVVVDVSHEDPEVKFNWYVDGVEVHNAKTKPR | |
| | | EEQYNSTYRVVSVITVLHQDWLNGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALENHYTQKSLSLSPGGGGSGGGGSFSQQIYGVVYGNVTFHV | |
| | | PSNVPLKEVLWKKQKDKVAELENSEFRAESSFKNRVYLDTVSGSLTIYNLTSSDEDEYEM | |
| | | ESPNITDTMKFFLYVLESLPSPILTCALINGSIEVQCKIPEHYNSHRGLIMYSWDCPMEQ | |
| | | CKRNSTSIYFKMENDLPQKIQCTLSNPLENTTSSIILTTCIPSSCHSRHRY | |
| | CD3 | EVQLLESGGGLVQPGGSLKLSCAASGFTFNTYAMNWVRKAPGKGLEWVARIRSKYNNYAT | 696 |
| | heavy | YYADSVKDRFTISRDDSKNTAYLQMNNLKTEDTAVYYCVRHGNFGNSYVSWFAYWGQGTD | |
| | chain | VTVSSASTKGPSVFPLAPSSKSTSGGTAALGCLVKDYFPEPVTVSWNSGALTSGVHTFPA | |
| | with a K | CLQSSGLYSLSSVVTVPSSSIGTQTYICNVNHKPSNTKVDKKVEPKSSDKTHTCPPCPAP | |
| | residue | EAAGAPSVELFPPKPKQTLMISRTPEVTCVVVDVSHEDPEVKFNWYVDGVEVHNAKTKPR | |
| | prior to | EEQYNSTYRVVSVLTVLHQDWINGKEYKCKVSNKALPAPIEKTISKAKGQPREPQVCTLP | |
| | Linker | PSREEMTKNQVSLSCAVKGFYPSDIAVEWESNGQPENNYKTTPPVLDSDGSFFLVSKLTV | |
| | (H1) | DKSRWQQGNVFSCSVMHEALHNHYTQKSLSLSPGKGGGGSGGGGSFSQQIYGVVYGNVTF | |
| | | HVPSNVPLKEVLWKKQKDKVAELENSEFRAFSSFKNRVYLDTVSGSLTIYNLTSSDEDEY | |
| | | EMESPNITDTMKFFLYVLESLPSPILTCALINGSIEVQCMIPERYNSHRGLIMYSWDCPM | |
| | | EQCKRNSTSIYFKMENDLPQKIQCTLSNPLENTTSSIILTTCIPSSGHSRHRY | |
| EIP0707 | huMHR | DIQMTQSPSTLSASVGDRVTITCRASENIYSYLAWYQKKPGKAPKLEVNNAKTLAEGVPS | 334 |
| | light | RFSGSGSGTEFTLTISSLQPDDFATYYCQHHYGSPWTFGQGTKVEIKRTVAAPSVFIFPP | |
| | chain | SDEQLKSGTASVVCLUNNFYPREAKVQWKVDNALQSGNSQESVTEQDSKDSTYSLSSTLR | |
| | (L2) | LSKADYEKHKVYACEVTHQGLSSPVTKSFNRGEC | |
| | huMHR | EVQLVQSGAEVKKPGESLKISCKASGYSFTDYTMNWVRDMPGKGLEWIGLINPYNGVTSY | 335 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTEPAVLQSS | |
| | (H2) | GLYSLSSVVTVPSSSLGTQTYICNVNHKPSNTKVDKKVEPKSCDKTHTCPPCPAPEAAGA | |
| | | PSVFLFPPKPKDTLMISRTPEVTCVVVDVSHEDPEVKFNWYVDGVEVHNAKTKPREEQYN | |
| | | STYRVVSVLTVLHQDWINGKEYKCKVSNKALPAPIEKTISKAKGQPREPQVYTUPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSLSPG | |
| | CD3 | ELVVTQEPSLTVSPGGTVTLTCRSSTGAVTTSNYANWVQDKPGQAPRGLIGDINKRAPGT | 336 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNTYAMNWVRKAPGKGLEWVARIRSKYNNYAT | 337 |
| | Heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGCLVKDYFPEPVTVSWNSGALTSGVHTFPA | |
| | (H1) | CLQSSGLYSLSSVVTVPSSSLGTQTYICNVNHKPSNTKVDKKVEPKSSDKTHTCPPCPAP | |
| | | EAAGAPSVFLEPPKPKDTLMISRTPEVTCVVVDVSHEDPEVKENWYVDGVEVHNAKTKPR | |
| | | EEQYNSTYRVVSVLTVLHQDWLNGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFIVSKITV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLTSSDEDEYEM | |
| | | ESPNITDTMKFFLYVLESLPSPILTCALINGSIEVQCMIPEHYNSHRGLIMYSWDCPMEQ | |
| | | CKRNSTSIYFKMENDLPQKIQCTLSNPLENTTSSIILTTCIPSSGHSRHRY | |
| EIP0717 | huMHR | DIQMTQSPSTLSASVGDRVTITCRASENIYSYLAWYQKKPGKAPKLEVNNAKTLAEGVPS | 342 |
| | light | RFSGSGSGTEFTLTISSLQPDDFATYYCQHHYGSPWTFGQGTKVEIKRTVAAPSVFIFPP | |
| | chain | SDEQLKSGTASVVCLUNNFYPREAKVQWKVDNALQSGNSQESVTEQDSKDSTYSLSSTLR | |
| | (L2) | LSKADYEKHKVYACEVTHQGLSSPVTKSENRGEC | |
| | huMHR | EVQLVQSGAEVKKPGESLKISCKASGYSFTDYTMNWVRDMPGKGLEWIGLINPYNGVTSY | 343 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTEPAVLQSS | |
| | (H2) | GLYSLSSVVTVPSSSLGTQTYICNVNHKPSNTKVDKKVEPKSCDKTHTCPPCPAPEAAGA | |
| | | PSVFLFPPKPKDTLMISRTPEVTCVVVDVSHEDPEVKFNWYVDGVEVHNAKTKPREEQYN | |
| | | STYRVVSVLTVLHQDWINGKEYKCKVSNKALPAPIEKTISKAKGQPREPQVYTLPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSLSPG | |
| | CD3 | ELVVTQEPSLTVSPGGTVTLTCRSSTGAVTTSNYANWVQDKPGQAPRGLIGGTNKRAPGT | 344 |
| | light | PARFSGSLLGGKAALTLSGVQPEDEAEYYCALWYSQLWVEGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNTYAMNWVRKAPGKGLEWVARIRSKYNNYAT | 345 |
| | Heavy | YYADSVKDRFTISRDDSKNTAYLQMNNLKTEDTAVYYCVRHGNFGDDYVSWFAYWGQGTL | |
| | chain | VTVSSASTKGPSVFPLAPSSKSTSGGTAALGCLVKDYFPEPVTVSWNSGALTSGVATEPA | |
| | (H1) | CLQSSGLYSLSSVVTVPSSSLGTQTYICNVNHKPSNTKVDKKVEPKSSDKTHTCPPCPAP | |
| | | EAAGAPSVFLEPPKPKDTAMISRTPEVTCVVVDVSHEDPEVKFNWYVDGVEVHNAKTKPR | |
| | | EEQYNSTYRVVSVLTVLHQDWINGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLISSDEDEYEM | |
| | | ESPNITDTMKFFLYVLESLPSPILTCALINGSIEVQCMIPEHYNSHRGLIMYSWDCPMEQ | |
| | | CKRNSTSIYFKMENDLPQKIQCTLSNPLENTISSIILTICIPSSGHSRHRY | |
| | CD3 | EVQLLESGGGLVQPGGSLKLSCAASGFTFNTYAMNWVRKAPGKGLEWVARIRSKINNYAT | 346 |
| | heavy | YYADSVKDRFTISRDDSKNTAYLQMNNLKTEDTAVYYCVRHGNFGDDYVSWFAYWGQGTL | |
| | chain | VTVSSASTKGPSVFPLAPSSKSTSGGTAALGCLVKDYFPEPVTVSWNSGALTSGVHTFPA | |
| | with a C- | CLQSSGLYSLSSVVTVPSSSLGTQTYICNVNHKPSNTKVDKKVEPKSSDKTHTCPPCPAP | |
| | terminal | EAAGAPSVFLFPPKPKDTLMISRTPEVTCVVVDVSHEDPEVKFNWYVDGVEVHNAKTKPR | |
| | K | EEQYNSTYRVVSVITVLHQDWLNGKEYKCKVSNKALPAPIEKTISKAKGQPREPQVCTLP | |
| | residue | PSREEMTKNQVSLSCAVKGFYPSDIAVEWESNGQPENNYKTTPPVLDSDGSFFLVSKLTV | |
| | (H1) | DKSRWQQGNVFSCSVMHEALHNHYTQKSLSLSPGKGGGGSGGGGSFSQQTYGVVYGNVTF | |
| | | HVPSNVPLKEVLWKKQKDKVAELENSEFRAFSSFKNRVYLDTVSGSLTIYNLISSDEDEY | |
| | | EMESPNITDTMKFFLYVLESLPSPTLICALINGSIEVQCMIPEHYNSHRGLIMYSWDCPM | |
| | | EQCKENSTSIYFKMENDLPQKIQCTLSNPLENTTSSIILTTCIPSSGHSRHRY | |
| EIP0699 | huMHR | DIQMTQSPSTLSASVGDRVTITCRASENIYSYLAWYQKKPGKAPKLLVNNAKTLAEGVPS | 351 |
| | light | RFSGSGSGTEFTLTISSLQPDDFATYYCQHHYGSPWTFGQGTKVEIKRTVAAPSVFIFPP | |
| | chain | SDEQLKSGTASVVCLLNNFYPREAKVQWKVDNALQSGNSQESVTEQDSKDSTYSLSSTLR | |
| | (L2) | LSKADYEKHKVYACEVTHQGLSSPVTKSFNRGEC | |
| | huMHR | EVQLVQSGAEVKKPGESLKISCKASGYSFTDYTMNWVRDMPGKGLEWIGLINPYNGVTSY | 352 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTFPAVLQSS | |
| | (H2) | GLYSLSSVVTVPSSSLGTQTYICNVNHKPSNTKVDKKVEPKSCDKTHTCPPCPAPEAAGA | |
| | | PSVELFPPKPKDTUMISRTPEVTQVVVDVSHEDPEVKFNWYVQGVEVHNAKTKPREEQYN | |
| | | STYRVVSVLTVLHQDWLNGKEYKCKVSNKALPAPIEKTISKAKGQPREPQVYTLPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSUSPG | |
| | CD180 | EVQLVQSGAEVKKPGESLKISCKASGYSFTDYTMNWVRDMPGKGLEWIGLINPYNGVTSY | 694 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTFPAVLQSS | |
| | with K | GLYSLSSVVTVPSSSLGTQTYICNVNHKPSNTKVDKKVEPKSCDKTHTCPPCPAPEAAGA | |
| | residue | PSVFLFPPKPKDTLMISRTPEVTCVVVDVSHEDPEVKFNWYVDGVEVHNAKTKPREEQYN | |
| | at last | STYRVVSVLTVLHQDWINGKEYKCKVSNKALPAPIEKTISKAKGQPREPQVYTLPPCREE | |
| | position | MIKNQVSLWCLVKGFYPSDIAVEWESNGQPENNYKTIPPVLDSDGSFFLYSKLTVDKSRW | |
| | (H2) | QQGNVFSCSVMHEALHNHYTQKSLSLSPGK | |
| | CD3 | ELVVTQEPSLTVSPGGTVTLTCRSSTGAVTTSNYANWVQDKPGQAPRGLIGGTNKRAPGT | 353 |
| | light | PARFSGSLLGGKAALTLSGVQPEDEAEYYCALWYSNLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNDYAMNWVRKAPGKGLEWVARIRSKYNNYAT | 354 |
| | Heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGCLVKDYFPEPVTVSWNSGALTSGVHTEPA | |
| | (H1) | CLQSSGLYSLSSVVTVPSSSLGTQTYICNVNHKPSNTKVDKKVEPKSSDKTHTCPPCPAP | |
| | | EAAGAPSVFLEPPKPKDTIMISRTPEVTCVVVDVSHEDPEVKENWYVDGVEVHNAKTKPR | |
| | | EEQYNSTYRVVSVLTVLHQDWINGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFRV | |
| | | PSNVPLKEVLWKKQKDKVAELENSEFRAFSSFKNRVYLDTVSGSLTIYNLISSDEDEYEM | |
| | | ESPNITDTMKFFLYVLESLPSPTLTCALINGSIEVQCMIPEHYNSHRGLIMYSWDCPMEQ | |
| | | CKRNSTSIYFKMENDLPQKIQCTLSNPLENTISSIILTICIPSSGHSRHRY | |
| | CD3 | EVQLLESGGGLVQPGGSLKLSCAASGFTFNDYAMNWVRKAPGKGLEWVARIRSKYNNYAT | 355 |
| | heavy | YYADSVKDRFTISRDDSKNTAYLQMNNLKTEDTAVYYCVRHGNFGNSYVSWFAYWGQGTL | |
| | chain | VTVSSASTKGPSVFPLAPSSKSTSGGTAALGCLVKDYFPEPVTVSWNSGALTSGVHTFPA | |
| | with a C- | CLQSSGLYSLSSVVTVPSSSLGTQTYICNVNHKPSNTKVDKKVEPKSSDKTHTCPPCPAP | |
| | terminal | EAAGAPSVFLFPPKPKDTLMISRTPEVTCVVVDVSHEDPEVKFNWYVDGVEVHNAKTKPR | |
| | K | EEQYNSTYRVVSVLTVLHQDWINGKEYKCKVSNKALPAPIEKTISKAKGQPREPQVCTLP | |
| | residue | PSREEMTKNQVSLSCAVKGFYPSDIAVEWESNGQPENNYKTTPPVLDSDGSFFLVSKLTV | |
| | (H1) | DKSRWQQGNVFSCSVMHEALHNHYTQKSLSLSPGKGGGGSGGGGSFSQQIYGVVYGNVTF | |
| | | HVPSNVPLKEVLWKKQKDKVAELENSEFRAFSSFKNRVYLDTVSGSLTIYNLTSSDEDEY | |
| | | EMESPNITDTMKFFLYVLESLPSPILTCALINGSIEVQCMIPEHYNSHRGLIMYSWDCPM | |
| | | EQCKRNSTSIYFKMENDLPQKIQCTLSNPLENTTSSIILTTCIPSSGHSRHRY | |
| EIP0709 | huMHR | DIQMTQSPSTLSASVGDRVTITCRASENIYSYLAWYQKKPGKAPKLLVNNAKTLAEGVPS | 360 |
| | light | RFSGSGSGTEFTLTISSLQPDDFATYYCQHHYGSPWTFGQGTKVEIKRTVAAPSVFIFPP | |
| | chain | SDEQLKSGTASVVCLLNNFYPREAKVQWKVDNALQSGNSQESVTEQDSKDSTYSLSSTLR | |
| | (L2) | LSKADYEKHKVYACEVTHQGLSSPVTKSFNRGEC | |
| | huMHR | EVQLVQSGAEVKKPGESLKISCKASGYSFTDYTMNWVRDMPGKGLEWIGLINPYNGVTSY | 361 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTEPAVLQSS | |
| | (H2) | GLYSLSSVVTVPSSSLGTQTYICNVNHKPSNTKVDKKVEPKSCDKTHTCPPCPAPEAAGA | |
| | | PSVFLEPPKPKDTLMISRTPEVTCVVVDVSHEDPEVKENWYVDGVEVHNAKTKPREEQYN | |
| | | STYRVVSVLTVLHQDWINGKEYKCKVSNKALPAPIEKTISKAKGQPREPQVYTLPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSLSPG | |
| | CD 3 | ELVVTQEPSLTVSPGGTVTLTCRSSTGAVTTSNYANWVQDKPGQAPRGLIGGTNKRAPGT | 362 |
| | light | PARFSGSLLGGKAALTLSGVQPEDEAEYYCALWYSQLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSUTPEQWKSHRSYSCQVTHEGSTVEKTVAPTESS | |
| | CD3 | EVQLLESGGGLVQPGGSLKLSCAASGFTFNTYAMNWVRKAPGKGLEWVAKIRSKYNNYAT | 363 |
| | Heavy | YYADSVKDRFTISRDDSKNTAYLQMNNLKTEDTAVYYCVRHGNFGDSYVSWFAYWGQGTL | |
| | chain | VTVSSASTKGPSVFPLAPSSKSTSGGTAALGCLVKDYFPEPVTVSWNSGALTSGVHTFPA | |
| | (H1) | CLQSSGLYSLSSVVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVDGVEVHNAKTKPR | |
| | | EEQYNSTYRVVSVITVLHQDWLNGKEYKCKVSNKALPAPIEKTISKAKGQPREPQVCTLP | |
| | | PSREEMTKNQVSLSCAVKGFYPSDIAVEWESNGQPENNYKITPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLTSSDEDEYEM | |
| | | ESPNITDIMKFFLYVLESLPSPILTCALINGSIEVQCMIPEHYNSHRGLIMYSWDCPMEQ | |
| | | CKRNSTSIYFKMENDLPQKIQCTLSNPLENTTSSIILTTCIPSSGHSRHRY | |
| | CD3 | EVQLLESGGGLVQPGGSLKLSCAASGFTFNTYAMNWVRKAPGKGLEWVAKIRSKYNNYAT | 364 |
| | heavy | YYADSVKDRFTISRDDSKNTAYLQMNNLKTEDTAVYYCVRHGNFGDSYVSWFAYWGQGTL | |
| | chain | VTVSSASTKGPSVFPLAPSSKSTSGGTAALGCLVKDYFPEPVIVSWNSGALTSGVHIFPA | |
| | with a C- | CLQSSGLYSLSSVVTVPSSSLGTQTYICNVNHKPSNTKVDKKVEPKSSDKTHTCPPCPAP | |
| | terminal | EAAGAPSVFLFPPKPKDTIMISRTPEVTCVVVDVSHEDPEVKENWYVDGVEVHNAKTKPR | |
| | K | EEQYNSTYRVVSVLIVLHQDWLNGKEYKCKVSNKALPAPIEKTISKAKGQPREPQVCTLP | |
| | residue | PSREEMTKNQVSLSCAVKGFYPSDIAVEWESNGQPENNYKTTPPVLDSDGSFFLVSKLTV | |
| | (H1) | DKSRWQQGNVFSCSVMHEALENHYTQKSLSLSPGKGGGGSGGGGSFSQQIYGVVYGNVTF | |
| | | HVPSNVPLKEVLWKKQKDKVAELENSEFRAFSSFKNRVYLDTVSGSLTIYNLISSDEDEY | |
| | | EMESPNITDIMKFFLYVLESLPSPILTCALINGSIEVQCMIPEHYNSHRGLIMYSWDCPM | |
| | | EQCKANSTSIYFKMENDLPQKIQCTLSNPLENTISSIILTICIPSSGHSRHRY | |
| EIP0746 | huMHR | DIQMTQSPSTLSASVGDRVTITCRASENIYSYLAWYQKKPGKAPKLEVNNAKTLAEGVPS | 369 |
| | light | RFSGSGSGTEFTLTISSLQPDDFATYYCQHHYGSPWTFGQGTKVEIKRTVAAPSVFIFPP | |
| | chain | SDEQLKSGTASVVCLENNFYPREAKVQWKVDNALQSGNSQESVTEQDSKDSTYSLSSTLR | |
| | (L2) | LSKADYEKHKVYACEVTHQGLSSPVTKSFNRGEC | |
| | huMHR | EVQLVQSGAEVKKPGESLKISCKASGYSFTDYTMNWVRDMPGKGLEWIGLINPYNGVTSY | 370 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTFPAVLQSS | |
| | (H2) | GLYSLSSVVTVPSSSLGTQTYICNVNHKPSNTKVDKKVEPKSCDKTHTCPPCPAPEAAGA | |
| | | PSVFLFPPKPKDTLMISRTPEVTCVVVDVSHEDPEVKFNWYVDGVEVHNAKTKPREEQYN | |
| | | STYRVVSVLTVLHQDWINGKEYKCKVSNKALPAPIEKTISKAKGQPREPQVYTLPPCREE | |
| | | MIKNQVSLWCLVKGFYPSDIAVEWESNGQPENNYKTIPPVLDSDGSFFLYSKLTVDKSRW | |
| | | QQGNVFSCSVMHEALHNHYTQKSLSLSPG | |
| | CD3 | ELVVTQEPSLTVSPGGTVTLTCRSSTGAVTTSNYANWVQDKPGQAPRGLIGGTNKRAPGT | 371 |
| | light | PARFSGSLLGGKAALTLSGVQPEDEAEYYCALWYSQLWVEGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSLTPEQWKSHRSYSCQVTHEGSTVEKTVAPTESS | |
| | CD3 | EVQLLESGGGLVQPGGSLKLSCAASGFTFNTYAMNWVRKAPGKGLEWVARIRSKYNNYAT | 372 |
| | Heavy | YYADSVKDRFTISRDDSKNTAYLQMNNLKTEDTAVYYCVRHGSFGNSYVSWFAYWGQGTL | |
| | chain | VTVSSASTKGPSVFPLAPSSKSTSGGTAALGCLVKDYFPEPVTVSWNSGALTSGVHTEPA | |
| | (H1) | CLQSSGLYSLSSVVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKENWYVDGVEVHNAKTKPR | |
| | | EEQYNSTYRVVSVLTVLHQDWINGKEYKCKVSNKALPAPIEKTISKAKGQPREPQVCTLP | |
| | | PSREEMTKNQVSLSCAVKGFYPSDIAVEWESNGQPENNYKTTPPVLDSDGSFFLVSKLTV | |
| | | DKSRWQQGNVFSCSVMHEALHNHYTQKSLSLSPGGGGSGGGGSFSQQIYGVVYGNVTFKV | |
| | | PSNVPLKEVLWKKQKDKVAELENSEFRAFSSFKNRVYLDTVSGSLTIYNLISSDEDEYEM | |
| | | ESPNITDIMKFFLYVLESLPSPILTCALINGSIEVQCMIPEHYNSHRGLIMYSWDCPMEQ | |
| | | CKRNSTSIYFKMENDLPQKIQCTLSNPLENTISSIILTICIPSSGHSRHRY | |
| | CD3 | EVQLLESGGGLVQPGGSLKLSCAASGFTFNTYAMNWVRKAPGKGLEWVARIRSKYNNYAT | 373 |
| | heavy | YYADSVKDRFTISRDDSKNTAYLQMNNLKTEDTAVYYCVRHGSPGNSYVSWFAYWGQGTL | |
| | chain | VTVSSASTKGPSVFPLAPSSKSTSGGTAALGCLVKDYFPEPVTVSWNSGALTSGVHTFPA | |
| | with a C- | CLQSSGLYSLSSVVTVPSSSLGTQTYICNVNHKPSNTKVDKKVEPKSSDKTHTCPPCPAP | |
| | terminal | EAAGAPSVFLFPPKPKDTLMISRTPEVTCVVVDVSHEDPEVKFNWYVDGVEVHNAKTKPR | |
| | K | EEQYNSTYRVVSVLTVLHQDWINGKEYKCKVSNKALPAPIEKTISKAKGQPREPQVCTLP | |
| | residue | PSREEMTKNQVSLSCAVKGFYPSDIAVEWESNGQPENNYKTTPPVLDSDGSFFLVSKUTV | |
| | (H1) | DKSRWCQGNVFSCSVMHEALHNHYTQKSLSLSPGKGGGGSGGGGSFSQQIYGVVYGNVTF | |
| | | HVPSNVPLKEVLWKKQKDKVAELENSEFRAFSSFKNRVYLDTVSGSLTIYNLESSDEDEY | |
| | | EMESPNITDIMKFFLYVLESLPSPILTCALINGSIEVQCMIPEHYNSHRGLIMYSWDCPM | |
| | | EQCKRNSTSIYFKMENDLPQKIQCTLSNPLFNTTSSIILTTCIPSSGHSRHRY | |
| EIP0752 | huMHR | DIQMTQSPSTLSASVGDRVTITCRASENIYSYLAWYQKKPGKAPKLLVNNAKTLAEGVPS | 378 |
| | light | RFSGSGSGTEFTLTISSLQPDDFATYYCQHHYGSPWTFGQGTKVEIKRTVAAPSVFIFPP | |
| | chain | SDEQLKSGTASVVCLLNNFYPREAKVQWKVDNALQSGNSQESVTEQDSKDSTYSLSSTLR | |
| | (L2) | LSKADYEKHKVYACEVTHQGLSSPVTKSFNRGEC | |
| | huMHR | EVQLVQSGAEVKKPGESLKISCKASGYSFTDYTMNWVRDMPGKGLEWIGLINPYNGVTSY | 379 |
| | heavy | NPNFQGQATLSVDKSISTAYLQWSSLKASDTAMYYCARSGDYDRNYFDYWGQGTTLTVSS | |
| | chain | ASTKGPSVFPLAPSSKSTSGGTAALGCLVDDYFPEPVTVSWNSGALTSGVHTEPAVLQSS | |
| | (H2) | GLYSLSSVVTVPSSSLGTQTYICNVNHKPSNTKVDKKVEPKSCDKTHTCPPCPAPEAAGA | |
| | | PSVFLEPPKPKDTLMISRTPEVTCVVVDVSHEDPEVKFNWYVDGVEVHNAKTKPREEQYN | |
| | | STYRVVSVLTVLHQDWINGKEYKCKVSNKALPAPIEKTISKAKGQPREPQVYTLPPCREE | |
| | | MTKNQVSLWCLVKGFYPSDIAVEWESNGQPENNYKTTPPVLDSDGSFFLYSKLTVDKSRW | |
| | | QQGNVFSCSVMHEALHNHYTQKSLSLSPG | |
| | CD 3 | ELVVTQEPSLTVSPGGTVTLTCRSSTGAVTTSNYANWVQDKPGQAPRGLIGGTNKRAPGT | 380 |
| | light | PARFSGSLLGGKAALTLSGVQPEDEAEYYCALWYSQLWVFGGGTKLTVLGQPKANPTVTL | |
| | chain | FPPSSEELQANKADLVCLISDFYPGAVTVAWKADGSPVKAGVETCKPSKQSNNKYAASSY | |
| | (L1) | LSUTPEQWKSHRSYSCQVTHEGSTVEKTVAPTESS | |
| | CD3 | EVQLLESGGGLVQPGGSLKLSCAASGFTFNTYAMNWVRKAPGKGLEWVARIRSKYNNYAT | 381 |
| | Heavy | YYADSVKDRFTISRDDSKNTAYLQMNNLKTEDTAVYYCVRHGTFGNSYVSWFAYWGQGTL | |
| | chain | VTVSSASTKGPSVFPLAPSSKSTSGGTAALGCLVKDYFPEPVTVSWNSGALTSGVHTSPA | |
| | (H1) | CLQSSGLYSLSSVVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVDGVEVHNAKTKPR | |
| | | EEQYNSTYRVVSVITVLHQDWLNGKEYKCKVSNKALPAPIEKTISKAKGQPREPQVCTLP | |
| | | PSREEMTKNQVSLSCAVKGFYPSDIAVEWESNGQPENNYKITPPVLDSDGSFFLVSKLTV | |
| | | DKSRWQQGNVFSCSVMHEALHNHYTQKSLSLSPGGGGSGGGGSFSQQIYGVVYGNVTFHV | |
| | | PSNVPLKEVLWKKQKDKVAELENSEFRAFSSFKNRVYLDTVSGSLTIYNLTSSDEDEYEM | |
| | | ESPNITDIMKFFLYVLESLPSPILTCALINGSIEVQCMIPEHYNSHRGLIMYSWDCPMEQ | |
| | | CKRNSTSIYFKMENDLPQKIQCTLSNPLENTTSSIILTTCIPSSGHSRHRY | |
| | CD3 | EVQLLESGGGLVQPGGSLKLSCAASGFTFNTYAMNWVRKAPGKGLEWVARIRSKYNNYAT | 382 |
| | heavy | YYADSVKDRFTISRDDSKNTAYLQMNNLKTEDTAVYYCVRHGTFGNSYVSWFAYWGQGTL | |
| | chain | VTVSSASTKGPSVFPLAPSSKSTSGGTAALGCLVKDYFPEPVIVSWNSGALTSGVHTSPA | |
| | with a C- | CLQSSGLYSLSSVVTVPSSSLGTQTYICNVNHKPSNTKVDKKVEPKSSDKTHTCPPCPAP | |
| | terminal | EAAGAPSVFLFPPKPKDTIMISRTPEVTCVVVDVSHEDPEVKENWYVDGVEVHNAKTKPR | |
| | K | EEQYNSTYRVVSVLTVLHQDWLNGKEYKCKVSNKALPAPIEKTISKAKGQPREPQVCTLP | |
| | residue | PSREEMTKNQVSLSCAVKGFYPSDIAVEWESNGQPENNYKTTPPVLDSDGSFFLVSKLTV | |
| | (H1) | DKSRWQQGNVFSCSVMHEALHNHYTQKSLSLSPGKGGGGSGGGGSFSQQIYGVVYGNVTF | |
| | | HVPSNVPLKEVLWKKQKDKVAELENSEFRAFSSFKNRVYLDTVSGSLTIYNLTSSDEDEY | |
| | | EMESPNITDTMKFFLYVLESLPSPILTCALINGSIEVQCMIPEHYNSHRGLIMYSWDCPM | |
| | | EQCKRNSTSIYFKMENDLPQKIQCTLSNPLENTYSSIILTICIPSSGHSRHRY | |

Exemplary, CD3ε×CD180 bispecific antibodies with a C-terminus fusion peptide of the invention include EIP1042, EIP1043, EIP1044, EIP1056, EIP1057, EIP0716, EIP0707, EIP0717, EIP0699, EIP0709, EIP0746 and EIP0752.

Bispecific antibodies EIP1042, EIP1043, EIP1044, EIP1056, EIP1057, and EIP0699 comprise a first antigen binding domain that binds CD3ε which comprising a VH1, comprising a VH1_{CDR1} having the amino acid sequence of SEQ ID NO: 30; a VH1_{CDR2} having the amino acid sequence of SEQ ID NO: 34, and a VH1_{CDR3} having the amino acid sequence of SEQ ID NO: 37, and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

Bispecific antibody EIP0716 comprises a first antigen binding domain that binds CD3ε which comprising a VH1, comprising a VH1_{CDR1} having the amino acid sequence of SEQ ID NO: 29: a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

Bispecific antibody EIP0707 comprises a first antigen binding domain that binds CD3ε which comprising a VH1, comprising a VH1_{CDR1} having the amino acid sequence of SEQ ID NO: 29: a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 44; and a VL1_{CDR3} having the amino acid sequence of SEQ ID NO: 45.

Bispecific antibody EIP0717 comprises a first antigen binding domain that binds CD3ε which comprising a VH1, comprising a VH1_{CDR1} having the amino acid sequence of SEQ ID NO: 29: a VH1_{CDR2} having the amino acid sequence of SEQ ID NO: 34; and a VH1_{CDR3} having the amino acid sequence of SEQ ID NO: 39; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

Bispecific antibody EIP0709 comprises a first antigen binding domain that binds CD3ε which comprising a VH1, comprising a VH1_{CDR1} having the amino acid sequence of SEQ ID NO: 29: a VH1_{CDR2} having the amino acid sequence of SEQ ID NO: 35; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

Bispecific antibody EIP0746 comprises a first antigen binding domain that binds CD3ε which comprising a VH1, comprising a VH1_{CDR1} having the amino acid sequence of SEQ ID NO: 29: a VH1_{CDR2} having the amino acid sequence of SEQ ID NO: 36; and a VH1_{CDR3} having the amino acid sequence of SEQ ID NO: 40; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

Bispecific antibody EIP0752 comprises a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 41, and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

Bispecific antibodies EIP1042, EIP1043, EIP1044, EIP1056, EIP1057, EIP0716, EIP0707, EIP0717, EIP0699, EIP0709, EIP0746 and EIP0752 comprise a second antigen binding domain that binds CD180 comprising a VH2, comprising VH2_{CDR1} having the amino acid sequence of SEQ ID NO: 210; a VH2_{CDR2} having the amino acid sequence of SEQ ID NO: 212; and a VH2_{CDR3} having the amino acid sequence of SEQ ID NO: 214; and a VL2, having VL2_{CDR1} having the amino acid sequence of SEQ ID NO: 222; a VL2_{CDR2} having the amino acid sequence of SEQ ID NO: 223; and a VL2_{CDR3} having the amino acid sequence of SEQ ID NO: 224.

In some embodiments, the bispecific antibody EIP1042 comprises a VH1 comprising the amino acid sequence of SEQ ID NO: 17, a VL1 comprising the amino acid sequence of SEQ ID NO: 22, a VH2 comprising the amino acid sequence of SEQ ID NO: 197, and a VL2 comprising the amino acid sequence of SEQ ID NO: 196.

In some embodiments, the bispecific antibody EIP1042 comprises a H1 comprising the amino acid sequence of SEQ ID NO: 305, a L1 comprising the amino acid sequence of SEQ ID NO: 304, a H2 comprising the amino acid sequence of SEQ ID NO: 303, and a L2 comprising the amino acid sequence of SEQ ID NO: 302.

In some embodiments, the bispecific antibody EIP1043 comprises a VH1 comprising the amino acid sequence of SEQ ID NO: 17, a VL1 comprising the amino acid sequence of SEQ ID NO: 22, a VH2 comprising the amino acid sequence of SEQ ID NO: 199, and a VL2 comprising the amino acid sequence of SEQ ID NO: 198.

In some embodiments, the bispecific antibody EIP1043 comprises a H1 comprising the amino acid sequence of SEQ ID NO: 309, a L1 comprising the amino acid sequence of SEQ ID NO: 308, a H2 comprising the amino acid sequence of SEQ ID NO: 307, and a L2 comprising the amino acid sequence of SEQ ID NO: 306.

In some embodiments, the bispecific antibody EIP1044 comprises a VH1 comprising the amino acid sequence of SEQ ID NO: 17, a VL1 comprising the amino acid sequence of SEQ ID NO: 22, a VH2 comprising the amino acid sequence of SEQ ID NO: 201, and a VL2 comprising the amino acid sequence of SEQ ID NO: 200.

In some embodiments, the bispecific antibody EIP1044 comprises a H1 comprising the amino acid sequence of SEQ ID NO: 313, a L1 comprising the amino acid sequence of SEQ ID NO: 312, a H2 comprising the amino acid sequence of SEQ ID NO: 311, and a L2 comprising the amino acid sequence of SEQ ID NO: 310.

In some embodiments, the bispecific antibody EIP1056 comprises a VH1 comprising the amino acid sequence of SEQ ID NO: 17, a VL1 comprising the amino acid sequence of SEQ ID NO: 22, a VH2 comprising the amino acid sequence of SEQ ID NO: 203, and a VL2 comprising the amino acid sequence of SEQ ID NO: 202.

In some embodiments, the bispecific antibody EIP1056 comprises a H1 comprising the amino acid sequence of SEQ ID NO: 317, a L1 comprising the amino acid sequence of SEQ ID NO: 316, a H2 comprising the amino acid sequence of SEQ ID NO: 315, and a L2 comprising the amino acid sequence of SEQ ID NO: 314.

In some embodiments, the bispecific antibody EIP1057 comprises a VH1 comprising the amino acid sequence of SEQ ID NO: 17, a VL1 comprising the amino acid sequence of SEQ ID NO: 22, a VH2 comprising the amino acid sequence of SEQ ID NO: 205, and a VL2 comprising the amino acid sequence of SEQ ID NO: 204.

In some embodiments, the bispecific antibody EIP1057 comprises a H1 comprising the amino acid sequence of SEQ ID NO: 321, a L1 comprising the amino acid sequence of SEQ ID NO: 320, a H2 comprising the amino acid sequence of SEQ ID NO: 319, and a L2 comprising the amino acid sequence of SEQ ID NO: 318.

In some embodiments, the bispecific antibody EIP0716 comprises a VH1 comprising the amino acid sequence of SEQ ID NO: 13, a VL1 comprising the amino acid sequence of SEQ ID NO: 22, a VH2 comprising the amino acid sequence of SEQ ID NO: 203, and a VL2 comprising the amino acid sequence of SEQ ID NO: 202.

In some embodiments, the bispecific antibody EIP0716 comprises a H1 comprising the amino acid sequence of SEQ ID NO: 329, a L1 comprising the amino acid sequence of SEQ ID NO: 328, a H2 comprising the amino acid sequence of SEQ ID NO: 327, and a L2 comprising the amino acid sequence of SEQ ID NO: 326.

In some embodiments, the bispecific antibody EIP0707 comprises a VH1 comprising the amino acid sequence of SEQ ID NO: 13, a VL1 comprising the amino acid sequence of SEQ ID NO: 27, a VH2 comprising the amino acid sequence of SEQ ID NO: 203, and a VL2 comprising the amino acid sequence of SEQ ID NO: 202.

In some embodiments, the bispecific antibody EIP0707 comprises a H1 comprising the amino acid sequence of SEQ ID NO: 337, a L1 comprising the amino acid sequence of SEQ ID NO: 336, a H2 comprising the amino acid sequence of SEQ ID NO: 335, and a L2 comprising the amino acid sequence of SEQ ID NO: 334.

In some embodiments, the bispecific antibody EIP0717 comprises a VH1 comprising the amino acid sequence of SEQ ID NO: 16, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0717 comprises a H1 comprising the amino acid sequence of SEQ ID NO: 345, a L1 comprising the amino acid sequence of SEQ ID NO: 344, a H2 comprising the amino acid sequence of SEQ ID NO: 343, and a L2 comprising the amino acid sequence of SEQ ID NO: 342.

In some embodiments, the bispecific antibody EIP0699 comprises a VH1 comprising the amino acid sequence of SEQ ID NO: 17, a VL1 comprising the amino acid sequence of SEQ ID NO: 22, a VH2 comprising the amino acid sequence of SEQ ID NO: 203, and a VL2 comprising the amino acid sequence of SEQ ID NO: 202.

In some embodiments, the bispecific antibody EIP0699 comprises a H1 comprising the amino acid sequence of SEQ ID NO: 354, a L1 comprising the amino acid sequence of SEQ ID NO: 353, a H2 comprising the amino acid sequence of SEQ ID NO: 352, and a L2 comprising the amino acid sequence of SEQ ID NO: 351.

In some embodiments, the bispecific antibody EIP0709 comprises a VH1 comprising the amino acid sequence of SEQ ID NO: 18, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0709 comprises a H1 comprising the amino acid sequence of SEQ ID NO: 363, a L1 comprising the amino acid sequence of SEQ ID NO: 362, a H2 comprising the amino acid sequence of SEQ ID NO: 361, and a L2 comprising the amino acid sequence of SEQ ID NO: 360.

In some embodiments, the bispecific antibody EIP0746 comprises a VH1 comprising the amino acid sequence of SEQ ID NO: 19, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0746 comprises a H1 comprising the amino acid sequence of SEQ ID NO: 372, a L1 comprising the amino acid sequence of SEQ ID NO: 371, a H2 comprising the amino acid sequence of SEQ ID NO: 370, and a L2 comprising the amino acid sequence of SEQ ID NO: 369.

In some embodiments, the bispecific antibody EIP0752 comprises a VH1 comprising the amino acid sequence of SEQ ID NO: 20, a VL1 comprising the amino acid sequence of SEQ ID NO: 26, a VH2 comprising the amino acid sequence of SEQ ID NO: 203, and a VL2 comprising the amino acid sequence of SEQ ID NO: 202.

In some embodiments, the bispecific antibody EIP0752 comprises a H1 comprising the amino acid sequence of SEQ ID NO: 381, a L1 comprising the amino acid sequence of SEQ ID NO: 380, a H2 comprising the amino acid sequence of SEQ ID NO: 379, and a L2 comprising the amino acid sequence of SEQ ID NO: 368.

Any one of the anti-CD3/anti-CD180 bispecific antibodies shown above in Table 11.5 can be further modified by substituting any one of the anti-CD3ε antigen binding regions with any one of the anti-CD3ε binding regions shown in Tables 9.1, 9.2 and 9.3. For example, the anti-CD3ε antigen binding regions of bispecific antibody “EIP1042” can be substituted with any one of the anti-CD3ε binding regions shown in Tables 9.1, 9.2 and 9.3 to produce the bispecific antibodies of the invention. Exemplary antibodies are shown in Table 11.4 and Table 11.5.

Exemplary Bispecific Antibodies that Bind to CD3 and BCMA

Provided herein are bispecific antibodies comprising a first antigen binding domain that binds a first antigen (e.g. CD3ε) and a second antigen binding domain that binds to a CD180 In some embodiments the bispecific antibody has the following structure: a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2).

In some embodiments, the bispecific antibody of the disclosure comprises a first antigen binding domain (e.g. binding to CD3ε) comprising: a) a heavy chain variable region (VH1) comprising a VH complementarity determining region 1 (VH1_{CDR1}), a VH complementarity determining region 2 (VH1_{CDR2}) and a VH complementarity determining region 3 (VH1_{CDR3}); and b) a light chain variable region (VL) comprising a VL complementarity determining region 1 (VL1_{CDR1}), a VL complementarity determining region 2 (VL1_{CDR2}) and a VL complementarity determining region 3 (VL1_{CDR3}); and a second antigen binding domain (e.g. binding to CD180) comprising: a) a heavy chain variable region (VH2) comprising a VH complementarity determining region 1 (VH2_{CDR1}), a VH complementarity determining region 2 (VH2_{CDR2}) and a VH2 complementarity determining region 3 (VH2_{CDR3}); and b) a light chain variable region (VL2) comprising a VL complementarity determining region 1 (VL2_{CDR1}), a VL complementarity determining region 2 (VL2_{CDR2}) and a VL complementarity determining region 3 (VL2_{CDR3}).

Exemplary Bispecific Antibodies that Bind to Cd3 and Cd180

Provided herein are bispecific antibodies comprising a first antigen binding domain that binds a first antigen (e.g. CD3ε) and a second antigen binding domain that binds to a BCMA. In some embodiments the bispecific antibody has the following structure: a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2).

In some embodiments, the bispecific antibody of the disclosure comprises a first antigen binding domain (e.g. binding to CD3ε) comprising: a) a heavy chain variable region (VH1) comprising a VH complementarity determining region 1 (VH1_{CDR1}), a VH complementarity determining region 2 (VH1_{CDR2}) and a VH complementarity determining region 3 (VH1_{CDR3}); and b) a light chain variable region (VL) comprising a VL complementarity determining region 1 (VL1_{CDR1}), a VL complementarity determining region 2 (VL1_{CDR2}) and a VL complementarity determining region 3 (VL1_{CDR3}); and a second antigen binding domain (e.g. binding to BCMA) comprising: a) a heavy chain variable region (VH2) comprising a VH complementarity determining region 1 (VH2_{CDR1}), a VH complementarity determining region 2 (VH2_{CDR2}) and a VH2 complementarity determining region 3 (VH2_{CDR3}); and b) a light chain variable region (VL2) comprising a VL complementarity determining region 1 (VL2_{CDR1}), a VL complementarity determining region 2 (VL2_{CDR2}) and a VL complementarity determining region 3 (VL2_{CDR3}).

Exemplary BCMA variable heavy chain variable region and light chain variable region are shown below:

BCMA_VL
(SEQ ID NO: 681)
DIQMTQSPSSLSASVGDRVTITCSASQDISNYLNWYQKKPGKAPKLLIYY
TSNLHSGVPSRFSGSGSGTDFTLTISSLQPEDFATYYCQQYRKLPWTFGQ
GTKLEIK BCMA_VL
BCMA_VH
(SEQ ID NO: 682)
QVQLVQSGAEVKKPGSSVKVSCKASGGTFSNYWMHWVRDAPGOGLEWMGA
TYRGHSDTYYNQKFKGRVTITADKSTSTAYMELSSLRSEDTAVYYCARGA
IYDGYDVLDNWGQGTLVTVSS

In some embodiments, the bispecific antibody EIP0506 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0506-2 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0506-3 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0506-4 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0506 comprises a H1 comprising the amino acid sequence of SEQ ID NO: 488, a L1 comprising the amino acid sequence of SEQ ID NO: 141, a H2 comprising the amino acid sequence of SEQ ID NO: 676, and a L2 comprising the amino acid sequence of SEQ ID NO: 675.

In some embodiments, the bispecific antibody EIP0506-2 comprises a H1 comprising the amino acid sequence of SEQ ID NO: 488, a L1 comprising the amino acid sequence of SEQ ID NO: 141, a H2 comprising the amino acid sequence of SEQ ID NO: 677, and a L2 comprising the amino acid sequence of SEQ ID NO: 675.

In some embodiments, the bispecific antibody EIP0506-3 comprises a H1 comprising the amino acid sequence of SEQ ID NO: 588, a L1 comprising the amino acid sequence of SEQ ID NO: 141, a H2 comprising the amino acid sequence of SEQ ID NO: 676, and a L2 comprising the amino acid sequence of SEQ ID NO: 675.

In some embodiments, the bispecific antibody EIP0506-4 comprises a H1 comprising the amino acid sequence of SEQ ID NO: 588, a L1 comprising the amino acid sequence of SEQ ID NO: 141, a H2 comprising the amino acid sequence of SEQ ID NO: 677, and a L2 comprising the amino acid sequence of SEQ ID NO: 675.

In some embodiments, the CD3×BCMA bispecific antibodies of the invention have a CD58 fusion peptide (SEQ ID NO: 49) fused indirectly at the C-terminus of the first heavy chain polypeptide (H1) using linker-1 (SEQ ID NO: 52). In some embodiments, the CD3×BCMA bispecific antibodies of the invention have a CD58v* fusion peptide (SEQ ID NO: 50) fused indirectly at the C-terminus of the first heavy chain polypeptide (H1) using linker-1 (SEQ ID NO: 52).

In some embodiments, the CD3×BCMA bispecific antibodies of the invention have a CD58 fusion peptide (SEQ ID NO: 49) fused indirectly at the C-terminus of the first heavy chain polypeptide (H1) using linker-2 (SEQ ID NO: 53). In some embodiments, the CD3×BCMA bispecific antibodies of the invention have a CD58v* fusion peptide (SEQ ID NO: 50) fused indirectly at the C-terminus of the first heavy chain polypeptide (H1) using linker-2 (SEQ ID NO: 53).

In some embodiments, the CD3×BCMA bispecific antibodies of the invention have a CD58 fusion peptide (SEQ ID NO: 49) fused indirectly at the C-terminus of the first heavy chain polypeptide (H1) using linker-3 (SEQ ID NO: 54). In some embodiments, the CD3×BCMA bispecific antibodies of the invention have a CD58v* fusion peptide (SEQ ID NO: 50) fused indirectly at the C-terminus of the first heavy chain polypeptide (H1) using linker-3 (SEQ ID NO: 54).

Exemplary, CD3ε×BCMA bispecific antibodies with a C-terminus fusion peptide of the invention include EIP0534.

In some embodiments, the bispecific antibody EIP0534 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0534-2 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0534-3 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0534-4 comprises a VH1 comprising the amino acid sequence of SEQ ID NO: 13, a VL1 comprising the amino acid sequence of SEQ ID NO: 22, a VH2 comprising the amino acid sequence of SEQ ID NO: 682, and a VL2 comprising the amino acid sequence of SEQ ID NO: 681.

In some embodiments, the bispecific antibody EIP0534 comprises a H1 comprising the amino acid sequence of SEQ ID NO: 488, a L1 comprising the amino acid sequence of SEQ ID NO: 141, a H2 comprising the amino acid sequence of SEQ ID NO: 679, and a L2 comprising the amino acid sequence of SEQ ID NO: 678.

In some embodiments, the bispecific antibody EIP0534-2 comprises a H1 comprising the amino acid sequence of SEQ ID NO: 488, a L1 comprising the amino acid sequence of SEQ ID NO: 141, a H2 comprising the amino acid sequence of SEQ ID NO: 680, and a L2 comprising the amino acid sequence of SEQ ID NO: 678.

In some embodiments, the bispecific antibody EIP0534-3 comprises a H1 comprising the amino acid sequence of SEQ ID NO: 588, a L1 comprising the amino acid sequence of SEQ ID NO: 141, a H2 comprising the amino acid sequence of SEQ ID NO: 679, and a L2 comprising the amino acid sequence of SEQ ID NO: 678.

In some embodiments, the bispecific antibody EIP0534-4 comprises a H1 comprising the amino acid sequence of SEQ ID NO: 588, a L1 comprising the amino acid sequence of SEQ ID NO: 141, a H2 comprising the amino acid sequence of SEQ ID NO: 680, and a L2 comprising the amino acid sequence of SEQ ID NO: 678.

Exemplary anti-CD3ε and anti-BCMA bispecific antibodies include those shown in Table 12.1.

TABLE 12.1
Exemplary Bispecific Antibodies that bind to CD38 and BCMA

| | | | |
|---|---|---|---|
| EIP0506 | BCMA light | DIQMTQSPSSLSASVGDRVTITCSASQDISNYLNWYQKKPGKAPKLLIYYTS | SEQ ID |
| | chain (H2) | NLHSGVPSRFSGSGSGTDFTLTISSLQPEDFATYYCQQYRKLPWTFGQGTKE | NO: 675 |
| | | EIKRTVAAPSVFIFPPSDEQLKSGTASVVCLLNNFYPREAKVOWKVDNALQS | |
| | | GNSQESVTEQDSKDSTYSLSSTLRLSKADYEKHKVYACEVTHQGLSSPVTKS | |
| | | FNRGEC | |
| | BCMA heavy | QVQLVQSGAEVKKPGSSVKVSCKASGGTFSNYWMHWVRDAPGQGLEWMGATY | SEQ ID |
| | chain (H2) | RGHSDTYYNQKFKGRVTITADKSTSTAYMELSSLRSEDTAVYYCARGAIYDG | NO: 676 |
| | | YDVLDNWGQGTLVTVSSASTKGPSVFPLAPSSKSTSGGTAALGCLVDDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPEAAGAPSVFLFPPKPKDTIMISRT | |
| | | PEVTCVVVDVSHEDPEVKFNWYVDGVEVHNAKTKPREEQYNSTYRVVSVLIV | |
| | | LHQDWINGKEYKCKVSNKALPAPIEKTISKAKGQPREPQVYTLPPCREEMTK | |
| | | NQVSLWCLVKGFYPSDIAVEWESNGQPENNYKTTPPVLDSDGSFFLYSKLTV | |
| | | DKSRWQQGNVESCSVMREALHNHYTQKSLSLSPG | |
| | BCMA heavy | QVQLVQSGAEVKKPGSSVKVSCKASGGTFSNYWMHWVRDAPGQGLEWMGATY | SEQ ID |
| | chain with | RGHSDTYYNQKFKGRVTITADKSTSTAYMELSSLRSEDTAVYYCARGAIYDG | NO: 677 |
| | K residue | YDVLDNWGQGTLVTVSSASTKGPSVFPLAPSSKSTSGGTAALGCLVDDYFPE | |
| | at last | PVTVSWNSGALTSGVHIFPAVLQSSGLYSLSSVVTVPSSSLGTQTYICNVNH | |
| | position | KPSNTKVDKKVEPKSCDKTHTCPPCPAPEAAGAPSVFLFPPKPKDTLMISRT | |
| | (H2) | PEVTCVVVDVSHEDPEVKENWYVDGVEVHNAKTKPREEQYNSTYRVVSVLTV | |
| | | LHQDWLNGKEYKCKVSNKALPAPIEKTISKAKGQPREPQVYTLPPCREEMTK | |
| | | NQVSLWCLVKGFYPSDIAVEWESNGQPENNYKTTPPVLDSDGSFFLYSKLTV | |
| | | DKSRWQQGNVFSCSVMHEALHNHYTQKSLSLSPGK | |
| | CD3 light | ELVVTQEPSLTVSPGGTVTLTCRSSTGAVTTSNYANWVQDKPGQAPRGLIGG | SEQ ID |
| | chain (L1) | INKRAPGTPARFSGSLLGGKAALTLSGVQPEDEAEYYCALWYSNLWVEGGGT | NO: 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: 488 |
| | | GNSYVSWFAYWGQGTLVTVSSASTKGPSVFPLAPSSKSTSGGTAALGQLVKD | |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKENWYVDGVEVANAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPG | |
| | CD3 heavy | EVQLLESGGGLVQPGGSLKLSCAASGFTFNTYAMNWVRKAPGKGLEWVARIR | SEQ ID |
| | chain with | SKYNNYATYYADSVKDRFTISRDDSKNTAYLQMNNLKTEDTAVYYCVRHGNF | NO: 588 |
| | K residue | GNSYVSWFAYWGQGTLVTVSSASTKGPSVFPLAPSSKSTSGGTAALGCLVKD | |
| | at last | YFPEPVTVSWNSGALISGVHTFPACLQSSGLYSLSSVVTVPSSSLGTQTYIC | |
| | position | NVNHKPSNTKVDKKVEPKSSDKTHTCPPCPAPEAAGAPSVFLFPPKPKDTLM | |
| | (H1) | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGK | |

TABLE 12.2
Exemplary Bispecific Antibodies that bind to CD3s and BCMA
with a C-terminus CD58 fusion peptide

| | | | |
|---|---|---|---|
| KIP0534 | BCMA light | EIVLTQSPATLSLSPGERATLSCRASQSVSSYLAWYQKKPGQAPRLETYDAS | SEQ ID |
| | chain (L2) | NRATGIPARFSGSGSGTDFTLTISSLEPEDFAVYYCQQRSNWPITFGQGTRL | NO: 678 |
| | | EIKRTVAAPSVFIFPPSDEQLKSGTASVVCLLNNFYPREAKVQWKVDNALQS | |
| | | GNSQESVTEQDSKDSTYSLSSTLRLSKADYEKHKVYACEVTHQGLSSPVTKS | |
| | | FNRGEC | |
| | BCMA heavy | EVQLVESGGGLVQPGRSLRLSCAASGFTFNDYAMHWVRDAPGKGLEWVSTIS | SEQ ID |
| | chain (H2) | WNSGSIGYADSVKGRFTISRDNAKKSLYLQMNSLRAEDTALYYCAKDIQYGN | NO: 679 |
| | | YYYGMDVWGQGTTVTVSSASTKGPSVFPLAPSSKSTSGGTAALGCLVQDYFP | |
| | | EPVTVSWNSGALTSGVHTFPAVLQSSGLYSLSSVVTVPSSSLGTQTYICNVN | |
| | | HKPSNTKVDKKVEPKSCDKTHTCPPCPAPEAAGAPSVFLFPPKPKDTLMISR | |
| | | TPEVTCVVVDVSHEDPEVKFNWYVDGVEVHNAKTKPREEQYNSTYRVVSVLT | |
| | | VLHQDWINGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKUT | |
| | | VDKSRWQQGNVFSCSVMHEALHNHYTQKSLSLSPG | |
| | BCMA heavy | EVQLVESGGGLVQPGRSLRLSCAASGFTFNDYAMHWVRDAPGKGLEWVSTIS | SEQ ID |
| | chain with | WNSGSIGYADSVKGRFTISRDNAKKSLYLQMNSLRAEDTALYYCAKDIQYGN | NO: 680 |
| | K residue | YYYGMDVWGQGTTVTVSSASTKGPSVFPLAPSSKSTSGGTAALGQLVQDYFP | |
| | at last | EPVTVSWNSGALTSGVHTFPAVLQSSGLYSLSSVVTVPSSSLGTQTYICNVN | |
| | position | HKPSNTKVDKKVEPKSCDKTHTCPPCPAPEAAGAPSVFLFPPKPKDTLMISR | |
| | (H2) | TPEVTCVVVDVSHEDPEVKENWYVDGVEVHNAKTKPREEQYNSTYRVVSVET | |
| | | VLHQDWINGKEYKCKVSNKALPAPIEKTISKAKGQPREPQVYTLPPCREEMT | |
| | | KNQVSLWCLVKGFYPSDIAVEWESNGQPENNYKTTPPVLDSDGSFFLYSKIT | |
| | | VDKSRWQQGNVFSCSVMHEALHNHYTQKSLSLSPGK | |
| | CD3 Light | ELVVTQEPSLTVSPGGTVTLTCRSSTGAVITSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: 141 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: 506 |
| | | GNSYVSWFAYWGQGTLVTVSSASTKGPSVFPLAPSSKSTSGGTAALGQLVKD | |
| | | YFPEPVTVSWNSGALISGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKENWYVDGVEVANAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSGGGGS | |
| | | FSQQIYGVVYGNVTFHVPSNVPLKEVLWKKQKDKVAELENSEFRAFSSEKNR | |
| | | VYLDTVSGSLTIYNLTSSDEDEYEMESPNITDTMKFFLYVLESLPSPTLTCA | |
| | | LINGSIEVQCMIPEHYNSHRGLIMYSWDCPMEQCKRNSTSIYEKMENDLPQK | |
| | | IQCTLSNPLENTISSIILTTCIPSSGHSRHRY | |
| | CD3 heavy | EVQLLESGGGLVQPGGSLKLSCAASGFTFNTYAMNWVRKAPGKGLEWVARIR | SEQ ID |
| | chain with | SKYNNYATYYADSVKDRFTISRDDSKNTAYLQMNNLKTEDTAVYYCVRHGNF | NO: 600 |
| | a K | GNSYVSWFAYWGQGTLVTVSSASTKGPSVFPLAPSSKSTSGGTAALGCLVKD | |
| | residue | YFPEPVTVSWNSGALTSGVHTFPACLQSSGLYSLSSVVTVPSSSLGTQTYIC | |
| | prior to | NVNHKPSNTKVDKKVEPKSSDKTHTCPPCPAPEAAGAPSVFLFPPKPKDTLM | |
| | linker | ISRTPEVTCVVVQVSHEDPEVKENWYVDGVEVHNAKTKPREEQYNSTYRVVS | |
| | (H1) | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGGGGG | |
| | | GSFSQQIYGVVYGNVTFHVPSNVPLKEVLWKKQKDKVAELENSEFRAFSSEK | |
| | | NRVYLDTVSGSLTIYNLISSDEDEYEMESPNITDTMKFFLYVLESLPSPILT | |
| | | CALINGSIEVQCMIPEHYNSHRGLIMYSWDCPMEQCKRNSTSIYFKMENDLP | |
| | | QKIQCTLSNPLENTTSSIILTTCIPSSGHSRHRY | |

Table 13 show exemplary bispecific antibodies of the disclosure and bispecific antibodies with a C-terminus fusion peptide.

TABLE 13
Exemplary Bispecific Antibodies of the Disclosure

| EIP | Target antigen on | Target antigen | Fusion |
|---|---|---|---|
| number | CAR T cell | on a tumor cell | peptide |
| EIP0506 | CD3; | BCMA; Belantamab | n/a |
| | CD3-01 (SP34) | | |
| EIP0534 | CD3; | BCMA; Belantamab | CD58 |
| | CD3-01 (SP34) | | |
| EIP0961 | CD3; CD3-A9 | CD20; ofatumumab | n/a |
| EIP0929 | CD3; CD3-A9 | CD20; ofatumumab | CD58 |
| EIP0698 | CD3; CD3-A6 | CD180; humanized | n/a |
| | | MHR73-11 | |
| EIP0699 | CD3; CD3-A6 | CD180; humanized | CD58 |
| | | MHR73-11 | |
| EIP0696 | CD3; | CD180; humanized | n/a |
| | CD3-01 (SP34) | MHR73-11 | |
| EIP0716 | CD3; | CD180; humanized | CD58 |
| | CD3-01 (SP34) | MHR73-11 | |

### Methods of Production

Various procedures known within the art may be used for the production of polyclonal or monoclonal antibodies directed against a given target, such as, for example, CD20, a disease associated antigen or other target, or against derivatives, fragments, analogs homologs or orthologs thereof (See, for example, Antibodies: A Laboratory Manual, Harlow E, and Lane D, 1988, Cold Spring Harbor Laboratory Press, Cold Spring Harbor, NY, incorporated herein by reference).

Antibodies are purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

In some embodiments, the antibodies of the invention are monoclonal antibodies. Monoclonal antibodies are generated, for example, by using the procedures set forth in the Examples provided herein. Antibodies are also generated, e.g., by immunizing BALB/c mice with combinations of cell transfectants expressing high levels of a given target on their surface. Hybridomas resulting from myeloma/B cell fusions are then screened for reactivity to the selected target.

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Monoclonal antibodies of the invention include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)2 or other antigen-binding subsequences of antibodies) that are principally comprised of the sequence of a human immunoglobulin, and contain minimal sequence derived from a non-human immunoglobulin. Humanization is performed, e.g., by following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies also comprise, e.g., residues which are found neither in the recipient antibody nor in the imported CDR or framework sequences. In general, the humanized antibody includes substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also includes at least a portion of an immunoglobulin constant region (Fc), typically that of a human immunoglobulin (Jones et al., 1986; Riechmann et al., 1988; and Presta. Curr. Op. Struct. Biol., 2:593-596 (1992)).

Fully human antibodies are antibody molecules in which the entire sequence of both the light chain and the heavy chain, including the CDRs, arise from human genes. Such antibodies are termed “human antibodies”, or “fully human antibodies” herein. Monoclonal antibodies can be prepared by using trioma technique; the human B-cell hybridoma technique (see Kozbor, et al., 1983 Immunol Today 4: 72); and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An example of such a nonhuman animal is a mouse termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds specifically to the relevant epitope with high affinity are disclosed in PCT publication WO 99/53049.

The antibody can be expressed by a vector containing a DNA segment encoding the single chain antibody described above.

These can include vectors, liposomes, naked DNA, adjuvant-assisted DNA, gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems. D. Glover, Ed. (Oxford Univ. Press, Oxford England) (1995); Geller, A. I. et al., Proc Natl. Acad. Sci. U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cell's cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, (Ca)₂(PO₄)₃ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

Bispecific antibodies are antibodies that have binding specificities for at least two different antigens. In the present case, one of the binding specificities is for a first target such as CD3ε or any fragment thereof. The second binding target is a disease associated antigen such as CD20 or any fragment thereof.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Bispecific and/or monovalent antibodies of the invention can be made using any of a variety of art-recognized techniques, including those disclosed in co-pending application WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However, it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “r bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof (See, e.g., Antibodies: A Laboratory Manual, Harlow E, and Lane D, 1988, Cold Spring Harbor Laboratory Press, Cold Spring Harbor, NY, incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogenic non-human animals. For example, see U.S. Pat. Nos. 6,075,181 and 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”). Alternatively, a library can be prepared in yeast as protein or peptide fusions to a cell wall protein on the surface of yeast cells and encoding DNA sequences contained within the yeast cells (i.e. “yeast display library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Although not strictly impossible, the serendipitous identification of different antibodies having the same heavy chain variable domain but directed against different antigens is highly unlikely. Indeed, in most cases the heavy chain contributes largely to the antigen binding surface and is also the most variable in sequence. In particular the CDR3 on the heavy chain is the most diverse CDR in sequence, length and structure. Thus, two antibodies specific for different antigens will almost invariably carry different heavy chain variable domains.

The methods disclosed in co-pending application WO 2012/023053 overcomes this limitation and greatly facilitates the isolation of antibodies having the same heavy chain variable domain by the use of antibody libraries in which the heavy chain variable domain is the same for all the library members and thus the diversity is confined to the light chain variable domain. Such libraries are described, for example, in co-pending applications WO 2010/135558 and WO 2011/084255, each of which is hereby incorporated by reference in its entirety. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention. The bispecific antibodies of the invention can be of different Isotypes and their Fc portion can be modified in order to alter the bind properties to different Fc receptors and in this way modify the effectors functions of the antibody as well as it pharmacokinetic properties. Numerous methods for the modification of the Fc portion have been described and are applicable to antibodies of the invention. (see for example Strohl, W R Curr Opin Biotechnol 2009 (6):685-91: U.S. Pat. No. 6,528,624; PCT/US2009/0191199 filed Jan. 9, 2009). The methods of the invention can also be used to generate bispecific antibodies and antibody mixtures in a F(ab′)2 format that lacks the Fc portion.

The common heavy chain and two different light chains are co-expressed into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%. However, it is likely that different light chains are expressed at different levels and/or do not assemble with the same efficiency. Therefore, a means to modulate the relative expression of the different polypeptides is used to compensate for their intrinsic expression characteristics or different propensities to assemble with the common heavy chain. This modulation can be achieved via promoter strength, the use of internal ribosome entry sites (IRES) featuring different efficiencies or other types of regulatory elements that can act at transcriptional or translational levels as well as acting on mRNA stability. Different promoters of different strength could include CMV (Immediate-early Cytomegalovirus virus promoter); EF1-1α (Human elongation factor 1α-subunit promoter); Ubc (Human ubiquitin C promoter); SV40 (Simian virus 40 promoter). Different IRES have also been described from mammalian and viral origin. (See e.g., Hellen CU and Sarnow P. Genes Dev 2001 15: 1593-612). These IRES can greatly differ in their length and ribosome recruiting efficiency. Furthermore, it is possible to further tune the activity by introducing multiple copies of an IRES (Stephen et al. 2000 Proc Natl Acad Sci USA 97: 1536-1541). The modulation of the expression can also be achieved by multiple sequential transfections of cells to increase the copy number of individual genes expressing one or the other light chain and thus modify their relative expressions. The Examples provided herein demonstrate that controlling the relative expression of the different chains is critical for maximizing the assembly and overall yield of the bispecific antibody.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

Other suitable purification methods include those disclosed in co-pending application PCT/IB2012/003028, filed on Oct. 19, 2012, published as WO2013/088259, the contents of which are hereby incorporated by reference in their entirety.

In other embodiments of producing bispecific antibodies, antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example. Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface includes at least a part of the CH3 region of an antibody constant domain. In this method, one or more small amino acid side chains from the interface of the first antibody molecule are replaced with larger side chains (e.g., tyrosine or tryptophan).

Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64). FcγRII (CD32) and FcγRill (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope of the present invention. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, so as to enhance, e.g., the effectiveness of the antibody in treating cancer and/or other diseases and disorders associated with aberrant CD20 expression and/or activity. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies of the invention. (See, for example, “Conjugate Vaccines”. Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiamides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6-[3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propionamide]hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA. 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

### Methods of Use

Any of the bispecific antibodies of the disclosure (e.g. anti-CD3ε and anti-CD20 antibody; anti-CD3ε and anti-CD20 antibody with CD58 fusion peptide) an may be used in therapeutic methods. In one aspect, bispecific antibody may be for use as a medicament is provided. In further aspects, a bispecific antibody for use in treating or delaying progression of a cell proliferative disorder (e.g., cancer) e.g., esophageal cancer or an adenocarcinoma) or an autoimmune disorder (e.g., arthritis) is provided. In certain embodiments, a bispecific antibody for use in a method of treatment is provided. In certain embodiments, the invention provides a bispecific antibody for use in a method of treating an individual having a cell proliferative disorder comprising administering to the individual an effective amount of the bispecific antibody. In one such embodiment, the method further comprises administering to the individual an effective amount of at least one additional therapeutic agent, for example, as described below. In further embodiments, the invention provides a bispecific antibody for use in enhancing immune function in an individual having a cell proliferative disorder. In certain embodiments, the invention provides a bispecific antibody for use in a method of enhancing immune function in an individual having a cell proliferative disorder comprising administering to the individual an effective of the bispecific antibody to activate effector cells (e.g., T cells, e.g., CD8+ and/or CD4+ T cells), expand (increase) an effector cell population, reduce a target cell population, and/or kill a target cell (e.g., target tumor cell). An “individual” according to any of the above embodiments may be a human.

In a further aspect, the invention provides for the use of bispecific antibodies of the disclosure (e.g. anti-CD3ε and anti-CD20 antibody; anti-CD3ε and anti-CD20 antibody with CD58 fusion peptide) in the manufacture or preparation of a medicament. In one embodiment, the medicament is for treatment of a cell proliferative disorder (e.g., cancer, e.g., esophageal cancer or an adenocarcinoma) or an autoimmune disorder (e.g., arthritis). In a further embodiment, the medicament is for use in a method of treating a cell proliferative disorder or an autoimmune disorder comprising administering to an individual having a cell proliferative disorder or an autoimmune disorder an effective amount of the medicament. In one such embodiment, the method further comprises administering to the individual an effective amount of at least one additional therapeutic agent, for example, as described below. In a further embodiment, the medicament is for activating effector cells (e.g., T cells, e.g., CD8+ and/or CD4+ T cells), expanding (increasing) an effector cell population, reducing a target cell (e.g., a cell expressing CD20) population, and/or killing target cells (e.g., target tumor cells) in the individual. In a further embodiment, the medicament is for use in a method of enhancing immune function in an individual having a cell proliferative disorder or an autoimmune disorder comprising administering to the individual an amount effective of the medicament to activate effector cells (e.g., T cells, e.g., CD8+ and/or CD4+ T cells), expand (increase) an effector cell population, reduce a target cell (e.g., a cell expressing CD20) population, and/or kill a target cell (e.g., target tumor cell). An “individual” according to any of the above embodiments may be a human.

In a further aspect, the invention provides a method for treating a cell proliferative disorder (e.g., cancer). In one embodiment, the method comprises administering to an individual having such a cell proliferative disorder an effective amount of an bispecific antibody. In one such embodiment, the method further comprises administering to the individual an effective amount of at least one additional therapeutic agent, for example, as described below. An “individual” according to any of the above embodiments may be a human. In a further aspect, the invention provides a method for enhancing immune function in an individual having a cell proliferative disorder or an autoimmune disorder in an individual having a cell proliferative disorder or an autoimmune disorder. In one embodiment, the method comprises administering to the individual an effective amount of a bispecific antibody to activate effector cells (e.g., T cells, e.g., CD8+ and/or CD4+ T cells), expand (increase) an effector cell population, reduce a target cell (e.g., a cell expressing CD20) population, and/or kill a target cell (e.g., target tumor cell).

In another aspect, the invention provides a method for treating a cancer that expresses CD20. In some embodiments, the cancer that expressed CD20 is a hematological cancer, such as a B cell cancer (for example, mature B-cell lymphoma). In some embodiments, the method of treating a cancer comprises administering an effective amount of the bispecific antibody of the disclosure (e.g. anti-CD3ε and anti-CD20 antibody; anti-CD3ε and anti-CD20 antibody with CD58 fusion peptide). In a further aspect of the embodiment, the mature B-cell lymphoma is a Non-Hodgkin's Lymphoma (NHL). In a further aspect of the embodiment, the NHL is selected from the group comprising: germinal-center B-cell-like (GCB) DLBCL, activated B-cell like (ABC) DLBCL, follicular lymphoma (FL), mantle cell lymphoma (CL), acute myeloid leukemia (AML), chronic lymphoid leukemia (CLL), marginal zone lymphoma (ZL), small lymphocytic leukemia (SLL), lymphoplasmacytic lymphoma (LL), Waldenstrom macroglobulinemia (W), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, f Heavy chain disease, μ Heavy chain disease, Plasma cell myeloma. Solitary plasmacytoma of bone, [Extraosseous plasmacytoma, Extranodal marginal zone lymphoma of mucosa-associated lymphoid tissue (MALT lymphoma), Nodal marginal zone lymphoma, Pediatric nodal marginal zone lymphoma. Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation. Lymphomatoid granulomatosis, Primary mediastinal (thymic) large B-cell lymphoma. Intravascular large B-cell lymphoma, ALK-positive large B-cell lymphoma, Plasmablastic lymphoma, Large B-cell lymphoma arising in HHV8-associated multicentric Castleman disease. Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma. In a preferred embodiment of the invention, the method comprises treating a cancer comprising germinal-center B-cell like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), or Burkitt's lymphoma (BL).

In a further aspect, the invention provides pharmaceutical formulations comprising any of the bispecific antibodies provided herein, e.g., for use in any of the above therapeutic methods. In one embodiment, a pharmaceutical formulation comprises any of the bispecific antibodies provided herein and a pharmaceutically acceptable carrier. In another embodiment, a pharmaceutical formulation comprises any of the bispecific antibodies provided herein and at least one additional therapeutic agent, for example, as described herein.

Antibodies of the invention can be used either alone or in combination with other agents in a therapy. For instance, an antibody of the invention may be co-administered with at least one additional therapeutic agent. In certain embodiments, an additional therapeutic agent is a chemotherapeutic agent, growth inhibitory agent, cytotoxic agent, agent used in radiation therapy, anti-angiogenesis agent, apoptotic agent, anti-tubulin agent, or other agent, such as a epidermal growth factor receptor (EGFR) antagonist (e.g., a tyrosine kinase inhibitor), HER1/EGFR inhibitor (e.g., erlotinib (Tarceva™), platelet derived growth factor inhibitor (e.g., Gleevec™ (Imatinib Mesylate)), a COX-2 inhibitor (e.g., celecoxib), interferon, cytokine, antibody other than the anti-CD3 antibody of the invention, such as an antibody that bind to one or more of the following targets ErbB2, ErbB3, ErbB4, PDGFR-beta, BlyS, APRIL, BCMA VEGF, or VEGF receptor(s), TRAIL/Apo2, PD-1 (e.g. Nivolumab, Pembrolizumab, Cemiplimab), PD-L1 (Atezolizumab, Avelumab, Durvalumab), PD-L2, or another bioactive or organic chemical agent. In some embodiments, the invention provides a method wherein the additional therapeutic agent is a glucocorticoid. In one embodiment, the glucocorticoid is dexamethasone.

In some aspects, the additional therapeutic agent is a monoclonal anti-CD20 antibody. Exemplary anti-CD20 antibodies include but are not limited to rituximab (RITUXAN®), obinutuzumab (GAZYVA®), tocilizumab (ACTEMRA®/RoACTEMRA®), ocrelizumab (OCREVUS®), ofatumumab (KESIMPTA®), ibritumomab (ZEVALIN®). In some embodiments, the additional therapeutic agent is rituximab.

In some aspects, the additional therapeutic agent is a checkpoint inhibitor. The term “inhibition” or “inhibitor” includes a reduction in a certain parameter, e.g., an activity, of a given molecule, e.g., an immune checkpoint inhibitor. For example, inhibition of an activity, e.g., an activity of, e.g., PD-1. PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGFR beta, of at least 5%, 10%, 20%, 30%, 40% or more is included by this term. The level of inhibition need not be 100%.

In some aspects, the checkpoint inhibitor is a PD-1 inhibitor. In some aspects, the PD-1 inhibitor is an anti-PD1 antibody. In some aspects, the PD-1 inhibitor is an anti PD-1 monoclonal antibody. Exemplary anti-PD-1 monoclonal antibodies include, but are not limited to cemiplimab (Libtayo), nivolumab (Opdivo), pembrolizumab (Keytruda). In some aspects, the checkpoint inhibitor is a PD-L1 inhibitor. Exemplary PD-L1 inhibitors include but are not limited to avelumab (Bavencio), durvalumab (Imfinzi) and atezolizumab (Tecentriq).

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the bispecific antibody and administration of an additional therapeutic agent occur within about one month, or within about one, two or three weeks, or within about one, two, three, four, five, or six days, of each other. Bispecific antibodies of the disclosure can also be used in combination with radiation therapy.

In some aspects, the additional therapeutic agent is a chimeric antigen receptor (CAR) T cell therapy. In some embodiments, the CAR-T cell therapy specifically binds CD19. Exemplary CAR-T cell therapies that specifically bind CD19 include but are not limited to BREYANZI® (lisocabtagene maraleucel), TECARTUS™ (brexucabtagene autoleucel), KYMRIAH™ (tisagenlecleucel), or YESCARTA™ (axicabtagene ciloleucel). In some embodiments, the CAR-T cell therapy specifically binds BCMA.

Exemplary CAR-T therapies that specifically bind BCMA include but are not limited to ABECMA® (idecabtagene vicleucel), or CARVYKTI™ (ciltacabtagene autoleucel). In some embodiments, the CAR-T cell therapy specifically binds CD22.

An antibody of the invention (and/or any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In some embodiments, the antibody is administered by subcutaneous administration. In some embodiments, an anti-CD3ε antibody administered by subcutaneous injection exhibits a less toxic response in a patient than the same anti-CD3ε antibody administered by intravenous injection. Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

As a general proposition, the therapeutically effective amount of the bispecific antibody administered to human will be in the range of about 0.01 to about 100 mg/kg of patient body weight whether by one or more administrations. In some embodiments, the antibody used is about 0.01 to about 45 mg/kg, about 0.01 to about 40 mg kg, about 0.01 to about 35 mg/kg, about 0.01 to about 30 mg/kg, about 0.01 to about 25 mg/kg, about 0.01 to about 20 mg/kg, about 0.01 to about 15 mg kg, about 0.01 to about 10 mg/kg, about 0.01 to about 5 mg/kg, or about 0.01 to about 1 mg/kg administered daily, for example. In one embodiment, an bispecific antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg kg, or 10 mg/kg (or any combination thereof) may be administered to the patient. Such doses may be administered intermittently, for example, every week or every three weeks (e.g., such that the patient receives from about two to about twenty, or, for example, about six doses of the bispecific antibody). An initial higher loading dose, followed by one or more lower doses may be administered. The progress of this therapy is easily monitored by conventional techniques and assays.

In some embodiments, the methods may further comprise an additional therapy. The additional therapy may be radiation therapy, surgery, chemotherapy, gene therapy, DMA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy may be a separate administration of one or more of the therapeutic agents described above.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000). Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include an antibody of the invention, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present invention also provides methods of treating or alleviating a symptom associated with a cancer. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the immune-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

Antibodies directed against a target such as CD3ε. CD20, or a combination thereof (or a fragment thereof), may be used in methods known within the art relating to the localization and/or quantitation of these targets, e.g., for use in measuring levels of these targets within appropriate physiological samples, for use in diagnostic methods, for use in imaging the protein, and the like). In a given embodiment, antibodies specific any of these targets, or derivative, fragment, analog or homolog thereof, that contain the antibody derived antigen binding domain, are utilized as pharmacologically active compounds (referred to hereinafter as “Therapeutics”).

An antibody of the invention can be used to isolate a particular target using standard techniques, such as immunoaffinity, chromatography or immunoprecipitation. Antibodies of the invention (or a fragment thereof) can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies or a fragment thereof of the invention can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences. Vol. 4), 1991, M. Dekker. New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles. e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of a given target (or a protein fragment thereof) in a sample. In some embodiments, the antibody contains a detectable label. Antibodies are polyclonal, or more preferably, monoclonal. An intact antibody, or a fragment thereof (e.g., Fab, scFv, or F_{(ab)2}) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled.

Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, dAb (domain antibody), single chain, F_{ab}, F_{ab′} and F_{(ab)2} fragments, scFvs, and an F_{ab} expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, IgG₄ and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen binding region” or “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” Various methods are known in the art for numbering the amino acids sequences of antibodies and identification of the complementary determining regions. For example, the Kabat numbering system (See Kabat, E. A., et al., Sequences of Protein of immunological interest. Fifth Edition, US Department of Health and Human Services, US Government Printing Office (1991)) or the IMGT numbering system (See IMGT®, the international ImMunoGeneTics information system*. Available online: http://www.imgt.org/). The IMGT numbering system is routinely used and accepted as a reliable and accurate system in the art to determine amino acid positions in coding sequences, alignment of alleles, and to easily compare sequences in immunoglobulin (IG) and T-cell receptor (TR) from all vertebrate species. The accuracy and the consistency of the IMGT data are based on IMGT-ONTOLOGY, the first, and so far unique, ontology for immunogenetics and immunoinformatics (See Lefranc. M. P. et al., Biomolecules, 2014 December; 4(4), 1102-1139). IMGT tools and databases run against IMGT reference directories built from a large repository of sequences. In the IMGT system the IG V-DOMAIN and IG C-DOMAIN are delimited taking into account the exon delimitation, whenever appropriate. Therefore, the availability of more sequences to the IMGT database, the IMGT exon numbering system can be and “is used” by those skilled in the art reliably to determine amino acid positions in coding sequences and for alignment of alleles. Additionally, correspondences between the IMGT unique numbering with other numberings (i.e., Kabat) are available in the IMGT Scientific chart (See Lefranc. M. P. et al., Biomolecules, 2014 December; 4(4), 1102-1139).

The term “hypervariable region” or “variable region” refers to the amino acid residues of an antibody that are typically responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system: Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop” VCDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally, the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is the to specifically bind an antigen when the dissociation constant is ≤1 μM; e.g., ≤100 nM, preferably ≤10 nM and more preferably ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is the to specifically bind to its target, when the equilibrium binding constant (K_{d}) is ≤1 μM, e.g., ≤100 nM, preferably ≤10 nM, and more preferably ≤1 nM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the heavy chain immunoglobulin molecules, and nucleic acid molecules encoding the light chain immunoglobulin molecules described herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source. e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules, and the light chain immunoglobulin molecules described herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to affect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

As used herein, the twenty conventional amino acids and their abbreviations follow conventional usage. See Immunology—A Synthesis (2nd Edition, E. S. Golub and D. R. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%,, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton. Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Te, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

### EXAMPLES

### Example 1: Materials and Methods

### T Cell Isolation and Activation

T cells were isolated from human PBMCs using StemCell Human T Cell Isolation Kit. T cells were activated with StemCell ImmunoCult Human CD3/CD28 T Cell Activator (in StemCell SFEM II with 10% FBS and 5 ng/mL Peprotech IL-2). Activation was done for 2-3 days followed by CAR-T generation and then expansion in StemCell SFEM II with 10% FBS supplemented with 5 ng/mL Peprotech IL-2. T cells were cultured at 37° C. with 5% CO₂.

### CAR-T Generation

T cells activated with Immunocult (and 5 ng/mL IL-2) were washed and diluted to 0.625 million/mL. After addition of polybrene (5 ug/mL final), BPS Biosciences anti-CD19 CAR Lentivirus (CD19 ScFv-CD8-4-1BB-CD3) was added to these cells at a MOI of 20. After 10 minutes at RT, cells were centrifuged at 800×g and 32 C for 2 hours. Following spinofection, cells were diluted 5× in fresh media. Two days later, cells were selected using 0.5 ug/mL puromycin. Two-three days later, cells were further selected with 1 μg/mL puromycin. Cells were further expanded in StemCell SFEM II with 10% FBS and 5 ng/mL IL-2 until their viability returned to >95%. To produce NT (non-transduced) T cells, cells were treated in the same way as the resultant CAR-T cells but did not receive virus nor puromycin selection.

### Cell Lines

Jeko-1 cells, previously engineered with GFP-Luc by lentivirus from BPS Biosciences, were grown in RPMI supplemented with 20% FBS at 37 C with 5% CO2. All co-culture assays with CAR-T cells were carried out in RPMI supplemented with 10% FBS at 37° C. with 5% CO2.

### CRISPR-Mediated Editing

To assemble RNP complexes, sgRNAs (3 per gene) synthesized by IDT were added to IDT Recombinant CRISPR Cas9 Alt-R S.p. HiFi Nuclease V3 in a 2.5 to 1 ratio and incubated for 15 minutes at RT. Jeko-1 cells were washed twice in RPMI, then resuspend in Lonza SG Cell Buffer before being added to the RNP Complexes. A Lonza 4D-Nucleofector was used to electroporate the Jeko-1 cells using the DS-104 program. After electroporation, the cells were rested. The following guides were used to generate KO cell lines: AAVS1 (AAVS1_1: GGGACCACCTTATATTCCCA (SEQ ID NO: 700), AAVS1_2: GCGGCTCCAATTCGGAAGTG (SEQ ID NO: 701), AAVS1_3: CACGGGGAGGATCCGCTCAG (SEQ ID NO: 702)), CD19 (CD19_1: GCCGAGCAGTGATCTCCAGG (SEQ ID NO: 703), CD19_2: CCCCTGGACACAGAGTCAGG (SEQ ID NO: 704), CD19_3: CACAGCGTTATCTCCCTCTG (SEQ ID NO: 705)), and CD20 (CD20_1: TTCCAGAAATGGCAGCAAAG (SEQ ID NO: 706), CD20_2: TTCATAATCTGGACAGCCTG (SEQ ID NO: 707), CD20_3: ACCATAATGCCTCCCCAGAG (SEQ ID NO: 708)). To isolate CD19 or CD20 negative cells, the StemCell EasySep Human APC Positive Selection II Kit was used in negative selection mode in combination with Biolegend APC anti-CD19 or anti-CD20 antibody. Two cycles of isolation were necessary to result in a CD19-KO Jeko-1 and then a CD19-KO and CD20-KO Jeko-1 population.

### Tumor Killing Assays

Tumor killing assay were completed as follows, AAVS1 knockout, CD19 knockout, or CD19/CD20 knockout Jeko-1 cells co-cultured with either non-transduced (NT) or CD19 targeted chimeric antigen receptor expressing T lymphocytes at an effector T cell (E) to tumor cell (T) ratio of 1 to 3 with 10 K T cells and 30 K tumor cells, 1 to 5 with 10 K T cells and 50 K tumor cells, or 1 to 10 with 10 K T cells and 100 K tumor cells in the presence of biologics as specified. After 2 days of incubation at 37 degrees, green fluorescent tumor cells were measured by flow cytometry. Tumor cells flow cytometry data were analyzed by FlowJo and graphed with GraphPad Prism. For comparison purposes, the cells were normalized to the average of cells remaining in co-culture with no biologics.

### Analytical/Flow Cytometry

A BD Biosciences FAC Symphony A5 instrument was used for analytical flow cytometry. For tumor-T cell co-cultures, the live cells with GFP were gated for tumor cell counting. To assess surface levels of CD19 (SJ26C1), CD20 (2H7), BCMA (19F2), and CD180 (MHR73.11) on Jeko-1 cells, antibodies conjugated with BUV395, BV421, PE, and APC were used. To assess surface levels of CD3 (UCHT1), CD4 (OKT4), CD8 (SK1), CD28 (CD28.2), and CD2 (RPA-2.10) on T cells, antibodies conjugated with BUV395, BV421, FITC, PE, and APC were used. The correct isotype controls conjugated with the same fluorophore were also used.

### Example 2: Production of CD19-Knockout Jeko-1 Cells

The present application demonstrates that bispecific antibodies can effectively retarget anti-CD19 CAR-T cells to CD19-negative tumor cells, stimulating an T cell mediated immune response to a tumor cell population and resulting in enhanced tumor cell killing.

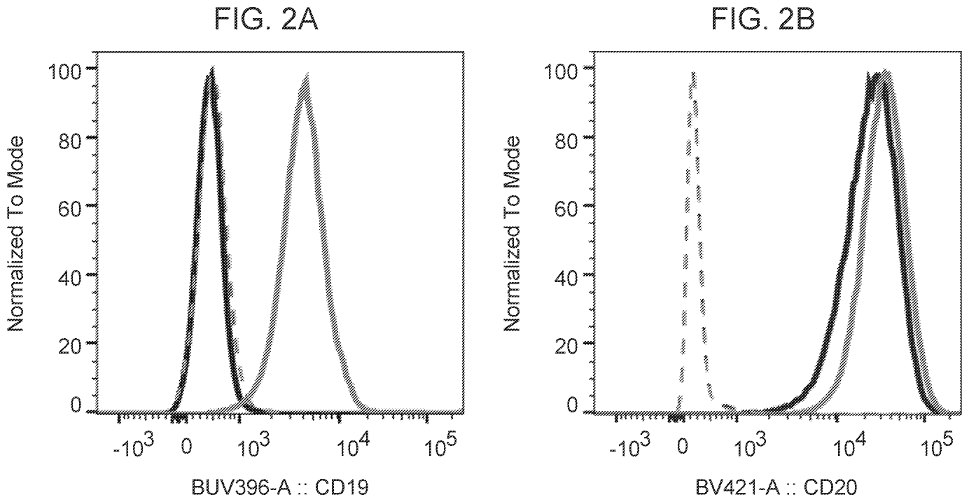

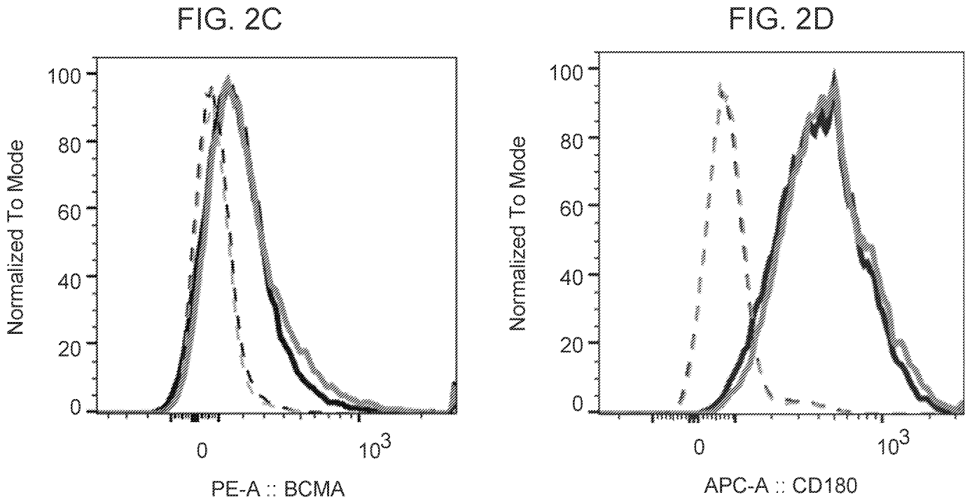

Jeko-1 cells engineered with green fluorescent protein and firefly luciferase to simplify measurements of cell numbers by flow cytometry were used to generate negative control and CD19 knock cell models. Three sgRNAs per gene targeting either CD19 or the AAVS1 locus with the latter serving as a non-gene targeted control were delivered by electroporation as pre-complexed Cas9 ribonucleotide complexes. Following electroporation cells underwent two rounds of negative bead-based selection using the StemCell EasySep Human APC Positive Selection Kit. After selection, Jeko-1 GFP-Luc AAVS1-KO (Control-KO) and Jeko-1 GFP-Luc CD19-KO cells were expanded and each cell population was assessed for target levels. As shown in FIG. 2A, CD19-KO Jeko-1 cells (“CD19-KO”) were efficiently knocked out for CD19, as the CD19-KO peak (black peak) shifted to the left compared to the Control-KO peak (gray peak) and resembled the “Control-KO isotype” peak (dashed line). The levels of CD20, BCMA, and CD180 in the CD19-KO cells (black line) were comparable to the levels of CD20 in the Control-KO cells (gray pline) (FIG. 2B), indicating that both CD19-KO and Control-KO Jeko-1 cells express surface CD20, BCMA, and CD180.

### Example 3: CAR-T Cells are not Effective if there is Antigen Loss; CAR-T Cells Increase and Retain their CD2 and CD3 Levels

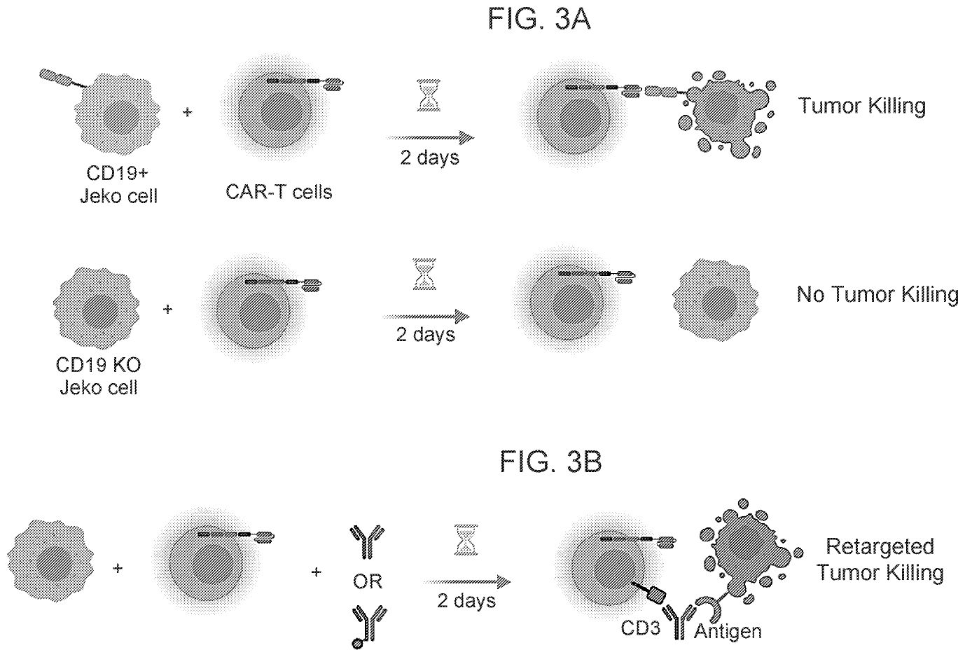

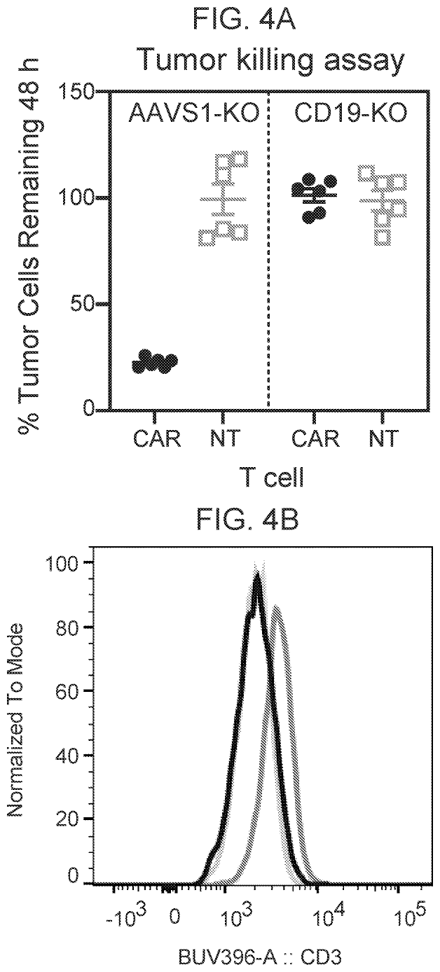

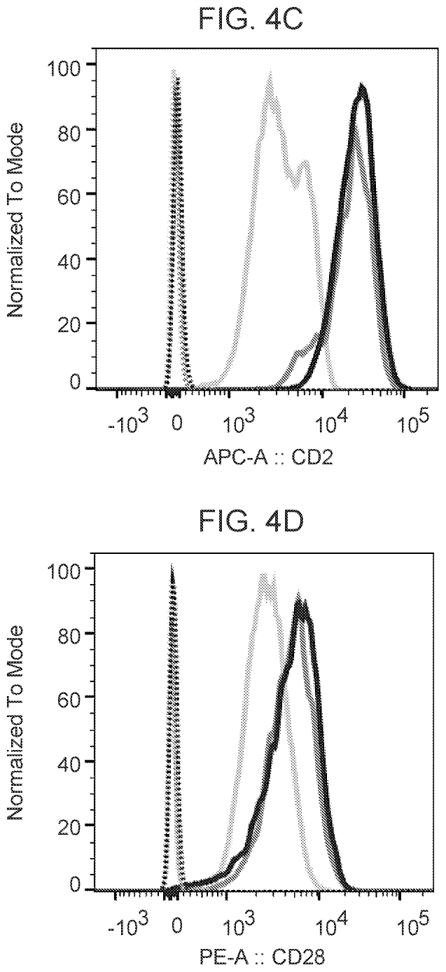

When anti-CD19 CAR-T cells are co-cultured with control-KO Jeko-1 cells, they kill effectively but when they are co-cultured with CD19-KO Jeko-1 cells, they can no longer kil the tumor cells (FIG. 3A). Anti-CD19 CAR-T cells (“CAR”) and non-transduced (“NT”) T cells were co-cultured with Control-KO and CD19-KO Jeko-1 cells at an E:T ratio of 1:3. While anti-CD19 CAR-T cells (“CAR”) efficiently kill CD19-positive Jeko-1 cells (FIG. 4A, left panel), there is no tumor killing seen when anti-CD19 CAR-T cells are co-cultured with CD19-KO Jeko-1 cells (FIG. 4, right panel). These CAR-T cells retain CD3 levels as compared to naïve T cells (FIG. 4B). Interestingly, CAR-T cells have much higher levels of CD2 than naive T cells (FIG. 4C). In contrast, CAR-T cells have only slightly higher CD28 levels than naïve T cells (FIG. 4D). As cells experience activation and exhaustion, their CD28 levels decrease while their CD2 levels increase. Therefore, a bispecific antibody that engages CD2 for costimulation may be more effective than one that engages CD28. CAR-T cells have sufficient CD3 and CD2 levels to be engaged by bispecific antibodies that target one or both of those receptors.

### Example 4: Bispecific Antibodies Retarget CD19-Directed CAR-T Cells and Promote Effective Killing of CD19-Negative Tumor Cells

Even though no CAR-T-mediated killing occurs in CD19-KO cells, when bispecific antibodies comprising a first antigen binding region that binds to the CAR-T cell and a second antigen binding region that binds to the tumor cell are added to the co-culture, the anti-CD19 CAR-T cells can be retargeted to the CD19-negative tumor cells inducing tumor cell killing, as depicted in the schematic in FIG. 3B.

A E:T ratio of 1:3 was used for further assessment with other tumor antigens.

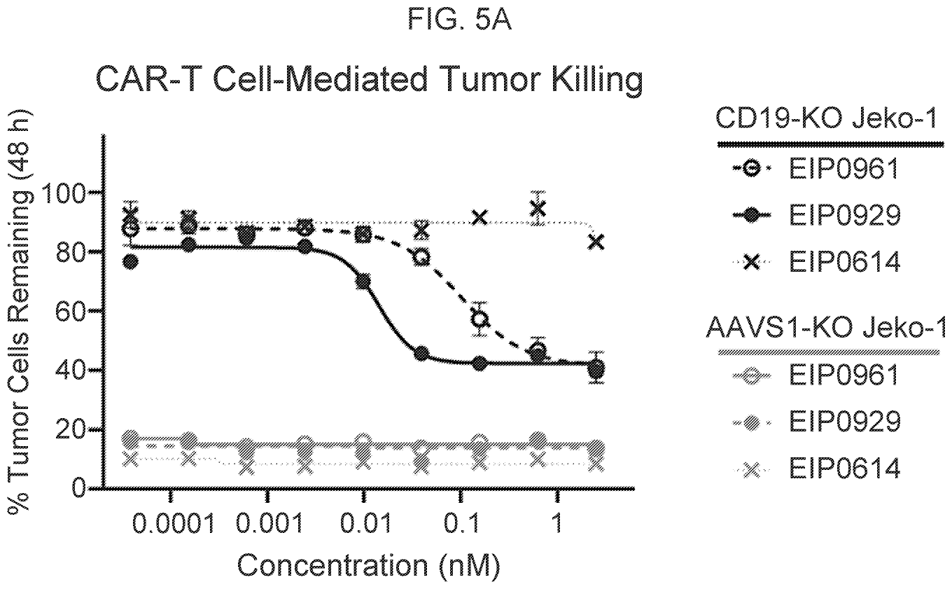

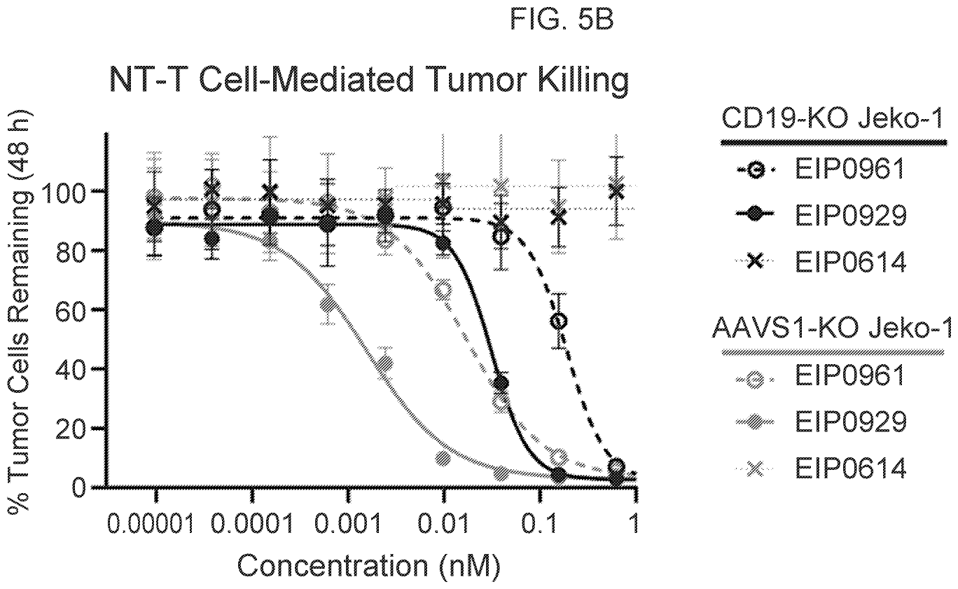

When the anti-CD20/anti-CD3 antibodies were assayed at half-log dilutions in a co-culture assay at an E:T ratio of 1:3, the CAR-T retargeting to anti-CD19 Jeko-1 cells was also observed. While the Control AAVS1-KO Jeko-1 cells were efficiently killed at all concentrations of antibody (due to killing through the CAR), the anti-CD20/anti-CD3/CD58 fusion (EIP0929) bispecific antibody more efficiently killed the CD19-KO cells as compared to the anti-CD20/anti-CD3 bispecific (EIP0961) antibody or the non-tumor-targeting control (EIP0614), as shown in FIG. 5A. Both antibodies were active with NT-T cells for both Control-KO and CD19-KO Jeko-1 cells as expected (FIG. 5B).

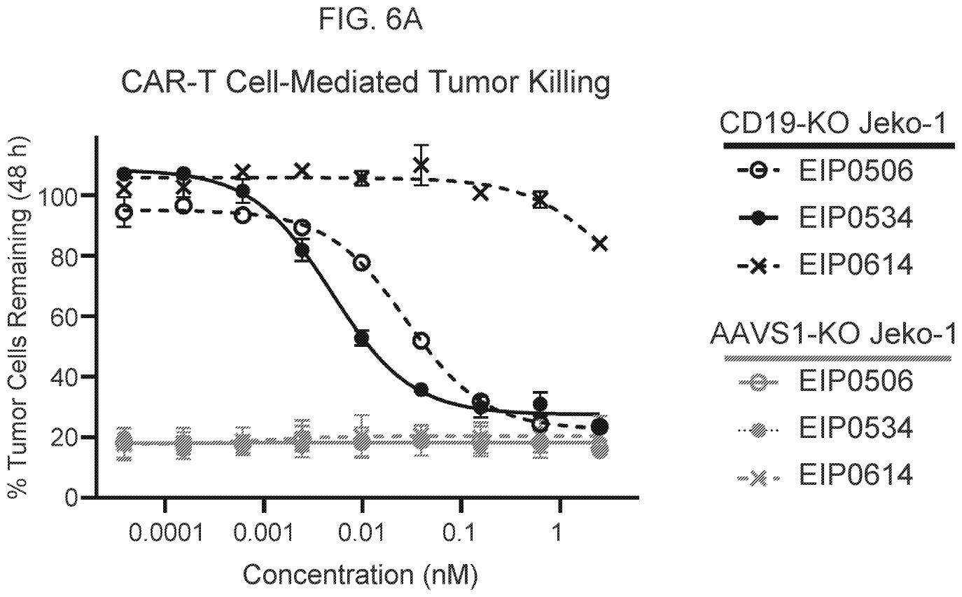

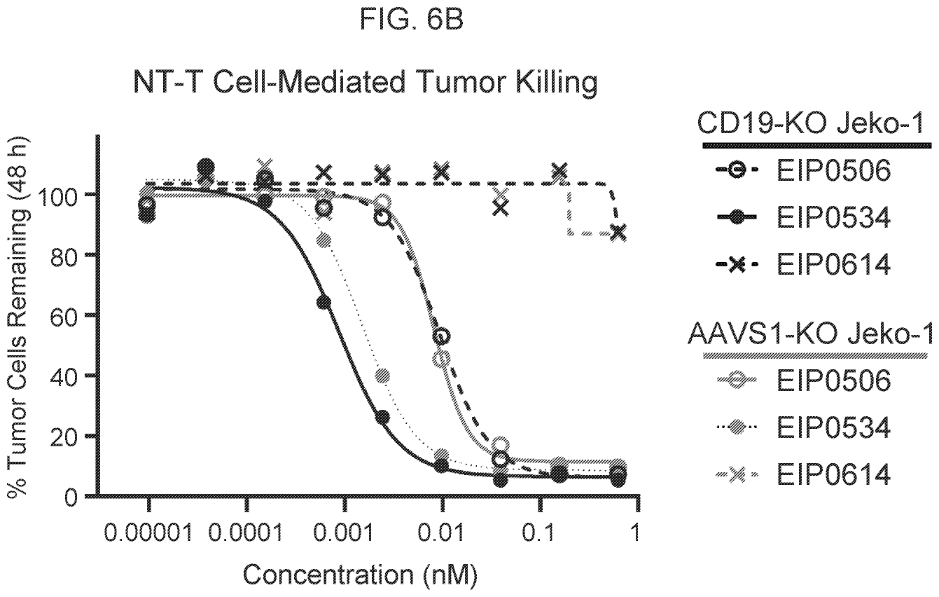

When the anti-BCMA/anti-CD3 antibodies were assayed at half-log dilutions in a co-culture assay at an E:T ratio of 1:3, the CAR-T retargeting to anti-CD19 Jeko-1 cells was also observed. While the Control AAVS1-KO Jeko-1 cells were efficiently killed at all concentrations of antibody (due to killing through the CAR), the anti-BCMA/anti-CD3/CD58 fusion (EIP0534) bispecific antibody more efficiently killed the CD19-KO cells as compared to the anti-BCMA/anti-CD3 bispecific (EIP0506) antibody or the non-tumor-targeting control (EIP0614), as shown in FIG. 6A. Both antibodies were active with NT-T cells for both Control-KO and CD19-KO Jeko-1 cells as expected (FIG. 6B).

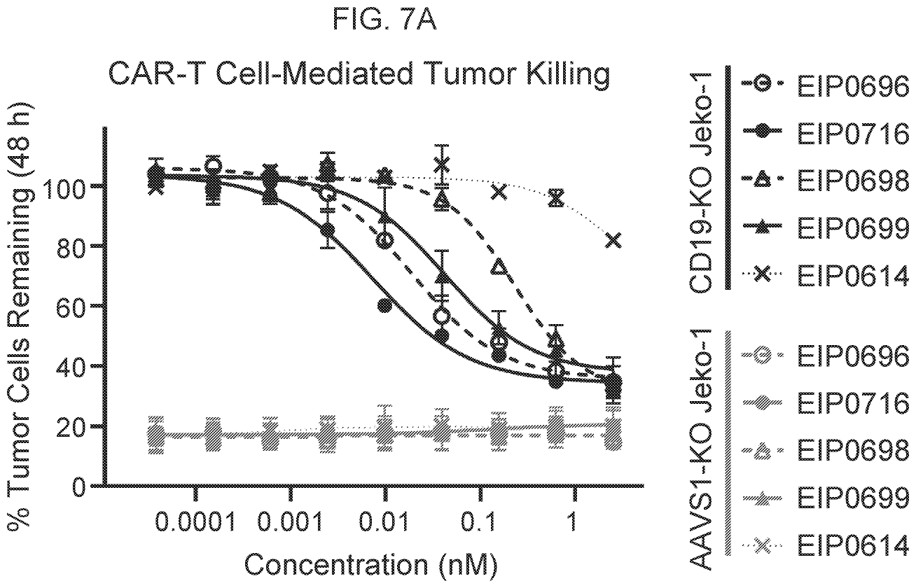

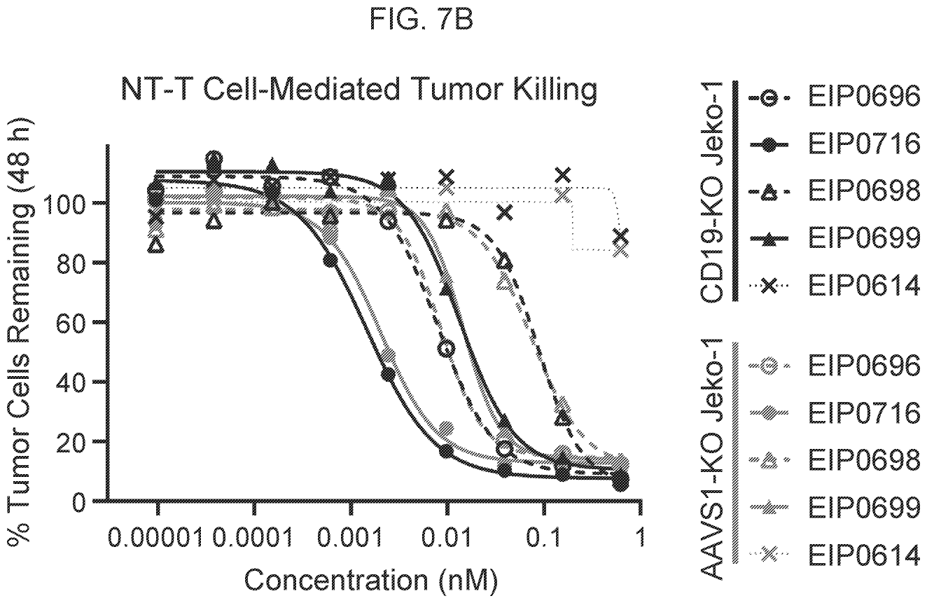

When the anti-CD180/anti-CD3 antibodies were assayed at half-log dilutions in a co-culture assay at an E:T ratio of 1:3, the CAR-T retargeting to anti-CD19 Jeko-1 cells was also observed. While the Control AAVS1-KO Jeko-1 cells were efficiently killed at all concentrations of antibody (due to killing through the CAR), the anti-CD180/anti-CD3/CD58 fusion (EIP0716 and EIP0699) bispecific antibodies more efficiently killed the CD19-KO cells as compared to the anti-CD180/anti-CD3 bispecific (EIP0696 and EIP0698) antibody or the non-tumor-targeting control (EIP0614), as shown in FIG. 7A. Both antibodies were active with NT-T cells for both Control-KO and CD19-KO Jeko-1 cells as expected (FIG. 7B).

### Example 5: Bispecific Antibodies can Increase Effectiveness of CAR-T Cells Even without Antigen Loss

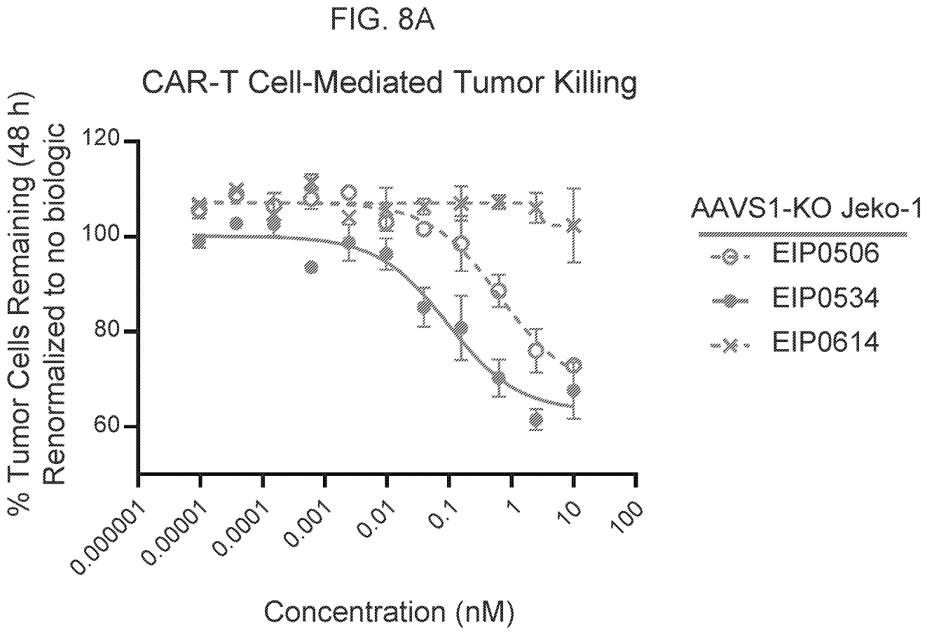

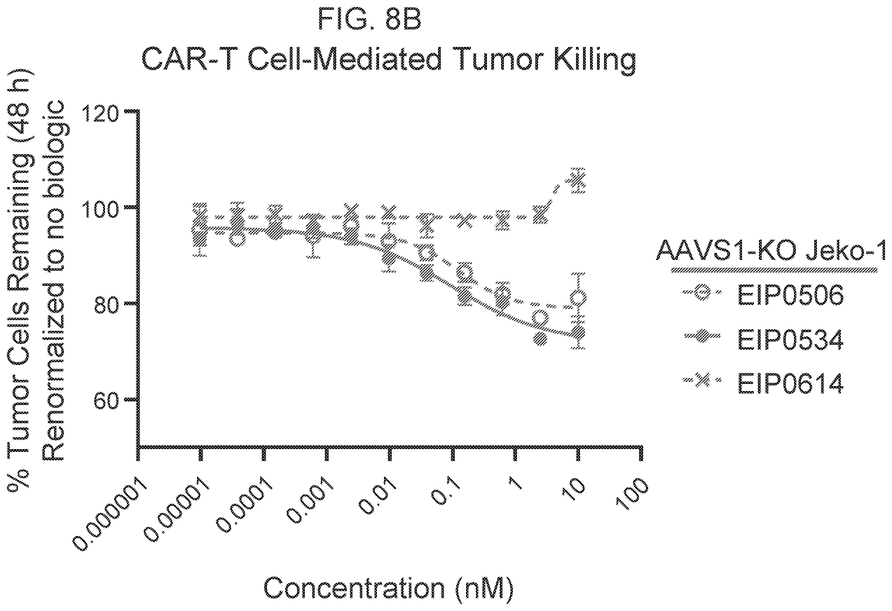

While the largest effect of bispecific antibodies is in cells that mimic CD19-loss, the improvement with bispecific antibodies can also be seen for control tumor cells at low E:T ratios. When we renormalize to the no biologic controls (thereby, just looking at the tumor cells remaining after CAR-T cell-mediated killing), both the anti-BCMA/anti-CD3/CD58 fusion (EIP0534) bispecific antibody and the anti-BCMA/anti-CD3 bispecific (EIP0506) antibody more efficiently killed the control cells as compared to the non-tumor-targeting control (EIP0614). The effect can be seen for both E:T ratio of 1:5 (FIG. 8A) and 1:10 (FIG. 8B). Since patient retained CAR-T cells are normally small in count, these E:T ratios are a closer mimic to what is found in these patients. Even if no antigen loss is experience by the tumor cells, bispecific antibodies can improve tumor killing.

### Example 6: Production of Double CD19- and CD20-Knockout Jeko-1 Cells and Tumor Retargeting with Anti-CD180 Antibodies

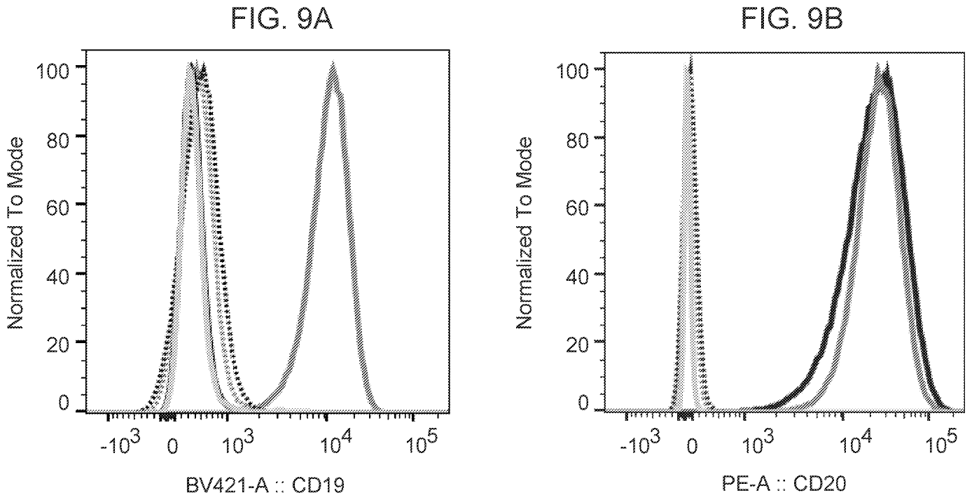

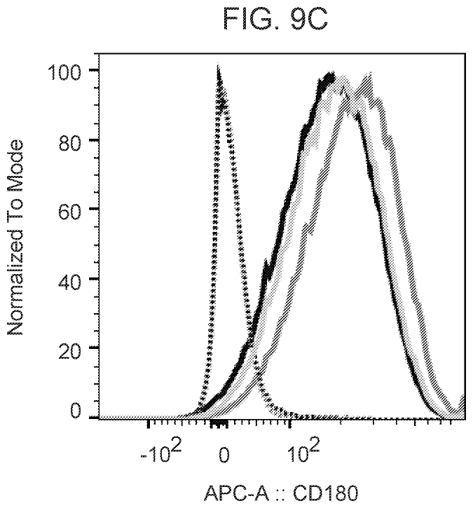

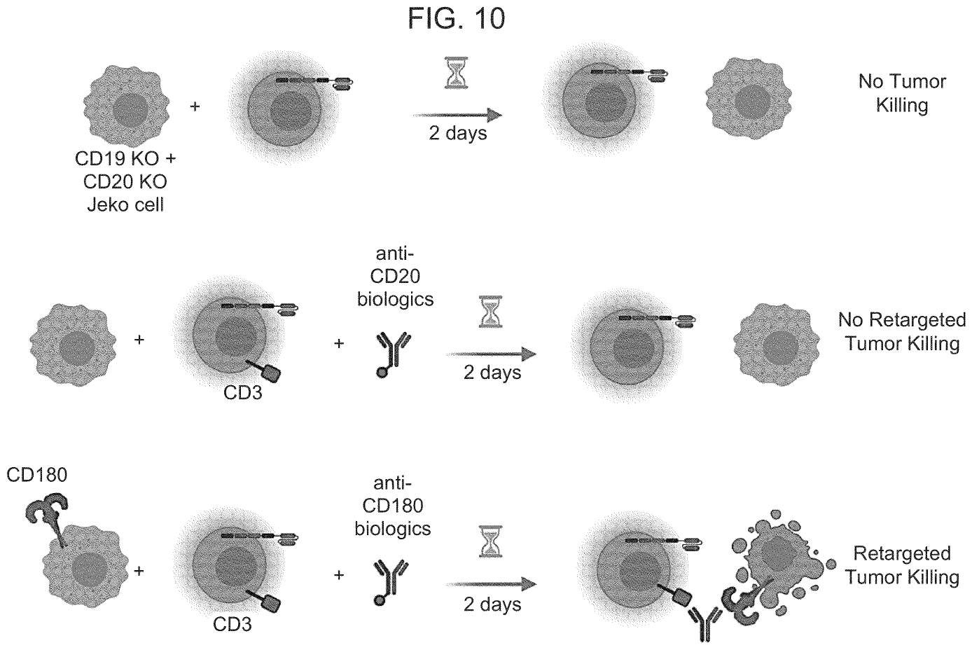

One potential mechanism of antigen loss is B cell class switching which could affect not only CD19 levels but also CD20 levels. To mimic this type of antigen loss, we engineered CD20-KO in the CD19-KO background of Jeko-1 GFP-Luc cells as described above to create double CD19 and CD20 knockout cells. We assessed the receptor levels of these cells as we did previously and found that while CD19 (FIG. 9A) and CD20 (FIG. 9B) were at isotype-levels in the double knockout CD19 and CD20 Jeko-1 cells, levels of another tumor antigen, CD180 (FIG. 9C), were unchanged. In the schematic in FIG. 10, we show that both CAR-T cells (upper panel) and anti-CD20/anti-CD3 biologics (middle panel) would not be effective in targeting or retargeting CAR-T cells for tumor killing. However, by using anti-CD180/anti-CD3 antibodies (FIG. 9; lower panel), we can maintain retargeted CAR-T tumor killing.

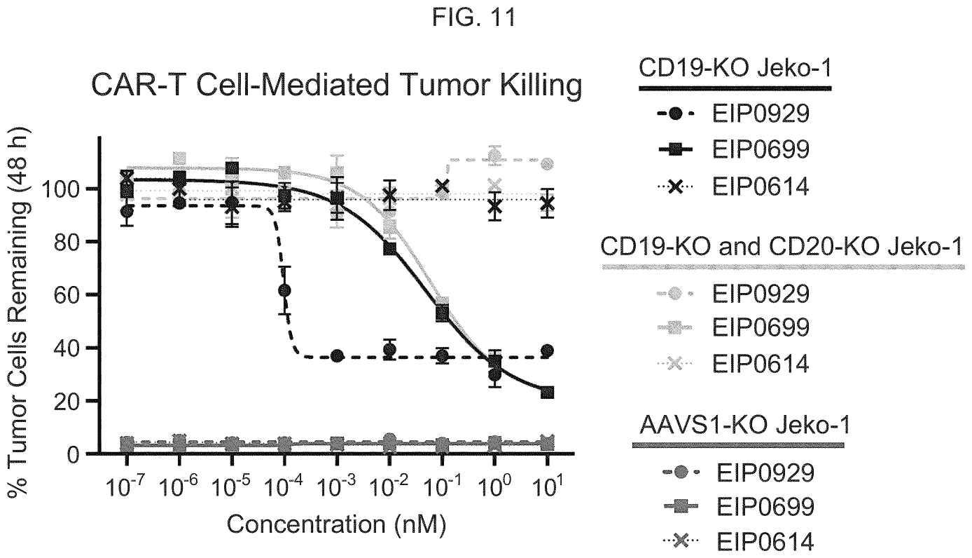

Indeed, CD19 knockout Jeko-1 cells can be effectively killed by either the anti-CD20/anti-CD3/CD58 fusion (EIP0929) bispecific antibody or the anti-CD180/anti-CD3/CD58 fusion (EIP0699) bispecific antibody (FIG. 11, black lines). However, double CD19 and CD20 knockout Jeko-1 cells can only be killed by the anti-CD180/anti-CD3/CD58 fusion (EIP0699) bispecific antibody (FIG. 11, light gray lines). Therefore, by targeting an antigen that is maintained, CAR-T function and retargeting can be more robust over a variety of antigen loss scenarios.

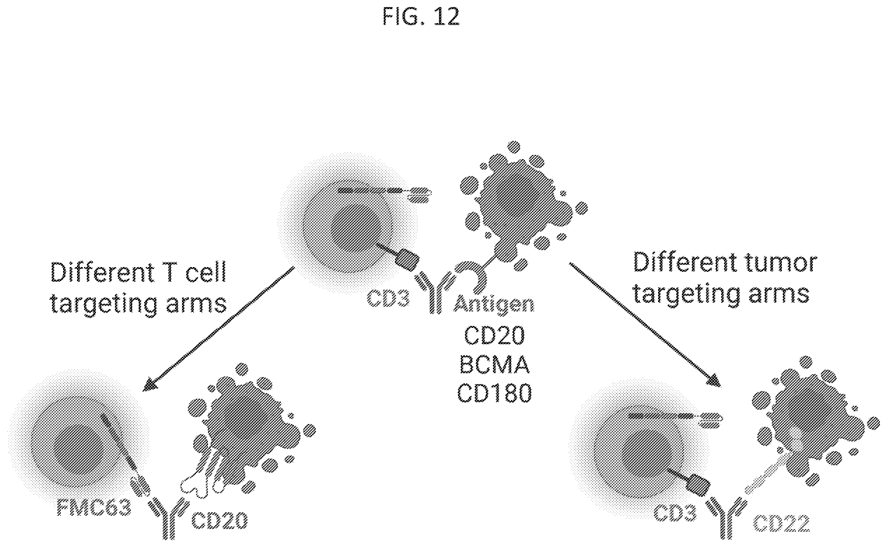

Herein we show retargeting of FMC63 anti-CD19 CAR-T cells with anti-CD20/anti-CD3 biologics, anti-BCMA/anti-CD3 biologics, and anti-CD180/anti-CD3 biologics. However this idea can be extended to other biologic molecules that can retarget CAR-T cells to available tumor antigens and T cell targets. As depicted in the schematic in FIG. 12, either the T cell targeting arm (e.g., CD3-directed arm) and/or the tumor cell targeting arm (e.g., CD20-, BCMA-, and CD180-targeting arm) can be varied to obtain the most ideal retargeting antibody. Of interest is a biologic targeting FMC63 (or any other CAR or CAR domain) itself or the CAR hinge as signaling through the CAR seems to result in more robust T cells than signaling through the T cell receptor (by way of anti-CD3 targeting antibodies). Other T cell antigens other than CD3 may also be targeted. Other tumor targets especially those associated with hematologic malignancies, such as CD22, could also prove to be efficacious. Even further, retargeting can also be applied to solid tumor cancers using different CAR molecules and correspondingly varied antigens of interest for the tumor targeting arm. The biologics shown here-in are particularly effective because they target receptors that are maintained and even increased on the surface of T cells such as CD2. We also show that these biologics can re-target T cells even at very low E:T ratios as is usually the case because few CAR-T cells are maintained long term in patients. Overall, this strategy can complement and enhance T-cell mediated tumor killing in patients that received CAR-T cells and may be losing effectiveness due to antigen loss or decrease of CAR-T cell numbers.

### EMBODIMENTS

Additional embodiments of the disclosure include the following:

Embodiment 1. A method of T cell re-targeting to a target tumor cell in a subject in need thereof comprising administering to the subject a population of modified T cells and a bispecific antibody, wherein the bispecific antibody comprises a first antigen binding region that binds to the modified T cell and a second antigen binding region that binds to the tumor cell.

Embodiment 2. A method for stimulating an T cell mediated immune response to a tumor cell population, the method comprising administering a population of modified T cells and a bispecific antibody, wherein the bispecific antibody comprises a first antigen binding region that binds to the modified T cell and a second antigen binding region that binds to the tumor cell.

Embodiment 3. A method of treating a cancer in a subject in need thereof comprising administering to the subject a population of modified T cells and a bispecific antibody, wherein the bispecific antibody comprises a first antigen binding region that binds to the modified T cell and a second antigen binding region that binds to the tumor cell.

Embodiment 4. The method of any one of embodiments 1-3, further comprising administering a modified T cell activation agent.

Embodiment 5. The method of any one of embodiments 1-4, wherein the bispecific antibody first antigen binding region binds to CD3 expressed on the modified T cell.

Embodiment 6. The method of any one of embodiments 1-5, wherein the modified T cell comprises a chimeric antigen receptor (CAR).

Embodiment 7. The method of embodiment 6, wherein the CAR binds to a disease-associated antigen on a tumor cell.

Embodiment 8. The method of embodiment 7, wherein the disease-associated antigen is CD19.

Embodiment 9. The method of any one of embodiments 6-8, wherein the bispecific antibody first antigen binding region binds to the CAR expressed on the modified T cell.

Embodiment 10. The method of any of embodiments 1-9, wherein the bispecific antibody second antigen binding region binds to a disease-associated antigen on a tumor cell.

Embodiment 11. The method of embodiment 10, wherein the disease-associated antigen is selected from the group consisting of CD20, BCMA, CD79A, CD79B, CD22, and CD180.

### INCORPORATION BY REFERENCE

Throughout this application various publications, patents, and/or patent applications are referenced. The disclosures of the publications, patents and/or patent applications are hereby incorporated by reference in their entireties into this application in order to more fully describe the state of the art to which this disclosure pertains.

1. A method of T cell re-targeting to a tumor cell population in a subject, the method comprising administering to the subject a bispecific antibody,
wherein the subject was previously administered a population of modified T cells that bind a first target antigen on the tumor cell,
wherein the bispecific antibody comprises a first antigen binding region that binds to a target antigen on the modified T cell and a second antigen binding region that binds to a second target antigen on the tumor cell.

2. A method for stimulating a T cell mediated immune response to a tumor cell population in a subject, the method comprising administering to the subject a bispecific antibody,
wherein the subject was previously administered a population of modified T cells that bind a first target antigen on the tumor cell,
wherein the bispecific antibody comprises a first antigen binding region that binds to a target antigen on the modified T cell and a second antigen binding region that binds to a second target antigen on the tumor cell.

3. A method of treating a cancer in a subject, the method comprising administering to the subject a bispecific antibody,
wherein the subject was previously administered a population of modified T cells that bind a first target antigen on the tumor cell,
wherein the bispecific antibody comprises a first antigen binding region that binds to a target antigen on the modified T cell and a second antigen binding region that binds to a second target antigen on the tumor cell.

4. A method of T cell re-targeting to a tumor cell population in a subject, the method comprising administering to the subject a population of modified T cells that bind a first target antigen on the tumor cell and a bispecific antibody,
wherein the bispecific antibody comprises a first antigen binding region that binds to a target antigen on the modified T cell and a second antigen binding region that binds to a second target antigen on the tumor cell.

5. A method for stimulating a T cell mediated immune response to a tumor cell population in a subject, the method comprising administering to the subject a population of modified T cells that bind a first target antigen on the tumor cell and a bispecific antibody,
wherein the bispecific antibody comprises a first antigen binding region that binds to a target antigen on the modified T cell and a second antigen binding region that binds to a second target antigen on the tumor cell.

6. A method of treating a cancer in a subject, the method comprising administering to the subject a population of modified T cells that bind a first target antigen on the tumor cell and a bispecific antibody,
wherein the bispecific antibody comprises a first antigen binding region that binds to a target antigen on the modified T cell and a second antigen binding region that binds to a second target antigen on the tumor cell.

7. The method of any one of claims 1-6, wherein the subject is resistant, inadequately responsive to, or relapsed following the administration of a population of modified T cells that bind a first target antigen on the tumor cell.

8. The method of claim 1-7, wherein the first target antigen is no longer expressed on the tumor cell following the administration of the population of modified T cells that bind a first target on the tumor cell.

9. The method of any one of claims 1-8, wherein the first target antigen on the tumor cell is expressed at a low level.

10. The method of any one of claims 1-9, wherein the first target antigen on the tumor cell is expressed at a lower level than the second target antigen on the tumor cell.

11. The method of any one of claims 1-10, wherein the first target antigen on the tumor cell and the second target antigen on the tumor cell is expressed at an equal level.

12. The method of any one of claims 1-11, wherein the administration of the bispecific antibody results in higher levels of tumor cell killing in comparison to the level of tumor cell killing from administration of a population of modified T cells alone.

13. The method of claim 12, wherein the level of tumor cell killing is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater than the level of tumor cell killing from administration of a population of modified T cells alone.

14. The method of any one of claims 1-13, wherein the administration of the bispecific antibody increases an immune response against the tumor cell population in comparison to an immune response from administration of the population of modified T cells alone.

15. The method of claim 14, wherein the immune response is T cell activation and/or T cell proliferation of the modified T cell and/or a natural (NT)-T cell.

16. The method of claim 14, wherein the immune response is modified T-cell mediated tumor killing.

17. The method of claim 14, wherein the immune response is natural (NT)-T cell mediated tumor killing.

18. The method of claim 14, wherein the immune response is modified T-cell mediated tumor killing and natural (NT)-T cell mediated tumor killing.

19. The method of any one of claims 14-18, wherein the increased immune response is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater against a tumor cell in comparison to the immune response from administration of the population of modified T cells alone.

20. The method of any one of claims 1-19, wherein the target antigen on the modified T cell is CD3.

21. The method of any one of claims 1-19, wherein the target antigen on the modified T cell is an antigen binding region of a chimeric antigen receptor.

22. The method of any one of claims 1-19, wherein the target antigen on the modified T cell is a linker region of a chimeric antigen receptor.

23. The method of any one of claims 1-22, wherein the first target antigen on the tumor cell and the second target antigen on the tumor cell are the same.

24. The method of claim 23, wherein the first target antigen on the tumor cell and the second target antigen on the tumor cell is CD19, BCMA or CD22.

25. The method of any one of claims 1-22, wherein the first target antigen on the tumor cell and the second target antigen on the tumor cell are different.

26. The method of claim 25, wherein the first target antigen on the tumor cell is CD19, BCMA or CD22.

27. The method of any one of claims 25-26, wherein the second target antigen on the tumor cell is CD20, CD180, BCMA, CD79A, CD79B or CD22.

28. The method of any one of claims 1-27, wherein the bispecific antibody comprises a first antigen binding region that binds to CD3 and a second antigen binding region that binds to CD19.

29. The method of any one of claims 1-27, wherein the bispecific antibody comprises a first antigen binding region that binds to CD3 and a second antigen binding region that binds to CD20.

30. The method of any one of claims 1-27, wherein the bispecific antibody comprises a first antigen binding region that binds to CD3 and a second antigen binding region that binds to CD180.

31. The method of any one of claims 1-27, wherein the bispecific antibody comprises a first antigen binding region that binds to CD3 and a second antigen binding region that binds to BCMA.

32. The method of any one of claims 1-31, wherein the modified T cell is a CAR T cell.

33. The method of any one of claims 1-32, wherein the bispecific antibody has the following structure:
a first heavy chain polypeptide (H1) comprising a variable region (VH1), and a constant region (CH1) having a constant region 1 domain (CH1_H1), a hinge region (H1H), a constant region 2 domain (CH1_H2) and a constant region 3 domain (CH1_H3); and a first light chain polypeptide (L1) comprising a variable region (VL1) and a constant region (CL1); and a second heavy chain polypeptide (H2) comprising a variable region (VH2), and a constant region (CH2) having a constant region 1 domain (CH2_H1), a hinge region (H2H), a constant region 2 domain (CH2_H2) and a constant region 3 domain (CH2_H3); and second light chain polypeptide (L2) comprising a variable region (VL2) and a constant region (CL2), and wherein
i) the amino acid at position 39 (Kabat numbering) of the VH1 is a K and the amino acid at position 38 (Kabat numbering) of the VL1 is a D;
ii) the amino acid at position 147 (EU numbering) of the CH1_H1 is a K and the amino acid at position 131 (EU numbering) of the CL1 is a D;
iii) the amino acid at position 173 (EU numbering) of the CH1_H1 is a C and the amino acid at position 162 (EU numbering) of the CL1 is a C;
iv) the amino acid at position 220 (EU numbering) in the H1H is a S and the amino acid at position 214 (EU numbering) of the CL1 is a S;
v) the amino acid at position 39 (Kabat numbering) of the VH2 is a D and the amino acid at position 38 (Kabat numbering) of the VL2 is a K; and
vi) the amino acid at position 147 (EU numbering) of the CH2_H1 is a D and the amino acid at position 180 (EU numbering) of the CL2 is a R.

34. The method of claim 33, wherein:
i) the amino acid at position 87 (Kabat numbering) of the VH1 and/or VH2 is a G, and
ii) the amino acid at position 45 (Kabat numbering) of the VL1 and/or VL2 is a W.

35. The method of any one of claims 33-34, wherein:
i) the CH1_H3 has a C at position 349, an S at position 366, an A at position 368 and a V at position 407 (EU numbering); and the CH2_H3 has a C at position 354 and a W at position 366 (EU numbering);
ii) the CH2_H3 has a C at position 349, an S at position 366, an A at position 368 and a V at position 407 (EU numbering); and the CH1_H3 has a C at position 354 and a W at position 366 (EU numbering);
iii) the CH1_H3 has a C at position 354, an S at position 366, an A at position 368 and a V at position 407 (EU numbering); and the CH2_H3 has a C at position 349 and a W at position 366 (EU numbering); or
iv) the CH2_H3 has a C at position 354, an S at position 366, an A at position 368 and a V at position 407 (EU numbering); and the CH1_H3 has a C at position 349 and a W at position 366 (EU numbering).

36. The method of any one of claims 33-35, wherein the amino acid at position 447 (EU numbering) of the CH1_H3 and/or of the CH2_H3 is deleted.

37. The method of any one of claims 33-36, wherein:
i) the H1H and/or the H2H has an A at positions 234 and 235 (EU numbering);
ii) the H1H and/or the H2H has an A at positions 234, 235 and 237 (EU numbering); or
iii) the H1H and/or the H2H has an A at positions 234 and 235 and G at position 329 (EU numbering).

38. The method of any one of claims 33-37, wherein
i) the CH1_H3 and/or the CH2_H3 has an A at position 297 (EU numbering);
ii) the CH1_H3 and/or the CH2_H3 has a G at position 297 (EU numbering); or
iii) the CH1_H3 and/or the CH2_H3 has a S at position 297 (EU numbering).

39. The method of any one of claims 33-38, wherein
the CH1_H3 and/or the CH2_H3 has an S at position 331 (EU numbering).

40. The method of any one of claims 33-39, wherein a polypeptide is fused to the N-terminus or the C-terminus of the first heavy chain polypeptide or the second heavy chain polypeptide of the bispecific antibody.

41. The method of claim 40, wherein the polypeptide is fused to the C-terminus of the first heavy chain polypeptide of the bispecific antibody.

42. The method of any one of claims 40-41, wherein the polypeptide is fused via a linker peptide.

43. The method of claim 42, wherein the linker peptide comprises the amino acid sequence of SEQ ID NO: 53.

44. The method of any one of claims 40-43, wherein the polypeptide comprises a CD58 or a fragment thereof.

45. The method of claim 44, wherein the CD58 comprises the amino acid sequence of SEQ ID NO: 49.

46. The method of any one of claims 1-45, wherein the bispecific antibody is an IgG1 or an IgG4 antibody.

47. The method of any one of claims 1-46, wherein the bispecific antibody is a monoclonal antibody, a chimeric antibody or a humanized antibody.

48. The method of any one of claims 1-47, further comprising administering a modified T cell activation agent.


SEQUENCE LISTING (713 sequences)

SEQ ID NO: 1-12 - skipped (12 entries)

SEQ ID NO: 13 - protein, 125 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 14 - protein, 125 aa, Homo sapiens
EVQLLESGGGLVQPGGSLKLSCAASGFTFNTYAMNWVRDAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 15 - protein, 125 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSS

SEQ ID NO: 16 - protein, 125 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSS

SEQ ID NO: 17 - protein, 125 aa, Homo sapiens
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 18 - protein, 125 aa, Homo sapiens
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSS

SEQ ID NO: 19 - protein, 125 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSS

SEQ ID NO: 20 - protein, 125 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSS

SEQ ID NO: 21 - protein, 125 aa, Homo sapiens
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 22 - protein, 109 aa, Homo sapiens
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVL

SEQ ID NO: 23 - protein, 109 aa, Homo sapiens
ELVVTQEPSLTVSPGGTVTLTCRSSTGAVTTSNYANWVQKKPGQAPRGLIGGTNKRAPGTPARFSGSLLGGKAALTLSGVQPEDEAEYYCALWYSNLWVFGGGTKLTVL

SEQ ID NO: 24 - protein, 109 aa, Homo sapiens
ELVVTQEPSLTVSPGGTVTLTCRSSTGAVTTSNYANWVQEKPGQAPRGLIGGTNKRAPGTPARFSGSLLGGKAALTLSGVQPEDEAEYYCALWYSNLWVFGGGTKLTVL

SEQ ID NO: 25 - protein, 109 aa, Homo sapiens
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWDSNLWVFGGGTKLTVL

SEQ ID NO: 26 - protein, 109 aa, Homo sapiens
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSQLWVFGGGTKLTVL

SEQ ID NO: 27 - protein, 109 aa, Homo sapiens
ELVVTQEPSLTVSPGGTVTLTCRSSTGAVTTSNYANWVQDKPGQAPRGLIGDTNKRAPGTPARFSGSLLGGKAALTLSGVQPEDEAEYYCALWYSNLWVFGGGTKLTVL

SEQ ID NO: 28 - protein, 109 aa, Homo sapiens
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSDLWVFGGGTKLTVL

SEQ ID NO: 29 - protein, 5 aa, Homo sapiens
TYAMN

SEQ ID NO: 30 - protein, 5 aa, Homo sapiens
DYAMN

SEQ ID NO: 31 - protein, 5 aa, Homo sapiens
TYAMD

SEQ ID NO: 32 - protein, 7 aa, Homo sapiens
GFTFNTY

SEQ ID NO: 33 - protein, 7 aa, Homo sapiens
GFTFNDY

SEQ ID NO: 34 - protein, 19 aa, Homo sapiens
RIRSKYNNYATYYADSVKD

SEQ ID NO: 35 - protein, 19 aa, Homo sapiens
KIRSKYNNYATYYADSVKD

SEQ ID NO: 36 - protein, 8 aa, Homo sapiens
RSKYNNYA

SEQ ID NO: 37 - protein, 14 aa, Homo sapiens
HGNFGNSYVSWFAY

SEQ ID NO: 38 - protein, 14 aa, Homo sapiens
HGNFGDSYVSWFAY

SEQ ID NO: 39 - protein, 14 aa, Homo sapiens
HGNFGDDYVSWFAY

SEQ ID NO: 40 - protein, 14 aa, Homo sapiens
HGSFGNSYVSWFAY

SEQ ID NO: 41 - protein, 14 aa, Homo sapiens
HGTFGNSYVSWFAY

SEQ ID NO: 42 - protein, 14 aa, Homo sapiens
RSSTGAVTTSNYAN

SEQ ID NO: 43 - protein, 7 aa, Homo sapiens
GTNKRAP

SEQ ID NO: 44 - protein, 7 aa, Homo sapiens
DTNKRAP

SEQ ID NO: 45 - protein, 9 aa, Homo sapiens
ALWYSNLWV

SEQ ID NO: 46 - protein, 9 aa, Homo sapiens
ALWDSNLWV

SEQ ID NO: 47 - protein, 9 aa, Homo sapiens
ALWYSQLWV

SEQ ID NO: 48 - protein, 9 aa, Homo sapiens
ALWYSDLWV

SEQ ID NO: 49 - protein, 188 aa, Homo sapiens
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 50 - protein, 94 aa, Homo sapiens
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLE

SEQ ID NO: 51 - skipped

SEQ ID NO: 52 - protein, 5 aa, Homo sapiens
GGGGS

SEQ ID NO: 53 - protein, 10 aa, Homo sapiens
GGGGSGGGGS

SEQ ID NO: 54 - protein, 15 aa, Homo sapiens
GGGGSGGGGSGGGGS

SEQ ID NO: 55-140 - skipped (86 entries)

SEQ ID NO: 141 - protein, 215 aa, Homo sapiens
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKADLVCLISDFYPGAVTVAWKADGSPVKAGVETCKPSKQSNNKYAASSYLSLTPEQWKSHRSYSCQVTHEGSTVEKTVAPTESS

SEQ ID NO: 142-148 - skipped (7 entries)

SEQ ID NO: 149 - protein, 215 aa, Homo sapiens
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSQLWVFGGGTKLTVLGQPKANPTVTLFPPSSEELQANKADLVCLISDFYPGAVTVAWKADGSPVKAGVETCKPSKQSNNKYAASSYLSLTPEQWKSHRSYSCQVTHEGSTVEKTVAPTESS

SEQ ID NO: 150-152 - skipped (3 entries)

SEQ ID NO: 153 - protein, 215 aa, Homo sapiens
ELVVTQEPSLTVSPGGTVTLTCRSSTGAVTTSNYANWVQDKPGQAPRGLIGDTNKRAPGTPARFSGSLLGGKAALTLSGVQPEDEAEYYCALWYSNLWVFGGGTKLTVLGQPKANPTVTLFPPSSEELQANKADLVCLISDFYPGAVTVAWKADGSPVKAGVETCKPSKQSNNKYAASSYLSLTPEQWKSHRSYSCQVTHEGSTVEKTVAPTESS

SEQ ID NO: 154-159 - skipped (6 entries)

SEQ ID NO: 160 - protein, 107 aa, Homo sapiens
DIQMTQSPASLSASVGETVTITCRASENIYSYLAWYQQKQGKSPQLLVNNAKTLAEGVPSRFSGSGSGTQFSLKINSLQPEDFGSYYCQHHYGSPWTFGGGTELEIK

SEQ ID NO: 161 - protein, 120 aa, Homo sapiens
EVQLQQSGPELVKPGASMKISCKASGYSFTDYTMNWVKQSHGKNLEWIGLINPYNGVTSYNPNFQGKATLTVDKSSSTAYMELLSLTSEDSAVYYCARSGDYDRNYFDYWGQGTTLTVSS

SEQ ID NO: 162 - protein, 107 aa, Homo sapiens
DIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIK

SEQ ID NO: 163 - protein, 118 aa, Homo sapiens
EVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSS

SEQ ID NO: 164 - protein, 107 aa, Homo sapiens
DIQMTQSPSTLSASVGDRVTITCRASENIYSYLAWYQQKPGKAPKLLVNNAKTLAEGVPSRFSGSGSGTEFTLTISSLQPDDFATYYCQHHYGSPWTFGQGTKVEIK

SEQ ID NO: 165 - protein, 120 aa, Homo sapiens
EVQLVQSGAEVKKPGSSVKVSCKASGYSFTDYTMNWVRQAPGQGLEWIGLINPYNGVTSYNPNFQGRATLTVDKSTSTAYMELSSLRSEDTAVYYCARSGDYDRNYFDYWGQGTLVTVSS

SEQ ID NO: 166 - protein, 107 aa, Homo sapiens; identical to SEQ ID NO: 164

SEQ ID NO: 167 - protein, 120 aa, Homo sapiens; identical to SEQ ID NO: 165

SEQ ID NO: 168 - protein, 107 aa, Homo sapiens; identical to SEQ ID NO: 164

SEQ ID NO: 169 - protein, 120 aa, Homo sapiens
QVQLVQSGAEVKKPGASVKVSCKASGYTFTDYTMNWVRQAPGQGLEWIGLINPYNGVTSYNPNFQGRVTLTVDKSTSTAYMELSSLRSEDTAVYYCARSGDYDRNYFDYWGQGTLVTVSS

SEQ ID NO: 170 - protein, 107 aa, Homo sapiens
DIQMTQSPSSLSASVGDRVTITCRASENIYSYLAWYQQKPGKAPKLLVNNAKTLAEGVPSRFSGSGSGTDFTLTISSLQPEDFATYYCQHHYGSPWTFGGGTELEIK

SEQ ID NO: 171 - protein, 120 aa, Homo sapiens
QVQLVQSGAEVKKPGSSVKVSCKASGYSFTDYTMNWVRQAPGQGLEWIGLINPYNGVTSYNPNFQGRATLTVDKSTSTAYMELSSLRSEDTAVYYCARSGDYDRNYFDYWGQGTTLTVSS

SEQ ID NO: 172 - protein, 107 aa, Homo sapiens
DIQMTQSPSSLSASVGDRVTITCRASENIYSYLAWYQQKPGKAPKLLINNAKTLQSGVPSRFSGSGSGTDFTLTISSLQPEDFATYYCQHHYGSPWTFGGGTELEIK

SEQ ID NO: 173 - protein, 120 aa, Homo sapiens
QVQLVQSGAEVKKPGASVKVSCKASGYTFTDYTMNWVRQAPGQGLEWIGIINPYNGVTSYNPNFQGRVTLTVDKSTSTVYMELSSLRSEDTAVYYCARSGDYDRNYFDYWGQGTLVTVSS

SEQ ID NO: 174 - protein, 107 aa, Homo sapiens; identical to SEQ ID NO: 164

SEQ ID NO: 175 - protein, 120 aa, Homo sapiens; identical to SEQ ID NO: 173

SEQ ID NO: 176 - protein, 107 aa, Homo sapiens; identical to SEQ ID NO: 170

SEQ ID NO: 177 - protein, 120 aa, Homo sapiens; identical to SEQ ID NO: 173

SEQ ID NO: 178 - protein, 107 aa, Homo sapiens
DIQMTQSPSSLSASVGDRVTITCRASENIYSYLAWYQQKPGKAPKLLVNNAKTLAEGVPSRFSGSGSGTEFTLTISSLQPEDFATYYCQHHYGSPWTFGGGTELEIK

SEQ ID NO: 179 - protein, 120 aa, Homo sapiens; identical to SEQ ID NO: 173

SEQ ID NO: 180 - protein, 107 aa, Homo sapiens; identical to SEQ ID NO: 178

SEQ ID NO: 181 - protein, 120 aa, Homo sapiens
EVQLVQSGAEVKKPGESLKISCKASGYSFTDYTMNWVRQMPGKGLEWIGLINPYNGVTSYNPNFQGQATLSVDKSISTAYLQWSSLKASDTAMYYCARSGDYDRNYFDYWGQGTTLTVSS

SEQ ID NO: 182 - protein, 107 aa, Homo sapiens; identical to SEQ ID NO: 164

SEQ ID NO: 183 - protein, 120 aa, Homo sapiens; identical to SEQ ID NO: 181

SEQ ID NO: 184 - protein, 107 aa, Homo sapiens
DIQMTQSPSSLSASVGDRVTITCRASENIYSYLAWYQQKPGKAPKLLVNNAKTLAEGVPSRFSGSGSGTDFTFTISSLQPEDIATYYCQHHYGSPWTFGGGTELEIK

SEQ ID NO: 185 - protein, 120 aa, Homo sapiens
EVQLVESGGGLVQPGGSLRLSCAASGYSFTDYTMNWVRQAPGKGLEWIGLINPYNGVTSYNPNFQGRATLSVDKSKNSAYLQMNSLKTEDTAVYYCARSGDYDRNYFDYWGQGTTLTVSS

SEQ ID NO: 186 - protein, 107 aa, Homo sapiens
DIQMTQSPSSLSASVGDRVTITCRASENIYSYLAWYQQKPGKAPKLLINNAKTLAEGVPSRFSGSGSGTDFTLTISSLQPEDFATYYCQHHYGSPWTFGGGTELEIK

SEQ ID NO: 187 - protein, 120 aa, Homo sapiens; identical to SEQ ID NO: 185

SEQ ID NO: 188 - protein, 107 aa, Homo sapiens
DIQMTQSPSSLSASVGDRVTITCRASENIYSYLAWYQQKPGKAPKLLINNAKTLAEGVPSRFSGSGSGTDFTLTISSLQPEDFATYYCQHHYGSPWTFGGGTKVEIK

SEQ ID NO: 189 - protein, 120 aa, Homo sapiens; identical to SEQ ID NO: 185

SEQ ID NO: 190 - protein, 107 aa, Homo sapiens; identical to SEQ ID NO: 184

SEQ ID NO: 191 - protein, 120 aa, Homo sapiens
EVQLVESGGGLVQPGGSLRLSCAASGFTFTDYTMNWVRQAPGKGLEWVGLINPYNGVTSYNPNFQGRFTISVDKSKNSAYLQMNSLKTEDTAVYYCARSGDYDRNYFDYWGQGTLVTVSS

SEQ ID NO: 192 - protein, 107 aa, Homo sapiens; identical to SEQ ID NO: 186

SEQ ID NO: 193 - protein, 120 aa, Homo sapiens; identical to SEQ ID NO: 191

SEQ ID NO: 194 - protein, 107 aa, Homo sapiens; identical to SEQ ID NO: 188

SEQ ID NO: 195 - protein, 120 aa, Homo sapiens; identical to SEQ ID NO: 191

SEQ ID NO: 196 - protein, 107 aa, Homo sapiens
DIQMTQSPSTLSASVGDRVTITCRASENIYSYLAWYQKKPGKAPKLLVNNAKTLAEGVPSRFSGSGSGTEFTLTISSLQPDDFATYYCQHHYGSPWTFGQGTKVEIK

SEQ ID NO: 197 - protein, 120 aa, Homo sapiens
QVQLVQSGAEVKKPGASVKVSCKASGYTFTDYTMNWVRDAPGQGLEWIGLINPYNGVTSYNPNFQGRVTLTVDKSTSTAYMELSSLRSEDTAVYYCARSGDYDRNYFDYWGQGTLVTVSS

SEQ ID NO: 198 - protein, 107 aa, Homo sapiens
DIQMTQSPSSLSASVGDRVTITCRASENIYSYLAWYQKKPGKAPKLLVNNAKTLAEGVPSRFSGSGSGTDFTLTISSLQPEDFATYYCQHHYGSPWTFGGGTELEIK

SEQ ID NO: 199 - protein, 120 aa, Homo sapiens
QVQLVQSGAEVKKPGSSVKVSCKASGYSFTDYTMNWVRDAPGQGLEWIGLINPYNGVTSYNPNFQGRATLTVDKSTSTAYMELSSLRSEDTAVYYCARSGDYDRNYFDYWGQGTTLTVSS

SEQ ID NO: 200 - protein, 107 aa, Homo sapiens
DIQMTQSPSSLSASVGDRVTITCRASENIYSYLAWYQKKPGKAPKLLVNNAKTLAEGVPSRFSGSGSGTEFTLTISSLQPEDFATYYCQHHYGSPWTFGGGTELEIK

SEQ ID NO: 201 - protein, 120 aa, Homo sapiens
EVQLVQSGAEVKKPGESLKISCKASGYSFTDYTMNWVRDMPGKGLEWIGLINPYNGVTSYNPNFQGQATLSVDKSISTAYLQWSSLKASDTAMYYCARSGDYDRNYFDYWGQGTTLTVSS

SEQ ID NO: 202 - protein, 107 aa, Homo sapiens; identical to SEQ ID NO: 196

SEQ ID NO: 203 - protein, 120 aa, Homo sapiens; identical to SEQ ID NO: 201

SEQ ID NO: 204 - protein, 107 aa, Homo sapiens; identical to SEQ ID NO: 196

SEQ ID NO: 205 - protein, 120 aa, Homo sapiens
EVQLVQSGAEVKKPGSSVKVSCKASGYSFTDYTMNWVRDAPGQGLEWIGLINPYNGVTSYNPNFQGRATLTVDKSTSTAYMELSSLRSEDTAVYYCARSGDYDRNYFDYWGQGTLVTVSS

SEQ ID NO: 206-209 - skipped (4 entries)

SEQ ID NO: 210 - protein, 5 aa, Homo sapiens
DYTMN

SEQ ID NO: 211 - protein, 7 aa, Homo sapiens
GYTFTDY

SEQ ID NO: 212 - protein, 17 aa, Homo sapiens
LINPYNGVTSYNPNFQG

SEQ ID NO: 213 - protein, 6 aa, Homo sapiens
NPYNGV

SEQ ID NO: 214 - protein, 11 aa, Homo sapiens
SGDYDRNYFDY

SEQ ID NO: 215 - protein, 7 aa, Homo sapiens
GYSFTDY

SEQ ID NO: 216 - protein, 17 aa, Homo sapiens
IINPYNGVTSYNPNFQG

SEQ ID NO: 217 - protein, 7 aa, Homo sapiens
GFTFTDY

SEQ ID NO: 218 - protein, 5 aa, Homo sapiens
GYTMN

SEQ ID NO: 219 - protein, 7 aa, Homo sapiens
GYSFTGY

SEQ ID NO: 220 - protein, 17 aa, Homo sapiens
LINPYNGVTSYNQKFKD

SEQ ID NO: 221 - protein, 9 aa, Homo sapiens
DYNYDYFDY

SEQ ID NO: 222 - protein, 11 aa, Homo sapiens
RASENIYSYLA

SEQ ID NO: 223 - protein, 7 aa, Homo sapiens
NAKTLAE

SEQ ID NO: 224 - protein, 9 aa, Homo sapiens
QHHYGSPWT

SEQ ID NO: 225 - protein, 7 aa, Homo sapiens
NAKTLQS

SEQ ID NO: 226 - protein, 214 aa, Homo sapiens
DIQMTQSPSTLSASVGDRVTITCRASENIYSYLAWYQKKPGKAPKLLVNNAKTLAEGVPSRFSGSGSGTEFTLTISSLQPDDFATYYCQHHYGSPWTFGQGTKVEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 227 - protein, 449 aa, Homo sapiens
QVQLVQSGAEVKKPGASVKVSCKASGYTFTDYTMNWVRDAPGQGLEWIGLINPYNGVTSYNPNFQGRVTLTVDKSTSTAYMELSSLRSEDTAVYYCARSGDYDRNYFDYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 215 aa, Homo sapiens; identical to SEQ ID NO: 141

SEQ ID NO: 229 - protein, 454 aa, Homo sapiens
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 230 - protein, 214 aa, Homo sapiens
DIQMTQSPSSLSASVGDRVTITCRASENIYSYLAWYQKKPGKAPKLLVNNAKTLAEGVPSRFSGSGSGTDFTLTISSLQPEDFATYYCQHHYGSPWTFGGGTELEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 231 - protein, 449 aa, Homo sapiens
QVQLVQSGAEVKKPGSSVKVSCKASGYSFTDYTMNWVRDAPGQGLEWIGLINPYNGVTSYNPNFQGRATLTVDKSTSTAYMELSSLRSEDTAVYYCARSGDYDRNYFDYWGQGTTLTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 232 - protein, 215 aa, Homo sapiens; identical to SEQ ID NO: 141

SEQ ID NO: 233 - protein, 454 aa, Homo sapiens; identical to SEQ ID NO: 229

SEQ ID NO: 234 - protein, 214 aa, Homo sapiens
DIQMTQSPSSLSASVGDRVTITCRASENIYSYLAWYQKKPGKAPKLLVNNAKTLAEGVPSRFSGSGSGTEFTLTISSLQPEDFATYYCQHHYGSPWTFGGGTELEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 235 - protein, 449 aa, Homo sapiens
EVQLVQSGAEVKKPGESLKISCKASGYSFTDYTMNWVRDMPGKGLEWIGLINPYNGVTSYNPNFQGQATLSVDKSISTAYLQWSSLKASDTAMYYCARSGDYDRNYFDYWGQGTTLTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 236 - protein, 215 aa, Homo sapiens; identical to SEQ ID NO: 141

SEQ ID NO: 237 - protein, 454 aa, Homo sapiens; identical to SEQ ID NO: 229

SEQ ID NO: 238 - protein, 214 aa, Homo sapiens; identical to SEQ ID NO: 226

SEQ ID NO: 239 - protein, 449 aa, Homo sapiens; identical to SEQ ID NO: 235

SEQ ID NO: 240 - protein, 215 aa, Homo sapiens; identical to SEQ ID NO: 141

SEQ ID NO: 241 - protein, 454 aa, Homo sapiens; identical to SEQ ID NO: 229

SEQ ID NO: 242 - protein, 214 aa, Homo sapiens; identical to SEQ ID NO: 226

SEQ ID NO: 243 - protein, 449 aa, Homo sapiens
EVQLVQSGAEVKKPGSSVKVSCKASGYSFTDYTMNWVRDAPGQGLEWIGLINPYNGVTSYNPNFQGRATLTVDKSTSTAYMELSSLRSEDTAVYYCARSGDYDRNYFDYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 244 - protein, 215 aa, Homo sapiens; identical to SEQ ID NO: 141

SEQ ID NO: 245 - protein, 454 aa, Homo sapiens; identical to SEQ ID NO: 229

SEQ ID NO: 246 - protein, 214 aa, Homo sapiens; identical to SEQ ID NO: 226

SEQ ID NO: 247 - protein, 449 aa, Homo sapiens; identical to SEQ ID NO: 235

SEQ ID NO: 248 - protein, 215 aa, Homo sapiens; identical to SEQ ID NO: 141

SEQ ID NO: 249 - protein, 454 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 250-253 - skipped (4 entries)

SEQ ID NO: 254 - protein, 214 aa, Homo sapiens; identical to SEQ ID NO: 226

SEQ ID NO: 255 - protein, 449 aa, Homo sapiens; identical to SEQ ID NO: 235

SEQ ID NO: 256 - protein, 215 aa, Homo sapiens; identical to SEQ ID NO: 153

SEQ ID NO: 257 - protein, 454 aa, Homo sapiens; identical to SEQ ID NO: 249

SEQ ID NO: 258-261 - skipped (4 entries)

SEQ ID NO: 262 - protein, 214 aa, Homo sapiens; identical to SEQ ID NO: 226

SEQ ID NO: 263 - protein, 449 aa, Homo sapiens; identical to SEQ ID NO: 235

SEQ ID NO: 264 - protein, 215 aa, Homo sapiens; identical to SEQ ID NO: 149

SEQ ID NO: 265 - protein, 454 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 266-269 - skipped (4 entries)

SEQ ID NO: 270 - protein, 214 aa, Homo sapiens; identical to SEQ ID NO: 226

SEQ ID NO: 271 - protein, 449 aa, Homo sapiens; identical to SEQ ID NO: 235

SEQ ID NO: 272 - protein, 215 aa, Homo sapiens; identical to SEQ ID NO: 141

SEQ ID NO: 273 - protein, 454 aa, Homo sapiens; identical to SEQ ID NO: 229

SEQ ID NO: 274-277 - skipped (4 entries)

SEQ ID NO: 278 - protein, 214 aa, Homo sapiens; identical to SEQ ID NO: 226

SEQ ID NO: 279 - protein, 449 aa, Homo sapiens; identical to SEQ ID NO: 235

SEQ ID NO: 280 - protein, 215 aa, Homo sapiens; identical to SEQ ID NO: 149

SEQ ID NO: 281 - protein, 454 aa, Homo sapiens
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 282-285 - skipped (4 entries)

SEQ ID NO: 286 - protein, 214 aa, Homo sapiens; identical to SEQ ID NO: 226

SEQ ID NO: 287 - protein, 449 aa, Homo sapiens; identical to SEQ ID NO: 235

SEQ ID NO: 288 - protein, 215 aa, Homo sapiens; identical to SEQ ID NO: 149

SEQ ID NO: 289 - protein, 454 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 290-293 - skipped (4 entries)

SEQ ID NO: 294 - protein, 214 aa, Homo sapiens; identical to SEQ ID NO: 226

SEQ ID NO: 295 - protein, 449 aa, Homo sapiens; identical to SEQ ID NO: 235

SEQ ID NO: 296 - protein, 215 aa, Homo sapiens; identical to SEQ ID NO: 149

SEQ ID NO: 297 - protein, 454 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 298-301 - skipped (4 entries)

SEQ ID NO: 302 - protein, 214 aa, Homo sapiens; identical to SEQ ID NO: 226

SEQ ID NO: 303 - protein, 449 aa, Homo sapiens; identical to SEQ ID NO: 227

SEQ ID NO: 304 - protein, 215 aa, Homo sapiens; identical to SEQ ID NO: 141

SEQ ID NO: 305 - protein, 651 aa, Homo sapiens
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 306 - protein, 214 aa, Homo sapiens; identical to SEQ ID NO: 230

SEQ ID NO: 307 - protein, 449 aa, Homo sapiens; identical to SEQ ID NO: 231

SEQ ID NO: 308 - protein, 215 aa, Homo sapiens; identical to SEQ ID NO: 141

SEQ ID NO: 309 - protein, 651 aa, Homo sapiens; identical to SEQ ID NO: 305

SEQ ID NO: 310 - protein, 214 aa, Homo sapiens; identical to SEQ ID NO: 234

SEQ ID NO: 311 - protein, 449 aa, Homo sapiens; identical to SEQ ID NO: 235

SEQ ID NO: 312 - protein, 215 aa, Homo sapiens; identical to SEQ ID NO: 141

SEQ ID NO: 313 - protein, 651 aa, Homo sapiens; identical to SEQ ID NO: 305

SEQ ID NO: 314 - protein, 214 aa, Homo sapiens; identical to SEQ ID NO: 226

SEQ ID NO: 315 - protein, 449 aa, Homo sapiens; identical to SEQ ID NO: 235

SEQ ID NO: 316 - protein, 215 aa, Homo sapiens; identical to SEQ ID NO: 141

SEQ ID NO: 317 - protein, 651 aa, Homo sapiens; identical to SEQ ID NO: 305

SEQ ID NO: 318 - protein, 214 aa, Homo sapiens; identical to SEQ ID NO: 226

SEQ ID NO: 319 - protein, 449 aa, Homo sapiens; identical to SEQ ID NO: 243

SEQ ID NO: 320 - protein, 215 aa, Homo sapiens; identical to SEQ ID NO: 141

SEQ ID NO: 321 - protein, 651 aa, Homo sapiens; identical to SEQ ID NO: 305

SEQ ID NO: 322-325 - skipped (4 entries)

SEQ ID NO: 326 - protein, 214 aa, Homo sapiens; identical to SEQ ID NO: 226

SEQ ID NO: 327 - protein, 449 aa, Homo sapiens; identical to SEQ ID NO: 235

SEQ ID NO: 328 - protein, 215 aa, Homo sapiens; identical to SEQ ID NO: 141

SEQ ID NO: 329 - protein, 651 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 330-333 - skipped (4 entries)

SEQ ID NO: 334 - protein, 214 aa, Homo sapiens; identical to SEQ ID NO: 226

SEQ ID NO: 335 - protein, 449 aa, Homo sapiens; identical to SEQ ID NO: 235

SEQ ID NO: 336 - protein, 215 aa, Homo sapiens; identical to SEQ ID NO: 153

SEQ ID NO: 337 - protein, 651 aa, Homo sapiens; identical to SEQ ID NO: 329

SEQ ID NO: 338-341 - skipped (4 entries)

SEQ ID NO: 342 - protein, 214 aa, Homo sapiens; identical to SEQ ID NO: 226

SEQ ID NO: 343 - protein, 449 aa, Homo sapiens; identical to SEQ ID NO: 235

SEQ ID NO: 344 - protein, 215 aa, Homo sapiens; identical to SEQ ID NO: 149

SEQ ID NO: 345 - protein, 651 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 346 - protein, 653 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 347-350 - skipped (4 entries)

SEQ ID NO: 351 - protein, 214 aa, Homo sapiens; identical to SEQ ID NO: 226

SEQ ID NO: 352 - protein, 449 aa, Homo sapiens; identical to SEQ ID NO: 235

SEQ ID NO: 353 - protein, 215 aa, Homo sapiens; identical to SEQ ID NO: 141

SEQ ID NO: 354 - protein, 651 aa, Homo sapiens; identical to SEQ ID NO: 305

SEQ ID NO: 355 - protein, 653 aa, Homo sapiens
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 356-359 - skipped (4 entries)

SEQ ID NO: 360 - protein, 214 aa, Homo sapiens; identical to SEQ ID NO: 226

SEQ ID NO: 361 - protein, 449 aa, Homo sapiens; identical to SEQ ID NO: 235

SEQ ID NO: 362 - protein, 215 aa, Homo sapiens; identical to SEQ ID NO: 149

SEQ ID NO: 363 - protein, 651 aa, Homo sapiens
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 364 - protein, 653 aa, Homo sapiens
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 365-368 - skipped (4 entries)

SEQ ID NO: 369 - protein, 214 aa, Homo sapiens; identical to SEQ ID NO: 226

SEQ ID NO: 370 - protein, 449 aa, Homo sapiens; identical to SEQ ID NO: 235

SEQ ID NO: 371 - protein, 215 aa, Homo sapiens; identical to SEQ ID NO: 149

SEQ ID NO: 372 - protein, 651 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 373 - protein, 653 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 374-377 - skipped (4 entries)

SEQ ID NO: 378 - protein, 214 aa, Homo sapiens; identical to SEQ ID NO: 226

SEQ ID NO: 379 - protein, 449 aa, Homo sapiens; identical to SEQ ID NO: 235

SEQ ID NO: 380 - protein, 215 aa, Homo sapiens; identical to SEQ ID NO: 149

SEQ ID NO: 381 - protein, 651 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 382 - protein, 653 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 383-390 - skipped (8 entries)

SEQ ID NO: 391 - protein, 214 aa, Homo sapiens; identical to SEQ ID NO: 226

SEQ ID NO: 392 - protein, 449 aa, Homo sapiens
EVQLVQSGAEVKKPGESLKISCKASGYSFTDYTMNWVRDMPGKGLEWIGLINPYNGVTSYNPNFQGQATLSVDKSISTAYLQWSSLKASDTAMYYCARSGDYDRNYFDYWGQGTTLTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 393 - protein, 215 aa, Homo sapiens; identical to SEQ ID NO: 141

SEQ ID NO: 394 - protein, 454 aa, Homo sapiens; identical to SEQ ID NO: 249

SEQ ID NO: 395-398 - skipped (4 entries)

SEQ ID NO: 399 - protein, 215 aa, Homo sapiens
EIVLTQSPGTLSLSPGERATFSCRSSHSIRSRRVAWYQKKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 400 - protein, 456 aa, Homo sapiens
QVQLVQSGAEVKKPGASVKVSCQASGYRFSNFVIHWVRDAPGQRQEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 401 - protein, 215 aa, Homo sapiens; identical to SEQ ID NO: 141

SEQ ID NO: 402 - protein, 454 aa, Homo sapiens; identical to SEQ ID NO: 249

SEQ ID NO: 403 - protein, 456 aa, Homo sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 404 - protein, 215 aa, Homo sapiens
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 405 - protein, 217 aa, Homo sapiens
DIQMTQSPSSLSASVGDRVTITCTSSQSLFNVRSQKNYLAWYQQKPGKAPKLLIYWASTRESGVPSRFSGSGSGTDFTLTISSLQPEDFATYYCKQSYDLFTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 406 - protein, 454 aa, Homo sapiens
EVQLVESGGGLVQPGGSLRLSCAASGFTFSDYYMTWVRQAPGKGLEWVAFIRNQARGYTSDHNPSVKGRFTISRDNAKNSLYLQMNSLRAEDTAVYYCARDRPSYYVLDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 407 - protein, 215 aa, Homo sapiens; identical to SEQ ID NO: 399

SEQ ID NO: 408 - protein, 456 aa, Homo sapiens; identical to SEQ ID NO: 400

SEQ ID NO: 409 - protein, 215 aa, Homo sapiens; identical to SEQ ID NO: 149

SEQ ID NO: 410 - protein, 651 aa, Homo sapiens; identical to SEQ ID NO: 345

SEQ ID NO: 411-418 - skipped (8 entries)

SEQ ID NO: 419 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 420 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 421-429 - skipped (9 entries)

SEQ ID NO: 430 - protein, 11 aa, Homo sapiens
RASEKIYSYLA

SEQ ID NO: 431 - protein, 7 aa, Homo sapiens
NAKTLAE

SEQ ID NO: 432 - protein, 9 aa, Homo sapiens
QHHFGSPRT

SEQ ID NO: 433 - protein, 12 aa, Homo sapiens
RSSHSIRSRRVA

SEQ ID NO: 434 - protein, 7 aa, Homo sapiens
GVSNRAS

SEQ ID NO: 435 - protein, 9 aa, Homo sapiens
QVYGASSYT

SEQ ID NO: 436-449 - skipped (14 entries)

SEQ ID NO: 450 - protein, 115 aa, Homo sapiens
DIVMTQTPLSLPVTPGEPASISCRSSKSLLHSNGITYLYWYLKKPGQSPQLLIYQMSNLVSGVPDRFSGSGSGTDFTLKISRVEAEDVGVYYCAQNLELPYTFGGGTKVEIKRTV

SEQ ID NO: 451 - protein, 119 aa, Homo sapiens
EVQLVQSGAEVKKPGSSVKVSCKASGYAFSYSWINWVRDAPGQGLEWMGRIFPGDGDTDYNGKFKGRVTITADKSTSTAYMELSSLRSEDTAVYYCARNVFDGYWLVYWGQGTLVTVSS

SEQ ID NO: 452 - protein, 110 aa, Homo sapiens
EIVLTQSPATLSLSPGERATLSCRASQSVSSYLAWYQKKPGQAPRLLIYDASNRATGIPARFSGSGSGTDFTLTISSLEPEDFAVYYCQQRSNWPITFGQGTRLEIKRTV

SEQ ID NO: 453 - protein, 122 aa, Homo sapiens
EVQLVESGGGLVQPGRSLRLSCAASGFTFNDYAMHWVRDAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 454 - protein, 5 aa, Homo sapiens
YSWIN

SEQ ID NO: 455 - protein, 7 aa, Homo sapiens
GYAFSYS

SEQ ID NO: 456 - protein, 18 aa, Homo sapiens
RIFPGDGDTDYNGKFKGG

SEQ ID NO: 457 - protein, 6 aa, Homo sapiens
FPGDGD

SEQ ID NO: 458 - protein, 10 aa, Homo sapiens
NVFDGYWLVY

SEQ ID NO: 459 - protein, 5 aa, Homo sapiens
DYAMH

SEQ ID NO: 460 - protein, 17 aa, Homo sapiens
TISWNSGSIGYADSVKG

SEQ ID NO: 461 - protein, 6 aa, Homo sapiens
SWNSGS

SEQ ID NO: 462 - protein, 13 aa, Homo sapiens
DIQYGNYYYGMDV

SEQ ID NO: 463 - protein, 16 aa, Homo sapiens
RSSKSLLHSNGITYLY

SEQ ID NO: 464 - protein, 7 aa, Homo sapiens
QMSNLVS

SEQ ID NO: 465 - protein, 9 aa, Homo sapiens
AQNLELPYT

SEQ ID NO: 466 - protein, 11 aa, Homo sapiens
RASQSVSSYLA

SEQ ID NO: 467 - protein, 7 aa, Homo sapiens
DASNRAT

SEQ ID NO: 468 - protein, 9 aa, Homo sapiens
QQRSNWPIT

SEQ ID NO: 469-470 - skipped (2 entries)

SEQ ID NO: 471 - protein, 214 aa, Homo sapiens
EIVLTQSPATLSLSPGERATLSCRASQSVSSYLAWYQKKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 472 - protein, 451 aa, Homo sapiens
EVQLVESGGGLVQPGRSLRLSCAASGFTFNDYAMHWVRDAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 473 - protein, 454 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 474 - skipped

SEQ ID NO: 475 - protein, 451 aa, Homo sapiens; identical to SEQ ID NO: 472

SEQ ID NO: 476 - protein, 454 aa, Homo sapiens; identical to SEQ ID NO: 281

SEQ ID NO: 477 - skipped

SEQ ID NO: 478 - protein, 451 aa, Homo sapiens; identical to SEQ ID NO: 472

SEQ ID NO: 479 - protein, 454 aa, Homo sapiens; identical to SEQ ID NO: 229

SEQ ID NO: 480 - skipped

SEQ ID NO: 481 - protein, 451 aa, Homo sapiens; identical to SEQ ID NO: 472

SEQ ID NO: 482 - protein, 454 aa, Homo sapiens; identical to SEQ ID NO: 265

SEQ ID NO: 483 - skipped

SEQ ID NO: 484 - protein, 451 aa, Homo sapiens; identical to SEQ ID NO: 472

SEQ ID NO: 485 - protein, 454 aa, Homo sapiens; identical to SEQ ID NO: 249

SEQ ID NO: 486 - skipped

SEQ ID NO: 487 - protein, 451 aa, Homo sapiens; identical to SEQ ID NO: 472

SEQ ID NO: 488 - protein, 454 aa, Homo sapiens; identical to SEQ ID NO: 249

SEQ ID NO: 489 - skipped

SEQ ID NO: 490 - protein, 451 aa, Homo sapiens; identical to SEQ ID NO: 472

SEQ ID NO: 491 - protein, 651 aa, Homo sapiens; identical to SEQ ID NO: 372

SEQ ID NO: 492 - skipped

SEQ ID NO: 493 - protein, 451 aa, Homo sapiens; identical to SEQ ID NO: 472

SEQ ID NO: 494 - protein, 651 aa, Homo sapiens
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 495 - skipped

SEQ ID NO: 496 - protein, 451 aa, Homo sapiens; identical to SEQ ID NO: 472

SEQ ID NO: 497 - protein, 651 aa, Homo sapiens; identical to SEQ ID NO: 305

SEQ ID NO: 498 - skipped

SEQ ID NO: 499 - protein, 451 aa, Homo sapiens; identical to SEQ ID NO: 472

SEQ ID NO: 500 - protein, 651 aa, Homo sapiens; identical to SEQ ID NO: 345

SEQ ID NO: 501 - skipped

SEQ ID NO: 502 - protein, 451 aa, Homo sapiens; identical to SEQ ID NO: 472

SEQ ID NO: 503 - protein, 452 aa, Homo sapiens
EVQLVESGGGLVQPGRSLRLSCAASGFTFNDYAMHWVRDAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 504 - skipped

SEQ ID NO: 505 - protein, 451 aa, Homo sapiens; identical to SEQ ID NO: 472

SEQ ID NO: 506 - protein, 651 aa, Homo sapiens; identical to SEQ ID NO: 329

SEQ ID NO: 507 - protein, 219 aa, Homo sapiens
DIVMTQTPLSLPVTPGEPASISCRSSKSLLHSNGITYLYWYLKKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 508 - protein, 448 aa, Homo sapiens
EVQLVQSGAEVKKPGSSVKVSCKASGYAFSYSWINWVRDAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 509 - protein, 454 aa, Homo sapiens; identical to SEQ ID NO: 473

SEQ ID NO: 510 - protein, 219 aa, Homo sapiens; identical to SEQ ID NO: 507

SEQ ID NO: 511 - protein, 448 aa, Homo sapiens; identical to SEQ ID NO: 508

SEQ ID NO: 512 - protein, 454 aa, Homo sapiens; identical to SEQ ID NO: 281

SEQ ID NO: 513 - protein, 219 aa, Homo sapiens; identical to SEQ ID NO: 507

SEQ ID NO: 514 - protein, 448 aa, Homo sapiens; identical to SEQ ID NO: 508

SEQ ID NO: 515 - protein, 454 aa, Homo sapiens; identical to SEQ ID NO: 229

SEQ ID NO: 516 - protein, 219 aa, Homo sapiens; identical to SEQ ID NO: 507

SEQ ID NO: 517 - protein, 448 aa, Homo sapiens; identical to SEQ ID NO: 508

SEQ ID NO: 518 - protein, 454 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 519 - protein, 219 aa, Homo sapiens; identical to SEQ ID NO: 507

SEQ ID NO: 520 - protein, 448 aa, Homo sapiens; identical to SEQ ID NO: 508

SEQ ID NO: 521 - protein, 454 aa, Homo sapiens; identical to SEQ ID NO: 249

SEQ ID NO: 522 - protein, 219 aa, Homo sapiens; identical to SEQ ID NO: 507

SEQ ID NO: 523 - protein, 448 aa, Homo sapiens; identical to SEQ ID NO: 508

SEQ ID NO: 524 - protein, 454 aa, Homo sapiens; identical to SEQ ID NO: 249

SEQ ID NO: 525 - protein, 219 aa, Homo sapiens; identical to SEQ ID NO: 507

SEQ ID NO: 526 - protein, 448 aa, Homo sapiens; identical to SEQ ID NO: 508

SEQ ID NO: 527 - protein, 651 aa, Homo sapiens; identical to SEQ ID NO: 372

SEQ ID NO: 528 - protein, 219 aa, Homo sapiens; identical to SEQ ID NO: 507

SEQ ID NO: 529 - protein, 448 aa, Homo sapiens; identical to SEQ ID NO: 508

SEQ ID NO: 530 - protein, 651 aa, Homo sapiens; identical to SEQ ID NO: 494

SEQ ID NO: 531 - protein, 219 aa, Homo sapiens; identical to SEQ ID NO: 507

SEQ ID NO: 532 - protein, 448 aa, Homo sapiens; identical to SEQ ID NO: 508

SEQ ID NO: 533 - protein, 651 aa, Homo sapiens; identical to SEQ ID NO: 305

SEQ ID NO: 534 - protein, 219 aa, Homo sapiens; identical to SEQ ID NO: 507

SEQ ID NO: 535 - protein, 448 aa, Homo sapiens; identical to SEQ ID NO: 508

SEQ ID NO: 536 - protein, 651 aa, Homo sapiens; identical to SEQ ID NO: 345

SEQ ID NO: 537 - protein, 219 aa, Homo sapiens; identical to SEQ ID NO: 507

SEQ ID NO: 538 - protein, 448 aa, Homo sapiens; identical to SEQ ID NO: 508

SEQ ID NO: 539 - protein, 651 aa, Homo sapiens; identical to SEQ ID NO: 329

SEQ ID NO: 540 - protein, 219 aa, Homo sapiens; identical to SEQ ID NO: 507

SEQ ID NO: 541 - protein, 448 aa, Homo sapiens; identical to SEQ ID NO: 508

SEQ ID NO: 542 - protein, 651 aa, Homo sapiens; identical to SEQ ID NO: 329

SEQ ID NO: 543 - protein, 215 aa, Homo sapiens; identical to SEQ ID NO: 399

SEQ ID NO: 544 - protein, 456 aa, Homo sapiens; identical to SEQ ID NO: 400

SEQ ID NO: 545 - protein, 651 aa, Homo sapiens; identical to SEQ ID NO: 345

SEQ ID NO: 546 - protein, 213 aa, Homo sapiens
DIQMTQSPSSLSASVGDRVTITCRASSSVSYMHWYQKKPGKAPKPLIYAPSNLASGVPSRFSGSGSGTDFTLTISSLQPEDFATYYCQQWSENPPTFGQGTKVEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 547 - protein, 451 aa, Homo sapiens
EVQLVESGGGLVQPGGSLRLSCAASGYTFTSYNMHWVRDAPGKGLEWVGAIYPGNGDTSYNQKEKGRFTISVDKSKNTLYLQMNSLRAEDTAVYYCARVVYYSNSYWYFDVWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 548 - protein, 454 aa, Homo sapiens; identical to SEQ ID NO: 249

SEQ ID NO: 549 - protein, 651 aa, Homo sapiens; identical to SEQ ID NO: 329

SEQ ID NO: 550 - protein, 219 aa, Homo sapiens
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 551 - protein, 447 aa, Homo sapiens
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLLSPG

SEQ ID NO: 552 - protein, 232 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 553 - protein, 673 aa, Homo sapiens
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 554 - protein, 452 aa, Homo sapiens; identical to SEQ ID NO: 503

SEQ ID NO: 555 - protein, 452 aa, Homo sapiens; identical to SEQ ID NO: 503

SEQ ID NO: 556 - protein, 452 aa, Homo sapiens; identical to SEQ ID NO: 503

SEQ ID NO: 557 - protein, 452 aa, Homo sapiens; identical to SEQ ID NO: 503

SEQ ID NO: 558 - protein, 452 aa, Homo sapiens; identical to SEQ ID NO: 503

SEQ ID NO: 559 - protein, 452 aa, Homo sapiens; identical to SEQ ID NO: 503

SEQ ID NO: 560 - protein, 449 aa, Homo sapiens
EVQLVQSGAEVKKPGSSVKVSCKASGYAFSYSWINWVRDAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 561 - protein, 449 aa, Homo sapiens; identical to SEQ ID NO: 560

SEQ ID NO: 562 - protein, 449 aa, Homo sapiens; identical to SEQ ID NO: 560

SEQ ID NO: 563 - protein, 449 aa, Homo sapiens; identical to SEQ ID NO: 560

SEQ ID NO: 564 - protein, 449 aa, Homo sapiens; identical to SEQ ID NO: 560

SEQ ID NO: 565 - protein, 449 aa, Homo sapiens; identical to SEQ ID NO: 560

SEQ ID NO: 566 - protein, 452 aa, Homo sapiens; identical to SEQ ID NO: 503

SEQ ID NO: 567 - protein, 452 aa, Homo sapiens; identical to SEQ ID NO: 503

SEQ ID NO: 568 - protein, 452 aa, Homo sapiens; identical to SEQ ID NO: 503

SEQ ID NO: 569 - protein, 452 aa, Homo sapiens; identical to SEQ ID NO: 503

SEQ ID NO: 570 - protein, 452 aa, Homo sapiens; identical to SEQ ID NO: 503

SEQ ID NO: 571 - protein, 452 aa, Homo sapiens; identical to SEQ ID NO: 503

SEQ ID NO: 572 - protein, 452 aa, Homo sapiens; identical to SEQ ID NO: 503

SEQ ID NO: 573 - protein, 452 aa, Homo sapiens; identical to SEQ ID NO: 503

SEQ ID NO: 574 - protein, 452 aa, Homo sapiens; identical to SEQ ID NO: 503

SEQ ID NO: 575 - protein, 452 aa, Homo sapiens; identical to SEQ ID NO: 503

SEQ ID NO: 576 - protein, 452 aa, Homo sapiens; identical to SEQ ID NO: 503

SEQ ID NO: 577 - protein, 452 aa, Homo sapiens; identical to SEQ ID NO: 503

SEQ ID NO: 578 - protein, 457 aa, Homo sapiens
QVQLVQSGAEVKKPGASVKVSCQASGYRFSNFVIHWVRDAPGQRQEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 579 - protein, 457 aa, Homo sapiens; identical to SEQ ID NO: 578

SEQ ID NO: 580 - protein, 452 aa, Homo sapiens
EVQLVESGGGLVQPGGSLRLSCAASGYTFTSYNMHWVRDAPGKGLEWVGAIYPGNGDTSYNQKEKGRFTISVDKSKNTLYLQMNSLRAEDTAVYYCARVVYYSNSYWYFDVWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 581 - protein, 452 aa, Homo sapiens; identical to SEQ ID NO: 580

SEQ ID NO: 582 - protein, 448 aa, Homo sapiens
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLLSPGK

SEQ ID NO: 583 - protein, 455 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 584 - protein, 455 aa, Homo sapiens
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 585 - protein, 455 aa, Homo sapiens
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 586 - protein, 455 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 587 - protein, 455 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 588 - protein, 455 aa, Homo sapiens; identical to SEQ ID NO: 587

SEQ ID NO: 589 - protein, 455 aa, Homo sapiens; identical to SEQ ID NO: 583

SEQ ID NO: 590 - protein, 455 aa, Homo sapiens; identical to SEQ ID NO: 584

SEQ ID NO: 591 - protein, 455 aa, Homo sapiens; identical to SEQ ID NO: 585

SEQ ID NO: 592 - protein, 455 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 593 - protein, 455 aa, Homo sapiens; identical to SEQ ID NO: 587

SEQ ID NO: 594 - protein, 455 aa, Homo sapiens; identical to SEQ ID NO: 587

SEQ ID NO: 595 - protein, 653 aa, Homo sapiens; identical to SEQ ID NO: 373

SEQ ID NO: 596 - protein, 653 aa, Homo sapiens
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 597 - protein, 653 aa, Homo sapiens; identical to SEQ ID NO: 355

SEQ ID NO: 598 - protein, 653 aa, Homo sapiens; identical to SEQ ID NO: 346

SEQ ID NO: 599 - protein, 653 aa, Homo sapiens
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 600 - protein, 653 aa, Homo sapiens; identical to SEQ ID NO: 599

SEQ ID NO: 601 - protein, 653 aa, Homo sapiens; identical to SEQ ID NO: 373

SEQ ID NO: 602 - protein, 653 aa, Homo sapiens; identical to SEQ ID NO: 596

SEQ ID NO: 603 - protein, 653 aa, Homo sapiens; identical to SEQ ID NO: 355

SEQ ID NO: 604 - protein, 653 aa, Homo sapiens; identical to SEQ ID NO: 346

SEQ ID NO: 605 - protein, 653 aa, Homo sapiens; identical to SEQ ID NO: 599

SEQ ID NO: 606 - protein, 653 aa, Homo sapiens; identical to SEQ ID NO: 599

SEQ ID NO: 607 - protein, 653 aa, Homo sapiens; identical to SEQ ID NO: 346

SEQ ID NO: 608 - protein, 653 aa, Homo sapiens; identical to SEQ ID NO: 355

SEQ ID NO: 609 - protein, 653 aa, Homo sapiens; identical to SEQ ID NO: 599

SEQ ID NO: 610 - protein, 674 aa, Homo sapiens
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 611 - protein, 457 aa, Homo sapiens; identical to SEQ ID NO: 578

SEQ ID NO: 612 - protein, 653 aa, Homo sapiens; identical to SEQ ID NO: 346

SEQ ID NO: 613 - protein, 457 aa, Homo sapiens; identical to SEQ ID NO: 578

SEQ ID NO: 614 - protein, 653 aa, Homo sapiens; identical to SEQ ID NO: 355

SEQ ID NO: 615 - protein, 452 aa, Homo sapiens; identical to SEQ ID NO: 580

SEQ ID NO: 616 - protein, 452 aa, Homo sapiens; identical to SEQ ID NO: 580

SEQ ID NO: 617 - protein, 653 aa, Homo sapiens; identical to SEQ ID NO: 599

SEQ ID NO: 618 - protein, 448 aa, Homo sapiens; identical to SEQ ID NO: 582

SEQ ID NO: 619 - protein, 674 aa, Homo sapiens; identical to SEQ ID NO: 610

SEQ ID NO: 620 - protein, 5 aa, Homo sapiens
ADAAP

SEQ ID NO: 621 - protein, 11 aa, Homo sapiens
ADAAPTVSIFP

SEQ ID NO: 622 - skipped

SEQ ID NO: 623 - protein, 12 aa, Homo sapiens
ADAAPTVSIFPP

SEQ ID NO: 624 - protein, 6 aa, Homo sapiens
AKTTAP

SEQ ID NO: 625 - protein, 13 aa, Homo sapiens
AKTTAPSVYPLAP

SEQ ID NO: 626 - protein, 17 aa, Homo sapiens
AKTTPKLEEGEFSEARV

SEQ ID NO: 627 - protein, 9 aa, Homo sapiens
AKTTPKLGG

SEQ ID NO: 628 - protein, 6 aa, Homo sapiens
AKTTPP

SEQ ID NO: 629 - protein, 13 aa, Homo sapiens
AKTTPPSVTPLAP

SEQ ID NO: 630 - protein, 6 aa, Homo sapiens
ASTKGP

SEQ ID NO: 631 - protein, 13 aa, Homo sapiens
ASTKGPSVFPLAP

SEQ ID NO: 632 - protein, 26 aa, Homo sapiens
ASTKGPSVFPLAPASTKGPSVFPLAP

SEQ ID NO: 633 - protein, 14 aa, Homo sapiens
EGKSSGSGSESKST

SEQ ID NO: 634 - protein, 15 aa, Homo sapiens
GEGESGEGESGEGES

SEQ ID NO: 635 - protein, 20 aa, Homo sapiens
GEGESGEGESGEGESGEGES

SEQ ID NO: 636 - protein, 15 aa, Homo sapiens
GEGGSGEGGSGEGGS

SEQ ID NO: 637 - protein, 15 aa, Homo sapiens
GENKVEYAPALMALS

SEQ ID NO: 638 - protein, 15 aa, Homo sapiens
GGEGSGGEGSGGEGS

SEQ ID NO: 639 - protein, 15 aa, Homo sapiens
GGGESGGEGSGEGGS

SEQ ID NO: 640 - protein, 15 aa, Homo sapiens
GGGESGGGESGGGES

SEQ ID NO: 641 - protein, 50 aa, Homo sapiens
  REPEAT 1..5: note = single repeat of Gly4Ser
  VARIANT 6..50: note = up to 9 copies of Gly4Ser can be deleted
GGGGSGGGGSGGGGSGGGGSGGGGSGGGGSGGGGSGGGGSGGGGSGGGGS

SEQ ID NO: 642 - protein, 10 aa, Homo sapiens
GGGGSGGGGS

SEQ ID NO: 643 - protein, 15 aa, Homo sapiens
GGGGSGGGGSGGGGS

SEQ ID NO: 644 - protein, 20 aa, Homo sapiens
GGGGSGGGGSGGGGSGGGGS

SEQ ID NO: 645 - protein, 15 aa, Homo sapiens
GGGKSGGGKSGGGKS

SEQ ID NO: 646 - protein, 15 aa, Homo sapiens
GGGKSGGKGSGKGGS

SEQ ID NO: 647 - protein, 15 aa, Homo sapiens
GGKGSGGKGSGGKGS

SEQ ID NO: 648 - protein, 5 aa, Homo sapiens
GGSGG

SEQ ID NO: 649 - protein, 9 aa, Homo sapiens
GGSGGGGSG

SEQ ID NO: 650 - protein, 13 aa, Homo sapiens
GGSGGGGSGGGGS

SEQ ID NO: 651 - protein, 15 aa, Homo sapiens
GHEAAAVMQVQYPAS

SEQ ID NO: 652 - protein, 15 aa, Homo sapiens
GKGGSGKGGSGKGGS

SEQ ID NO: 653 - protein, 15 aa, Homo sapiens
GKGKSGKGKSGKGKS

SEQ ID NO: 654 - protein, 20 aa, Homo sapiens
GKGKSGKGKSGKGKSGKGKS

SEQ ID NO: 655 - protein, 15 aa, Homo sapiens
GKPGSGKPGSGKPGS

SEQ ID NO: 656 - protein, 20 aa, Homo sapiens
GKPGSGKPGSGKPGSGKPGS

SEQ ID NO: 657 - protein, 15 aa, Homo sapiens
GPAKELTPLKEAKVS

SEQ ID NO: 658 - protein, 12 aa, Homo sapiens
GSAGSAAGSGEF

SEQ ID NO: 659 - protein, 14 aa, Homo sapiens
IRPRAIGGSKPRVA

SEQ ID NO: 660 - protein, 18 aa, Homo sapiens
KESGSVSSEQLAQFRSLD

SEQ ID NO: 661 - protein, 15 aa, Homo sapiens
KTTPKLEEGEFSEAR

SEQ ID NO: 662 - protein, 6 aa, Homo sapiens
QPKAAP

SEQ ID NO: 663 - protein, 13 aa, Homo sapiens
QPKAAPSVTLFPP

SEQ ID NO: 664 - protein, 27 aa, Homo sapiens
RADAAAAGGGGSGGGGSGGGGSGGGGS

SEQ ID NO: 665 - protein, 12 aa, Homo sapiens
RADAAAAGGPGS

SEQ ID NO: 666 - protein, 6 aa, Homo sapiens
RADAAP

SEQ ID NO: 667 - protein, 9 aa, Homo sapiens
RADAAPTVS

SEQ ID NO: 668 - protein, 6 aa, Homo sapiens
SAKTTP

SEQ ID NO: 669 - protein, 18 aa, Homo sapiens
SAKTTPKLEEGEFSEARV

SEQ ID NO: 670 - protein, 10 aa, Homo sapiens
SAKTTPKLGG

SEQ ID NO: 671 - protein, 14 aa, Homo sapiens
STAGDTHLGGEDFD

SEQ ID NO: 672 - protein, 5 aa, Homo sapiens
TVAAP

SEQ ID NO: 673 - protein, 12 aa, Homo sapiens
TVAAPSVFIFPP

SEQ ID NO: 674 - protein, 24 aa, Homo sapiens
TVAAPSVFIFPPTVAAPSVFIFPP

SEQ ID NO: 675 - protein, 214 aa, Homo sapiens
DIQMTQSPSSLSASVGDRVTITCSASQDISNYLNWYQKKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 676 - protein, 450 aa, Homo sapiens
QVQLVQSGAEVKKPGSSVKVSCKASGGTFSNYWMHWVRDAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 677 - protein, 451 aa, Homo sapiens
QVQLVQSGAEVKKPGSSVKVSCKASGGTFSNYWMHWVRDAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 678 - protein, 214 aa, Homo sapiens; identical to SEQ ID NO: 471

SEQ ID NO: 679 - protein, 451 aa, Homo sapiens; identical to SEQ ID NO: 472

SEQ ID NO: 680 - protein, 452 aa, Homo sapiens; identical to SEQ ID NO: 503

SEQ ID NO: 681 - protein, 107 aa, Homo sapiens
DIQMTQSPSSLSASVGDRVTITCSASQDISNYLNWYQKKPGKAPKLLIYYTSNLHSGVPSRFSGSGSGTDFTLTISSLQPEDFATYYCQQYRKLPWTFGQGTKLEIK

SEQ ID NO: 682 - protein, 121 aa, Homo sapiens
QVQLVQSGAEVKKPGSSVKVSCKASGGTFSNYWMHWVRDAPGQGLEWMGATYRGHSDTYYNQKFKGRVTITADKSTSTAYMELSSLRSEDTAVYYCARGAIYDGYDVLDNWGQGTLVTVSS

SEQ ID NO: 683-689 - skipped (7 entries)

SEQ ID NO: 690 - protein, 450 aa, Homo sapiens
EVQLVQSGAEVKKPGESLKISCKASGYSFTDYTMNWVRDMPGKGLEWIGLINPYNGVTSYNPNFQGQATLSVDKSISTAYLQWSSLKASDTAMYYCARSGDYDRNYFDYWGQGTTLTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 691 - protein, 455 aa, Homo sapiens; identical to SEQ ID NO: 587

SEQ ID NO: 692 - protein, 450 aa, Homo sapiens; identical to SEQ ID NO: 690

SEQ ID NO: 693 - protein, 455 aa, Homo sapiens; identical to SEQ ID NO: 585

SEQ ID NO: 694 - protein, 450 aa, Homo sapiens; identical to SEQ ID NO: 690

SEQ ID NO: 695 - protein, 450 aa, Homo sapiens; identical to SEQ ID NO: 690

SEQ ID NO: 696 - protein, 653 aa, Homo sapiens; identical to SEQ ID NO: 599

SEQ ID NO: 697-699 - skipped (3 entries)

SEQ ID NO: 700 - DNA, 20 bp, Homo sapiens
  source 1..20: mol_type = other DNA
gggaccaccttatattccca

SEQ ID NO: 701 - DNA, 20 bp, Homo sapiens
  source 1..20: mol_type = other DNA
gcggctccaattcggaagtg

SEQ ID NO: 702 - DNA, 20 bp, Homo sapiens
  source 1..20: mol_type = other DNA
cacggggaggatccgctcag

SEQ ID NO: 703 - DNA, 20 bp, Homo sapiens
  source 1..20: mol_type = other DNA
gccgagcagtgatctccagg

SEQ ID NO: 704 - DNA, 20 bp, Homo sapiens
  source 1..20: mol_type = other DNA
cccctggacacagagtcagg

SEQ ID NO: 705 - DNA, 20 bp, Homo sapiens
  source 1..20: mol_type = other DNA
cacagcgttatctccctctg

SEQ ID NO: 706 - DNA, 20 bp, Homo sapiens
  source 1..20: mol_type = other DNA
ttccagaaatggcagcaaag

SEQ ID NO: 707 - DNA, 20 bp, Homo sapiens
  source 1..20: mol_type = other DNA
ttcataatctggacagcctg

SEQ ID NO: 708 - DNA, 20 bp, Homo sapiens
  source 1..20: mol_type = other DNA
accataatgcctccccagag

SEQ ID NO: 709 - protein, 250 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 710 - protein, 187 aa, synthetic construct
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 711 - protein, 187 aa, synthetic construct
  VARIANT 1: note = F or S
  VARIANT 9: note = V or K
  VARIANT 21: note = V or Q
  VARIANT 58: note = V or K
  VARIANT 85: note = T or S
  VARIANT 93: note = L or G
XSQQIYGVXYGNVTFHVPSNXPLKEVLWKKQKDKVAELENSEFRAFSSFKNRVYLDTXSGSLTIYNLTSSDEDEYEMESPNITDXMKFFLYVXESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 712 - protein, 44 aa, synthetic construct
SQQIYGVVYGNVTFHVPSNVPLKEVLWKKQKDKVAELENSEFRA

SEQ ID NO: 713 - protein, 44 aa, synthetic construct
  VARIANT 8: note = V or K
  VARIANT 20: note = V or Q
SQQIYGVXYGNVTFHVPSNXPLKEVLWKKQKDKVAELENSEFRA